SEQUENCE LISTING (123 sequences)

SEQ ID NO: 1 - DNA, 25 bp, synthetic construct
  misc_feature 1..25: note = Artificial
  source 1..25: mol_type = other DNA
tttaccgggaattgtcgaattaggc

SEQ ID NO: 2 - DNA, 16 bp, synthetic construct
  misc_feature 1..16: note = Artificial
  source 1..16: mol_type = other DNA
gtgatctgaccgccgg

SEQ ID NO: 3 - DNA, 24 bp, synthetic construct
  misc_feature 1..24: note = Artificial
  source 1..24: mol_type = other DNA
tttgccttggattttccacttgca

SEQ ID NO: 4 - DNA, 23 bp, synthetic construct
  misc_feature 1..23: note = Artificial
  source 1..23: mol_type = other DNA
gagttggattcgtggccgcgcgg

SEQ ID NO: 5 - DNA, 23 bp, synthetic construct
  misc_feature 1..23: note = Artificial
  source 1..23: mol_type = other DNA
tggtgggagatccatctgcgagg

SEQ ID NO: 6 - DNA, 25 bp, synthetic construct
  misc_feature 1..25: note = Artificial
  source 1..25: mol_type = other DNA
tttctcatactcgttggctcgttgc

SEQ ID NO: 7 - DNA, 20 bp, synthetic construct
  misc_feature 1..20: note = Artificial
  source 1..20: mol_type = other DNA
cccatcctccagtgctacgg

SEQ ID NO: 8 - DNA, 28 bp, synthetic construct
  misc_feature 1..28: note = Artificial
  source 1..28: mol_type = other DNA
tttaggacctctagaggctacttctgcc

SEQ ID NO: 9 - DNA, 27 bp, synthetic construct
  misc_feature 1..27: note = Artificial
  source 1..27: mol_type = other DNA
tttgataatagctatgaagattttgtg

SEQ ID NO: 10 - DNA, 30 bp, synthetic construct
  misc_feature 1..30: note = Artificial
  source 1..30: mol_type = other DNA
gccgaccctttggccacgtggcaatatata

SEQ ID NO: 11 - DNA, 30 bp, synthetic construct
  misc_feature 1..30: note = Artificial
  source 1..30: mol_type = other DNA
gccgacgccacgtggcaagccgcctatata

SEQ ID NO: 12 - DNA, 20 bp, synthetic construct
  misc_feature 1..20: note = Artificial
  source 1..20: mol_type = other DNA
tgtctcgccgccgcgggaaa

SEQ ID NO: 13 - DNA, 36 bp, synthetic construct
  misc_feature 1..36: note = Artificial
  source 1..36: mol_type = other DNA
tgtctcgccgccgcgggaaaagccgacgcgggaaaa

SEQ ID NO: 14 - DNA, 36 bp, synthetic construct; identical to SEQ ID NO: 13
  misc_feature 1..36: note = Artificial
  source 1..36: mol_type = other DNA

SEQ ID NO: 15 - DNA, 39 bp, synthetic construct
  misc_feature 1..39: note = Artificial
  source 1..39: mol_type = other DNA
gctcaagccgacgcgggaaaggccgacgccgccgccgac

SEQ ID NO: 16 - DNA, 30 bp, synthetic construct
  misc_feature 1..30: note = Artificial
  source 1..30: mol_type = other DNA
cctttgtatatagccgacgccgacgccgcc

SEQ ID NO: 17 - DNA, 24 bp, synthetic construct
  misc_feature 1..24: note = Artificial
  source 1..24: mol_type = other DNA
cctttgtatatagccgacgccgac

SEQ ID NO: 18 - DNA, 30 bp, synthetic construct; identical to SEQ ID NO: 16
  misc_feature 1..30: note = Artificial
  source 1..30: mol_type = other DNA

SEQ ID NO: 19 - DNA, 36 bp, synthetic construct
  misc_feature 1..36: note = Artificial
  source 1..36: mol_type = other DNA
gcatgccgaccctttggccacgtggcaatatatatt

SEQ ID NO: 20 - DNA, 36 bp, synthetic construct
  misc_feature 1..36: note = Artificial
  source 1..36: mol_type = other DNA
atcgaatatatattgccacgtggccaaagggtcggc

SEQ ID NO: 21 - DNA, 130 bp, synthetic construct
  misc_feature 1..130: note = Artificial
  source 1..130: mol_type = other DNA
cgagagagccgtctccctcttcgcttctcctctcccccccgtgatctgacgccgacgccacgtggcaagccgcctatatacgccggcgacgaccccctcccaccacccgccgccgccgccgcgcccccgc

SEQ ID NO: 22 - DNA, 42 bp, synthetic construct
  misc_feature 1..42: note = Artificial
  source 1..42: mol_type = other DNA
acttaatgtctcgccgccgcgggaaaagccgacgcgggaaaa

SEQ ID NO: 23 - DNA, 42 bp, synthetic construct
  misc_feature 1..42: note = Artificial
  source 1..42: mol_type = other DNA
aagtttttcccgcgtcggcttttcccgcggcggcgagacatt

SEQ ID NO: 24 - DNA, 136 bp, synthetic construct
  misc_feature 1..136: note = Artificial
  source 1..136: mol_type = other DNA
cggcggcgtcgtcgtcttcctcctcttccccccgagttggattcgtggcctgtctcgccgccgcgggaaaagccgacgcgggaaaacggcggatgggaggggaggagggaatggcggcggggaggaagaagcgggt

SEQ ID NO: 25 - DNA, 136 bp, synthetic construct
  misc_feature 1..136: note = Artificial
  source 1..136: mol_type = other DNA
gatacaggtagagagaggtgagaaggcagtggtgggagatccatcttgtctcgccgccgcgggaaaagccgacgcgggaaaagcgaggtgcctccatggctgagaaggagggcaacctcgacgccgtcctcaagga

SEQ ID NO: 26 - DNA, 39 bp, synthetic construct; identical to SEQ ID NO: 15
  misc_feature 1..39: note = Artificial
  source 1..39: mol_type = other DNA

SEQ ID NO: 27 - DNA, 39 bp, synthetic construct
  misc_feature 1..39: note = Artificial
  source 1..39: mol_type = other DNA
gagcgtcggcggcggcgtcggcctttcccgcgtcggctt

SEQ ID NO: 28 - DNA, 124 bp, synthetic construct
  misc_feature 1..124: note = Artificial
  source 1..124: mol_type = other DNA
acctgccactcgcccactcctcctcctcccccatcctccagtgcctttgtatatagccgacgccgacgccgccctacggtgtcacgcagccatggcgctccgcacgctggcctcgaggaaaacc

SEQ ID NO: 29 - DNA, 36 bp, synthetic construct
  misc_feature 1..36: note = Artificial
  source 1..36: mol_type = other DNA
ctgcaacctttgtatatagccgacgccgacgccgcc

SEQ ID NO: 30 - DNA, 36 bp, synthetic construct
  misc_feature 1..36: note = Artificial
  source 1..36: mol_type = other DNA
gcagggcggcgtcggcgtcggctatatacaaaggtt

SEQ ID NO: 31 - DNA, 36 bp, synthetic construct
  misc_feature 1..36: note = Artificial
  source 1..36: mol_type = other DNA
cctttgtatatagccgacgccgacgccgccgggcga

SEQ ID NO: 32 - DNA, 36 bp, synthetic construct
  misc_feature 1..36: note = Artificial
  source 1..36: mol_type = other DNA
ccggcggcgtcggcgtcggctatatacaaaggtcgc

SEQ ID NO: 33 - DNA, 36 bp, synthetic construct
  misc_feature 1..36: note = Artificial
  source 1..36: mol_type = other DNA
atgcaatatatattgccacgtggccaaagggtcggc

SEQ ID NO: 34 - DNA, 34 bp, synthetic construct
  misc_feature 1..34: note = Artificial
  source 1..34: mol_type = other DNA
aaaagccgacgccacgtggcaagccgcctatata

SEQ ID NO: 35 - DNA, 34 bp, synthetic construct
  misc_feature 1..34: note = Artificial
  source 1..34: mol_type = other DNA
tttttatataggcggcttgccacgtggcgtcggc

SEQ ID NO: 36 - DNA, 42 bp, synthetic construct
  misc_feature 1..42: note = Artificial
  source 1..42: mol_type = other DNA
cactaatgtctcgccgccgcgggaaaagccgacgcgggaaaa

SEQ ID NO: 37 - DNA, 42 bp, synthetic construct
  misc_feature 1..42: note = Artificial
  source 1..42: mol_type = other DNA
agtgttttcccgcgtcggcttttcccgcggcggcgagacatt

SEQ ID NO: 38 - DNA, 40 bp, synthetic construct
  misc_feature 1..40: note = Artificial
  source 1..40: mol_type = other DNA
gtattgtctcgccgccgcgggaaaagccgacgcgggaaaa

SEQ ID NO: 39 - DNA, 40 bp, synthetic construct
  misc_feature 1..40: note = Artificial
  source 1..40: mol_type = other DNA
atacttttcccgcgtcggcttttcccgcggcggcgagaca

SEQ ID NO: 40 - DNA, 40 bp, synthetic construct
  misc_feature 1..40: note = Artificial
  source 1..40: mol_type = other DNA
ttattgtctcgccgccgcgggaaaagccgacgcgggaaaa

SEQ ID NO: 41 - DNA, 40 bp, synthetic construct
  misc_feature 1..40: note = Artificial
  source 1..40: mol_type = other DNA
ataattttcccgcgtcggcttttcccgcggcggcgagaca

SEQ ID NO: 42 - DNA, 39 bp, synthetic construct; identical to SEQ ID NO: 15
  misc_feature 1..39: note = Artificial
  source 1..39: mol_type = other DNA

SEQ ID NO: 43 - DNA, 39 bp, synthetic construct; identical to SEQ ID NO: 27
  misc_feature 1..39: note = Artificial
  source 1..39: mol_type = other DNA

SEQ ID NO: 44 - DNA, 34 bp, synthetic construct
  misc_feature 1..34: note = Artificial
  source 1..34: mol_type = other DNA
acgcggcggcgtcggcgtcggctatatacaaagg

SEQ ID NO: 45 - DNA, 34 bp, synthetic construct
  misc_feature 1..34: note = Artificial
  source 1..34: mol_type = other DNA
gcgtcctttgtatatagccgacgccgacgccgcc

SEQ ID NO: 46 - DNA, 36 bp, synthetic construct; identical to SEQ ID NO: 29
  misc_feature 1..36: note = Artificial
  source 1..36: mol_type = other DNA

SEQ ID NO: 47 - DNA, 36 bp, synthetic construct; identical to SEQ ID NO: 30
  misc_feature 1..36: note = Artificial
  source 1..36: mol_type = other DNA

SEQ ID NO: 48 - DNA, 36 bp, synthetic construct
  misc_feature 1..36: note = Artificial
  source 1..36: mol_type = other DNA
cgtgcctttgtatatagccgacgccgacgccgccgg

SEQ ID NO: 49 - DNA, 40 bp, synthetic construct
  misc_feature 1..40: note = Artificial
  source 1..40: mol_type = other DNA
cacgccggcggcgtcggcgtcggctatatacaaaggtcgc

SEQ ID NO: 50 - DNA, 25 bp, synthetic construct
  misc_feature 1..25: note = Artificial
  source 1..25: mol_type = other DNA
tgcaattggagccatcttttctgcg

SEQ ID NO: 51 - DNA, 24 bp, synthetic construct
  misc_feature 1..24: note = Artificial
  source 1..24: mol_type = other DNA
aatgcctgcggatagtggttaccc

SEQ ID NO: 52 - DNA, 26 bp, synthetic construct
  misc_feature 1..26: note = Artificial
  source 1..26: mol_type = other DNA
tgttacattccctcaggtgcttcgtg

SEQ ID NO: 53 - DNA, 26 bp, synthetic construct
  misc_feature 1..26: note = Artificial
  source 1..26: mol_type = other DNA
tgtcaccagcaacctttcgaacatca

SEQ ID NO: 54 - DNA, 25 bp, synthetic construct
  misc_feature 1..25: note = Artificial
  source 1..25: mol_type = other DNA
tggcaattggaaaagaaacagggcg

SEQ ID NO: 55 - DNA, 25 bp, synthetic construct
  misc_feature 1..25: note = Artificial
  source 1..25: mol_type = other DNA
tgggaatgtagggctatccactggt

SEQ ID NO: 56 - DNA, 25 bp, synthetic construct
  misc_feature 1..25: note = Artificial
  source 1..25: mol_type = other DNA
ccaaaatcctcatcaatggcccagc

SEQ ID NO: 57 - DNA, 23 bp, synthetic construct
  misc_feature 1..23: note = Artificial
  source 1..23: mol_type = other DNA
actgggccacactacaatcctgc

SEQ ID NO: 58 - DNA, 25 bp, synthetic construct
  misc_feature 1..25: note = Artificial
  source 1..25: mol_type = other DNA
actacaactcacaggatgcagcagc

SEQ ID NO: 59 - DNA, 40 bp, synthetic construct
  misc_feature 1..40: note = Artificial
  source 1..40: mol_type = other DNA
atagcacaatgccgaccctttggccacgtggcaatatata

SEQ ID NO: 60 - DNA, 40 bp, synthetic construct
  misc_feature 1..40: note = Artificial
  source 1..40: mol_type = other DNA
ctattatatattgccacgtggccaaagggtcggcattgtg

SEQ ID NO: 61 - DNA, 40 bp, synthetic construct
  misc_feature 1..40: note = Artificial
  source 1..40: mol_type = other DNA
tcggcacaatgccgacgccacgtggcaagccgcctatata

SEQ ID NO: 62 - DNA, 40 bp, synthetic construct
  misc_feature 1..40: note = Artificial
  source 1..40: mol_type = other DNA
ccgatatataggcggcttgccacgtggcgtcggcattgtg

SEQ ID NO: 63 - DNA, 70 bp, synthetic construct
  misc_feature 1..70: note = Artificial
  source 1..70: mol_type = other DNA
ctcttgacgcacaatcccacttgtctctatatagccgccgcgggaaaagccgacgcgggaaaatatataa

SEQ ID NO: 64 - DNA, 65 bp, synthetic construct
  misc_feature 1..65: note = Artificial
  source 1..65: mol_type = other DNA
agagttatatattttcccgcgtcggcttttcccgcggcggctatatagagacaagtgggattgtg

SEQ ID NO: 65 - DNA, 73 bp, synthetic construct
  misc_feature 1..73: note = Artificial
  source 1..73: mol_type = other DNA
gtaactgacgcacaatcccacttgtctctatatagccgccgcgggaaaagccgacgcgggaaaatatataagc

SEQ ID NO: 66 - DNA, 73 bp, synthetic construct
  misc_feature 1..73: note = Artificial
  source 1..73: mol_type = other DNA
ttacgcttatatattttcccgcgtcggcttttcccgcggcggctatatagagacaagtgggattgtgcgtcag

SEQ ID NO: 67 - DNA, 64 bp, synthetic construct
  misc_feature 1..64: note = Artificial
  source 1..64: mol_type = other DNA
catccacaatcccactacctcgtgtctcgccgccgcgggaaaagccgacgcgggaaaatatata

SEQ ID NO: 68 - DNA, 64 bp, synthetic construct
  misc_feature 1..64: note = Artificial
  source 1..64: mol_type = other DNA
gatgtatatattttcccgcgtcggcttttcccgcggcggcgagacacgaggtagtgggattgtg

SEQ ID NO: 69 - DNA, 55 bp, synthetic construct
  misc_feature 1..55: note = Artificial
  source 1..55: mol_type = other DNA
acaccacaatacctcgtgtctcgccgacgcgggaaaggccgacgccgccgccgac

SEQ ID NO: 70 - DNA, 55 bp, synthetic construct
  misc_feature 1..55: note = Artificial
  source 1..55: mol_type = other DNA
gtgtgtcggcggcggcgtcggcctttcccgcgtcggcgagacacgaggtattgtg

SEQ ID NO: 71 - DNA, 58 bp, synthetic construct
  misc_feature 1..58: note = Artificial
  source 1..58: mol_type = other DNA
atgacacaatcccactcctttgtatatagccgacgccgacgccgccacctcgtgtctc

SEQ ID NO: 72 - DNA, 58 bp, synthetic construct
  misc_feature 1..58: note = Artificial
  source 1..58: mol_type = other DNA
tcatgagacacgaggtggcggcgtcggcgtcggctatatacaaaggagtgggattgtg

SEQ ID NO: 73 - DNA, 58 bp, synthetic construct
  misc_feature 1..58: note = Artificial
  source 1..58: mol_type = other DNA
tctccacaatcccactcctttgtatatagccgacgccgacgccgccacctcgtgtctc

SEQ ID NO: 74 - DNA, 58 bp, synthetic construct
  misc_feature 1..58: note = Artificial
  source 1..58: mol_type = other DNA
gagagagacacgaggtggcggcgtcggcgtcggctatatacaaaggagtgggattgtg

SEQ ID NO: 75 - DNA, 58 bp, synthetic construct
  misc_feature 1..58: note = Artificial
  source 1..58: mol_type = other DNA
acgccacaatcccactcctttgtatatagccgacgccgacgccgccacctcgtgtctc

SEQ ID NO: 76 - DNA, 58 bp, synthetic construct
  misc_feature 1..58: note = Artificial
  source 1..58: mol_type = other DNA
gcgtgagacacgaggtggcggcgtcggcgtcggctatatacaaaggagtgggattgtg

SEQ ID NO: 77 - DNA, 70 bp, synthetic construct
  misc_feature 1..70: note = Artificial
  source 1..70: mol_type = other DNA
tttccctcgtgtctcatgacgcacaatcccactcctttgtatatagccgacgccgacgccgccgtatata

SEQ ID NO: 78 - DNA, 70 bp, synthetic construct
  misc_feature 1..70: note = Artificial
  source 1..70: mol_type = other DNA
gaaatatatacggcggcgtcggcgtcggctatatacaaaggagtgggattgtgcgtcatgagacacgagg

SEQ ID NO: 79 - DNA, 10 bp, synthetic construct
  misc_feature 1..10: note = Artificial
  source 1..10: mol_type = other DNA
gccacgtggc

SEQ ID NO: 80 - DNA, 11 bp, synthetic construct
  misc_feature 1..11: note = Artificial
  source 1..11: mol_type = other DNA
taagagccgcc

SEQ ID NO: 81 - DNA, 11 bp, synthetic construct
  misc_feature 1..11: note = Artificial
  source 1..11: mol_type = other DNA
cctcgtgtctc

SEQ ID NO: 82 - DNA, 36 bp, synthetic construct
  misc_feature 1..36: note = Artificial
  source 1..36: mol_type = other DNA
cacaatgccgaccctttggccacgtggcaatatata

SEQ ID NO: 83 - DNA, 36 bp, synthetic construct
  misc_feature 1..36: note = Artificial
  source 1..36: mol_type = other DNA
cacaatgccgacgccacgtggcaagccgcctatata

SEQ ID NO: 84 - DNA, 67 bp, synthetic construct
  misc_feature 1..67: note = Artificial
  source 1..67: mol_type = other DNA
ctgacgcacaatcccacttgtctctatatagccgccgcgggaaaagccgacgcgggaaaatatataa

SEQ ID NO: 85 - DNA, 67 bp, synthetic construct; identical to SEQ ID NO: 84
  misc_feature 1..67: note = Artificial
  source 1..67: mol_type = other DNA

SEQ ID NO: 86 - DNA, 60 bp, synthetic construct
  misc_feature 1..60: note = Artificial
  source 1..60: mol_type = other DNA
cacaatcccactacctcgtgtctcgccgccgcgggaaaagccgacgcgggaaaatatata

SEQ ID NO: 87 - DNA, 51 bp, synthetic construct
  misc_feature 1..51: note = Artificial
  source 1..51: mol_type = other DNA
cacaatacctcgtgtctcgccgacgcgggaaaggccgacgccgccgccgac

SEQ ID NO: 88 - DNA, 54 bp, synthetic construct
  misc_feature 1..54: note = Artificial
  source 1..54: mol_type = other DNA
cacaatcccactcctttgtatatagccgacgccgacgccgccacctcgtgtctc

SEQ ID NO: 89 - DNA, 54 bp, synthetic construct; identical to SEQ ID NO: 88
  misc_feature 1..54: note = Artificial
  source 1..54: mol_type = other DNA

SEQ ID NO: 90 - DNA, 54 bp, synthetic construct; identical to SEQ ID NO: 88
  misc_feature 1..54: note = Artificial
  source 1..54: mol_type = other DNA

SEQ ID NO: 91 - DNA, 66 bp, synthetic construct
  misc_feature 1..66: note = Artificial
  source 1..66: mol_type = other DNA
cctcgtgtctcatgacgcacaatcccactcctttgtatatagccgacgccgacgccgccgtatata

SEQ ID NO: 92 - protein, 231 aa, Oryza sativa
  source 1..231: note = subspecies japonica
MALRTLASRKTLAAAALPLAAAAAARGVTTVALPDLPYDYGALEPAISGEIMRLHHQKHHATYVANYNKALEQLDAAVAKGDAPAIVHLQSAIKFNGGGHVNHSIFWNNLKPISEGGGDPPHAKLGWAIDEDFGSFEALVKKMSAEGAALQGSGWVWLALDKEAKKLSVETTANQDPLVTKGANLVPLLGIDVWEHAYYLQYKNVRPDYLSNIWKVMNWKYAGEVYENATA

SEQ ID NO: 93 - protein, 164 aa, Oryza sativa
  source 1..164: note = subspecies japonica
MAGKAGGLKGVALIGGAGGNSAVAGALHFFQDPSTGYTEVRGRVTGLAPGLHGFHIHSFGDTTNGCNSTGPHFNPHNKSHGAPSDDERHVGDLGNIVANKDGVADIFIKDLQISLSGPHSILGRAVVVHADSDDLGRGGHELSKTTGNAGARIGCGIIGLRSAV

SEQ ID NO: 94 - protein, 60 aa, Oryza sativa
  source 1..60: note = subspecies japonica
MVKAVVVLGSSEIVKGTIHFVQEGDGPTTVTGSVSGLKPGLHGFHIHALGDTTNGCMSTG

SEQ ID NO: 95 - protein, 1148 aa, Oryza sativa
  source 1..1148: note = subspecies japonica
MDNPEAEPDDAVLFVGVSLVLGIASRHLLRGTRVPYTVALLVLGVALGSLEFGTKHGMGKLGAGIRIWANINPDLLLAVFLPALLFESSFSMEIHQIKKCMAQMVLLAGPGVLISTFFLGSALKLTFPYNWNWKTSLLLGGLLSATDPVAVVALLKELGASKKLSTIIEGESLMNDGTAIVVYQLFYRMVLGRTFDAGSIIKFLSEVSLGAVALGLAFGIASVLWLGFIFNDTIIEIALTLAVSYIAFFTAQDALEVSGVLTVMTLGMFYAAFAKTAFKGDSQQSLHHFWEMVAYIANTLIFILSGVVIADGVLENNVHFERHGASWGFLLLLYVFVQISRILVVVILYPLLRHFGYGLDLKEATILVWAGLRGAVALSLSLSVKRASDAVQTHLKPVDGTMFVFFTGGIVFLTLIFNGSTTQFLLHLLGMDRLAATKLRILNYTKYEMLNKALEAFGDLRDDEELGPPADWVTVKKYITCLNDLDDEPVHPHAVSDRNDRMHTMNLRDIRVRLLNGVQAAYWGMLEEGRITQTTANILMRSVDEAMDLVPTQELCDWKGLRSNVHFPNYYRFLQMSRLPRRLITYFTVERLESGCYICAAFLRAHRIARRQLHDFLGDSEVARIVIDESNAEGEEARKFLEDVRVTFPQVLRVLKTRQVTYSVLTHLSEYIQNLQKTGLLEEKEMAHLDDALQTDLKKFKRNPPLVKMPRVSDLLNTHPLVGALPAAMRDPLLSSTKETVKGHGTILYREGSRPTGIWLVSIGVVKWTSQRLSSRHSLDPILSHGSTLGLYEVLIGKPYICDMITDSVVHCFFIEAEKIEQLRQSDPSIEIFLWQESALVVARLLLPMMFEKMATHELRVLITERSTMNIYIKGEEIELEQNFIGILLEGFLKTKNQTLITPPGLLLPPNADLNLFGLESSAINRIDYCYTAPSYQVEARARILFVEIGRPEIEADLQRSASLISQTLELPRTQSKEHSGLLSWPESFRKSRGAQNGASLTEIRDHPASFSARALQLSMYGSMINDMKSGQGQGQRRQRHRHTKASSNKAHSSSYPRVPSRSSNTQRPLLSVQSEGANMTTARQAAAAGASLPPEPEEAGRRRRRQRKAIEEDEDNSSDESAGEEVIVRVDSPSMLTFRQPSSAADR

SEQ ID NO: 96 - protein, 1142 aa, Oryza sativa
  source 1..1142: note = subspecies japonica
MDNPEAEPDDAVLFVGVSLVLGIASRHLLRGTRVPYTVALLVLGVALGSLEFGTKHGMGKLGAGIRIWANINPDLLLAVFLPALLFESSFSMEIHQIKKCMAQMVLLAGPGVLISTFFLGSALKLTFPYNWNWKTSLLLGGLLSATDPVAVVALLKELGASKKLSTIIEGESLMNDGTAIVVYQLFYRMVLGRTFDAGSIIKFLSEVSLGAVALGLAFGIASVLWLGFIFNDTIIEIALTLAVSYIAFFTAQDALEVSGVLTVMTLGMFYAAFAKTAFKGDSQQSLHHFWEMVAYIANTLIFILSGVVIADGVLENNVHFERHGASWGFLLLLYVFVQISRILVVVILYPLLRHFGYGLDLKEATILVWAGLRGAVALSLSLSVKRASDAVQTHLKPVDGTMFVFFTGGIVFLTLIFNGSTTQFLLHLLGMDRLAATKLRILNYTKYEMLNKALEAFGDLRDDEELGPPADWVTVKKYITCLNGLDDEPVHPHAVSDRNDRMHTMNLRDIRVRLLNGVQAAYWGMLEEGRITQTTANILMRSVDEAMDLVPTQELCDWKGLRSNVHFPNYYRFLQMSRLPRRLITYFTVERLESGCYICAAFLRAHRIARRQLHDFLGDSEVARIVIDESNAEGEEARKFLEDVRVTFPQVLRVLKTRQVTYSVLTHLSEYIQNLQKTGLLEEKEMAHLDDALQTDLKKFKRNPPLVKMPRVSDLLNTHPLVGALPAAMRDPLLSSTKETVKGHGTILYREGSRPTGIWLVSIGVVKWTSQRLSSRHSLDPILSHGSTLGLYEVLIGKPYICDMITDSVVHCFFIEAEKIEQLRQSDPSIEIFLWQESALVVARLLLPMMFEKMATHELRVLITERSTMNIYIKGEEIELEQNFIGILLEGFLKTKNQTLITPPGLLLPPNADLNLFGLESSAINRIDYCYTAPSYQVEARARILFVEIGRPEIEADLQRSASLISQTLELPRTQSKEHSETIQQLSGAQNGASLTEIRDHPASFSARALQLSMYGSMINDMKSGQGQGQRRQRHRHTKASSNKAHSSSYPRVPSSSSNTQRPLLSVQSEGANMTTARQAAAAGASLPPEPEEAGRRRRRQRKAIEEDEDNSSDESAGEEVIVRVDSPSMLTFRQPSSAADR

SEQ ID NO: 97 - protein, 535 aa, Oryza sativa
  source 1..535: note = subspecies japonica
MGMEVAAARLGALYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIGAIFSATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKITWRQQVVIWWAGLMRGAVSIALAYNKFTRSGHTQLHGNAIMITSTITVVLFSTMVFGMMTKPLIRLLLPASGHPVTSEPSSPKSLHSPLLTSMQGSDLESTTNIVRPSSLRMLLTKPTHTVHYYWRKFDDALMRPMFGGRGFVPFSPGSPTEQSHGGR

SEQ ID NO: 98 - protein, 535 aa, Oryza sativa
  source 1..535: note = subspecies japonica
MGMEVAAARLGPLYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIRAIFPATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKRTWRQQVVIWWAGLMRGAVSIALAYNKFTRSGHTQLHGNAIMITSTITVVLWSTMVFGMMTKPLIRLLLPASGHPVTSEPSSPKSLHSPLLTRMQGSDLESTTNIVRPSSLRMLLTKPTHTVHYYWRKFDDALMRPMFGGRGFVPFSPGSPTEQSHGGR

SEQ ID NO: 99 - protein, 530 aa, Oryza sativa
  source 1..530: note = subspecies japonica
MTSIYHDFIHNKLQSFGRIGRYFVNFVVLAHRFIALHIHPFWIQLSYFLLISILGSVLLMFLKPSNPEFRPGYIDMLFLSTSALTLSSLITIEMEVLSSSQIVVITLLMLLGGEVFVSFLGLMLRLNHKHNPEFSGDKVSSVPIELDTINSASTVISCEELQLEAAIPEVPSSTIKDLKRSKRLRWFLGFVVFSYFVVIHVAGFLLVLWYISRVSSAKAPLKKKGINIALFSFSVTVSSFANVGLVPTNENMAIFSKNPGLLLLFIGQILAGNTLYPLFLRLLIWFLGKVTKLRELKLMIKNPEELQYDYLLPKLPTAFLASTVIGLMASLVTLFGAVDWNSSVFDGLSSYQKIINALFMAVNARHSGENSIDCSLIAPAVLVLFIILMYLPPSTTFALSNGDEKTANKKAKRKLGLVVQNLAFSQLACISVFVIVAFITERSRLRNDPLNFSALNMIFEIISAYGNVGLSTGYSCSRLQKLHPGSICQDKPYSLSGWWSDEGKLLLVFVMLYGRLKAFTKGTGEYWRLW

SEQ ID NO: 100 - protein, 300 aa, Oryza sativa
  source 1..300: note = subspecies japonica
MGGEEGMAAGRKKRVGRYEVGRTIGQGTFAKVKFAVDADTGAAVAMKVLDKDTILNHRMLHQIKREISIMKIVRHPNIVRLNEVLAGKTKIYIILELITGGELFDKIARQGKLRENEARKYFQQLIDAINYCHSKGVYHRDLKPENLLLDSRGNLKVSDFGLSTLAQKGVGLLHTTCGTPNYVAPEVLSNNGYDGSAADVWSCGVILYVLMAGYLPFEEDDLPTLYDKITAGQFSCPYWFSPGATSLIHRILDPNPKTRITIEQIREDTWFKKTYVAIKRGEDENVDLDDVQAVFDNIED

SEQ ID NO: 101 - protein, 956 aa, Oryza sativa
  source 1..956: note = subspecies japonica
MAEDKGGLDAVLKESVDLENIPIEEVFQNLKCCRQGLTSEEAQLRLQLFGPNKLEEKEESKFLKFLGFMWNPLSWVMEAAAIMAIALANGGGKPPDWQDFVGIITLLLINSTISFIEENNAGNAAAALMARLAPKAKVLRNGSWTEEEAAILVPGDIISIKLGDIIPADARLLEGDPLKIDQSALTGESLPATKGPGDGVYSGSTVKQGEIEAVVIATGVHTFFGKAAHLVDSTNQVGHFQKVLTAIGNFCICSIAVGMFVEIIVMYPIQHRPYRPGIDNLLVLLIGGIPIAMPTVLSVTMAIGSHRLSQQGAITKRMTAIEEMAGMDVLCSDKTGTLTLNKLTVDKNLIEIFERGVTQDQVILMAARASRTENQDAIDTAIVGMLADPKEARAGIQEVHFLPFNPTDKRTALTYIDSDGKMYRVSKGAPEQILNLAHNKTQIERRVHAVIDKFAERGLRSLAVAYQEVPDGRKESPGGPWRFVALLPLFDPPRHDSAETIRRALNLGVNVKMITGDQLAIGKETGRRLGMGTNMYPSSALLGQNKDESVAALPVDDLIEKADGFAGVFPEHKYEIVKRLQARKHICGMTGDGVNDAPALKKADIGIAVADATDAARSASDIVLTEPGLSVIISAVLTSRAIFQRMKNYTIYAVSITIRIVFGFMLLALIWEFDFPPFMVLIIAILNDGTIMTISKDLVKPSPLPDSWKLAEIFTTGVVLGGYLAMMTVIFFWAAYKTNFFPRIFHVESLEKTAQDDYQKLASAVYLQVSTISQALIFVTRSRSWSFIERPGFLLVFAFFVAQLIATLIAVYANWAFTSIKGIGWGWAGIVWLYNLVFYFPLDIIKFLIRYALSGKAWDLVIEQRIAFTRKKDFGKEERELKWAHAHRTLHGLQPPDAKPFPEKTGYSELNQMAEEAKRRAEIARLRELHTLKGHVESVVKLKGLDIDTIHQSYTV

SEQ ID NO: 102 - protein, 620 aa, Oryza sativa
  source 1..620: note = subspecies japonica
MSYDRVTTFEDSEKESEYGYVRKVSGPVVVADGMGGAAMYELVRVGNDNLIGEIIRLEGDSATIQVYEETAGLMVNDPVLRTRKPLSVELGPGILGNIFDGIQRPLKTIAIKSGDVYIPRGVSVPALDKDQLWDFEPKKLGVGDAITGGDLYATVFENTLMKHHVALPPGSMGKISYIAPAGQYSLQDTVLELEFQGIKKQFTMLQTWPVRSPRPVSSKLAADTPLLTGQRVLDALFPSVLGGTCAIPGAFGCGKTVISQALSKYSNSEAVVYVGCGERGNEMAEVLMDFPQLTMTLPDGREESVMKRTTLVANTSNMPVAAREASIYTGITIAEYFRDMGYNVSMMADSTSRWAEALREISGRLAEMPADSGYPAYLAARLASFYERAGKVKCLGSPDRTGSVTIVGAVSPPGGDFSDPVTSATLSIVQVFWGLDKKLAQRKHFPSVNWLISYSKYSKALESFYEKFDQDFIDIRTKAREVLQREDDLNEIVQLVGKDALAESDKITLETAKLLREDYLAQNAFTPYDKFCPFYKSVWMMRNIIHFNTLANQAVERAANADGQKITYSVIKHRMGDLFYRLVSQKFEDPAEGEDVLVAKFQKLYDDLTTGFRNLEDEAR

SEQ ID NO: 103 - protein, 488 aa, Oryza sativa
MGLVKDGAADLEEGTLEIGMEYRTVSGVAGPLVILDKVKGPKYQEIVNIRLGDGTTRRGQVLEVDGEKAVVQVFEGTSGIDNKYTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKSLEKGKHAEGGEDDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRSGSITQIPILTMPNDDITHPTPDLTGYITEGQIYIDRQLHNRQIYPPINVLPSLSRLMKSAIGEGMTRRDHSDVSNQLYANYAIGKDVQAMKAVVGEEALSSEDLLYLEFLDKFERKFVTQGAYDTRNIFQSLDLAWTLLRIFPRELLHRIPAKTLDQYYSRDATH

SEQ ID NO: 104 - protein, 328 aa, Leymus chinensis
MAASLQAAATLMPAKIGGRASSARPSSHVARAFGVDAGARITCSLQSDIREVASKCAEAAKMAGFALATSALLVSGASAEGAPKRLTFDEIQSKTYMEVKGTGTANQCPTIDGGVDSFPFKAGKYEMKKFCLEPTSFTVKAEGIQKNEPPAFQKTKLMTRLTYTLDEMEGPLEVGADGTLKFEEKDGIDYAAVTVQLPGGERVPFLFTVKQLVATGKPESFSGPFLVPSYRGSSFLDPKGRGGSTGYDNAVALPAGGRGDEEELAKENVKNASSSTGNITLSVTKSKPETGEVIGVFERVQPSDTDLGAKAPKDVKIQGVWYAQLESN

SEQ ID NO: 105 - protein, 142 aa, Oryza sativa
  source 1..142: note = subspecies japonica
VVNASSSEASSDEKNVTRRRLALLGAGALATGLLKSSSAYAEEVPKNYKSYVDSKDGYSYLYPADWRDFDFLGHDSAFKDRNVALQCVRVGFIPTTKTDIRDLGPMDEAIFNLVNNVYAAPNQIPTVYDMQERTVHGKNYWT

SEQ ID NO: 106 - protein, 217 aa, Oryza sativa
  source 1..217: note = subspecies indica
MAQAMASMTGLSQGVQLPAGPRRAGGRSRLAVVRADAAAADVQTGRRAVLGLVATGIAGGALAQAALAEAAKPIKLGPPPPPSGGLPGTLNSDQARDTDLPLRERFYLQPLPPAEAAARAKESAQDIINLKPLIEKKQWPFVRDDLRLRASYLRYDLKTVINSKPKDEKKGLKDLTGKLFATIDGLDHAAKIKSPEEAEKYYTLTKSALGDVLAKLG

SEQ ID NO: 107 - protein, 505 aa, Oryza sativa
  source 1..505: note = subspecies japonica
MEGAGRDGNPLGGYRIGKTLGIGSFGKVKIAEHILTGHKVAIKILNRRKIKSMEMEEKVKREIKILRLFMHPHIIRLYEVIDTPADIYVVMEYVKSGELFDYIVEKGRLQEEEARRFFQQIISGVEYCHRNMVVHRDLKPENLLLDSKCNVKIADFGLSNVMRDGHFLKTSCGSPNYAAPEVISGKLYAGPEVDVWSCGVILYALLCGTLPFDDENIPNLFKKIKGGIYTLPSHLSPLARDLIPRMLVVDPMKRITIREIREHQWFTVGLPRYLAVPPPDTAQQVKKLDDETLNDVINMGFDKNQLIESLHKRLQNEATVAYYLLLDNRLRTTSGYLGAEFHESMESSLAQVTPAETPNSATDHRQHGHMESPGFGLRHHFAADRKWALGLQSRAHPREIITEVLKALQELNVCWKKIGHYNMKCRWSPSFPSHESMMHNNHGFGAESAIIETDDSEKSTHTVKFEIQLYKTRDEKYLLDLQRVSGPQLLFLDLCSAFLTQLRVL

SEQ ID NO: 108 - protein, 505 aa, Oryza sativa; identical to SEQ ID NO: 107
  source 1..505: note = subspecies japonica

SEQ ID NO: 109 - protein, 503 aa, Oryza sativa
MEGAGSDGNPLGGYRIGKTLGIGSFGKVKIAEHILTGHKVAIKILNRRKSMEMEEKVKREIKILRLFMHPHIIRLYEVIDTPADIYVVMEYVKSGELFDYDVEKGRLQEEEARRFFQQIISGVEYCHRNMVVRRDLKPENLLLDSKCNVKIADFGLSNVMRDGHFLKTSCGSPNYAAPEVISGKLYAGPEVDVWSCGVILYALLCGTLPFDDENIPNLFKNIKGGIYTLPSHLSPLGRDLIPRMLVVDPMKRITIREIREHQWFTVGLPRYLAVPPPDTAQQVKKLDDETQNDVINMGFDKNQLIESLHKRLQNEATVAYYLLLDNRLRTTSGYLGAEFHESMVSSLAQVTPAETPNSATDHRQHGHMESPGFGLRHHFAADRKWALGLQSRAHPREIITEVLKALQELNVCWKKIGHHNMKCRWSPSFPSHESMMHNNHGFGAASAMIETDDSEKSTHTVKFEIQLYKTRDEKYFLDLQRLSGPQLLFLDLCSAFLTQLRVL

SEQ ID NO: 110 - protein, 505 aa, Oryza sativa; identical to SEQ ID NO: 107
  source 1..505: note = subspecies japonica

SEQ ID NO: 111 - DNA, 36 bp, synthetic construct; identical to SEQ ID NO: 19
  misc_feature 1..36: note = Artificial
  source 1..36: mol_type = other DNA

SEQ ID NO: 112 - protein, 497 aa, Arabidopsis thaliana
MAMLGFYVTFIFFLVCLFTYFFLQKKPQGQPILKNWPFLRMLPGMLHQIPRIYDWTVEVLEATNLTFYFKGPWLSGTDMLFTADPRNIHHILSSNFGNYPKGPEFKKIFDVLGEGILTVDFELWEEMRKSNHALFHNQDFIELSVSSNKSKLKEGLVPFLDNAAQKNIIIELQDVFQRFMFDTSSILMTGYDPMSLSIEMLEVEFGEAADIGEEAIYYRHFKPVILWRLQNWIGIGLERKMRTALATVNRMFAKIISSRRKEEISRAKTEPYSKDALTYYMNVDTSKYKLLKPNKDKFIRDVIFSLVLAGRDTTSSVLTWFFWLLSKHPQVMAKLRHEINTKFDNEDLEKLVYLHAALSESMRLYPPLPFNHKSPAKPDVLPSGHKVDANSKIVICIYALGRMRSVWGEDALDFKPERWISDNGGLRHEPSYKFMAFNSGPRTCLGKNLALLQMKMVALEIIRNYDFKVIEGHKVEPIPSILLRMKHGLKVTVTKKI

SEQ ID NO: 113 - protein, 1084 aa, Oryza sativa
MAGNEWINGYLEAILDSGGAAGGGGGGGGGGGGGGGGGGGGGGGGVDPRSPAAGAASPRGPHMNFNPTHYFVEEVVKGVDESDLHRTWIKVVATRNARERSTRLENMCWRIWHLARKKKQLELEGILRISARRKEQEQVRRETSEDLAEDLFEGEKADTVGELAQQDTPMKKKFQRNFSELTVSWSDENKEKKLYIVLISLHGLVRGDNMELGRDSDTGGQVKYVVELARALAMMPGVYRVDLFTRQVSSPEVDWSYGEPTEMLTSGSTDGEGSGESAGAYIVRIPCGPRDKYLRKEALWPYLQEFVDGALAHILNMSKALGEQVSNGKLVLPYVIHGHYADAGDVAALLSGALNVPMVLTGHSLGRNKLEQIMKQGRMSKEEIDSTYKIMRRIEGEELALDAAELVITSTRQEIDEQWGLYDGFDVKLEKVLRARARRGVSCHGRFMPRMVVIPPGMDFSSVVVPEDTSDGDDGKDFEIASPRSLPPIWAEVMRFLTNPHKPMILALSRPDPKKNITTLVKAFGECRPLRELANLILIMGNRDDIDEMSAGNASVLTTVLKLIDKYDLYGSVAFPKHHKQSDVPEIYRLTGKMKGVFINPALVEPFGLTLIEAAAHGLPIVATKNGGPVDIKNALNNGLLVDPHDQHAIADALLKLVADKNLWQECRKNGLRNIQLYSWPEHCRTYLTRIAGCRIRNPRWLMDTPADAAAEEEEALEDSLMDVQDLSLRLSIDGERGSSMNDAPSSDPQDSVQRIMNKIKRSSPADTDGAKIPAEAAATATSGAMNKYPLLRRRRRLFVIAVDCYGDDGSASKRMLQVIQEVFRAVRSDSQMSRISGFALSTAMPLPETLKLLQLGKIPPTDFDALICGSGSEVYYPSTAQCVDAGGRLRPDQDYLLHINHRWSHDGAKQTIAKLAHDGSGTNVEPDVESCNPHCVSFFIKDPNKVRTIDEMRERVRMRGLRCHLMYCRNATRLQVVPLLASRSQALRYLFVRWGLSVGNMYLIVGEHGDTDHEEMLSGLHKTVIIRGVTEKGSEQLVRSSGSYQREDVVPSESPLIAFTKGDLKADEIMRALKEVTKAASGM

SEQ ID NO: 114 - protein, 716 aa, Oryza sativa
MASVDPSRSFVRDVKRVIIKVGTAVVSRQDGRLALGRVGALCEQVKELNSLGYEVILVTSGAVGVGRQRLRYRKLVNSSFADLQKPQMELDGKACAAVGQSGLMALYDMLFNQLDVSSSQLLVTDSDFENPKFREQLTETVESLLDLKVIPIFNENDAISTRKAPYEDSSGIFWDNDSLAGLLALELKADLLILLSDVDGLYSGPPSEPSSKIIHTYIKEKHQQEITFGDKSRVGRGGMTAKVKAAVLASNSGTPVVITSGFENRSILKVLHGEKIGTLFHKNANLWESSKDVSTREMAVAARDCSRHLQNLSSEERKKILLDVADALEANEDLIRSENEADVAAAQVAGYEKPLVARLTIKPGKIASLAKSIRTLANMEDPINQILKKTEVADDLVLEKTSCPLGVLLIVFESRPDALVQIASLAIRSGNGLLLKGGKEAIRSNTILHKVITDAIPRNVGEKLIGLVTTRDEIADLLKLDDVIDLVIPRGSNKLVSQIKASTKIPVLGHADGICHVYIDKSADMDMAKHIVMDAKIDYPAACNAMETLLVHKDLMKSPGLDDILVALKTEGVNIYGGPIAHKALGFPKAVSFHHEYSSMACTVEFVDDVQSAIDHIHRYGSAHTDCIVTTDDKVAETFLRRVDSAAVFHNASTRFSDGARFGLGAEVGISTGRIHARGPVGVEGLLTTRWILRGRGQVVNGDKDVVYTHKSLPLQ

SEQ ID NO: 115 - DNA, 25 bp, synthetic construct
  misc_feature 1..25: note = Artificial
  source 1..25: mol_type = other DNA
tttaccgggaattgtcgaattaggc

SEQ ID NO: 116 - DNA, 25 bp, synthetic construct
  misc_feature 1..25: note = Artificial
  source 1..25: mol_type = other DNA
tttaaaattcgctggaggaaaaaaa

SEQ ID NO: 117 - DNA, 24 bp, synthetic construct
  misc_feature 1..24: note = Artificial
  source 1..24: mol_type = other DNA
tttgccttggattttccacttgca

SEQ ID NO: 118 - DNA, 25 bp, synthetic construct
  misc_feature 1..25: note = Artificial
  source 1..25: mol_type = other DNA
tttgggggggcttcgctgcctgaat

SEQ ID NO: 119 - DNA, 25 bp, synthetic construct
  misc_feature 1..25: note = Artificial
  source 1..25: mol_type = other DNA
tttgcaagcaagatttcctttgctt

SEQ ID NO: 120 - DNA, 25 bp, synthetic construct
  misc_feature 1..25: note = Artificial
  source 1..25: mol_type = other DNA
tttctcatactcgttggctcgttgc

SEQ ID NO: 121 - DNA, 25 bp, synthetic construct
  misc_feature 1..25: note = Artificial
  source 1..25: mol_type = other DNA
tttatgcggcatctgtctcgtgaac

SEQ ID NO: 122 - DNA, 25 bp, synthetic construct
  misc_feature 1..25: note = Artificial
  source 1..25: mol_type = other DNA
tttaggacctctagaggctacttct

SEQ ID NO: 123 - DNA, 25 bp, synthetic construct
  misc_feature 1..25: note = Artificial
  source 1..25: mol_type = other DNA
tttggtggactactcgatctctgcg

(19) United States
(12) Patent Application Publication
(10) Pub. No.: US 2025/0388921 A1
(43) Pub. Date: Dec. 25, 2025

(54) SALT TOLERANT PLANTS

(71) Applicants: Luke YOUNG, Kitchener (CA); Rory James HORNBY, Kitchener (CA); Alora Innovations Inc., San Francisco, CA (US)

(72) Inventors: Luke YOUNG, Kitchener (CA); Rory James HORNBY, Kitchener (CA)

(21) Appl. No.: 18/681,396

(22) PCT Filed: Aug. 4, 2022
(86) PCT No.: PCT/EP2022/072021

§ 371 Date: Feb. 5, 2024

(30) Foreign Application Priority Data
Aug. 4, 2021 (GB) 2111268.5
Aug. 11, 2021 (GB) 2111563.9

(51) Int. Cl. C12N 15/82; C07K 14/495; C12N 9/02; C12N 9/12; C12N 9/22; C12N 15/11

(52) U.S. Cl. CPC C12N 15/8273; C07K 14/495; C12N 9/0089; C12N 9/12; C12N 9/226; C12N 15/111; C12N 15/8207; C12N 15/8227; C12N 2310/20; C12Y 115/01001; C12Y 207/11001

(57) ABSTRACT

The invention provides engineered plants that are salt tolerant, methods for making these plants and uses of these plants.

### TECHNICAL FIELD

This invention relates to plants that are designed to be salt tolerant.

### BACKGROUND TO THE INVENTION

Soil salinity is one of the most severe problems in agriculture aside from drought. Approximately 20% of the world's cultivated land and nearly half of irrigated land are affected by salinity, which has become a serious threat to agricultural production limiting plant growth and productivity worldwide [1]. Absorption of excessive salt from saline soils inhibits both root and shoot growth, reduces reproductive activity and affects viability of plants. As a result, salinity is one of the major constraints in geographic range of crop cultivation globally, and, where it does not preclude growth of certain crops nonetheless substantially affects crop productivity. Additionally, salt accumulation as a result of excessive irrigation, improper drainage, or use of reclaimed water places existing agricultural areas at risk, especially as climate change increases irrigation needs in arid/semiarid regions.

Excessive salinity imposes two stress factors on plants: an osmotic component that results from the reduced water availability caused by an increase in osmotic pressure in the soil, and an ionic stress resulting from a solute imbalance, causing changes in the K⁺/Na⁺ ratio and increasing the concentration of Na⁺ and Cl⁻ in the cytosol. Sodium toxicity is caused mainly by the similarity of the Na⁺ and K⁺ ions to plant transporters and enzymes. Plant cells typically maintain a high K⁺/Na⁺ ratio in their cytosol with relatively high K⁺, in the order of 100-200 mM, and low Na⁺ , of about 1-10 mM [1].

Natural genetic variation in food crops provides limited opportunity for enhancement of salinity tolerance via crossbreeding strategies. Even relatively saline resistant crops such as rye and barley have threshold salinity values (ECes) well below that of saline water sources such as seawater. The limited repertoire of naturally saline tolerant plants also limits the applicability of crossbreeding strategies, as the plant species to be crossbred must generally be in the same genus or closely related genera.

The advent of programmable nucleases such as Cas endonucleases (e.g., Cas9, CpfI), Transcription activator-like effector nucleases (TALENs), and zinc finger nucleases (ZFNs) has improved the ability to make precise genomic edits in plant species; however, the exact genetic number of and identity of genetic edits to achieve a salinity resistance are not well-defined.

Previous attempts to create transgenic plants that are salt tolerant have employed strategies that express only a small number of genes. For example, EP 3409105 suggested inhibiting the function of PERK13 (Proline-rich extensin-like receptor kinase 13) to produce salt tolerant plants. Fan et al. suggested improving salinity tolerance by introducing Sesuvium portulacastrum SOS1 and AHA genes into Arabidopsis [2]. Both SOS1 and AHA genes encode plasma membrane proteins. This strategy therefore concentrated on controlling the intracellular ion concentration only with plasma membrane proteins. Khan et al. discuss various salt tolerant transgenic plants [3], but these were designed to express only genes that encode ion transporters. A further example of producing salt tolerant plants by altering the expression of an ion transporter is provided by Hossain et al. who produced transgenic tobacco that comprised a sodium/hydrogen antiporter gene [4].

There is a requirement in the art to provide salt tolerant plants that can grow at high salt concentrations, for example, in the sea.

### SUMMARY OF THE INVENTION

The inventors have successfully designed and produced plants that have improved salt tolerance. These plants were engineered to contain multiple different mechanisms for controlling the salt stress which collectively increase the tolerance of the plant to salinity. As a result, disclosed herein are salt tolerant plants. The plants may be transgenic plants and/or may include genes of interest operatively linked to an enhancer element. The transgenes, and/or genes of interest operatively linked to an enhancer element, result in improved salt tolerance by affecting multiple mechanisms as disclosed herein.

The invention provides an engineered plant comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element.

The invention also provides an engineered plant comprising at least two genes of interest, wherein the genes of interest comprise a gene that encodes a plasma membrane protein that controls the intracellular ion concentration and a gene that encodes a tonoplast protein that controls the intracellular ion concentration, wherein the gene that encodes a plasma membrane protein that controls the intracellular ion concentration is operatively linked to an enhancer element and the a gene that encodes a tonoplast protein that controls the intracellular ion concentration is operatively linked to an enhancer element

The invention also provides an engineered plant comprising at least three genes of interest, wherein each gene of interest is operatively linked to an enhancer element, wherein the engineered plant has increased salt tolerance compared to a plant of a same species without said genome modifications.

The invention also provides an engineered plant comprising at least three genes of interest wherein the genes of interest comprise a gene that encodes a plasma membrane protein that controls the intracellular ion concentration, a gene that encodes a tonoplast protein that controls the intracellular ion concentration and a gene that encodes an antioxidant, and wherein the gene that encodes a plasma membrane protein that controls the intracellular ion concentration is operatively linked to an enhancer element, the gene that encodes a tonoplast protein that controls the intracellular ion concentration is operatively linked to an enhancer element and the gene that encodes an antioxidant is operatively linked to an enhancer element.

The invention also provides an engineered rice plant comprising at least four genes of interest, wherein the genes of interest comprise a gene that encodes a plasma membrane ion transporter, a gene that encodes a vacuolar ion transporter, a gene that encodes a potassium transporter and a gene that encodes an antioxidant, and wherein the gene that encodes a plasma membrane ion transporter is operatively linked to an enhancer element, a gene that encodes a vacuolar ion transporter is operatively linked to an enhancer element, a gene that encodes a potassium transporter is operatively linked to an enhancer element and a gene that encodes an antioxidant is operatively linked to an enhancer element.

The invention also provides an engineered rice plant comprising at least four genes of interest, wherein the at least four genes of interest encode OsSOS1, OsNHX1, OsHKT1 and OsSODA1, wherein each gene of interest is operatively linked to an enhancer element. The invention also provides an engineered rice plant comprising at least four genes of interest, wherein the at least four genes of interest encode OsSOS1, OsNHX1, OsHKT1 and OSAHA3, wherein each gene of interest is operatively linked to an enhancer element.

The invention also provides an engineered rice plant comprising at least four genes of interest, wherein the at least four genes of interest encode OsSOS1, OsNHX1, OsHKT1 and OsSODA1, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising an amino acid sequence that has at least 95% sequence identity to SEQ ID NO: 92, and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element.

The invention also provides an engineered rice plant comprising at least four genes of interest, wherein the at least four genes of interest encode OsSOS1, OsNHX1, OsHKT1 and OsAHA3, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsAHA3 gene comprises a polynucleotide sequence that encodes a polypeptide comprising an amino acid sequence that has at least 95% sequence identity to SEQ ID NO: 101, and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

The invention also provides an engineered rice plant comprises at least eight genes of interest, wherein the at least eight genes of interest encode OsSOS1, OsSOS2, OsAHA3, OsVHA A, OsNHX1, OsHKT1, OsSODA1 and OsSOD2; wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.

The invention also provides an engineered rice plant comprising at least eight genes of interest, wherein the at least eight genes of interest encode OsSOS1, OsSOS2, OsAHA3, OsVHA A, OsNHX1, OsHKT1, OsSODA1 and OsSOD2 wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.

The invention also provides an engineered rice plant comprises at least eight genes of interest, wherein the at least eight genes of interest encode OsSOS1, OsSOS2, OsAHA3, OsVHA A, OsNHX1, OsHKT1, OsSODA1 and OsSODCC1; wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.

The invention also provides an engineered rice plant comprising at least eight genes of interest wherein the at least eight genes of interest encode OsSOS1, OsSOS2, OsAHA3, OsVHA A, OsNHX1, OsHKT1, OsSODA1 and OsSODCC1; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.

The invention also provides an engineered rice plant comprising at least eight genes of interest, wherein the eight genes of interest encode OsSOS1, OsSOS2, OsAHA3, OsVHA-A, OsNHX1, OsHKT1, OsSODA1 and OsSOD2; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; wherein the OsSOS1 gene is operably linked to an enhancer element comprising SEQ ID NO: 12, the OsSOS2 gene is operably linked to an enhancer element comprising SEQ ID NO: 13, the OsAHA3 gene is operably linked to an enhancer element comprising SEQ ID NO: 14, the OsVHA-A gene is operably linked to an enhancer element comprising SEQ ID NO: 11, the OsNHX1 gene is operably linked to an enhancer element comprising SEQ ID NO: 10, the OsHKT1 gene is operably linked to an enhancer element comprising SEQ ID NO: 15, the OsSODA1 gene is operably linked to an enhancer element comprising SEQ ID NO: 16 and the OsSOD2 gene is operably linked to an enhancer element comprising SEQ ID NO: 18. Also provided is such an engineered rice plant where the sequences defined in this paragraph are varied, e.g. such that any or all of the sequences specified have at least 95% sequence identity to the specified SEQ ID NO.

The invention also provides a plant part of any of the engineered plants disclosed herein.

The invention also provides a method of making the engineered plant, the plant part or the multicellular structure according to claims 1-82, the method comprising the steps of
i) introducing at least two enhancer elements as defined in the engineered plants according to claims 1-77 into a cell of a plant, wherein the enhancer elements integrate into the genome of the cell of the plant such that they are operatively linked to the genes of interest, and
ii) regenerating the cell to form an engineered plant, a plant part or a multicellular structure from the cell. The invention also provides a method of producing flour, wholemeal, starch or other product obtained from a seed from a plant disclosed herein.

The invention also provides the use of an engineered plant or plant part as disclosed herein as animal feed, or to produce feed for animal consumption or food for human consumption.

The inventors have successfully designed and produced plants that have improved salt tolerance that contain multiple different mechanisms for controlling the salt stress which collectively increase the tolerance of the plant to salinity. This approach can be achieved with engineered plants as set out in the paragraphs above, but it could also be achieved with transgenic plants as set out in the following paragraphs. These approaches could be used separately or in combination, all of which are disclosed herein. For example, the following embodiments that define the transgene could also define the corresponding gene of interest in the engineered plants. A transgenic plant as defined herein could have any one, or more, of the transgenes defined herein replaced by the corresponding gene of interest that is operatively linked to an enhancer element also defined herein.

The invention provides a transgenic plant comprising at least two transgenes wherein the at least two transgenes comprise a transgene that encodes a protein that controls the intracellular ion concentration and a transgene that encodes an antioxidant. In some embodiments, the protein that controls the intracellular ion concentration can be an ion transporter, a hydrogen exporting ATPase, a hydrogen exporting pyrophosphatase or a protein kinase. In further embodiments, the ion transporter, the hydrogen exporting ATPase and/or the hydrogen exporting pyrophosphatase can be a plasma membrane ion transporter, a plasma membrane hydrogen exporting ATPase and/or a plasma membrane hydrogen exporting pyrophosphatase. In some embodiments, the plasma membrane ion transporter can be SOS1. The SOS1 can be OsSOS1. In some embodiments, the OsSOS1 transgene comprises a polynucleotide sequence that encodes a polypeptide comprising an amino acid sequence that has at least 95% sequence identity to SEQ ID NO: 95 and/or 96. In some embodiments, the plasma membrane hydrogen exporting ATPase can be AHA3. The AHA3 can be OsAHA3. In some embodiments, the OsAHA3 transgene comprises a polynucleotide sequence that encodes a polypeptide comprising an amino acid sequence that has at least 95% sequence identity to SEQ ID NO: 101.

The ion transporter, the hydrogen exporting ATPase and/or the hydrogen exporting pyrophosphatase cane be tonoplast ion transporter, a tonoplast hydrogen exporting ATPase and/or a tonoplast hydrogen exporting pyrophosphatase. In some embodiments, the tonoplast ion transporter can be NHX1. The NHX1 can be OsNHX1. In some embodiments, the OsNHX1 transgene comprises a polynucleotide sequence that encodes a polypeptide comprising an amino acid sequence that has at least 95% sequence identity to SEQ ID NO: 97 and/or 98. The tonoplast hydrogen exporting ATPase can be VHA-A. In some embodiments, the VHA-A can be OsVHA-A. In some embodiments, the OsVHA-A transgene comprises a polynucleotide sequence that encodes a polypeptide comprising an amino acid sequence that has at least 95% sequence identity to SEQ ID NO: 102.

In some embodiments, the protein kinase can be a serine/threonine kinase. In some embodiments the serine/threonine kinase can be SOS2. The SOS2 can be OsSOS2. In some embodiments, the OsSOS2 transgene comprises a polynucleotide sequence that encodes a polypeptide comprising an amino acid sequence that has at least 95% sequence identity to SEQ ID NO: 100.

In some embodiments, the antioxidant can be a mitochondrial antioxidant or a cytoplasmic antioxidant. The transgenic plant can comprise at least two transgenes that encode antioxidants. For example, the transgenic plant can comprise a mitochondrial antioxidant and a cytoplasmic antioxidant. In some embodiments, the transgenic plant comprises at least three transgenes that encode antioxidants. In certain embodiments, the antioxidant(s) comprise(s) SODA1, SOD2 and/or SODCC1. The SODA1 can be OsSODA1. In some embodiments, the OsSODA1 transgene comprises a polynucleotide sequence that encodes a polypeptide comprising an amino acid sequence that has at least 95% sequence identity to SEQ ID NO: 92. The SOD2 can be OsSOD2. In some embodiments, the OsSOD2 transgene comprises a polynucleotide sequence that encodes a polypeptide comprising an amino acid sequence that has at least 95% sequence identity to SEQ ID NO: 93. The SODCC1 can be OsSODCC1. In some embodiments the OsSODCC1 transgene comprises a polynucleotide sequence that encodes a polypeptide comprising an amino acid sequence that has at least 95% sequence identity to SEQ ID NO: 94.

The invention also provides a transgenic plant comprising at least two transgenes wherein the at least two transgenes comprise a transgene that encodes a plasma membrane protein that controls the intracellular ion concentration and a transgene that encodes a tonoplast protein that controls the intracellular ion concentration. In some embodiments, the plasma membrane protein that controls the intracellular ion concentration can be a plasma membrane ion transporter, a plasma membrane hydrogen exporting ATPase or a plasma membrane hydrogen exporting pyrophosphatase. In some embodiments, the plasma membrane ion transporter can be SOS1. The SOS1 can be OsSOS1. In some embodiments, the OsSOS1 transgene comprises a polynucleotide sequence that encodes a polypeptide comprising an amino acid sequence that has at least 95% sequence identity to SEQ ID NO: 95 and/or 96. In some embodiments, the plasma membrane hydrogen exporting ATPase can be AHA3. The AHA3 can be OsAHA3. In some embodiments, the OsAHA3 transgene comprises a polynucleotide sequence that encodes a polypeptide comprising an amino acid sequence that has at least 95% sequence identity to SEQ ID NO: 101.

In some embodiments, the tonoplast protein that controls the intracellular ion concentration can be a tonoplast ion transporter, a tonoplast hydrogen exporting ATPase or a tonoplast hydrogen exporting pyrophosphatase.

In some embodiments, the tonoplast ion transporter can be NHX1. The NHX1 can be OsNHX1. In some embodiments, the OsNHX1 transgene can comprises a polynucleotide sequence that encodes a polypeptide comprising an amino acid sequence that has at least 95% sequence identity to SEQ ID NO: 97 and/or 98. The tonoplast hydrogen exporting ATPase can be VHA-A. In some embodiments, the VHA-A can be OsVHA-A. In some embodiments, the OsVHA-A transgene comprises a polynucleotide sequence that encodes a polypeptide comprising an amino acid sequence that has at least 95% sequence identity to SEQ ID NO: 102.

In some embodiments, the transgenic plant further comprises a transgene that encodes a protein kinase and/or a transgene that encodes an antioxidant. In some embodiments, the protein kinase can be a serine/threonine kinase. In some embodiments the serine/threonine kinase can be SOS2. The SOS2 can be OsSOS2. In some embodiments, the OsSOS2 transgene comprises a polynucleotide sequence that encodes a polypeptide comprising an amino acid sequence that has at least 95% sequence identity to SEQ ID NO: 100.

In some embodiments, the antioxidant can be a mitochondrial antioxidant or a cytoplasmic antioxidant. In certain embodiments, the antioxidant comprises any one of SODA1, SOD2 and SODCC1. The SODA1 can be OsSODA1. In some embodiments, the OsSODA1 transgene comprises a polynucleotide sequence that encodes a polypeptide comprising an amino acid sequence that has at least 95% sequence identity to SEQ ID NO: 92. The SOD2 can be OsSOD2. In some embodiments, the OsSOD2 transgene comprises a polynucleotide sequence that encodes a polypeptide comprising an amino acid sequence that has at least 95% sequence identity to SEQ ID NO: 93. The SODCC1 can be OsSODCC1. In some embodiments the OsSODCC1 transgene comprises a polynucleotide sequence that encodes a polypeptide comprising an amino acid sequence that has at least 95% sequence identity to SEQ ID NO: 94.

The invention also provides a transgenic plant comprising at least three transgenes, wherein the transgenic plant has increased salt tolerance compared to a plant of a same species without said genome modifications.

The invention also provides a transgenic plant comprising at least three transgenes wherein the at least three transgenes comprise a transgene that encodes a plasma membrane protein that controls the intracellular ion concentration, a transgene that encodes a tonoplast protein that controls the intracellular ion concentration and a transgene that encodes an antioxidant. In some embodiments, the plasma membrane protein and/or the tonoplast protein that controls the intracellular ion concentration can be an ion transporter, a hydrogen exporting ATPase or a hydrogen exporting pyrophosphatase. In some embodiments, the transgenic plant further comprises a transgene that encodes a protein kinase. The protein kinase can be a serine/threonine kinase. In some embodiments, the serine/threonine kinase can be SOS2.

The invention provides a transgenic rice plant comprises at least four transgenes, wherein the at least four transgenes comprise transgenes that encode OsSOS1, OsNHX1, OsHKT1 and OsSODA1. The invention also provides a transgenic rice plant comprising at least four transgenes, wherein the at least four transgenes comprise transgenes that encode OsSOS1, OsNHX1, OsHKT1 and OsSODA1, wherein the OsSOS1 transgene comprises a polynucleotide sequence that encodes a polypeptide comprising an amino acid sequence that has at least 95% sequence identity to SEQ ID NO: 95 and/or 96; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92.

In some embodiments, the transgenic plant comprises at least seven transgenes, wherein the at least seven transgenes comprise a transgene that encodes a plasma membrane ion transporter; a transgene that encodes a plasma membrane hydrogen exporting ATPase; a transgene that encodes a protein kinase; a transgene that encodes a vacuolar hydrogen exporting ATPase; a transgene that encodes a vacuolar sodium/proton transporter; a transgene that encodes a potassium transporter; and a transgene that encodes an antioxidant.

In some embodiments, the transgenic plant comprises at least eight transgenes, wherein the at least eight transgenes comprise a transgene that encodes a plasma membrane ion transporter; a transgene that encodes a plasma membrane hydrogen exporting ATPase; a transgene that encodes a protein kinase; a transgene that encodes a vacuolar hydrogen exporting ATPase; a transgene that encodes a vacuolar sodium/proton transporter; a transgene that encodes a potassium transporter; a transgene that encodes a first antioxidant and a transgene that encodes a second antioxidant. In some embodiments, the plasma membrane ion transporter can be SOS1. In certain embodiments, the SOS1 can be OsSOS1. In further specific embodiments, the OsSOS1 transgene comprises a polynucleotide sequence that encodes a polypeptide comprising an amino acid sequence that has at least 95% sequence identity to SEQ ID NO: 95 and/or 96. In some embodiments, the plasma membrane hydrogen exporting ATPase can be AHA3. In certain embodiments, the AHA3 can be OsAHA3. In further specific embodiments, the OsAHA3 transgene comprises a polynucleotide sequence that encodes a polypeptide comprising an amino acid sequence that has at least 95% sequence identity to SEQ ID NO: 101. In some embodiments, the protein kinase can be SOS2. In certain embodiments, the SOS2 can be OsSOS2. In further specific embodiments, the OsSOS2 transgene comprises a polynucleotide sequence that encodes a polypeptide comprising an amino acid sequence that has at least 95% sequence identity to SEQ ID NO: 100. In some embodiments, the vacuolar hydrogen exporting ATPase can be VHA-A. In certain embodiments, the VHA-A can be OsVHA-A. In further specific embodiments, the OsVHA-A transgene comprises a polynucleotide sequence that encodes a polypeptide comprising an amino acid sequence that has at least 95% sequence identity to SEQ ID NO: 102. In some embodiments, the vacuolar ion transporter can be NHX1. In certain embodiments, the NHX1 can be OsNHX1. In further specific embodiments, the OsNHX1 transgene comprises a polynucleotide sequence that encodes a polypeptide comprising an amino acid sequence that has at least 95% sequence identity to SEQ ID NO: 97 and/or 98. In some embodiments, the potassium transporter can be HKT1. In certain embodiments, the HKT1 can be OsHKT1. In further specific embodiments, the OsHKT1 transgene comprises a polynucleotide sequence that encodes a polypeptide comprising an amino acid sequence that has at least 95% sequence identity to SEQ ID NO: 99. In some embodiments, the first and the second antioxidants are SODA1, SOD2 and/or SODCC1. In certain embodiments, the SODA1 can be OsSODA1. In further specific embodiments, the OsSODA1 transgene comprises a polynucleotide sequence that encodes a polypeptide comprising an amino acid sequence that has at least 95% sequence identity to SEQ ID NO: 92. In certain embodiments, the SOD2 can be OsSOD2. In further specific embodiments, the OsSOD2 transgene comprises a polynucleotide sequence that encodes a polypeptide comprising an amino acid sequence that has at least 95% sequence identity to SEQ ID NO: 93. In certain embodiments, the SODCC1 can be OsSODCC1. In further specific embodiments, the OsSODCC1 transgene comprises a polynucleotide sequence that encodes a polypeptide comprising an amino acid sequence that has at least 95% sequence identity to SEQ ID NO: 94.

The invention provides a transgenic rice plant comprising at least eight transgenes, wherein the at least eight transgenes comprise transgenes that encode OsSOS1, OsSOS2, OsAHA3, OsVHA-A, OsNHX1, OsHKT1, OsSODA1 and OsSOD2. In a further aspect, the invention provides a transgenic rice plant comprising at least eight transgenes wherein the at least eight transgenes comprise transgenes that encode OsSOS1, OsSOS2, OsAHA3, OsVHA-A, OsNHX1, OsHKT1, OsSODA1 and OsSOD2; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93.

The invention provides a transgenic rice plant comprises at least eight transgenes, wherein the at least eight transgenes comprise transgenes that encode OsSOS1, OsSOS2, OsAHA3, OsVHA-A, OsNHX1, OsHKT1, OsSODA1 and OsSODCC1. In another aspect, the invention provides a transgenic rice plant comprising at least eight transgenes, wherein the at least eight transgenes comprise transgenes that encode OsSOS1, OsSOS2, OsAHA3, OsVHA-A, OsNHX1, OsHKT1, OsSODA1 and OsSODCC1; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSODCC1 transgene comprises a polynucleotide sequence that encodes a polypeptide comprising an amino acid sequence that has at least 95% sequence identity to SEQ ID NO: 94.

In some embodiments, the transgenic plant further comprises at least one additional transgene, wherein the at least one additional transgene encodes a protein that can be a cytochrome p450 (P450); an oxygen-evolving complex; a sucrose phosphate synthase; and/or a pyrroline carboxylate synthase. In some embodiments, the oxygen-evolving complex can be formed of the proteins PsbO, PsbP and PsbQ. In other embodiments, the oxygen-evolving complex can be formed of the proteins PsbO, PsbP, PsbQ, PsbU and PsbV. In other embodiments, the sucrose phosphate synthase can be sucrose phosphate synthase 1, sucrose phosphate synthase 2 or sucrose phosphate synthase 3. In some embodiments, the pyrroline carboxylate synthase can be delta-1-pyrroline-5-carboxylate synthase 1, or delta-1-pyrroline-5-carboxylate synthase 2. In some embodiments, the transgenic plant further comprises a transgene that encodes OSK1. In some embodiments, the transgenic plant does not contain a transgene that encodes PERK13.

In some embodiments, at least one of the transgenes is operably linked to at least one promoter. In certain embodiments, all of the transgenes are operably linked to a promoter. In some embodiments, the at least one promoter comprises at least 10, at least 20, or at least 30 nucleotides. In some embodiments, the at least one promoter can be within 150-500 nucleotides of the 5′ end of an open reading frame of the transgene. In some embodiments, the at least one promoter can be a root-specific promoter. In certain embodiments, all of the transgenes are operably linked to a root-specific promoter. In some embodiments, the at least one promoter comprises a DREB2A, gibberellin, ethylene or auxin promoter sequence or a combination thereof, optionally wherein all of the transgenes are operably linked to a promoter comprising a DREB2A, gibberellin, ethylene or auxin promoter sequence or a combination thereof. In some embodiments, the at least one promoter comprises at least 6 nucleotides from an promoter element from a DREB2A, gibberellin, ethylene or auxin promoter sequence or a combination thereof, optionally wherein all of the transgenes are operably linked to a promoter comprising an promoter element from a DREB2A, gibberellin, ethylene or auxin promoter sequence. In some embodiments, the at least one promoter comprises a TAF-1, TATA, E2F, G-BOX, or CAAT promoter sequence or a combination thereof, optionally wherein all of the transgenes are operably linked to a promoter comprising a TAF-1, TATA, E2F, G-BOX, or CAAT promoter sequence. In some embodiments, the at least one promoter comprises a sequence having at least 95% sequence identity to any one of SEQ ID NO: 10-18.

In some embodiments, each transgene may encode a separate protein, i.e. each protein introduced into the transgenic plant is encoded by a separate transgene.

In some embodiments, the transgenic plant can be an angiosperm. In some embodiments, the transgenic plant can be monocotyledonous or dicotyledonous. In certain embodiments, the transgenic plant can be a cereal crop. In further specific embodiments, the transgenic plant can be maize, rice, soybean, sugar cane, mung bean, quinoa, barley, oat, rye, sorghum, or wheat. In a certain embodiment, the transgenic plant can be a transgenic rice plant. In some embodiments, the transgenic plant can be a vegetable crop. In some embodiments, the transgenic plant can be from the genus a Brassica, Glycine, or Soja.

The invention also provides a plant part of the transgenic plant according to the invention. In some embodiments, the plant part can be a cell, a seed, a leaf, a shoot, a stem or a root. In certain embodiments, the plant part can be a seed or a cell. The invention also provides a multicellular structure comprising one or more plant cells according to the invention. In some embodiments, the multicellular structure can be a callus.

The invention also provides methods of making the transgenic plant, the plant part or the multicellular structure according to the invention, the method comprising the steps of: i) introducing the at least two transgenes as defined in the transgenic plants according to the invention into a cell of a plant, wherein the transgenes integrate into the genome of the cell of the plant, and ii) regenerating the cell to form a transgenic plant, a plant part or a multicellular structure from the cell. In some embodiments, the transgenes are introduced into the cell by particle bombardment, Agrobacterium mediated transformation or by protoplast transfection. In some embodiments, the methods involves: (a) inducing callus formation from a seed; (b) precipitating a polynucleotide sequence, a guide RNA and a nuclease onto a microcarrier; wherein the polynucleotide sequence comprises the at least two transgenes as described herein; (c) transforming the callus with the microcarriers using particle bombardment to generate a transformed callus wherein the polynucleotide sequence integrates into the genome of the transgenic plant, the plant part or the multicellular structure; (d) recovering the transformed callus to generate a multicellular structure according to the invention. In some embodiments, the multicellular structure is regenerated into a transgenic plant. In some embodiments, the polynucleotide sequence is stably integrated into the genome of the plant. In some embodiments, the nuclease is a Cas nuclease, Cpf1 nuclease, a TALEN or a zinc finger nucleases, optionally wherein the nuclease is Cas9 or Cpf1. In some embodiments, the polynucleotide is RNA, DNA or a plasmid, optionally wherein the polynucleotide is DNA.

In some embodiments, the transgenic plant, the plant part or the multicellular structure according to the invention is not produced by a process that involves homologous recombination and/or is not produced by an essentially biological process.

The invention also provides methods of producing flour, wholemeal, starch or other product obtained from a seed according of the invention. The invention also provides uses of a transgenic plant, a part thereof or a multicellular structure according to the invention as animal feed, or to produce feed for animal consumption or food for human consumption.

### BRIEF DESCRIPTION OF DRAWINGS

FIG. 1—The mechanism of Cpf1 (Cas12a), including the location of cut sites and genetic inserts. Inserts contain corresponding sequences to the overhangs plus the desired promoter-based sequence to direct expression of the chosen gene.

FIG. 2—Callus induction rates of the Rice varieties TH 3-5, Truong Giang, MHC2 and Ha Phat 3 with different concentrations of 2,4-D and BAP. The concentrations in each treatment are summarised in Table 12.

FIG. 3—Analysis of Java long, Se Zic, Agostano, Hunan and Dichroa rice varieties. FIG. 3a shows percentage of seed from the Java long, Se Zic, Agostano, Hunan and Dichroa Rice varieties that germinated. FIG. 3b shows the callus induction rates of the rice varieties with different concentrations of 2,4-D and BAP. The concentrations in each treatment are summarised in Table 12. FIG. 3c shows the shoot development of the rice varieties with the same treatments. The order of the bars from left to right are: Jaca long, Se Zic, Agostano, Hunan and Dichroa repeated, with a space between each set.

FIG. 4—Callus induction rates of the rice variety Hayayuki with different concentrations of 2,4-D and BAP. The concentrations in each treatment are summarised in Table 13.

FIG. 5—qRT-PCR analysis of engineered plants according to the invention. FIG. 5a shows the expression of four genes of interest in the leaf, while FIG. 5b shows the expression of four genes of interest in the root. WT denotes a wild type plant. Genotypes 1, 6, 9 and 10 are four different engineered plants according to the invention and the genes of interest are NHX1, SOS1, HKT1 and SODA1. The order of the bars from left to right are: NHX1; SOS1, HKT1 and SODA1 repeated, with a space between each set.

FIG. 6—The sequences added to the 5′ and 3′ ends of the DNA inserts. The promoter sequences within the 5′ inserts are underlined.

FIG. 7—The distribution of hormones throughout the life cycle of a plant. Presence of a particular hormone is indicated by the coloured boxes.

FIG. 8—An example of a yield vs salinity (ECe) plot.

FIG. 9—Bar graphs showing the gene expression of OsSOS1, OsSOS2, OsAHA3, OsVHA-A, OsNHX1, OsHKT1, OsSODA1 and OsSOD2 in engineered plants according to the invention. The gene expression is relative to both the reference gene, Actin-3, and to the wild-type plants. A positive value indicates an upregulation of the expression, a negative value indicates a down regulation of the gene expression. FIGS. 9a and 9b show the normalised gene expression grouped by the engineered plants. In FIG. 9b the data have been grouped by biological sample, whereas the data in FIG. 9a have not. FIG. 9c shows the normalised gene expression grouped by the genes of interest. The engineered plants with their corresponding genes of interest are summarized in Table 8.

FIGS. 9a and 9b the order of the bars from left to right are: Plant A —Os-VHA-A, SOS1, SOS2, OsAHA3, HKT1, SODA1, OsSOD2, space, Plant B—NHX1, Os-VHA-A, SOS2, OsAHA3, HKT1, SODA1, OsSOD2, space, Plant C—SOS1, SOS2, OsAHA3, HKT1, SODA1, OsSOD2, space, Plant D—NHX1, Os-VHA-A, SOS2, OsAHA3, HKT1, SODA1, OsSOD2, space, Plant E1 Os-VHA-A, SOS1, SOS2, OsAHA3, HKT1, SODA1, OsSOD2, space, Plant F Os-VHA-A, SOS2, OsAHA3, HKT1, SODA1, OsSOD2, space, Plant G—Os-VHA-A, SOS2, OsAHA3, HKT1, SODA1, OsSOD2, space, Plant I—SOS1, SOS2, OsAHA3, HKT1, SODA1, OsSOD2, space Plant 1—Os-VHA-A, SOS1, SOS2, OsAHA3, HKT1, SODA1, OsSOD2, space, Plant 9—Os-VHA-A, SOS2, OsAHA3, HKT1, SODA1, OsSOD2, space, Wild type—NHX1; OsVHA-A; SOS1; SOD2; OsAHA3; HKT1; SODA1 and OsSOD2. FIG. 9c the order of the bars from left to right are: Plant A, Plant B, Plant C, Plant D, Plant E1, Plant F, Plant G, Plant I, Plant 1, Plant 9 and Wild type repeated.

FIG. 10a-j—Scatter plots mapping the normalized gene expression of engineered plants according to the invention against wild type plants (WT). The settings on the plot include a fold-change threshold of 2.00 with each sample containing two replicates. FIG. 10(a) is the scatter plot for engineered plant A; FIG. 10(b) is the scatter plot for engineered plant B; FIG. 10(c) is the scatter plot for engineered plant C; FIG. 10(d) is the scatter plot for engineered plant D; FIG. 10(e) is the scatter plot for engineered plant E1; FIG. 10(f) is the scatter plot for engineered plant F; FIG. 10(g) is the scatter plot for engineered plant I; FIG. 10(h) is the scatter plot for engineered plant G; FIG. 10(i) is the scatter plot for engineered plant 1; FIG. 10(j) is the scatter plot for engineered plant 9. The engineered plants with their corresponding genes of interest are summarized in Table 8.

FIG. 11—Images of wild type (WT) and engineered rice plants grown in saline media. The engineered plants with their corresponding genes of interest are summarized in Table 8.

FIGS. 12a-c—Images of wild type (WT) and engineered calli grown in saline media. The genes of interest in the engineered calli are OsNHX1, OsVHA-A, OsSOS1 & OsSOS2.

FIG. 13—Bar graph showing the gene expression of OsSOS1, OsSOS2, OsAHA3, OsVHA-A, OsNHX1, OsHKT1, OsSODA1 and OsSOD2 in engineered calli according to the invention. The gene expression is relative to both the reference gene, Actin-3, and to the wild-type plants. A positive value indicates an upregulation of the expression, a negative value indicates a down regulation of the gene expression. The genes of interest in the engineered calli are OsNHX1, OsVHA-A, OsSOS1 & OsSOS2. The order of the bars from left to right are: SOS1; SOD2; HKT1; NHX1; OsAHA3; OsVHA-A and SODA1 repeated, with a space between each set.

FIG. 14—Bar graph showing the gene expression of OsSOS1, OsSOS2, OsAHA3, OsVHA-A, OsNHX1, OsHKT1, OsSODA1 and OsSOD2 in wild-type calli when grown in medium with 0, 2, 4, 6, 8, 10, 12.5 & 15 g/L of sodium chloride. The gene expression is relative to both the reference gene, Actin-3, and to the wild-type plants. A positive value indicates an upregulation of the expression, a negative value indicates a down regulation of the gene expression. The order of the bars from left to right are: HKT1; NHX1; OsAHA3; OsVHA-A; SODA1 SOS1 and SOD2; repeated, with a space between each set.

FIG. 15a-o—Scatter plots mapping the normalized gene expression of engineered calli according to the invention against wild type plants (WT). The settings on the plot include a fold-change threshold of 2.00 with each sample containing two replicates. The genes of interest in the engineered calli are OsNHX1, OsVHA-A, OsSOS1 & OsSOS2.

FIG. 16a-e—Images of three engineered rice plants that are producing seeds. The engineered plants comprise the following genes of interest, OsNHX1, OsVHA-A, OsSOS1, OsSOS2, OsAHA3, OsHKT1, OsSODA1, and either OsSODCC1 or OsSOD2, wherein each gene of interest is operatively linked to an enhancer element. (A) The large plant on the left hand side of the image is a engineered plant according to the invention which is a Troung Giang rice variety, while the two plants on the right hand side are engineered plants according to the invention that are from the Hayayuki rice variety; (B) A closer image of the two engineered rice plants of the Hayayuki rice variety; (C) An image showing the panicles and seeds of an engineered rice plant of the Hayayuki rice variety; (D) An image of the engineered rice plant of the Troung Giang rice variety; (C) An image showing the panicles and seeds of an engineered rice plant of the Troung Giang rice variety.

FIG. 17—A bar graph showing the gene expression of OsSOS1, OsSOS2, OsAHA3, OsVHA-A, OsNHX1, OsHKT1, OsSODA1, OsSODCC1 and OsSOD2 in seedlings 13 and 14. Seedlings 13 and 14 were grown from seed harvested from an engineered rice plant of the Hayayuki rice variety. These seedlings were grown from seeds that were obtained from engineered plants according to the invention normalized relative to a wild-type rice plant.

FIG. 18—Scatter plots mapping the normalized gene expression of seedlings 13 and 14 normalized relative to a wild-type rice plant. The bars represent a four-fold increase in gene expression.

### DEFINITIONS

Unless specifically defined otherwise, all technical and scientific terms used herein shall be taken to have the same meaning as commonly understood by one of ordinary skill in the art (e.g., in cell culture, molecular genetics, plant molecular biology, protein chemistry, and biochemistry).

As used herein, the term “medium” generally refers to a natural or man-made substance in solid, semi-solid, or liquid form that can be used to grow a plant. Examples of suitable types of media that can be used in the present disclosure include soil, artificial or non-soil potted mix, water (e.g., for hydroponic growth formats), and agar.

“Salinity,” as used herein, generally refers to a measure of soluble salts in soil or water. “Salt,” as used herein, generally refers to any molecule comprised of a cation, such as sodium (Na⁺), potassium (K⁺), magnesium (Mg²⁺), or calcium (Ca²⁺), and an anion, such as chloride (Cl⁻), bicarbonate (HCO₃⁻), carbonate (CO₃²⁻), or sulfate (SO₄²⁻). Sodium chloride (NaCl) is the most common salt in groundwater and soils. The salinity of soil (or another growth medium) can be expressed: (a) as the salt concentration of the medium in terms of grams per liter (g/L) or (b) in terms of electric conductivity (EC, in deciSiemens per meter—dS/m, or in equivalent units milliMhos per centimeter—mmhos/cm or milliSiemens per centimeter—mS/cm). For soil, salinity can be measured in units of electrical conductivity of a saturated soil paste extract (ECₑ) taken from the root zone of a plant and averaged over time and depth. Soil paste extracts are soil samples that are brought up to their water saturation points (see, e.g., USDA. Diagnosis and improvement of saline and alkali soils. Agriculture Handbook No. 60. (1954), which is incorporated by reference herein). In some embodiments, electrical conductivities are measured on the vacuum-extracted and filtered water extracts from saturated soil paste extracts.

According to the USDA salinity laboratory, “saline” can be defined as a medium having an electrical conductivity of the saturated paste extract (ECₑ) of 4 dS/m or greater, “slightly saline” (or medium) can be defined as having an electrical conductivity of the saturated paste extract (ECₑ) of between 4 and 8, moderately saline can be defined as having an electrical conductivity of the saturated paste extract (ECₑ) of between 8 and 16, and severely saline can be defined as having an electrical conductivity of the saturated paste extract (ECₑ) of greater than 16; and seawater may have a salt concentration of 30 g/L and an EC of 50 dS/m. However, effects can occur on plants at salinity levels lower than 4 dS/m; so-called sensitive plants can exhibit growth problems at 0.75-1.5 dS/m and many plants can nonetheless experience growth rate decreases at 1.5-3.0 dS/m.

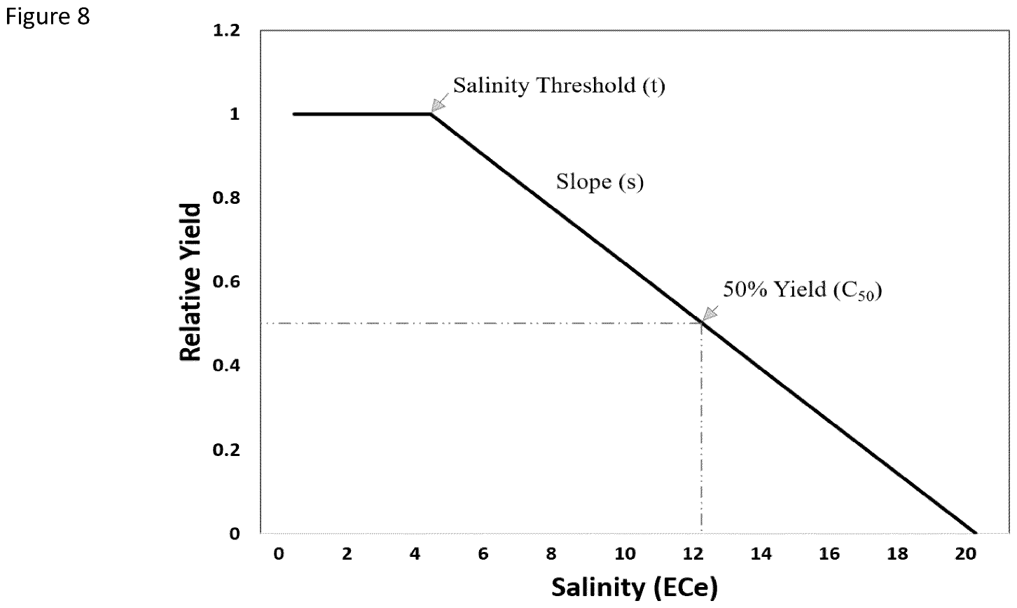

Salinity tolerance can be manifested by resistance to individual physicochemical stresses that combine to cause salinity stress such as ionic stress (which can be tested, e.g., by increased resistance to concentrations of LiCl) and/or osmotic stress (which can be tested, e.g., by increased resistance to concentrations of polyethylene glycol). The general effect of salinity on plants is to reduce the growth rate resulting in smaller leaves, shorter stature, fewer leaves, and/or decreased yield. Therefore in some embodiments, salinity tolerance can be assessed by reference to yield, mass, length, or growth rate of the whole plant, or by the yield, mass, length, or growth rate of particular parts of the plant (e.g., roots, leaves, shoots, and/or seeds). For example, a plant's tolerance to salinity can be described as a function of yield decline across a range of salt concentrations. In other embodiments, plant salt tolerance can be described using two parameters, the threshold (ECₜ), the electrical conductivity that is expected to cause the initial significant reduction in the maximum expected yield (Yₘₐₓ), and the slope (s). FIG. 8 depicts a diagram of an example yield vs salinity plot, showing the salinity threshold (ECₜ), Yₘₐₓ, and slope (s). In terms of ECₜ, plants with ECₜ of 0.9 dS/m or less can be considered salt sensitive, plants with ECₜ greater than 0.9 up to 1.4 dS/m can be considered moderately sensitive, plants with ECₜ greater than 1.4 up to 2.5 dS/m can be considered moderately tolerant, and plants with ECₜ greater than 2.5 dS/m can be considered tolerant. In other embodiments, salt tolerance can be expressed in terms of the electrical conductivity of the saturated paste extract (ECₑ) at which yield is reduced by 50% (C₅₀).

As used herein, the term “operably linked” generally means that a promoter sequence or enhancer sequence is positioned with respect to a transcribable or translatable polynucleotide sequence (i.e. the transgene or gene of interest) such that the regulatory element within the promoter sequence can affect the regulatory activity of the polynucleotide sequence. A promoter having transcriptional promoter activity, for example, can be located at any distance, including adjacent to or up to thousands of nucleotides away from, and upstream or downstream from the gene, which can be a minimal promoter element, and polynucleotide sequence to be transcribed, and still exert a detectable effect on the level of expression of an encoded reporter molecule.

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, +/−5%, +/−1%, or +/−0.5%, of the designated value.

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

With regard to a defined polypeptide, it will be appreciated that % identity figures higher than those provided above will encompass further specific embodiments. Thus, where applicable, in light of the minimum % identity figures, the polypeptide may comprise an amino acid sequence which is at least 60%, at least 65%, at least 70%, at least 75%, at least 76%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical or 100% identical to the relevant nominated SEQ ID NO.

The extent of similarity between two sequences can be based on percent sequence identity. “Sequence identity” herein means the extent to which two polynucleotide or amino acid sequences are invariant. “Sequence alignment” means the process of lining up two or more sequences to achieve maximal levels of identity for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

Exemplary methods are described in section 7.7.18 of ref. [5]. Alignments may be determined by the Smith-Waterman homology search algorithm using an affine gap search with a gap open penalty of 5 or 12 and a gap extension penalty of 2, BLOSUM matrix of 62. The Smith-Waterman homology search algorithm is disclosed in ref. [6].” In an alternative embodiment, the percentage identity of a polypeptide can be determined by GAP (Needleman and Wunsch, 1970) analysis (GCG program) with a gap creation penalty=5, and a gap extension penalty=0.3. The query sequence is at least 150 amino acids in length, and the GAP analysis aligns the two sequences over a region of at least 150 amino acids. The query sequence may be at least 500 amino acids in length, and the GAP analysis aligns the two sequences over a region of at least 500 amino acids. The query sequence may be at least 750 amino acids in length and the GAP analysis aligns the two sequences over a region of at least 750 amino acids. The query sequence may be at least 900 amino acids in length and the GAP analysis aligns the two sequences over a region of at least 900 amino acids. The GAP analysis may align two sequences over their entire length.

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

An enhancer element is a polynucleotide sequence that comprises or consists of a regulatory element(s) that is/are capable of altering the expression level of a gene of interest compared to the expression level of the gene of interest in a wild-type plant. For example, an enhancer element may increase the expression level of a gene of interest compared to a wild-type plant. The enhancer element may alter the expression level of the gene of interest at all times or at one or more stages of the plant's life cycle, for example when the plant is germinating. The enhancer element may result in the expression of the gene of interest at a different location in the plant compared to a wild-type plant.

The enhancer element can comprise a regulatory element(s) that is/are naturally present in the plant genome or can contain regulatory elements that are not naturally present in the plant genome. For example, the enhancer element may comprise a polynucleotide sequence obtained from or derived from a plant cell of the same species, or another plant cell of a different species, or a non-plant source, or a synthetic sequence. In certain embodiments, the enhancer element is not the promoter sequence that is naturally present in the plant genome for the corresponding gene of interest.

An enhancer element is incorporated into the genome of a plant by plant transformation methods that involve genetic engineering i.e. the enhancer element is not incorporated into the genome of the plant by an essentially biological means, such as crossing, interbreeding or selective breeding. As a result of such genomic alteration, the engineered plant is different from the related wild-type plant and has a trait that is not naturally found in the wild-type plant. In certain embodiments, the enhancer element(s) is/are stably incorporated into the genome of the plant.

The terms “gene of interest”, “gene” and “genes” refer to polynucleotide sequences that naturally occur in the genome of the plant, i.e. it is the endogenous gene within the plant.

The term “transgene” refers to a polynucleotide sequence that is incorporated into the genome of a plant by plant transformation methods that involve genetic engineering i.e. the transgene is not incorporated into the genome of the plant by an essentially biological means, such as crossing, interbreeding or selective breeding. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. The transgene may include polynucleotide sequences obtained from or derived from a plant cell of the same species, or another plant cell of a different species, or a non-plant source, or a synthetic sequence. As a result of such genomic alteration, the transgenic plant is different from the related wild-type plant and the transgenic trait is a trait not naturally found in the wild-type plant.

If the transgene in a transgenic plant is obtained from or derived from a plant cell of the same species as the transgenic plant, then the presence of the transgene means that an extra copy of the gene exists in the plant genome compared to a wild-type plant i.e. the transgenic plant comprises the endogenous gene and an extra copy of the gene, which is the transgene. The additional copy of the gene may be operably linked to the endogenous plant promoter or an artificial promoter that is different from the endogenous promoter. The presence of an artificial promoter may result in the expression levels or expression pattern of the transgene being different when compared to a wild-type plant. For example the expression level of the transgene may be increased or decreased compared to a wild-type plant. The additional copy of the gene may result in altered expression of the gene at some point in the plant's life cycle, for example when the plant is germinating. The additional copy of the gene may result in the expression of the gene in a different location in the plant compared to the endogenous gene. In certain embodiments, the transgenes are stably incorporated into the genome of the plant.

In some embodiments, the transgenic plant can comprise a transgene and a gene of interest that is operatively linked to an enhancer element. The transgene or gene of interest can any transgene or gene of interest as described herein. The transgene or gene of interest can encode the same protein or can encode different proteins. For example, the transgenic plant may comprise a transgene that encodes a protein that controls the intracellular ion concentration and a gene of interest that encodes an antioxidant; wherein the gene that encodes an antioxidant is operatively linked to an enhancer element.

All patent and literature references cited in the present specification are hereby incorporated by reference in their entirety.

### DISCLOSURE OF THE INVENTION

The improved salt tolerance disclosed herein can be achieved by a number of routes, including making a transgenic plant as disclosed herein and/or an engineered plant as disclosed herein. The improved salt tolerance can be provided by the increased gene and/or transgene expression, relative to wild-type, achieved as disclosed herein. For example, the increased expression, relative to wild-type, might be achieved by using a transgene(s) as disclosed herein and/or a gene of interest operatively linked to an enhancer element.

### Transgenes and Genes of Interest

The invention provides engineered plants that have been specifically designed to have enhancer elements that are operatively linked to genes of interest, such that the plants have increased salt tolerance. For example, the invention provides an engineered plant comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element.

The inventors have identified that it is advantageous to insert short enhancer elements into the genome of plants so that they are operatively linked to genes of interest, rather than inserting transgenes. This method for producing the engineered plants according to the invention requires less DNA to be inserted into the genome, which means that it is less invasive to the plant. Without wishing to be bound by any particular theory, the inventors think that the improved salt tolerance observed in the engineered plants of the invention may be due to the reduced amount of DNA that is inserted into the plant genome. In addition, plants that have been gene edited rather than inserting whole transgenes into their genome require shorter regulatory approval.

The invention provides transgenic plants that have been specifically designed and genetically engineered to comprise transgenes, such that the plants have increased salt tolerance. For example, the invention provides a transgenic plant comprising at least two transgenes, wherein the at least two transgenes comprise a transgene that encodes a protein that controls the intracellular ion concentration and a transgene that encodes an antioxidant.

The invention also provides engineered plants that have been specifically designed and genetically engineered to include an enhancer element that is operatively linked to a gene of interest, such that the plants have increased salt tolerance. For example, the invention provides an engineered plant comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element.

Both approaches, using transgenes and using genes of interest linked to enhancer elements, can be routes to the same outcome of increasing the expression of the transgene and/or the gene of interest. As such, the proteins encoded by the transgenes and genes of interest disclosed herein may be the same.

### Antioxidants

As the sodium concentration within a cell increases, the sodium ions interfere with metabolic reactions and cause an increase in reactive oxygen species and radicals, such as hydrogen peroxide, OH⁻ and oxygen. These reactive oxygen species and radicals can have various deleterious effects in the cell, such as damaging the DNA. Antioxidants are compounds that inhibit oxidation, which produces reactive oxygen species and radicals. Excess levels of chloride ions can also lead to an increase in reactive oxygen species and radicals. The inventors have advantageously identified that expressing at least one transgene that encodes an antioxidant in a transgenic plant increases salt tolerance of the transgenic plant. The inventors have advantageously identified that operatively linking an enhancer element to a gene of interest that encodes an antioxidant in an engineered plant increases salt tolerance of the engineered plant.

The antioxidant can be a mitochondrial antioxidant or a cytoplasmic antioxidant. In some embodiments, the antioxidant is superoxide dismutase 1 (SODA). In some embodiments, the antioxidant is superoxide dismutase 2 (SOD2). In some embodiments, the antioxidant is cytosolic superoxidase dismutase (SODCC1).

In some embodiments, the SODA1 is Oryza sativa SODA1 (OsSODA1). OsSODA1 can have the polypeptide sequence of Uniprot accession number Q43121 or SEQ ID NO: 92. In some embodiments, the transgene or gene of interest comprises a polynucleotide sequence that encodes a polypeptide comprising the amino acid sequence of Q43121, or the transgene can comprise an amino acid sequence that has at least 75%, 80%, 85%, 90%, 95%, 96%, 97%, 98%, 99%, 99.5% or 99.9% sequence identity to Q43121. In some embodiments, the transgene or gene of interest comprises a polynucleotide sequence that encodes a polypeptide comprising the amino acid sequence of SEQ ID NO: 92, or comprising an amino acid sequence that has at least 75%, 80%, 85%, 90%, 95%, 96%, 97%, 98%, 99%, 99.5% or 99.9% sequence identity to SEQ ID NO: 92.

In some embodiments, the SOD2 is Oryza sativa SOD2 (OsSOD2). OsSOD2 can have the polypeptide sequence of the Uniprot accession number Q10PW4 or SEQ ID NO: 93. In some embodiments, the transgene or gene of interest comprises a polynucleotide sequence that encodes a polypeptide comprising the amino acid sequence of Q10PW4, or the transgene can comprising amino acid sequence that has at least 75%, 80%, 85%, 90%, 95%, 96%, 97%, 98%, 99%, 99.5% or 99.9% sequence identity to Q10PW4. In some embodiments, the transgene or gene of interest comprises a polynucleotide sequence that encodes a polypeptide comprising the amino acid sequence of SEQ ID NO: 93, or comprising an amino acid sequence that has at least 75%, 80%, 85%, 90%, 95%, 96%, 97%, 98%, 99%, 99.5% or 99.9% sequence identity to SEQ ID NO: 93.

In some embodiments, the SODCC1 is Oryza sativa SODCC1 (OsSODCC1). OsSODCC1 can have the polypeptide sequence of the Uniprot accession number QODRV6 or SEQ ID NO 94. In some embodiments, the transgene or gene of interest comprises a polynucleotide sequence that encodes a polypeptide comprising the amino acid sequence of QODRV6, or the transgene can comprising amino acid sequence that has at least 75%, 80%, 85%, 90%, 95%, 96%, 97%, 98%, 99%, 99.5% or 99.9% sequence identity to QODRV6. In some embodiments, the transgene or gene of interest comprises a polynucleotide sequence that encodes a polypeptide comprising the amino acid sequence of SEQ ID NO: 94, or comprising an amino acid sequence that has at least 75%, 80%, 85%, 90%, 95%, 96%, 97%, 98%, 99%, 99.5% or 99.9% sequence identity to SEQ ID NO: 94.

In some embodiments, the transgenic plant can comprise at least two transgenes, at least three transgenes, at least four transgenes or at least five transgenes that encode an antioxidant. In certain embodiments, the transgenic plant can comprise at least two transgenes that encode antioxidants. The antioxidant can be a mitochondrial antioxidant and a cytoplasmic antioxidant. In certain embodiments, the transgenic plant can comprise at least two transgenes including a transgene that encodes a mitochondrial antioxidant and a transgene that encodes a cytoplasmic antioxidant. In some embodiments, the antioxidants can be a manganese superoxide dismutase and/or a CuZn superoxide dismutase. In certain embodiments, the transgenic plant can comprise at least two transgenes including transgenes that encode two antioxidants, wherein the two antioxidants are selected from the group consisting of SODA1 and SOD2; SODA1 and SODCC1; or SOD2 and SODCC1. In some embodiments, the transgenic plant comprises at least three transgenes including transgenes that encode SODA1, SOD2 and SODCC1. In certain embodiments, the transgenic plant comprises at least three transgenes including transgenes that encode OsSODA1, OsSOD2 and OsSODCC1.

In some embodiments, the engineered plant can comprise at least two genes of interest, at least three genes of interest, at least four genes of interest or at least five genes of interest that encode an antioxidant. In certain embodiments, the engineered plant can comprise at least two genes of interest that encode antioxidants. The antioxidant can be a mitochondrial antioxidant and a cytoplasmic antioxidant. In certain embodiments, the engineered plant can comprise at least two genes of interest including a gene of interest that encodes a mitochondrial antioxidant and a gene of interest that encodes a cytoplasmic antioxidant. In some embodiments, the antioxidants can be a manganese superoxide dismutase and/or a CuZn superoxide dismutase. In certain embodiments, the engineered plant can comprise at least two genes of interest including genes of interest that encode two antioxidants, wherein the two antioxidants are selected from the group consisting of SODA1 and SOD2; SODA1 and SODCC1; or SOD2 and SODCC1. In some embodiments, the engineered plant comprises at least three genes of interest including genes of interest that encode SODA1, SOD2 and SODCC1. In certain embodiments, the engineered plant comprises at least three genes of interest including genes of interest that encode OsSODA1, OsSOD2 and OsSODCC1. In each of the embodiments in this paragraph, the gene of interest is operatively linked to an enhancer element as disclosed herein.

Proteins that can Control the Intracellular Ion Concentration

Proteins that can control the intracellular ion concentration include ion transporters (e.g. a sodium or potassium transporter), hydrogen exporting ATPases, hydrogen exporting pyrophosphatases or protein kinases.

Plants use two strategies to maintain a low cytosolic sodium concentration. Sodium can be excluded from the cytosol to the apoplast or the extracellular space. Alternatively, sodium can be transported to the vacuole where it is stored, which is known as sodium compartmentation. The inventors have identified that exploiting both plasma membrane and tonoplast mechanisms for reducing the cytosolic sodium concentration can advantageously increase the stress tolerance of an engineered plant. Therefore, the invention provides an engineered plant comprising at least two genes of interest, wherein the genes of interest comprise a gene that encodes a plasma membrane protein that controls the intracellular ion concentration and a gene that encodes a tonoplast protein that controls the intracellular ion concentration, wherein the gene that encodes a plasma membrane protein that controls the intracellular ion concentration is operatively linked to an enhancer element and the a gene that encodes a tonoplast protein that controls the intracellular ion concentration is operatively linked to an enhancer element.

The invention also provides transgenic plants comprising at least two transgenes including a transgene that encodes a plasma membrane protein that controls the intracellular ion concentration and a tonoplast protein that controls the intracellular ion concentration.

### Ion Transporters

Ion transporters can transport ions, such as sodium or potassium across membranes either passively via an ion gradient or actively using energy from various sources-including adenosine triphosphate (ATP), sunlight, and other redox reactions. Ion transporters for use in the invention may be active ion transporters. Active ion transporter are able to remove ions from the cytosol of the plant cell against a concentration gradient.

Plasma membrane-bound sodium/hydrogen antiporters operate to exclude sodium from the cell. SOS1 (Salt Overly Sensitive 1) is a plasma membrane sodium/hydrogen antiporter that extrudes excessive sodium from the cytosol. Studies in several species have now shown that SOS1 is conserved in higher plants including both monocotyledonous and dicotyledonous plants [7]. SOS1 has also been was previously identified as essential for plant salt tolerance [8].

Sodium compartmentation can occur in the vacuole or the apoplast. The plant vacuoles play central roles in plant stress responses. The central vacuole, which can occupy more than 80% of the total plant cell volume, is separated from the surrounding cytosol by the tonoplast membrane that controls the passage of inorganic and organic solutes to and from the cytoplasm through a wide range of pumps, carriers, ion channels and receptors [1]. NHX1 (Na+/H+ antiporter 1) is a tonoplast (vacuolar) sodium/hydrogen ion exchanger.

In some embodiments, the ion transporter is a sodium/hydrogen antiporter. In some embodiments, the ion transporter is a plasma membrane sodium/hydrogen antiporter, such as SOS1. In some embodiments, the ion transporter is a sodium/hydrogen ion exchanger. In some embodiments, the ion transporter is a tonoplast sodium/hydrogen ion exchanger, such as NHX1. The inventors have identified that altering the expression of a tonoplast ion transporter in combination with a plasma membrane ion transporter using enhancer elements can advantageously increase salt tolerance, because sodium can be both excluded and compartmentalized from the cytosol.

Therefore, in certain embodiments, the engineered plant can comprise at least two genes of interest wherein the genes of interest comprise a gene that encodes a plasma membrane sodium/hydrogen antiporter and a gene that encodes a tonoplast sodium/hydrogen ion exchanger, wherein the a gene that encodes a plasma membrane sodium/hydrogen antiporter is operatively linked to an enhancer element and a gene that encodes a tonoplast sodium/hydrogen ion exchanger is operatively linked to an enhancer element. For example, the engineered plant may comprise genes that encode SOS1 and NHX1.

In certain embodiments, the transgenic plant can comprise at least two transgenes wherein the at least two transgenes comprise a transgene that encodes a plasma membrane sodium/hydrogen antiporter and a transgene that encodes a tonoplast sodium/hydrogen ion exchanger. For example, the transgenic plant may comprise transgenes that encode SOS1 and NHX1.

In some embodiments, the SOS1 is Oryza sativa SOS1 (OsSOS1). OsSOS1 can have the polypeptide sequence of the Uniprot accession number Q5ICN3; the Uniprot accession number Q7XBF9; SEQ ID NO: 95 and/or SEQ ID NO: 96. In some embodiments, the transgene or gene of interest comprises a polynucleotide sequence that encodes a polypeptide comprising the amino acid sequence of Q5ICN3 and/or Q7XBF9, or the transgene can comprises an amino acid sequence that has at least 75%, 80%, 85%, 90%, 95%, 96%, 97%, 98%, 99%, 99.5% or 99.9% sequence identity to Q5ICN3 and/or Q7XBF9. In some embodiments, the transgene comprises a polynucleotide sequence that encodes a polypeptide comprising the amino acid sequence of SEQ ID NO: 95, or comprising an amino acid sequence that has at least 75%, 80%, 85%, 90%, 95%, 96%, 97%, 98%, 99%, 99.5% or 99.9% sequence identity to SEQ ID NO: 95. In some embodiments, the transgene comprises a polynucleotide sequence that encodes a polypeptide comprising the amino acid sequence of SEQ ID NO: 96, or comprising an amino acid sequence that has at least 75%, 80%, 85%, 90%, 95%, 96%, 97%, 98%, 99%, 99.5% or 99.9% sequence identity to SEQ ID NO: 96.

In some embodiments, the NHX1 is Oryza sativa NHX1 (OsNHX1). OsNHX1 can have the polypeptide sequence of the Uniprot accession number Q9SXJ8; the Uniprot accession number Q6VVA7; SEQ ID NO: 97 and/or SEQ ID NO: 98. In some embodiments, the transgene or gene of interest comprises a polynucleotide sequence that encodes a polypeptide comprising the amino acid sequence of Q9SXJ8 and/or Q6VVA7, or the transgene can comprises an amino acid sequence that has at least 75%, 80%, 85%, 90%, 95%, 96%, 97%, 98%, 99%, 99.5% or 99.9% sequence identity to Q9SXJ8 and/or Q6VVA7. In some embodiments, the transgene comprises a polynucleotide sequence that encodes a polypeptide comprising the amino acid sequence of SEQ ID NO: 97, or comprising an amino acid sequence that has at least 75%, 80%, 85%, 90%, 95%, 96%, 97%, 98%, 99%, 99.5% or 99.9% sequence identity to SEQ ID NO: 97. In some embodiments, the transgene comprises a polynucleotide sequence that encodes a polypeptide comprising the amino acid sequence of SEQ ID NO: 98, or comprising an amino acid sequence that has at least 75%, 80%, 85%, 90%, 95%, 96%, 97%, 98%, 99%, 99.5% or 99.9% sequence identity to SEQ ID NO: 98.

High-affinity potassium transporters (HKTs) belong to an important class of integral membrane proteins that facilitate cation transport across the plasma membranes of plant cells. HKT1 (High-affinity potassium transporter 1) transports potassium and sodium out of the xylem of the plant and back into the cytoplasm of the cell. HKT1 has a slightly larger active site which makes it better suited to transporting sodium. HKT1 can be a transgene or a gene of interest because it is a powerful sodium transporter. Its inclusion in the engineered or transgenic plants of the invention is to lock significant salt levels in the roots and prevent salt entering the transport system of the plant, and therefore the rest of the plant. In some embodiments, the ion transporter is a high-affinity potassium transporter. In certain embodiments, the ion transporter is a plasma membrane high-affinity potassium transporter, such as HKT1.

In some embodiments, the HKT1 is Oryza sativa HKT1 (OsHKT1). OsHKT1 can have the polypeptide sequence of Uniprot accession number Q0D9S3 or SEQ ID NO: 99. In some embodiments, the transgene or gene of interest comprises a polynucleotide sequence that encodes a polypeptide comprising the amino acid sequence of Q0D9S3, or the transgene can comprises an amino acid sequence that has at least 75%, 80%, 85%, 90%, 95%, 96%, 97%, 98%, 99%, 99.5% or 99.9% sequence identity to Q0D9S3. In some embodiments, the transgene comprises a polynucleotide sequence that encodes a polypeptide comprising the amino acid sequence of SEQ ID NO: 99, or comprising an amino acid sequence that has at least 75%, 80%, 85%, 90%, 95%, 96%, 97%, 98%, 99%, 99.5% or 99.9% sequence identity to SEQ ID NO: 99.

### Protein Kinases

SOS1 function is regulated by SOS2 (salt overly sensitive 2). SOS2 is a serine/threonine protein kinase. Constitutive expression of SOS2 has been shown to constitutively activate SOS1 allowing for a continued salt transport out of the cell and increase salt tolerance [9]. The inventors have identified that altering the expression of a protein kinase using an enhancer element in combination with altering the expression of at least one ion transporter, such as a plasma membrane ion transporter using an enhancer element, can be advantageous, because the protein kinase can activate the ion transporter, which results in improved salt tolerance. In some embodiments, the protein kinase is a serine/threonine protein kinase, such as SOS2. In some embodiments, the SOS2 is Oryza sativa SOS2 (OsSOS2). OsSOS2 can have the polypeptide sequence of Uniprot accession number Q69Q47 or SEQ ID NO: 100. In some embodiments, the transgene or gene of interest comprises a polynucleotide sequence that encodes a polypeptide comprising the amino acid sequence of Q69Q47, or the transgene can comprises an amino acid sequence that has at least 75%, 80%, 85%, 90%, 95%, 96%, 97%, 98%, 99%, 99.5% or 99.9% sequence identity to Q69Q47. In some embodiments, the transgene comprises a polynucleotide sequence that encodes a polypeptide comprising the amino acid sequence of SEQ ID NO: 100, or comprising an amino acid sequence that has at least 75%, 80%, 85%, 90%, 95%, 96%, 97%, 98%, 99%, 99.5% or 99.9% sequence identity to SEQ ID NO: 100. In certain embodiments, the transgenic plant comprises at least two transgenes which encode SOS1 and SOS2.

### Hydrogen Exporting ATPases and Pyrophosphatases

The inventors have identified that altering the expression of sodium/hydrogen antiporters and hydrogen exporting ATPases and pyrophosphatases in engineered or transgenic plants is advantageous, because it ensures that a pH gradient exists across the plasma membrane and tonoplast. Sodium/hydrogen transporters use the pH gradient generated by hydrogen exporting ATPases and pyrophosphatases to transport sodium out of the cytosol. This pH gradient therefore helps to power sodium transport out of the cytoplasm and thereby improve the salt tolerance of the engineered or transgenic plant. For example, the AHA family are plasma membrane bound hydrogen exporting ATPases (H+−ATPases), which includes AHA1, AHA2 and AHA3. Their primary function is to transport hydrogen ions across the plasma membrane and out of the cell. This generates an electrochemical proton gradient and eventually a proton motive force. This proton motive force acts like a battery—hydrogen ions are forcefully pushed out of the cell, creating the electrochemical gradient that provides the energy to readily exchange hydrogen ions outside of the cell for sodium ions inside the cell.

Hydrogen exporting ATPases and pyrophosphatases are major components of the vacuolar membrane of plant cells [1]. VHA-A (Vacuolar-type H+−ATPase subunit A1) is a vacuolar ATPase which breaks down ATP to transport hydrogen ions into the vacuole. This creates a strong proton motive force, similar to the SOS1 and AHA3 cycle, across the tonoplast by building up a large amount of hydrogen ions inside the vacuole that can be exchanged for sodium ions. This is believed to advantageously increase the salt tolerance of the plant.

In some embodiments, the protein that can controls the intracellular ion concentration is a hydrogen exporting ATPase or a hydrogen exporting pyrophosphatase. In some embodiments, the protein that can controls the intracellular ion concentration is a hydrogen exporting ATPase. In some embodiments, the hydrogen exporting ATPase is a tonoplast hydrogen exporting ATPase or a plasma membrane hydrogen exporting ATPase.

In certain embodiments, the plasma membrane hydrogen exporting ATPase is AHA3. In some embodiments, the AHA3 is Oryza sativa AHA3 (OsAHA3). OsAHA3 can have the polypeptide sequence of Uniprot accession number Q8L6I3 or SEQ ID NO: 101. In some embodiments, the transgene or gene of interest comprises a polynucleotide sequence that encodes a polypeptide comprising the amino acid sequence of Q8L6I3, or the transgene can comprise an amino acid sequence that has at least 75%, 80%, 85%, 90%, 95%, 96%, 97%, 98%, 99%, 99.5% or 99.9% sequence identity to Q8L6I3. In some embodiments, the transgene comprises a polynucleotide sequence that encodes a polypeptide comprising the amino acid sequence of SEQ ID NO: 101, or comprising an amino acid sequence that has at least 75%, 80%, 85%, 90%, 95%, 96%, 97%, 98%, 99%, 99.5% or 99.9% sequence identity to SEQ ID NO: 101.

In certain embodiments, the tonoplast hydrogen exporting ATPase is VHA-A. In some embodiments, the VHA-A is Oryza sativa VHA-A (OsVHA-A). OsVHA-A can have the polypeptide sequence of Uniprot accession number Q651T8 or SEQ ID NO: 102. In some embodiments, the transgene or gene of interest comprises a polynucleotide sequence that encodes a polypeptide comprising the sequence of Q651T8, or the transgene can comprise an amino acid sequence that has at least 75%, 80%, 85%, 90%, 95%, 96%, 97%, 98%, 99%, 99.5% or 99.9% sequence identity to Q651T8. In some embodiments, the transgene comprises a polynucleotide sequence that encodes a polypeptide comprising the amino acid sequence of SEQ ID NO: 102, or comprising an amino acid sequence that has at least 75%, 80%, 85%, 90%, 95%, 96%, 97%, 98%, 99%, 99.5% or 99.9% sequence identity to SEQ ID NO: 102.

In other embodiments the tonoplast hydrogen exporting ATPase is VHA-B. In some embodiments, the VHA-B is Oryza sativa VHA-B (OsVHA-B). OsVHA-B can have the polypeptide sequence of Uniprot accession number Q7FV25 or SEQ ID NO: 103. In some embodiments, the transgene or gene of interest comprises a polynucleotide sequence that encodes a polypeptide comprising the sequence of Q7FV25, or the transgene an comprise an amino acid sequence that has at least 75%, 80%, 85%, 90%, 95%, 96%, 97%, 98%, 99%, 99.5% or 99.9% sequence identity to Q7FV25. In some embodiments, the transgene comprises a polynucleotide sequence that encodes a polypeptide comprising the amino acid sequence of SEQ ID NO: 103, or comprising an amino acid sequence that has at least 75%, 80%, 85%, 90%, 95%, 96%, 97%, 98%, 99%, 99.5% or 99.9% sequence identity to SEQ ID NO: 103.

In certain embodiments, the engineered plant comprises at least two genes of interest, wherein the at least two genes of interest, comprise a gene that encodes a hydrogen exporting ATPase and a gene that encodes an ion transporter (such as a sodium or potassium transporter). In some embodiments, the at least two genes of interest comprise a gene that encodes a plasma membrane hydrogen exporting ATPase and a gene that encodes a plasma membrane ion transporter (such as a sodium or potassium transporter). For example, the genes of interest may encode SOS1 and AHA3. In some embodiments, the genes encode a tonoplast hydrogen exporting ATPase and a tonoplast ion transporter (such as a sodium or potassium transporter). For example, the genes of interest may encode NHX1 and VHA-A. In further embodiments, the engineered plant comprises at least four genes of interest, wherein the at least four genes of interest, comprise a gene that encodes a plasma membrane hydrogen exporting ATPase, a gene that encodes a plasma membrane ion transporter, a gene that encodes a tonoplast hydrogen exporting ATPase and a gene that encodes a tonoplast ion transporter. For example, the genes of interest may encode SOS1, AHA3, NHX1 and VHA-A. In each of the embodiments in this paragraph, the gene of interest is operatively linked to an enhancer element as disclosed herein.

In certain embodiments, the transgenic plant comprises at least two transgenes, wherein the at least two transgenes comprise a transgene that encodes a hydrogen exporting ATPase and a transgene that encodes an ion transporter (such as a sodium or potassium transporter). In some embodiments, the at least two transgenes comprise a transgene that encodes a plasma membrane hydrogen exporting ATPase and a transgene that encodes a plasma membrane ion transporter (such as a sodium or potassium transporter). For example, the transgenes may encode SOS1 and AHA3.

In some embodiments, the transgenes encode a tonoplast hydrogen exporting ATPase and a tonoplast ion transporter (such as a sodium or potassium transporter). For example, the transgenes may encode NHX1 and VHA-A. In further embodiments, the transgenic plant comprises at least four transgenes, wherein the at least four transgenes comprise a transgene that encodes a plasma membrane hydrogen exporting ATPase, a transgene that encodes a plasma membrane ion transporter, a transgene that encodes a tonoplast hydrogen exporting ATPase and a transgene that encodes a tonoplast ion transporter. For example, the transgenes may encode SOS1, AHA3, NHX1 and VHA-A.

### Exemplary Engineered and Transgenic Plants of the Invention

The invention provides engineered plants comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element. The invention also provides transgenic plants comprising at least two transgenes wherein the at least two transgenes comprise a transgene that encodes a protein that controls the intracellular ion concentration and a transgene that encodes an antioxidant.

In some embodiments, the protein that controls the intracellular ion concentration can be an ion transporter (e.g. a sodium or potassium transporter), a hydrogen exporting ATPase, a hydrogen exporting pyrophosphatase or protein kinase. In some embodiments, the protein that controls the intracellular ion concentration is a plasma membrane protein, a cytosolic protein or a tonoplast protein.

In some embodiments, the engineered plant comprises two or more, three or more, four or more, five or more, six or more or seven or more, eight or more, nine or more, or ten or more genes of interest including genes that encode proteins that control the intracellular ion concentration. In particular embodiments, the engineered plant comprises eight or more genes of interest including six or more genes of interest that encode proteins that control the intracellular ion concentration and two or more genes of interest that encode antioxidants. In each of the embodiments in this paragraph and this entire section the gene of interest is operatively linked to an enhancer element as disclosed herein.

In some embodiments, the transgenic plant comprises two or more, three or more, four or more, five or more, six or more or seven or more, eight or more, nine or more, or ten or more transgenes including transgenes that encode proteins that control the intracellular ion concentration. In particular embodiments, the transgenic plant comprises eight or more transgenes including six or more transgenes that encode proteins that control the intracellular ion concentration and two or more transgenes that encode antioxidants.

In some embodiments, the engineered plant comprises two or more genes of interest that encode proteins that control the intracellular ion concentration, wherein the genes of interest are operatively linked to an enhancer element as disclosed herein. In some embodiments, the transgenic plant comprises two or more transgenes that encode proteins that control the intracellular ion concentration. In some embodiments, the transgenes or genes of interest encode (a) an ion transporter and a hydrogen exporting ATPase; (b) an ion transporter and a hydrogen exporting pyrophosphatase, (c) an ion transporter and a protein kinase, (d) a hydrogen exporting ATPase and a protein kinase, (e) a hydrogen exporting pyrophosphatase and a protein kinase, (f) two ion transporters. In particular embodiments, the transgenes or genes of interest encode an ion transporter and a hydrogen exporting ATPase or an ion transporter and a protein kinase. For example, the transgenes or genes of interest encode SOS1 and AHA3; SOS1 and SOS2; or NHX1 and VHA-A. In certain embodiments, the transgenes or genes of interest encode OsSOS1 and OsAHA3; OsSOS1 and OsSOS2; or OsNHX1 and OsVHA-A.

In some embodiments, the engineered plant comprises two or more genes of interest including genes that encode two plasma membrane proteins that control the intracellular ion concentration. In other embodiments, the engineered plant comprises two or more genes of interest including genes that encode two tonoplast proteins that control the intracellular ion concentration. In each of the embodiments in this paragraph, the gene of interest is operatively linked to an enhancer element as disclosed herein.

In some embodiments, the transgenic plant comprises two or more transgenes including transgenes that encode two plasma membrane proteins that control the intracellular ion concentration. In other embodiments, the transgenic plant comprises two or more transgenes including transgenes that encode two tonoplast proteins that control the intracellular ion concentration.

The inventors have identified that using both plasma membrane and tonoplast mechanisms to reduce the cytosolic sodium concentration can advantageously increase a plant's tolerance to salt. Therefore, the invention provides engineered and transgenic plants comprising at least two transgenes or genes of interest wherein a transgene or a gene of interest encodes a plasma membrane protein that controls the intracellular ion concentration and a transgene or a gene of interest that encodes a tonoplast protein that controls the intracellular ion concentration. In each of the embodiments in this paragraph the gene of interest is operatively linked to an enhancer element as disclosed herein.

For example, the engineered plant comprises two or more genes of interest including genes that encode a plasma membrane ion transporter and a tonoplast ion transport. In some embodiments, the engineered plant comprises two or more genes of interest including genes that encode a plasma membrane ion transporter and a tonoplast hydrogen exporting ATPase. In some embodiments, the engineered plant comprises two or more genes of interest including genes that encode a plasma membrane hydrogen exporting ATPase and a tonoplast ion transporter. In some embodiments, the engineered plant comprises two or more genes of interest including genes that encode a plasma membrane hydrogen exporting ATPase and a tonoplast hydrogen exporting ATPase. For example, the genes of interest encode SOS1 and NHX1; AHA3 and VHA-A; SOS1 and AHA3 or NHX1 and AHA3. In certain embodiments, the genes encode OsSOS1 and OsNHX1; OsAHA3 and OsVHA-A; OsSOS1 and OsAHA3 or OsNHX1 and OsAHA3. In each of the embodiments in this paragraph, the gene of interest is operatively linked to an enhancer element as disclosed herein.

For example, the transgenic plant comprises two or more transgenes including transgenes that encode a plasma membrane ion transporter and a tonoplast ion transport. In some embodiments, the transgenic plant comprises two or more transgenes including transgenes that encode a plasma membrane ion transporter and a tonoplast hydrogen exporting ATPase. In some embodiments, the transgenic plant comprises two or more transgenes including transgenes that encode a plasma membrane hydrogen exporting ATPase and a tonoplast ion transporter. In some embodiments, the transgenic plant comprises two or more transgenes including transgenes that encode a plasma membrane hydrogen exporting ATPase and a tonoplast hydrogen exporting ATPase. For example, the transgenes encode SOS1 and NHX1; AHA3 and VHA-A; SOS1 and AHA3 or NHX1 and AHA3. In certain embodiments, the transgenes encode OsSOS1 and OsNHX1; OsAHA3 and OsVHA-A; OsSOS1 and OsAHA3 or OsNHX1 and OsAHA3.

In some embodiments, the engineered plant can comprises at least three genes of interest that increase the salt tolerance of the engineered plant compared to a plant of a same species without said genome modifications, wherein each gene of interest is operatively linked to an enhancer element as disclosed herein. In some embodiments, the transgenic plant can comprises at least three transgenes that increase the salt tolerance of the transgenic plant compared to a plant of a same species without said genome modifications.

The engineered plant or transgenic plant can comprise at least three genes of interest or transgenes wherein the at least three genes of interest or transgenes comprise genes or transgenes that encode proteins that can be independently selected from the group consisting of a plasma membrane ion transporter, a plasma membrane hydrogen exporting ATPase and at least one antioxidant. In some embodiments, the transgenes encode a tonoplast ion transporter, a tonoplast hydrogen exporting ATPase and an antioxidant. In some embodiments, the genes or transgenes encode a plasma membrane ion transporter, a tonoplast ion transporter and a tonoplast hydrogen exporting ATPase. In some embodiments, the genes or transgenes encode a plasma membrane hydrogen exporting ATPase, a tonoplast ion transporter and a tonoplast ATPase. In some embodiments, the genes or transgenes encode a plasma membrane ion transporter, a plasma membrane hydrogen exporting ATPase and a tonoplast ion transporter. In some embodiments, the genes or transgenes encode a plasma membrane ion transporter, a plasma membrane hydrogen exporting ATPase and a tonoplast ion transporter. In some embodiments, the genes or transgenes encode a plasma membrane sodium transporter, a tonoplast sodium transporter and a potassium transporter. In some embodiments, the genes or transgenes encode a plasma membrane ion transporter, a plasma membrane hydrogen exporting ATPase and a protein kinase. In some embodiments, the genes or transgenes encode a tonoplast ion transporter, a tonoplast hydrogen exporting ATPase and a protein kinase. In each of the embodiments in this paragraph the gene of interest is operatively linked to an enhancer element as disclosed herein.

The engineered plant can comprise at least three genes of interest. In certain embodiments, the engineered plant comprises at least three genes of interest wherein the at least three genes of interest comprise a gene that encodes a plasma membrane protein that controls the intracellular ion concentration; a gene that encodes a tonoplast protein that controls the intracellular ion concentration and a gene that encodes an antioxidant. For example, the engineered plant can comprise at least three genes of interest wherein the at least three genes of interest comprise a gene that encodes a plasma membrane ion transporter; a gene that encodes a tonoplast ion transporter and a gene that encodes an antioxidant. In each of the embodiments in this paragraph, the gene of interest is operatively linked to an enhancer element as disclosed herein.

The transgenic plant can comprise at least three transgenes. In certain embodiments, the transgenic plant comprises at least three transgenes wherein the at least three transgenes comprise a transgene that encodes a plasma membrane protein that controls the intracellular ion concentration; a transgene that encodes a tonoplast protein that controls the intracellular ion concentration and a transgene that encodes an antioxidant. For example, the transgenic plant can comprise at least three transgenes wherein the at least three transgenes comprise a transgene that encodes a plasma membrane ion transporter; a transgene that encodes a tonoplast ion transporter and a transgene that encodes an antioxidant.

The engineered or transgenic plant can comprise at least four genes of interest or transgenes wherein the at least four genes of interest or transgenes comprise genes or transgenes that encode proteins that can be independently selected from the group consisting of an ion transporter (e.g. a sodium or potassium transporter), a hydrogen exporting ATPase, a hydrogen exporting pyrophosphatase, a protein kinase and at least one antioxidant.

In some embodiments, the genes of interest or transgenes encode a plasma membrane ion transporter, a plasma membrane ATPase, a protein kinase and an antioxidant. In some embodiments, the genes of interest or transgenes encode a tonoplast ion transporter, a tonoplast ATPase, a protein kinase and an antioxidant. In some embodiments, the genes of interest or transgenes encode a plasma membrane ion transporter, a tonoplast ion transporter, a protein kinase and an antioxidant. In some embodiments, the genes of interest or transgenes encode a plasma membrane hydrogen exporting ATPase, a tonoplast hydrogen exporting ATPase, a protein kinase and an antioxidant. In some embodiments, the genes of interest or transgenes encode a plasma membrane hydrogen exporting ATPase, a tonoplast ion transporter, a protein kinase and an antioxidant. In some embodiments, the genes of interest or transgenes encode a plasma membrane ion transporter, a tonoplast hydrogen exporting ATPase, a protein kinase and an antioxidant. In each of the embodiments in this paragraph the gene of interest is operatively linked to an enhancer element as disclosed herein.

In some embodiments, the genes of interest or transgenes encode a plasma membrane ion transporter, a plasma membrane ATPase, a potassium transporter and an antioxidant. In some embodiments, the genes of interest or transgenes encode a tonoplast ion transporter, a tonoplast hydrogen exporting ATPase, a potassium transporter and an antioxidant. In some embodiments, the genes of interest or transgenes encode a plasma membrane hydrogen exporting ATPase, a tonoplast hydrogen exporting ATPase, a potassium transporter and an antioxidant. In some embodiments, the genes of interest or transgenes encode a plasma membrane hydrogen exporting ATPase, a tonoplast ion transporter, a potassium transporter and an antioxidant. In some embodiments, the genes of interest or transgenes encode a plasma membrane ion transporter, a tonoplast hydrogen exporting ATPase, a potassium transporter and an antioxidant. In each of the embodiments in this paragraph the gene of interest is operatively linked to an enhancer element as disclosed herein.

In certain embodiments, the engineered plant comprises at least four genes of interest wherein the at least four genes of interest comprise genes that encode a plasma membrane ion transporter, a tonoplast ion transporter, a potassium transporter and an antioxidant. In certain embodiments, the genes of interest plant comprises at least four genes of interest wherein the at least four genes of interest comprise genes that encode SOS1 (a plasma membrane ion transporter), NHX1 (a tonoplast ion transporter), HKT1 (a potassium transporter) and SODA1 (an antioxidant). In some embodiments, the invention provides a genes of interest rice plant comprising at least four genes of interest wherein the at least four genes of interest encode OsSOS1, OsNHX1, OsHKT1 and OsSODA1. In each of the embodiments in this paragraph, the gene of interest is operatively linked to an enhancer element as disclosed herein.

In certain embodiments, the engineered plant comprises at least four genes of interest wherein the at least four genes of interest comprise genes that encode a plasma membrane ion transporter, a tonoplast ion transporter, a potassium transporter and a plasma membrane hydrogen exporting ATPase. In certain embodiments, the genes of interest plant comprises at least four genes of interest wherein the at least four genes of interest comprise genes that encode SOS1 (a plasma membrane ion transporter), NHX1 (a tonoplast ion transporter), HKT1 (a potassium transporter) and AHA3 (a plasma membrane hydrogen exporting ATPase). In some embodiments, the invention provides a genes of interest rice plant comprising at least four genes of interest wherein the at least four genes of interest encode OsSOS1, OsNHX1, OsHKT1 and OsAHA3. In each of the embodiments in this paragraph, the gene of interest is operatively linked to an enhancer element as disclosed herein.

In certain embodiments, the engineered plant comprises at least four genes of interest wherein the at least four genes of interest comprise genes that encode a plasma membrane ion transporter, a tonoplast ion transporter, a serine kinase and a tonoplast hydrogen exporting ATPase. In certain embodiments, the genes of interest plant comprises at least four genes of interest wherein the at least four genes of interest comprise genes that encode SOS1 (a plasma membrane ion transporter), NHX1 (a tonoplast ion transporter), SOS2 (a serine kinase) and VHA-A (a tonoplast hydrogen exporting ATPase). In some embodiments, the invention provides a genes of interest rice plant comprising at least four genes of interest wherein the at least four genes of interest encode OsSOS1, OsNHX1, OsSOS2 and OsVHA-A. In each of the embodiments in this paragraph, the gene of interest is operatively linked to an enhancer element as disclosed herein.

In certain embodiments, the transgenic plant comprises at least four transgenes wherein the at least four transgenes comprise transgenes that encode a plasma membrane ion transporter, a tonoplast ion transporter, a potassium transporter and an antioxidant. In certain embodiments, the transgenic plant comprises at least four transgenes wherein the at least four transgenes comprise transgenes that encode SOS1 (a plasma membrane ion transporter), NHX1 (a tonoplast ion transporter), HKT1 (a potassium transporter) and SODA1 (an antioxidant). In some embodiments, the invention provides a transgenic rice plant comprising at least four transgenes wherein the at least four transgenes comprise transgenes that encode OsSOS1, OsNHX1, OsHKT1 and OsSODA1.

In some embodiments, the genes of interest or transgenes encode a plasma membrane ion transporter, a plasma membrane hydrogen exporting ATPase, a tonoplast hydrogen exporting ATPase, and an antioxidant. In some embodiments, the engineered or transgenic plant comprises genes of interest or transgenes, wherein the genes of interest or transgenes encode a plasma membrane ion transporter, a plasma membrane hydrogen exporting ATPase, a tonoplast ion transporter, and an antioxidant. In some embodiments, the engineered or transgenic plant comprises genes of interest or transgenes, wherein the genes of interest or transgenes encode a plasma membrane ion transporter, a tonoplast ion transporter, a tonoplast hydrogen exporting ATPase, and an antioxidant. In some embodiments, the engineered or transgenic plant comprises genes of interest or transgenes, wherein the genes of interest or transgenes encode a plasma membrane hydrogen exporting ATPase, a tonoplast ion transporter, a tonoplast hydrogen exporting ATPase, and an antioxidant.

In some embodiments, the engineered or transgenic plant comprises at least four genes of interest or transgenes wherein the at least four genes of interest or transgenes comprise genes or transgenes that encode a plasma membrane ion transporter, a plasma membrane hydrogen exporting ATPase, a tonoplast ion transporter and a tonoplast hydrogen exporting ATPase.

In some embodiments, the engineered or transgenic plant can comprise at least five genes of interest or transgenes wherein the at least five genes of interest or transgenes comprise genes or transgenes that encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins.

In some embodiments, the genes or transgenes encode a plasma membrane ion transporter; a plasma membrane hydrogen exporting ATPase, a tonoplast hydrogen exporting ATPase, a tonoplast ion transporter and at least one antioxidant. For example, the genes or transgenes may encode SOS1, AHA-3, NHX1, VHA-A and SODA1. In some embodiments, the genes or transgenes encode a plasma membrane ion transporter; a plasma membrane hydrogen exporting ATPase, a protein kinase, a tonoplast ion transporter and an antioxidant. For example, the genes or transgenes may encode SOS1, AHA-3, NHX1, SOS2 and SODA1. In some embodiments, the genes or transgenes encode a plasma membrane ion transporter; a potassium transporter, a protein kinase, a tonoplast ion transporter and an antioxidant. For example, the genes or transgenes may encode SOS1, HKT1, NHX1, SOS2 and SODA1.

In some embodiments, the engineered or transgenic plant can comprise at least six genes of interest or transgenes wherein the at least six genes of interest or transgenes comprise genes or transgenes that encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In some embodiments, the transgenes encode a plasma membrane ion transporter; a plasma membrane hydrogen exporting ATPase, a protein kinase, a tonoplast hydrogen exporting ATPase, a tonoplast ion transporter and an antioxidant. For example, the genes of interest or transgenes encode SOS1, AHA-3, NHX1, VHA-A, SOS2 and SODA1. In some embodiments, the genes of interest or transgenes encode a plasma membrane ion transporter; a plasma membrane hydrogen exporting ATPase, potassium transporter, a tonoplast ion transporter, a protein kinase and an antioxidant. For example, the at least six genes of interest or transgenes encode SOS1, AHA-3, HKT1, NHX1, SOS2 and SODA1.

In some embodiments, the genes of interest or transgenes can encode an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In some embodiments, the genes of interest or transgenes encode a plasma membrane ion transporter; a plasma membrane hydrogen exporting ATPase, a protein kinase, a tonoplast hydrogen exporting ATPase, a tonoplast ion transporter and an antioxidant.

In some embodiments, the genes of interest or transgenes encode a plasma membrane ion transporter; a plasma membrane hydrogen exporting ATPase, a tonoplast hydrogen exporting ATPase, a tonoplast ion transporter, a protein kinase and an antioxidant. For example, the genes of interest or transgenes encode SOS1, AHA-3, NHX1, VHA-A, SOS2 and SODA1. In some embodiments, the genes of interest or transgenes encode a plasma membrane ion transporter; a plasma membrane hydrogen exporting ATPase, potassium transporter, a tonoplast ion transporter, a protein kinase and an antioxidant. For example, the six genes of interest or transgenes may encode SOS1, AHA-3, HKT1, NHX1, SOS2 and SODA1.

In some embodiments, the engineered or transgenic plant can comprise at least seven genes of interest or transgenes wherein the at least seven genes of interest or transgenes comprise genes or transgenes that encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In some embodiments, the transgenes encode a plasma membrane ion transporter; a plasma membrane hydrogen exporting ATPase, a tonoplast hydrogen exporting ATPase, a tonoplast ion transporter, a potassium transporter, a protein kinase and an antioxidant. For example, the seven genes of interest or transgenes may encode (a) SOS1, AHA-3, NHX1, VHA-A, HKT1, SOS2 and SODA1; (b) SOS1, AHA-3, NHX1, VHA-A, HKT1, SOS2 and SODCC1 or (c) SOS1, AHA-3, NHX1, VHA-A, HKT1, SOS2 and SOD2.

In some embodiments, the engineered plant can comprise at least eight genes of interest wherein the at least eight genes of interest encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In certain embodiments, the genes of interest encode a plasma membrane ion transporter; a plasma membrane hydrogen exporting ATPase, a tonoplast hydrogen exporting ATPase, a tonoplast ion transporter, a potassium transporter, a protein kinase and two antioxidants. In certain embodiments, the genes of interest encode SOS1, AHA-3, NHX1, VHA-A, HKT1, SOS2, SODA1 and SODCC1. In further specific embodiments, the genes of interest encode OsSOS1, OsAHA-3, OsNHX1, OsVHA-A, OsHKT1, OsSOS2, OsSODA1 and OsSODCC1. In alternative specific embodiments, the genes of interest encode SOS1, AHA-3, NHX1, VHA-A, HKT1, SOS2, SODA1 and SOD2. In further specific embodiments, the genes of interest encode OsSOS1, OsAHA-3, OsNHX1, OsVHA-A, OsHKT1, OsSOS2, OsSODA1 and OsSOD2. In each of the embodiments in this paragraph, the gene of interest is operatively linked to an enhancer element as disclosed herein.

In some embodiments, the transgenic plant can comprise at least eight transgenes wherein the at least eight transgenes comprise transgenes that encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In certain embodiments, the transgenes encode a plasma membrane ion transporter; a plasma membrane hydrogen exporting ATPase, a tonoplast hydrogen exporting ATPase, a tonoplast ion transporter, a potassium transporter, a protein kinase and two antioxidants. In certain embodiments, the transgenes encode SOS1, AHA-3, NHX1, VHA-A, HKT1, SOS2, SODA1 and SODCC1. In further specific embodiments, the transgenes encode OsSOS1, OsAHA-3, OsNHX1, OsVHA-A, OsHKT1, OsSOS2, OsSODA1 and OsSODCC1. In alternative specific embodiments, the transgenes encode SOS1, AHA-3, NHX1, VHA-A, HKT1, SOS2, SODA1 and SOD2. In further specific embodiments, OsSOS1, OsAHA-3, OsNHX1, OsVHA-A, OsHKT1, OsSOS2, OsSODA1 and OsSOD2.

In some embodiments, the engineered or transgenic plant comprises at least nine genes of interest or transgenes wherein the at least nine genes of interest or transgenes comprise genes or transgenes that can encode proteins that can be independently selected from the group consisting of an ion transporter (e.g. a sodium or potassium transporter), a hydrogen exporting ATPase, a hydrogen exporting pyrophosphatase, a protein kinase and at least one antioxidant. These encoded proteins can be plasma membrane proteins, cytosolic proteins or tonoplast proteins. In certain embodiments, the genes of interest or transgenes encode a plasma membrane ion transporter; a plasma membrane hydrogen exporting ATPase, a tonoplast hydrogen exporting ATPase, a tonoplast ion transporter, a potassium transporter, a protein kinase and three antioxidants. In certain embodiments, the genes of interest or transgenes encode SOS1, AHA-3, NHX1, VHA-A, HKT1, SOS2, SODA1, SODCC1 and SOD2.

### Additional Genes of Interest and Transgenes

In some embodiments, the engineered or transgenic plant comprises further genes of interest or transgenes that encode a cytochrome P450 (P450), an oxygen-evolving complex, a sucrose phosphate synthase and/or a pyrroline synthase. In each of the embodiments in the sections below the gene of interest is operatively linked to an enhancer element as disclosed herein.

### Cytochrome P450

Cytochrome P450 is an enzyme which catalyzes the rate-limiting step for wax production in leaves. By increasing the expression of this enzyme the level of wax in the leaves can be increased. This increase in wax prevents water loss between the mesophyll layer of plant leaves and the air. There is an osmotic disparity between the ocean air with high salt levels and the water potential inside the mesophyll air pockets. Therefore, the inclusion of a transgene that encodes a cytochrome P450 or targeting a gene of interest that is a cytochrome P450 may advantageously enable engineered or transgenic plants to grow in high salinity areas such as the ocean. Therefore in some embodiments, the engineered or transgenic plant further comprises a gene of interest or transgene that encodes a cytochrome P450 (P450). In some embodiments, the P450 is Oryza sativa P450 (OsP450). In some embodiments, the P450 is Arabidopsis thaliana P450, which has the UniProt Accession number Q9FVS9 or SEQ ID NO: 112. In some embodiments, the transgene or gene of interest comprises a polynucleotide sequence that encodes a polypeptide comprising the amino acid sequence of SEQ ID NO: 112, or the transgene comprises an amino acid sequence that has at least 75%, 80%, 85%, 90%, 95%, 96%, 97%, 98%, 99%, 99.5% or 99.9% sequence identity to SEQ ID NO: 112.

In some embodiments, the engineered or transgenic plant comprises gene of interest or transgenes that encode SOS1, SOS2, AHA3, VHA-A, NHX1, HKT1, SODA1, SODCC1 and P450. In some embodiments, the engineered or transgenic plant comprises gene of interest or transgenes that encode SOS1, SOS2, AHA3, VHA-A, NHX1, HKT1, SODA1, SOD2 and P450.

### Oxygen-Evolving Complex

The process of pumping hydrogen ions to many locations in the cell can be energy intensive. Constitutively producing three subunits from the Photosystem 2 hydrolase: PsbO, PsbP and PsbQ may help to improve the salt tolerance of the transgenic plant by increasing the amount of energy that is available to the plant cell. When combined to form a complete enzymatic unit, these subunits break down water molecules to produce hydrogen ions, electrons and oxygen. PsbU and PsbV may also be used in this system to only allow water to be reduced by the complete enzyme.

In some embodiments, the engineered or transgenic plant further comprises a gene of interest or transgene that encodes an oxygen-evolving complex. In some embodiments, the oxygen-evolving complex is formed of the proteins PsbO, PsbP and PsbQ. In some embodiments, the oxygen-evolving complex is formed of the proteins PsbO, PsbP, PsbQ, PsbU and PsbV. In some embodiments, the engineered or transgenic plant comprises genes of interest or transgenes that encode SOS1, SOS2, AHA3, VHA-A, NHX1, HKT1, SODA1, SODCC1, PsbO, PsbP and PsbQ. In some embodiments, the engineered or transgenic plant comprises genes of interest or transgenes that encode SOS1, SOS2, AHA3, VHA-A, NHX1, HKT1, SODA1, SOD2, PsbO, PsbP, PsbQ, PsbU and PsbV.

In some embodiments, the PsbO is Oryza sativa PsbO (OsPsbO). OsPsbO can have the polypeptide sequence of Uniprot accession number A5JV93 or SEQ ID NO: 104. In some embodiments, the gene of interest or transgene comprises a polynucleotide sequence that encodes a polypeptide comprising the amino acid sequence of A5JV93 and/or SEQ ID NO: 104, or the transgene can comprise an amino acid sequence that has at least 75%, 80%, 85%, 90%, 95%, 96%, 97%, 98%, 99%, 99.5% or 99.9% sequence identity to A5JV93 and/or SEQ ID NO: 104.

In some embodiments, the PsbP is Oryza sativa PsbP (OsPsbP). OsPsbP can have the polypeptide sequence of Uniprot accession number XP_002876377.1; QOKIW5; SEQ ID NO: 105. In some embodiments, the gene of interest or transgene comprises a polynucleotide sequence that encodes a polypeptide comprising the amino acid sequence of XP_002876377.1; QOKIW5; SEQ ID NO: 105, or the transgene can comprise an amino acid sequence that has at least 75%, 80%, 85%, 90%, 95%, 96%, 97%, 98%, 99%, 99.5% or 99.9% sequence identity to XP_002876377.1; QOKIW5; SEQ ID NO: 105.

In some embodiments, the PsbQ is Oryza sativa PsbQ (OsPsbQ). OsPsbQ can have the polypeptide sequence of Uniprot accession number P83646 or SEQ ID NO: 106. In some embodiments, the gene of interest or transgene comprises a polynucleotide sequence that encodes a polypeptide comprising the amino acid sequence of P83646 and/or SEQ ID NO: 106 or the transgene can comprise an amino acid sequence that has at least 75%, 80%, 85%, 90%, 95%, 96%, 97%, 98%, 99%, 99.5% or 99.9% sequence identity to P83646 and/or SEQ ID NO: 106. In some embodiments, the PsbU is Oryza sativa PsbU (OsPsbU).

### Sucrose Phosphate Synthase

The accumulation of osmoprotective compounds, such as sucrose and proline, is important for plants to adapt to high salinity. Sucrose is derived from hexose phosphates through the combined activities of UDP-glucose pyrophosphorylase, sucrose phosphate synthase and sucrose phosphatase. Sucrose phosphate synthase appears to be a major control point in sucrose formation [10]. It may therefore be advantageous to express sucrose phosphate synthases in the engineered or transgenic plants to protect against high levels of salt.

In some embodiments, the engineered or transgenic plant further comprises a gene of interest or transgene that encodes a sucrose phosphate synthase. In some embodiments, the sucrose phosphate synthase is sucrose phosphate synthase 1, sucrose phosphate synthase 2 or sucrose phosphate synthase 3. In some embodiments, the engineered or transgenic plant comprises genes of interest or transgenes that encode SOS1, SOS2, AHA3, VHA-A, NHX1, HKT1, SODA1, SODCC1 and sucrose phosphate synthase 1. In some embodiments, the engineered or transgenic plant comprises genes of interest or transgenes that encode SOS1, SOS2, AHA3, VHA-A, NHX1, HKT1, SODA1, SODCC1 and sucrose phosphate synthase 2. In some embodiments, the engineered or transgenic plant comprises genes of interest or transgenes that encode SOS1, SOS2, AHA3, VHA-A, NHX1, HKT1, SODA1, SODCC1 and sucrose phosphate synthase 3.

In some embodiments, the sucrose phosphate synthase 1 is Oryza sativa sucrose phosphate synthase 1, which has the UniProt Accession number Q0JGK4 or SEQ ID NO: 113. In some embodiments, the transgene or gene of interest comprises a polynucleotide sequence that encodes a polypeptide comprising the amino acid sequence of Q0JGK4 and/or SEQ ID NO: 113, or the transgene comprises an amino acid sequence that has at least 75%, 80%, 85%, 90%, 95%, 96%, 97%, 98%, 99%, 99.5% or 99.9% sequence identity to SEQ ID NO: 113.

In some embodiments, the sucrose phosphate synthase 1 is Oryza sativa sucrose phosphate synthase 1. In some embodiments, the sucrose phosphate synthase 2 is Oryza sativa sucrose phosphate synthase 2. In some embodiments, the sucrose phosphate synthase 3 is Oryza sativa sucrose phosphate synthase 3.

### Pyrroline Synthase

Several plants accumulate high proline levels in response to high salinity. Proline is thought to act as an osmoprotectant, by being a sink for energy to regulate redox potentials, or as a solute that protects macromolecules from denaturation. Under stress conditions proline accumulation is primarily due to de novo synthesis. In plants, proline can be synthesised from either glutamate or ornithine, but under salt stress the glutamate pathway is the primary route [11]. Delta-1-pyrroline-5-carboxylate synthase catalyzes the rate-limiting step in the biosynthesis of proline. There are two forms of this enzyme, delta-1-pyrroline-5-carboxylate synthase 1, or delta-1-pyrroline-5-carboxylate synthase 2, which are present in different cells and/or different subcellular locations. It may therefore be advantageous to express delta-1-pyrroline-5-carboxylate synthases in the engineered or transgenic plants to protect against high level of salt.

In some embodiments, the engineered or transgenic plant further comprises a gene of interest or transgene that encodes a pyrroline carboxylate synthase. In some embodiments, the pyrroline carboxylate synthase is delta-1-pyrroline-5-carboxylate synthase 1 or delta-1-pyrroline-5-carboxylate synthase 2. In some embodiments, the engineered or transgenic plant comprises genes of interest or transgenes that encode SOS1, SOS2, AHA3, VHA-A, NHX1, HKT1, SODA1, SODCC1 and delta-1-pyrroline-5-carboxylate synthase 1. In some embodiments, the engineered or transgenic plant comprises genes of interest or transgenes that encode SOS1, SOS2, AHA3, VHA-A, NHX1, HKT1, SODA1, SODCC1 and delta-1-pyrroline-5-carboxylate synthase 2.

In some embodiments, the delta-1-pyrroline-5-carboxylate synthase 1 is Oryza sativa delta-1-pyrroline-5-carboxylate synthase 1. In some embodiments, the delta-1-pyrroline-5-carboxylate synthase 2 is Oryza sativa delta-1-pyrroline-5-carboxylate synthase 2. In some embodiments, the Oryza sativa delta-1-pyrroline-5-carboxylate synthase 1 has the UniProt Accession number 004226 or SEQ ID NO: 114. In some embodiments, the transgene or gene of interest comprises a polynucleotide sequence that encodes a polypeptide comprising the amino acid sequence of 004226 and/or SEQ ID NO: 114, or the transgene comprises an amino acid sequence that has at least 75%, 80%, 85%, 90%, 95%, 96%, 97%, 98%, 99%, 99.5% or 99.9% sequence identity to SEQ ID NO: 114.

### OSK1

OSK1 regulates the expression of superoxide dismutases (such as SODCC1, SOD2 and SODA1). It may therefore be advantageous to express OSK1 in combination with superoxide dismutases in engineered or transgenic plants to protect against high level of salt. In some embodiments, the engineered or transgenic plant further comprises a gene of interest or a transgene that encodes OSK1. In certain embodiments, the engineered or transgenic plant comprises a gene of interest or a transgene that encodes OSK1 in combination with a gene of interest or a transgene that encodes a superoxide dismutase, such as SODCC1, SOD2 or SODA1. In some embodiments, the engineered or transgenic plant comprises genes of interest or transgenes which encode SOS1, SOS2, AHA3, VHA-A, NHX1, HKT1, SODA1, SODCC1 and OSK1. In some embodiments, the engineered or transgenic plant comprises genes of interest or transgenes which encode SOS1, SOS2, AHA3, VHA-A, NHX1, HKT1, SODA1, SOD2 and OSK1. In some embodiments, the engineered or transgenic plant comprises genes of interest or transgenes which encode SOS1, SOS2, AHA3, VHA-A, NHX1, HKT1, SODCC1, SOD2 and OSK1. In some embodiments, the engineered or transgenic plant comprises genes of interest or transgenes which encode SOS1, SOS2, AHA3, VHA-A, NHX1, HKT1, SODA1, SODCC1, SOD2 and OSK1. In some embodiments, the OSK1 is Oryza sativa OSK1 (OsOSK1), which can have the polypeptide sequence of Uniprot accession number Q9ZRJ1, QODGI1, Q9ZTF6 and/or Q852Q2. In some embodiments, the gene of interest or transgene comprises a polynucleotide sequence that encodes a polypeptide comprising the amino acid sequence of Q9ZRJ1, QODGI1, Q9ZTF6 or Q852Q2, or the transgene can comprise an amino acid sequence that has at least 75%, 80%, 85%, 90%, 95%, 96%, 97%, 98%, 99%, 99.5% or 99.9% sequence identity to Q9ZRJ1, QODGI1, Q9ZTF6 and/or Q852Q2.

In some embodiments, the OsPsbQ can have the polypeptide sequence of SEQ ID NO: 107, SEQ ID NO: 108, SEQ ID NO: 109 and/or SEQ ID NO: 110. In some embodiments, the gene of interest or transgene comprises a polynucleotide sequence that encodes a polypeptide comprising the amino acid sequence of SEQ ID NO: 107, SEQ ID NO: 108, SEQ ID NO: 109 and/or SEQ ID NO: 110 or the transgene can comprise an amino acid sequence that has at least 75%, 80%, 85%, 90%, 95%, 96%, 97%, 98%, 99%, 99.5% or 99.9% sequence identity to SEQ ID NO: 107, SEQ ID NO: 108, SEQ ID NO: 109 and/or SEQ ID NO: 110.

In some embodiments, the transgenic plant does not contain a transgene that encodes PERK13. In some embodiments, the gene of interest in an engineered plant is not PERK13.

### Promoters and Enhancer Elements

A promoter can be operably linked to a transgene to control its expression in the transgenic plant. The promoter can comprise at least 6, at least 7, at least 9, at least 10, at least 11, at least 12, at least 13, at least 14, at least 15, at least 16, at least 17, at least 18, at least 19, or at least 20 nucleotides, at least 30 nucleotides, at least 40 nucleotides or at least 50 nucleotides. The promoter can be located within at least about 50 nucleotides, at least about 75 nucleotides, at least about 100 nucleotides, at least about 125 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 600 nucleotides, at least about 700 nucleotides, at least about 800 nucleotides, at least about 900 nucleotides, or at least about 1000 nucleotides of the 5′ end of an open reading frame of transgene.

The promoter operably linked to a transgene can comprise a promoter sequence that is naturally present in the plant genome for the corresponding gene—i.e. if the transgene is OsSOS1, then the promoter can comprise the promoter sequence that is naturally present in the genome of a rice plant for OsSOS1. Alternatively, the promoter can be a specifically designed and/or an artificial promoter. The promoter can control the expression level of the transgene both spatially and temporally within the plant. For example, the promoter can be designed to specifically express the transgene in the shoot, leaf, seed or root of the plant. In some embodiments the promoter is a shoot specific promoter, leaf specific promoter, seed specific promoter or root specific promoter. In certain embodiments, the promoter can be a root specific promoter.

The promoter can be designed to specifically express the transgene in the seed, when the plant is germinating, when the plant is maturing, when the plant is flowering and when the plant develops fruit.

The inventors have identified that plants can be engineered so that the expression of endogenous salinity resistance genes can be altered by operably linking the enhancer elements described herein to endogenous salinity resistance genes. This can be achieved by inserting a enhancer element at the 5′ end of an open reading frame of a salt tolerance gene or inserting the enhancer element downstream of the 5′ end of an open reading frame within an intron of the endogenous salt tolerance gene. Enhancer elements are operably linked to each gene of interest to control their expression in the engineered plant. The Enhancer element can comprise at least 6, at least 7, at least 9, at least 10, at least 11, at least 12, at least 13, at least 14, at least 15, at least 16, at least 17, at least 18, at least 19, or at least 20 nucleotides, at least 30 nucleotides, at least 40 nucleotides or at least 50 nucleotides.

The enhancer element can be located within at least about 50 nucleotides, at least about 75 nucleotides, at least about 100 nucleotides, at least about 125 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 600 nucleotides, at least about 700 nucleotides, at least about 800 nucleotides, at least about 900 nucleotides, or at least about 1000 nucleotides of the 5′ end of an open reading frame of a salt tolerance gene. The salt tolerance gene can be SOS1, AHA-3, NHX1, VHA-A, HKT1, SOS1, SODCC and/or SOD2. The examples demonstrate that altering the expression of these endogenous salinity resistance genes can improve the salt tolerance of the plant. In some embodiments, operably linking enhancer elements to endogenous salinity resistance genes can involve (a) inducing callus formation from a seed of the plant; (b) transforming the callus with microcarriers using particle bombardment; (c) generating a transformed callus, wherein the microcarriers have adsorbed thereto at least two different DNA sequences comprising the enhancer element sequences that integrate into the genome such that at least two genes of interest are operatively linked to the enhancer element sequences. In some cases, the DNA sequences can comprise (i) 5′ and 3′ flanking homology arms or adapters corresponding to a 5′ region of each of the at least three salinity resistance genes; an internal sequence comprising a enhancer element (or at least 5, 6, 7, 8, 9, or 10 nucleotides from an enhancer element) which comprises DREB2A, or an ETH or AUX promoter sequences. Such a procedure can then comprise (c) recovering the transformed callus in growth medium to generate the multicellular structure comprising a plurality of engineered plant cells having improved salinity tolerance.

In some embodiments, the microcarriers have absorbed thereto programmable nucleases with specificity for a 5′ upstream region or an intronic region proximal to the 5′ end of an open reading frame of the genes of interest. The programmable nucleases can comprise a class II, type II or class II, type V Cas nuclease in complex with guide RNAs directed against a 5′ upstream region of the genes of interest or an intronic region proximal to the 5′ end of an open reading frame of the genes of interest. The programmable nucleases can comprise transcription activator-like (TAL) effector and nucleases (TALENs) with specificity for a 5′ region of the at least three salinity resistance genes or an intronic region proximal to the 5′ end of an open reading frame of the genes of interest. The programmable nucleases can comprise zinc finger nucleases (ZFN) with specificity for a 5′ region of the at least three salinity resistance genes or an intronic region proximal to the 5′ end of an open reading frame of the genes of interest.

Plant hormones (also known as phytohormones) are signaling molecules that can regulate plant growth and development. Plants produce a wide variety of hormones, including auxins, gibberellins, abscisic acid, cytokinins, salicylic acid, ethylene, jasmonates, brassinosteroids and peptides. In some embodiments, the enhancer element is hormone responsive. In some embodiments, a gene of interest encodes an ion transporter (e.g. a sodium or potassium transporter) and the gene of interest is operably linked to a hormone responsive enhancer element. In some embodiments, a gene of interest encodes a hydrogen exporting ATPase and the gene of interest is operably linked to a hormone responsive enhancer element. In some embodiments, the gene of interest encodes a hydrogen exporting pyrophosphatase and the gene of interest is operably linked to a hormone responsive enhancer element. In some embodiments, a gene of interest encodes a protein kinase and the gene of interest is operably linked to a hormone responsive enhancer element. In some embodiments, a gene of interest encodes an antioxidant and the gene of interest is operably linked to a hormone responsive enhancer element.

In some embodiments, the promoter is a hormone responsive promoter. In some embodiments, a transgene encodes an ion transporter (e.g. a sodium or potassium transporter) and the transgene is operably linked to a hormone responsive promoter. In some embodiments, a transgene encodes a hydrogen exporting ATPase and the transgene is operably linked to a hormone responsive promoter. In some embodiments, a transgene encodes a hydrogen exporting pyrophosphatase and the transgene is operably linked to a hormone responsive promoter. In some embodiments, a transgene encodes a protein kinase and the transgene is operably linked to a hormone responsive promoter. In some embodiments, a transgene encodes an antioxidant and the transgene is operably linked to a hormone responsive promoter.

The hormone responsive enhancer element or promoter may be responsive to abscisic acid (ABA), ethylene (ETH), gibberellin (GA) and auxin (AUX), or any combination thereof. In some embodiments, enhancer elements or promoters can include DREB2A, ETH, or AUX promoters, or any combination thereof. The enhancer element or promoter can comprise at least 6 nucleotides from DREB2A, ETH, or AUX promoter. The sequences of DREB2A, ETH, and AUX are shown in Table 1.

In some embodiments, a gene of interest encodes an ion transporter and the gene of interest is operably linked to an enhancer element that is responsive to abscisic acid, ethylene, gibberellin and auxin, or any combination thereof. In some embodiments, a gene of interest encodes a hydrogen exporting ATPase and the gene of interest is operably linked to an enhancer element that is responsive to abscisic acid, ethylene, gibberellin and auxin, or any combination thereof. In some embodiments, a gene of interest encodes a hydrogen exporting pyrophosphatase and the gene of interest is operably linked to an enhancer element that is responsive to abscisic acid, ethylene, gibberellin and auxin, or any combination thereof. In some embodiments, a gene of interest encodes a protein kinase and the gene of interest is operably linked to an enhancer element that is responsive to abscisic acid, ethylene, gibberellin and auxin, or any combination thereof. In some embodiments, a gene of interest encodes an antioxidant and the gene of interest is operably linked to an enhancer element that is responsive to abscisic acid, ethylene, gibberellin and auxin, or any combination thereof.

In certain embodiments, the gene of interest encodes SOS1 and the gene of interest is operably linked to an enhancer element that is responsive to abscisic acid, ethylene, gibberellin and auxin, or any combination thereof. In certain embodiments, the gene of interest encodes SOS2 and the gene of interest is operably linked to an enhancer element that is responsive to abscisic acid, ethylene, gibberellin and auxin, or any combination thereof. In certain embodiments, the gene of interest encodes NHK1 and the gene of interest is operably linked to an enhancer element that is responsive to abscisic acid, ethylene, gibberellin and auxin, or any combination thereof. In certain embodiments, the gene of interest encodes AHA3 and the gene of interest is operably linked to an enhancer element that is responsive to abscisic acid, ethylene, gibberellin and auxin, or any combination thereof. In certain embodiments, the gene of interest encodes HKT1 and the gene of interest is operably linked to an enhancer element that is responsive to abscisic acid, ethylene, gibberellin and auxin, or any combination thereof. In certain embodiments, the gene of interest encodes VHA-A and the gene of interest is operably linked to an enhancer element that is responsive to abscisic acid, ethylene, gibberellin and auxin, or any combination thereof. In certain embodiments, the gene of interest encodes SODA1 and the gene of interest is operably linked to an enhancer element that is responsive to abscisic acid, ethylene, gibberellin and auxin, or any combination thereof. In certain embodiments, the gene of interest encodes SOD2 and the gene of interest is operably linked to an enhancer element that is responsive to abscisic acid, ethylene, gibberellin and auxin, or any combination thereof. In certain embodiments, the gene of interest encodes SODCC1 and the gene of interest is operably linked to an enhancer element that is responsive to abscisic acid, ethylene, gibberellin and auxin, or any combination thereof.

In some embodiments, a transgene encodes an ion transporter and the transgene is operably linked to a promoter that is responsive to abscisic acid, ethylene, gibberellin and auxin, or any combination thereof. In some embodiments, a transgene encodes a hydrogen exporting ATPase and the transgene is operably linked to a promoter that is responsive to abscisic acid, ethylene, gibberellin and auxin, or any combination thereof. In some embodiments, a transgene encodes a hydrogen exporting pyrophosphatase and the transgene is operably linked to a promoter that is responsive to abscisic acid, ethylene, gibberellin and auxin, or any combination thereof. In some embodiments, a transgene encodes a protein kinase and the transgene is operably linked to a promoter that is responsive to abscisic acid, ethylene, gibberellin and auxin, or any combination thereof. In some embodiments, a transgene encodes an antioxidant and the transgene is operably linked to a promoter that is responsive to abscisic acid, ethylene, gibberellin and auxin, or any combination thereof.

In certain embodiments, the transgene encodes SOS1 and the transgene is operably linked to a promoter that is responsive to abscisic acid, ethylene, gibberellin and auxin, or any combination thereof. In certain embodiments, the transgene encodes SOS2 and the transgene is operably linked to a promoter that is responsive to abscisic acid, ethylene, gibberellin and auxin, or any combination thereof. In certain embodiments, the transgene encodes NHK1 and the transgene is operably linked to a promoter that is responsive to abscisic acid, ethylene, gibberellin and auxin, or any combination thereof. In certain embodiments, the transgene encodes AHA3 and the transgene is operably linked to a promoter that is responsive to abscisic acid, ethylene, gibberellin and auxin, or any combination thereof. In certain embodiments, the transgene encodes HKT1 and the transgene is operably linked to a promoter that is responsive to abscisic acid, ethylene, gibberellin and auxin, or any combination thereof. In certain embodiments, the transgene encodes VHA-A and the transgene is operably linked to a promoter that is responsive to abscisic acid, ethylene, gibberellin and auxin, or any combination thereof. In certain embodiments, the transgene encodes SODA1 and the transgene is operably linked to a promoter that is responsive to abscisic acid, ethylene, gibberellin and auxin, or any combination thereof. In certain embodiments, the transgene encodes SOD2 and the transgene is operably linked to a promoter that is responsive to abscisic acid, ethylene, gibberellin and auxin, or any combination thereof. In certain embodiments, the transgene encodes SODCC1 and the transgene is operably linked to a promoter that is responsive to abscisic acid, ethylene, gibberellin and auxin, or any combination thereof.

In some embodiments, the hormone responsive enhancer element or promoter is modulated by its proximity to a general transcription-enhancing element (e.g., fewer than 15, 14, 13, 12, 11, 10, 9, 8, 7, 6, 5, 4, 3, 2, or 1 nucleotides distance to). Such general transcription-enhancing elements include, but are not limited to, TAF-1, TATA, E2F, G-BOX, or CAAT sequences. The general transcription-enhancing elements can include any of the sequences outlined in Table 1 or any combination thereof.

In some embodiments, the enhancer element or promoter can comprise one or more of promoter elements selected from the group of DREB2A, GA, TAF-1, TATATA, TATA, ETH, ARE, E2F-1, CAAT, TGA, G-BOX or AUX COMP. The sequences for these elements are provided in Table 1. Other possible promoter elements that can be used in the enhancer elements or promoters are described in the PLACE database [12], and references [13], [14][15], [16], [17], [18], [19], [20], [21], [22], [23], [24], [25] and [26], which are incorporated herein by reference.

TABLE 1
Summary of promoter sequences

| Code | Promoter element | Sequence |
|---|---|---|
| DREB2A | Drought responsive element-binding | GCCGAC |
| | protein 2A | |
| GA | Gibberellin Acid Responsive Element | CCTTTG |
| TAF-1 | TAF-1 binding site | GCCACGTGGC (SEQ ID NO: 79) |
| TATATA | TATA box triplicate | TATATA/AATATAT |
| TATA | TATA box | TATA |
| ETH | Ethylene Responsive Element | TAAGAGCCGCC (SEQ ID NO: 80)/ |
| | | AGCCGCC |
| ARE | Auxin Responsive Element | TGTCTC |
| E2F-1 | E2F Binding Site | GCGGGAAA |
| CAAT | | GGCCAATCT/CACAAT/CCCACT |
| TGA | Auxin inducible element | TGACGTAA/CTGACG |
| G-BOX | Brassica specific bZIP TF binding site | ACACGTG(T/GC) |
| AUX COMP | D1 Auxin Responsive composite | CCTCGTGTCTC (SEQ ID NO: 81) |
| | element. | |

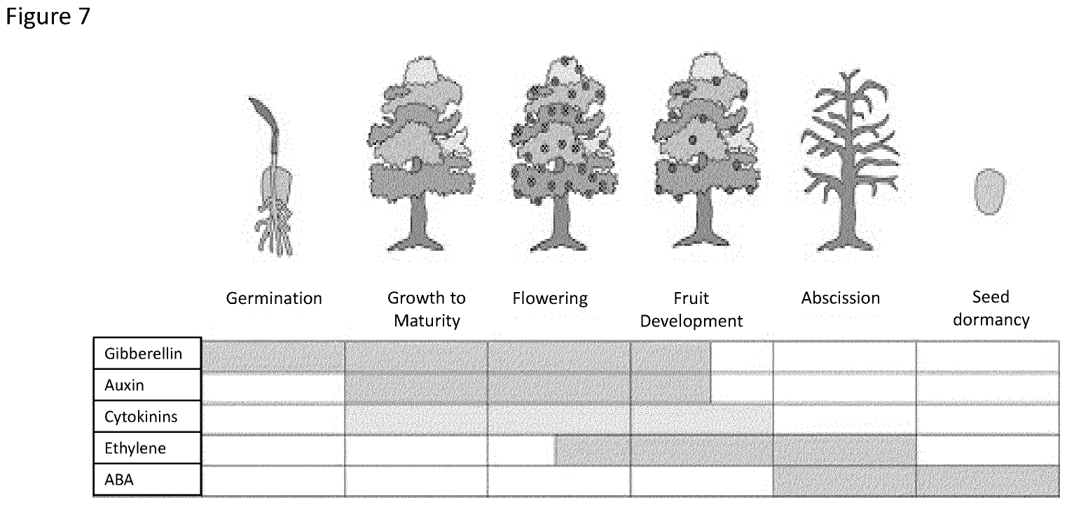

The distribution of hormones changes over time and spatially throughout the plant. FIG. 7 demonstrates how distribution of certain hormones vary through the plant life cycle. The inventors have found that the varying distribution of hormones within the plant can be exploited to control the expression of the genes of interest or transgenes. For example, it may be advantageous to express the genes of interest or transgenes specifically in the roots when the plant is exposed to salt stress instead of the whole plant, as the unnecessary expression of the genes of interest or transgenes in the rest of the plant or the non-saline exposed portions of the plant will have a negative energy cost.

For example, auxin is expressed very intensely at the tip of the root, but the expression reduces further up the root. Auxin is also heavily expressed on the outer edges of the root in comparison to the center of the root. Using this auxin distribution, the inventors have found that they can trigger the expression of the SOS1 mechanism (SOS1, SOS2 and AHA3) to be most active at the tip of the root, this results in most SOS1 being produced once the roots detect saltwater and a consistent SOS1 presence through saltwater immersion. It also creates a pathway for salt to travel down as it moves along the SOS1 distribution down to the tip of the root and away from the center of the plant/stem. In some embodiments, the SOS1 gene, a SOS2 gene and/or a AHA3 gene is operably linked to an enhancer element that comprises an auxin responsive promoter element. In some embodiments, a SOS1 transgene, a SOS2 transgene and/or a AHA3 transgene is operably linked to a promoter that comprises an auxin responsive promoter element.

The variation in the distribution of hormones throughout the life cycle of the plant can be exploited so that energy can be conserved when a particular enzyme is not needed. For example, if antioxidant enzymes are operably linked to a gibberellin promoter they will be expressed from germination through the vegetative and flowering portion of the plant's growth. This provides the plant with protection through the early days of the plant's life until a salt management system is established.

Multiple genes of interest transgenes can be operably linked to the same or similar enhancer elements or promoters to ensure that they are expressed in concert. For example, if the engineered plant contains more than one gene of interest that encodes an antioxidant, then each of the antioxidants can be operably linked to the same or a similar enhancer element to ensure that their expression is ubiquitous. In certain embodiments, the genes of interest or the transgenes that encode antioxidants are operably linked to the same enhancer element or promoter which comprises GA, TATA, DREB2A and ETH elements or which comprises the sequence of SEQ ID NO: 16-18.

SOS1, SOS2 and AHA3 act together as a synergistic group. Therefore, in certain embodiments, the engineered plant comprises genes of interest that encode SOS1, SOS2 and AHA3, and these genes of interest are operably linked to similar enhancer element to ensure that they are expressed at the same time. Therefore, in certain embodiments, the transgenic plant comprises transgenes that encode SOS1, SOS2 and AHA3, and these transgenes are operably linked to similar promoters to ensure that they are expressed at the same time. These three genes act in a synergistic group, therefore it is advantageous to use enhancer element or promoters that are similar. In certain embodiments, the engineered plant comprises genes of interest that encode OsSOS1, OsSOS2 and OsAHA3; wherein the OsSOS1 gene is operably linked to an enhancer element comprising SEQ ID NO: 12, the OsSOS2 gene is operably linked to an enhancer element comprising SEQ ID NO: 13, the OsAHA3 gene is operably linked to an enhancer element comprising SEQ ID NO: 14. In certain embodiments, the transgenic plant comprises transgenes that encode OsSOS1, OsSOS2 and OsAHA3; wherein the OsSOS1 transgene is operably linked to a promoter comprising SEQ ID NO: 12, the OsSOS2 transgene is operably linked to a promoter comprising SEQ ID NO: 13, the OsAHA3 transgene is operably linked to a promoter comprising SEQ ID NO: 14.

In some embodiments, the enhancer element can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 10-18. In some embodiments, the promoter can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 10-18, when the transgenic plant is Oryza sativa. In certain embodiments, the promoter has the sequence of SEQ ID NO 10-18, when the plant cell is Oryza sativa.

In certain embodiments, a OsSOS1 gene is operably linked to an enhancer element comprising SEQ ID NO: 12, a OsSOS2 gene is operably linked to an enhancer element comprising SEQ ID NO: 13, a OsAHA3 gene is operably linked to an enhancer element comprising SEQ ID NO: 14, a OsVHA-A gene is operably linked to an enhancer element comprising SEQ ID NO: 11, a OsNHX1 gene is operably linked to an enhancer element comprising SEQ ID NO: 10, a OsHKT1 gene is operably linked to an enhancer element comprising SEQ ID NO: 15, a OsSODA1 gene is operably linked to an enhancer element comprising SEQ ID NO: 16 and/or a OsSOD2 gene is operably linked to an enhancer element comprising SEQ ID NO: 18.

In certain embodiments, a OsSOS1 transgene is operably linked to a promoter comprising SEQ ID NO: 12, a OsSOS2 transgene is operably linked to a promoter comprising SEQ ID NO: 13, a OsAHA3 transgene is operably linked to a promoter comprising SEQ ID NO: 14, a OsVHA-A transgene is operably linked to a promoter comprising SEQ ID NO: 11, a OsNHX1 transgene is operably linked to a promoter comprising SEQ ID NO: 10, a OsHKT1 transgene is operably linked to a promoter comprising SEQ ID NO: 15, a OsSODA1 transgene is operably linked to a promoter comprising SEQ ID NO: 16 and/or a OsSOD2 transgene is operably linked to a promoter comprising SEQ ID NO: 18.

In some embodiments, the enhancer element can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 82-91 listed in Table 2, In some embodiments, the promoter can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 82-91 listed in Table 2, when the transgenic plant is a Brassica species.

In certain embodiments, a BoSOS1 gene is operably linked to an enhancer element comprising SEQ ID NO: 84, a BoSOS2 gene is operably linked to an enhancer element comprising SEQ ID NO: 85, a BoAHA3 gene is operably linked to an enhancer element comprising SEQ ID NO: 86, a BoVHA-A gene is operably linked to an enhancer element comprising SEQ ID NO: 83, a BoNHX1 gene is operably linked to an enhancer element comprising SEQ ID NO: 82, a BoHKT1 gene is operably linked to an enhancer element comprising SEQ ID NO: 87, a BoSODA1 gene is operably linked to an enhancer element comprising SEQ ID NO: 89, a BoSOD2 gene is operably linked to an enhancer element comprising SEQ ID NO: 88, a Bo CuZn SOD gene is operably linked to an enhancer element comprising SEQ ID NO: 90 and/or a BoOSK1 gene is operably linked to an enhancer element comprising SEQ ID NO: 91.

In certain embodiments, a BoSOS1 transgene is operably linked to a promoter comprising SEQ ID NO: 84, a BoSOS2 transgene is operably linked to a promoter comprising SEQ ID NO: 85, a BoAHA3 transgene is operably linked to a promoter comprising SEQ ID NO: 86, a BoVHA-A transgene is operably linked to a promoter comprising SEQ ID NO: 83, a BoNHX1 transgene is operably linked to a promoter comprising SEQ ID NO: 82, a BoHKT1 transgene is operably linked to a promoter comprising SEQ ID NO: 87, a BoSODA1 transgene is operably linked to a promoter comprising SEQ ID NO: 89, a BoSOD2 transgene is operably linked to a promoter comprising SEQ ID NO: 88, a Bo CuZn SOD transgene is operably linked to a promoter comprising SEQ ID NO: 90 and/or a BoOSK1 transgene is operably linked to a promoter comprising SEQ ID NO: 91.

TABLE 2
Promoter sequences that can be introduced to the 5′ end of
the transgenes from Brassica species

| Oryza sativa | | | |
|---|---|---|---|
| orthologue/ | Gene | Promoters | |
| kale gene | accession code | elements | Promoter sequence |
| NHX1/ | Bo9g010200 | CAAT.DREB.G | CACAATGCCGACCCTTTGGCCACG |
| Bo9g010200 | | A.TAF.TATATA | TGGCAATATATA(SEQ ID NO: 82) |
| Os-VHA- | Bo6g122400 | CAAT.TATA/ | CACAATGCCGACGCCACGTGGCAA |
| A/BoVHA-A | | TAF-1 + ETH | GCCGCCTATATA(SEQ ID NO: 83) |
| SOS1/ | Bo9g071470 | TGA + CAAT*2.A | CTGACGCACAATCCCACTTGTCTCT |
| BoSOS1 | | UX.ETH.E2F.DR | ATATAGCCGCCGCGGGAAAAGCCG |
| | | EB.E2F.TATAT | ACGCGGGAAAATATATAA |
| | | A | (SEQ ID NO: 84) |
| SOS2/ | Bo4g139520.1 | AUX/ETH + | CTGACGCACAATCCCACTTGTCTCT |
| BoSOS2 | | E2F-1(*2)+ | ATATAGCCGCCGCGGGAAAAGCCG |
| | | TATA + | ACGCGGGAAAATATATAA |
| | | DREB.TATATA | (SEQ ID NO: 85) |
| OsAHA3/ | Bo9g133110 | AUXCOMP.AU | CACAATCCCACTACCTCGTGTCTC |
| BoAHA3 | | X/ETH + E2F-1 | GCCGCCGCGGGAAAAGCCGACGC |
| | | (*2) + TATA + | GGGAAAATATATA |
| | | DREB | (SEQ ID NO: 86) |
| HKT1/ | Bo3g044660 | CAAT.ARE.DRE | CACAATACCTCGTGTCTCGCCGAC |
| BoHKT1 | | B2A x 3+ E2F-1 | GCGGGAAAGGCCGACGCCGCCGC |
| | | | CGAC (SEQ ID NO: 87) |
| [CuZn]SOD2/ | Bo5g137020 | CAAT + GA + | CACAATCCCACTCCTTTGTATATAG |
| Bo5g137020 | | TATA + G-BOX + | CCGACGCCGACGCCGCCACCTCGT |
| | | DREB2A + AUX | GTCTC (SEQ ID NO: 88) |
| SODA1/ | Bolg141360 | CAAT + GA + | CACAATCCCACTCCTTTGTATATAG |
| BoSODA1 | | TATA + G-BOX + | CCGACGCCGACGCCGCCACCTCGT |
| | | DREB2A + AUX | GTCTC (SEQ ID NO: 89) |
| CuZn SOD | Bo4g165150 | CAAT + GA + | CACAATCCCACTCCTTTGTATATAG |
| chloroplast/ | | TATA + G-box + | CCGACGCCGACGCCGCCACCTCGT |
| CuZn SOD | | DREB2A + AUX | GTCTC (SEQ ID NO: 90) |
| chloroplast | | | |
| OSK1/ | Bo4g138000 | AUX ENH.AUX + | CCTCGTGTCTCATGACGCACAATC |
| Bo4g138000 | | TGA + CAAT + | CCACTCCTTTGTATATAGCCGACG |
| | | GA + TATA + | CCGACGCCGCCGTATATA |
| | | G- | (SEQ ID NO: 91) |
| | | BOX/DREB2A + | |
| | | TATA | |

In certain embodiments, the invention provides an engineered rice plant comprising at least eight genes of interest, wherein the genes of interest encode OsSOS1, OsSOS2, OsAHA3, OsVHA-A, OsNHX1, OsHKT1, OsSODA1 and OsSOD2; wherein the OsSOS1 gene comprises a polynucleotide sequence that encodes a polypeptide comprising the amino acid sequence of Q5ICN3 or Q7XBF9; wherein the OsSOS2 gene comprises a polynucleotide sequence that encodes a polypeptide comprising the amino acid sequence of Q69Q47; wherein the OsAHA3 gene comprises a polynucleotide sequence that encodes a polypeptide comprising the amino acid sequence of Q8L6I3; wherein the OsVHA-A gene comprises a polynucleotide sequence that encodes a polypeptide comprising the amino acid sequence of Q651T8; wherein the OsNHX1 gene comprises a polynucleotide sequence that encodes a polypeptide comprising the amino acid sequence of Q9SXJ8 or Q6VVA7; wherein the OsHKT1 gene comprises a polynucleotide sequence that encodes a polypeptide comprising the amino acid sequence of Q0D9S3; wherein the OsSODA1 gene comprises a polynucleotide sequence that encodes a polypeptide comprising the amino acid sequence of Q43121 and wherein the OsSOD2 gene comprises a polynucleotide sequence that encodes a polypeptide comprising the amino acid sequence of Q10PW4;

wherein the OsSOS1 gene is operably linked to a promoter comprising SEQ ID NO: 12, the OsSOS2 gene is operably linked to a promoter comprising SEQ ID NO: 13, the OsAHA3 gene is operably linked to a promoter comprising SEQ ID NO: 14, the OsVHA-A gene is operably linked to a promoter comprising SEQ ID NO: 11, the OsNHX1 gene is operably linked to a promoter comprising SEQ ID NO: 10, the OsHKT1 gene is operably linked to a promoter comprising SEQ ID NO: 15, the OsSODA1 gene is operably linked to a promoter comprising SEQ ID NO: 16 and the OsSOD2 gene is operably linked to a promoter comprising SEQ ID NO: 18.

In certain embodiments, the invention provides a transgenic rice plant comprising at least eight transgenes, wherein the transgenes encode OsSOS1, OsSOS2, OsAHA3, OsVHA-A, OsNHX1, OsHKT1, OsSODA1 and OsSOD2;

wherein the OsSOS1 transgene comprises a polynucleotide sequence that encodes a polypeptide comprising the amino acid sequence of Q5ICN3 or Q7XBF9; wherein the OsSOS2 transgene comprises a polynucleotide sequence that encodes a polypeptide comprising the amino acid sequence of Q69Q47; wherein the OsAHA3 transgene comprises a polynucleotide sequence that encodes a polypeptide comprising the amino acid sequence of Q8L6I3; wherein the OsVHA-A transgene comprises a polynucleotide sequence that encodes a polypeptide comprising the amino acid sequence of Q651T8; wherein the OsNHX1 transgene comprises a polynucleotide sequence that encodes a polypeptide comprising the amino acid sequence of Q9SXJ8 or Q6VVA7; wherein the OsHKT1 transgene comprises a polynucleotide sequence that encodes a polypeptide comprising the amino acid sequence of Q0D9S3; wherein the OsSODA1 transgene comprises a polynucleotide sequence that encodes a polypeptide comprising the amino acid sequence of Q43121 and wherein the OsSOD2 transgene comprises a polynucleotide sequence that encodes a polypeptide comprising the amino acid sequence of Q10PW4;
wherein the OsSOS1 transgene is operably linked to a promoter comprising SEQ ID NO: 12, the OsSOS2 transgene is operably linked to a promoter comprising SEQ ID NO: 13, the OsAHA3 transgene is operably linked to a promoter comprising SEQ ID NO: 14, the OsVHA-A transgene is operably linked to a promoter comprising SEQ ID NO: 11, the OsNHX1 transgene is operably linked to a promoter comprising SEQ ID NO: 10, the OsHKT1 transgene is operably linked to a promoter comprising SEQ ID NO: 15, the OsSODA1 transgene is operably linked to a promoter comprising SEQ ID NO: 16 and the OsSOD2 transgene is operably linked to a promoter comprising SEQ ID NO: 18.

In certain embodiments, the invention provides an engineered rice plant comprising at least eight genes of interest, wherein the genes of interest encode OsSOS1, OsSOS2, OsAHA3, OsVHA-A, OsNHX1, OsHKT1, OsSODA1 and OsSODCC1;

wherein the OsSOS1 gene comprises a polynucleotide sequence that encodes a polypeptide comprising the amino acid sequence of Q5ICN3 or Q7XBF9; wherein the OsSOS2 gene comprises a polynucleotide sequence that encodes a polypeptide comprising the amino acid sequence of Q69Q47; wherein the OsAHA3 gene comprises a polynucleotide sequence that encodes a polypeptide comprising the amino acid sequence of Q8L6I3; wherein the OsVHA-A gene comprises a polynucleotide sequence that encodes a polypeptide comprising the amino acid sequence of Q651T8; wherein the OsNHX1 gene comprises a polynucleotide sequence that encodes a polypeptide comprising the amino acid sequence of Q9SXJ8 or Q6VVA7; wherein the OsHKT1 gene comprises a polynucleotide sequence that encodes a polypeptide comprising the amino acid sequence of Q0D9S3; wherein the OsSODA1 gene comprises a polynucleotide sequence that encodes a polypeptide comprising the amino acid sequence of Q43121 and wherein the OsSODCC1 gene comprises a polynucleotide sequence that encodes a polypeptide comprising the amino acid sequence of QODRV6;

wherein the OsSOS1 gene is operably linked to a promoter comprising SEQ ID NO: 12, the OsSOS2 gene is operably linked to a promoter comprising SEQ ID NO: 13, the OsAHA3 gene is operably linked to a promoter comprising SEQ ID NO: 14, the OsVHA-A gene is operably linked to a promoter comprising SEQ ID NO: 11, the OsNHX1 gene is operably linked to a promoter comprising EQ ID NO: 10, the OsHKT1 gene is operably linked to a promoter comprising SEQ ID NO: 15, the OsSODA1 gene is operably linked to a promoter comprising SEQ ID NO: 16 and the OsSODCC1 gene is operably linked to a promoter comprising SEQ ID NO: 17.

In certain embodiments, the invention provides a transgenic rice plant comprising at least eight transgenes, wherein the transgenes encode OsSOS1, OsSOS2, OsAHA3, OsVHA-A, OsNHX1, OsHKT1, OsSODA1 and OsSODCC1;

wherein the OsSOS1 transgene comprises a polynucleotide sequence that encodes a polypeptide comprising the amino acid sequence of Q5ICN3 or Q7XBF9; wherein the OsSOS2 transgene comprises a polynucleotide sequence that encodes a polypeptide comprising the amino acid sequence of Q69Q47; wherein the OsAHA3 transgene comprises a polynucleotide sequence that encodes a polypeptide comprising the amino acid sequence of Q8L6I3; wherein the OsVHA-A transgene comprises a polynucleotide sequence that encodes a polypeptide comprising the amino acid sequence of Q651T8; wherein the OsNHX1 transgene comprises a polynucleotide sequence that encodes a polypeptide comprising the amino acid sequence of Q9SXJ8 or Q6VVA7; wherein the OsHKT1 transgene comprises a polynucleotide sequence that encodes a polypeptide comprising the amino acid sequence of Q0D9S3; wherein the OsSODA1 transgene comprises a polynucleotide sequence that encodes a polypeptide comprising the amino acid sequence of Q43121 and wherein the OsSODCC1 transgene comprises a polynucleotide sequence that encodes a polypeptide comprising the amino acid sequence of QODRV6;

wherein the OsSOS1 transgene is operably linked to a promoter comprising SEQ ID NO: 12, the OsSOS2 transgene is operably linked to a promoter comprising SEQ ID NO: 13, the OsAHA3 transgene is operably linked to a promoter comprising SEQ ID NO: 14, the OsVHA-A transgene is operably linked to a promoter comprising SEQ ID NO: 11, the OsNHX1 transgene is operably linked to a promoter comprising EQ ID NO: 10, the OsHKT1 transgene is operably linked to a promoter comprising SEQ ID NO: 15, the OsSODA1 transgene is operably linked to a promoter comprising SEQ ID NO: 16 and the OsSODCC1 transgene is operably linked to a promoter comprising SEQ ID NO: 17.

In certain embodiments, the invention provides an engineered rice plant comprising at least eight genes of interest, wherein the genes of interest encode OsSOS1, OsSOS2, OsAHA3, OsVHA-A, OsNHX1, OsHKT1, OsSODA1 and OsSOD2;

wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93;

wherein the OsSOS1 gene is operably linked to a promoter comprising SEQ ID NO: 12, the OsSOS2 gene is operably linked to a promoter comprising SEQ ID NO: 13, the OsAHA3 gene is operably linked to a promoter comprising SEQ ID NO: 14, the OsVHA-A gene is operably linked to a promoter comprising SEQ ID NO: 11, the OsNHX1 gene is operably linked to a promoter comprising SEQ ID NO: 10, the OsHKT1 gene is operably linked to a promoter comprising SEQ ID NO: 15, the OsSODA1 gene is operably linked to a promoter comprising SEQ ID NO: 16 and the OsSOD2 gene is operably linked to a promoter comprising SEQ ID NO: 18

In certain embodiments, the invention provides a transgenic rice plant comprising at least eight transgenes, wherein the transgenes encode OsSOS1, OsSOS2, OsAHA3, OsVHA-A, OsNHX1, OsHKT1, OsSODA1 and OsSOD2;

wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93;

wherein the OsSOS1 transgene is operably linked to a promoter comprising SEQ ID NO: 12, the OsSOS2 transgene is operably linked to a promoter comprising SEQ ID NO: 13, the OsAHA3 transgene is operably linked to a promoter comprising SEQ ID NO: 14, the OsVHA-A transgene is operably linked to a promoter comprising SEQ ID NO: 11, the OsNHX1 transgene is operably linked to a promoter comprising SEQ ID NO: 10, the OsHKT1 transgene is operably linked to a promoter comprising SEQ ID NO: 15, the OsSODA1 transgene is operably linked to a promoter comprising SEQ ID NO: 16 and the OsSOD2 transgene is operably linked to a promoter comprising SEQ ID NO: 18.

### Engineered Plants, Engineered Plant Parts and Engineered Multicellular Structures, Transgenic Plants, Transgenic Plant Parts and Transgenic Multicellular Structures

### Engineered and Transgenic Plants

In some embodiments the engineered or transgenic plant can be angiosperms. Angiosperms include both monocotyledonous angiosperms and dicotyledonous angiosperms. Monocotyledonous angiosperms include, but are not limited to, cereal crops, such as Zea mays (maize), Oryza sativa (rice), the genus Saccharum (sugar cane), Hordeum vulgare (barley), millet, oat (Avena sativa), Secale cereale (rye), the genus sorghum (sorghum), the genus Triticum (wheat), or any combination thereof. Dicotyledonous angiosperms include vegetable crops, such as Brassica (e.g. Brassica oleracea, kale), Glycine (e.g. Glycine max—soybean), Vigna radiata (mung bean), Chenopodium quinoa (quinoa), Soja, or Solanum (e.g. Solanum tuberosum—tomato).

In certain embodiments, the engineered plant is an engineered rice plant. In certain embodiments, the transgenic plant is a transgenic rice plant. Possible rice varieties that can be used in the invention include: Agostano, Dichroa, Early Sutarsar, Hunan Early Dwarf, Java, Kendzo, Konosu #2, Kurumiwase, Kwanto Wase, Novelli Gigante, Okuro Mochi, Primanychskij, Sensho Tane, Venere Italian Black Rice, Zhe 733, Cho Seun Zo Saeng, Se Zic, Daido, Mamoriaka, Duborskian, Yukihikari, Hayayuki, Truong Giang, MHC-2, TH3-5, Ho Phat 3. In some embodiments, the engineered or transgenic plant is the rice variety Hayayuki, TH 3-5, Truong Giang, MHC2 and Ha Phat 3, Java long, Se Zic, Agostano, Hunan and Dichroa. In certain embodiments, the engineered rice plant is a rice variety Hayayuki engineered plant. In certain embodiments, the transgenic rice plant is a rice variety Hayayuki transgenic plant.

The gene of interest or transgenes described above can originate from any plant species. For example, the gene of interest or transgene may be a rice gene, a maize gene, a sugar cane gene, a barley gene, a millet gene, an oat gene, a rye gene, a sorghum gene, a wheat gene, a kale gene, a soybean gene, a mung bean gene, a quinoa gene, or a tomato gene.

In some embodiments, the transgenic plant comprises a transgene derived from the same species as the transgenic plant. In other embodiments, the transgenic plant comprises a transgene derived from a different species from the transgenic plant, such as an orthologue of a transgene present in the transgenic plant. Where the transgene is derived from the same species as the transgenic plant its insertion as a transgene allows for an additional copy of the gene to be present in the plant and/or the transgene is inserted under the control of a promoter which is different from the endogenous promoter which allows for the expression level or pattern to differ from that of the expression pattern or level of the endogenous gene.

In certain embodiments, the transgenic plant is a transgenic rice plant that comprises transgenes that are rice genes. In some embodiments, the transgenic plant is a transgenic maize plant that comprises transgenes that are maize genes. In some embodiments, the transgenic plant is a transgenic sugar cane plant that comprises transgenes that are sugar cane genes. In some embodiments, the transgenic plant is a transgenic barley plant that comprises transgenes that are barley genes. In some embodiments, the transgenic plant is a transgenic millet plant that comprises transgenes that are millet genes. In some embodiments, the transgenic plant is a transgenic oat plant that comprises transgenes that are oat genes. In some embodiments, the transgenic plant is a transgenic rye plant that comprises transgenes that are rye genes. In some embodiments, the transgenic plant is a transgenic sorghum plant that comprises transgenes that are sorghum genes. In some embodiments, the transgenic plant is a transgenic wheat plant that comprises transgenes that are wheat genes. In some embodiments, the transgenic plant is a transgenic kale plant that comprises transgenes that are kale genes. In some embodiments, the transgenic plant is a transgenic soybean plant that comprises transgenes that are soybean genes. In some embodiments, the transgenic plant is a transgenic mung bean plant that comprises transgenes that are mung bean genes. In some embodiments, the transgenic plant is a transgenic quinoa plant that comprises transgenes that are quinoa genes. In some embodiments, the transgenic plant is a transgenic tomato plant that comprises transgenes that are tomato genes.

The engineered plants, plant parts and multicellular structures are not produced by a process that involves homologous recombination. The engineered plants, plant parts and multicellular structures and the seeds according to the invention are not produced by an essentially biological process. The transgenic plants, plant parts and multicellular structures are not produced by a process that involves homologous recombination. The transgenic plants, plant parts and multicellular structures and the seeds according to the invention are not produced by an essentially biological process.

In some embodiments, the engineered or transgenic plant is capable of growing in a medium having a salt concentration greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 32 g/L, about 34 g/L, about 36 g/L, about 38 g/L, about 40 g/L, about 42 g/L, about 44 g/L, about 46 g/L, about 48 g/L, about 50 g/L. In certain embodiments, the engineered or transgenic plant is capable of growing in a medium having a salt concentration greater than about 10 g/L, 20 g/L or 35 g/L. In further certain embodiments, the engineered or transgenic plant is capable of growing in a medium having a salt concentration greater than about 35 g/L, because this is greater than ocean salinity.

The engineered and transgenic plants of the invention can have increased salinity tolerance compared to a wild type plant of the same species. In some embodiments, the engineered or transgenic plant has an elevated growth rate in a medium having a particular salt concentration or ECe compared to the growth rate of a wild type plant. The medium that the engineered or transgenic plant can grow in can have an electrical conductivity of a saturated paste extract (ECₑ) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some embodiments, the medium is liquid (e.g., for hydroponic growth strategies). In some embodiments, the medium is solid (e.g., soil, sand). In some embodiments, the medium is semi-solid.

In some embodiments, the engineered or transgenic plant has a growth rate in a saline medium that is equal to or greater than the growth rate in a non-saline medium. The saline medium can be “slightly saline” (e.g., having an electrical conductivity of the saturated paste extract (ECₑ) or liquid EC of between 4 and 8 dS/m), “moderately saline” (e.g., having an electrical conductivity of the saturated paste extract (ECₑ) or liquid EC of between 8 and 16 dS/m), or “severely saline” can be defined as having an electrical conductivity of the saturated paste extract (ECₑ) or liquid EC of greater than 16 dS/m. The non-saline medium can have an electrical conductivity of the saturated paste extract (ECₑ) or liquid EC of less than 4 dS/m, less than 3 dS/m, less than 2 dS/m, less than 1.7 dS/m, or less than 1.5 dS/m. In some embodiments, the growth rate in saline medium is at least 5%, 10%, 15%, 20%, 25%, 30%, 35%, 40%, 45%, 50%, 100%, 150%, 200% or more.

In some embodiments, the engineered or transgenic plant has a particular threshold salinity (ECₜ), or an elevated threshold salinity. In some embodiments, the engineered or transgenic plant has a threshold salinity of at least about 6 dS/m, at least about 6.5 dS/m, at least about 6.7 dS/m, at least about 7 dS/m, at least about 7.5 dS/m, at least about 8 dS/m, at least about 8.5 dS/m, at least about 9 dS/m, at least about 9.5 dS/m, at least about 10 dS/m, at least about 10.5 dS/m, at least about 11 dS/m, at least about 11.5 dS/m, at least about 12 dS/m, at least about 12.5 dS/m, at least about 13 dS/m, at least about 13.5 dS/m, at least about 14 dS/m, at least about 14.5 dS/m, at least about 15 dS/m, at least about 15.5 dS/m; at least about 16 dS/m, at least about 16.5 dS/m, at least about 17 dS/m, at least about 17.5 dS/m; at least about 18 dS/m, at least about 18.5 dS/m; at least about 19 dS/m, at least about 19.5 dS/m, at least about 20 dS/m, at least about 21 dS/m, at least about 22 dS/m, at least about 23 dS/m at least about 24 dS/m, at least about 25 dS/m, at least about 26 dS/m, at least about 27 dS/m, at least about 28 dS/m, at least about 29 dS/m, or at least about 30 dS/m, or more. In some embodiments, the elevated threshold salinity is assessed relative to a same plant species or cultivar without the genome edits. In some embodiments, the threshold salinity is elevated by at least about 1 dS/m, at least about 2 dS/m, at least about 3 dS/m, at least about 4 dS/m, at least about 5 dS/m, at least about 6 dS/m, at least about 7 dS/m, at least about 8 dS/m, at least about 9 dS/m, at least about 10 dS/m, at least about 11 dS/m, at least about 12 dS/m, at least about 13 dS/m, at least about 14 dS/m, or at least about 15 dS/m, or more.

In some embodiments, the engineered or transgenic plant has a particular slope (s) of a yield vs salinity (ECe) plot, or a decreased slope (s) of a yield vs salinity (ECe) plot. In some embodiments, the decreased slope is assessed relative to a same plant species or cultivar without the genome edits. In some embodiments, the slope is decreased by at least about 1% per dS/m, at least about 1.5% per dS/m, at least about 2.0% per dS/m, at least about 2.5% per dS/m, at least about 3.0% per dS/m, at least about 3.5% per dS/m, at least about 4.0% per dS/m, at least about 4.5% per dS/m, at least about 5.0% per dS/m, at least about 5.5% per dS/m, at least about 6.0% per dS/m, at least about 8% per dS/m, or at least about 10% per dS/m, or more. An example of a yield vs salinity (ECe) plot can be seen in FIG. 8.

In some embodiments, the engineered or transgenic plant has a particular starch content as a mature plant, or the seeds or root of the mature plant have can have a particular starch content. In some embodiments, the plant, root, fruit, or seeds of the mature plant can have at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

### Engineered and Transgenic Plant Parts

The invention also provides part of the engineered or transgenic plants of the invention. For example, the plant part can be a cell, a seed, a leaf, a shoot, a stem or a root. In certain embodiments, the plant part is a seed or a cell. The plant parts such as the seeds, may therefore comprise any of the combinations of genes of interest or transgenes as described above for the engineered or transgenic plants. For example, the seed comprising at least two genes of interest, wherein the first gene encodes a protein that controls the intracellular ion concentration and wherein the second gene encodes an antioxidant, wherein the gene that encodes a protein that controls the intracellular ion concentration is operatively linked to an enhancer element and the gene that encodes an antioxidant is operatively linked to an enhancer element. In other embodiments, the seed comprises at least two genes of interest, wherein the first and the second genes of interest encode proteins that control the intracellular ion concentration, wherein the first genes of interest encodes a plasma membrane protein that controls the intracellular ion concentration and wherein the second genes of interest encodes a tonoplast protein that controls the intracellular ion concentration, wherein the gene that encodes a plasma membrane protein that controls the intracellular ion concentration is operatively linked to an enhancer element and the gene that encodes a tonoplast protein is operatively linked to an enhancer element.

In other embodiments, the seed comprises at least four genes of interest, wherein the seed is a rice seed and wherein the genes of interest encode OsSOS1, OsNHX1, OsHKT1 and OsSODA1 and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element. In other embodiments, the seed comprises at least four genes of interest, wherein the seed is a rice seed and wherein the genes of interest encode OsSOS1, OsNHX1, OsHKT1 and OsAHA3 and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

For example, the seed comprising at least two transgenes, wherein the first transgene encodes a protein that controls the intracellular ion concentration and wherein the second transgene encodes an antioxidant. In other embodiments, the seed comprises at least two transgenes, wherein the first and the second transgenes encode proteins that control the intracellular ion concentration, wherein the first transgene encodes a plasma membrane protein that controls the intracellular ion concentration and wherein the second transgene encodes a tonoplast protein that controls the intracellular ion concentration. In other embodiments, the seed comprises at least four transgenes, wherein the seed is a rice seed and wherein the transgenes encode OsSOS1, OsNHX1, OsHKT1 and OsSODA1.

In some embodiments, the engineered rice seed comprises at least four genes of interest, wherein the genes of interest encode OsSOS1, OsNHX1, OsHKT1 and OsSODA1, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising an amino acid sequence that has at least 95% sequence identity to SEQ ID NO: 92, and

wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element.

In some embodiments, the engineered rice seed comprises at least four genes of interest, wherein the genes of interest encode OsSOS1, OsNHX1, OsHKT1 and OsAHA3, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsAHA3 gene comprises a polynucleotide sequence that encodes a polypeptide comprising an amino acid sequence that has at least 95% sequence identity to SEQ ID NO: 101, and

wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

In some embodiments, the engineered rice seed comprises at least four genes of interest, wherein the genes of interest encode OsSOS1, OsNHX1, OsSOS2 and OsVHA-A, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsSOS2 gene comprises a polynucleotide sequence that encodes a polypeptide comprising the amino acid sequence of SEQ ID NO: 100; wherein the OsVHA-A gene comprises a polynucleotide sequence that encodes a polypeptide comprising the amino acid sequence of SEQ ID NO: 102, wherein the gene that encodes OsSOS1 is operatively linked to an enhancer element; wherein the gene that encodes OsNHX1 is operatively linked to an enhancer element; wherein the gene that encodes OsSOS2 is operatively linked to an enhancer element and wherein the gene that encodes OsVHA-A is operatively linked to an enhancer element.

In some embodiments, the rice seed comprises at least four transgenes, wherein the transgenes encode OsSOS1, OsNHX1, OsHKT1 and OsSODA1, wherein the OsSOS1 transgene comprises a polynucleotide sequence that encodes a polypeptide comprising an amino acid sequence that has at least 95% sequence identity to SEQ ID NO: 95 and/or 96; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92.

In certain embodiments, the engineered rice seed comprises at least eight genes of interest, wherein the at least eight genes of interest encode OsSOS1, OsSOS2, OsAHA3, OsVHA-A, OsNHX1, OsHKT1, OsSODA1 and OsSOD2; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.

In certain embodiments, the rice seed comprises at least eight transgenes, wherein the transgenes encode OsSOS1, OsSOS2, OsAHA3, OsVHA-A, OsNHX1, OsHKT1, OsSODA1 and OsSOD2; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93.

In certain embodiments, the engineered rice seed comprising at least eight genes of interest wherein the at least eight genes of interest encode OsSOS1, OsSOS2, OsAHA3, OsVHA-A, OsNHX1, OsHKT1, OsSODA1 and OsSODCC1;

wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94; and

wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.

In other embodiments, the rice seed comprises at least eight transgenes wherein the transgenes encode OsSOS1, OsSOS2, OsAHA3, OsVHA A, OsNHX1, OsHKT1, OsSODA1 and OsSODCC1; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101;

wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSODCC1 transgene comprises a polynucleotide sequence that encodes a polypeptide comprising an amino acid sequence that has at least 95% sequence identity to SEQ ID NO: 94.

In some embodiments, the seed is capable of growth in a medium having a salt concentration greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, or about 30 g/L, about 32 g/L, about 34 g/L, about 36 g/L, about 38 g/L, about 40 g/L, about 42 g/L, about 44 g/L, about 46 g/L, about 48 g/L, about 50 g/L. In certain embodiments, the engineered or transgenic plant is capable of growing in a medium having a salt concentration greater than about 10 g/L, 20 g/L or 35 g/L. In further specific embodiments, the engineered or transgenic plant is capable of growing in a medium having a salt concentration greater than about 35 g/L, because this is greater than ocean salinity. The medium can have an electrical conductivity of a saturated paste extract (ECe) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some embodiments, the medium is liquid (e.g., for hydroponic growth strategies). In some embodiments, the medium is solid (e.g., soil, sand). In some embodiments, the medium is semi-solid. In some embodiments, the seed can have a starch concentration of at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

### Multicellular Structures

The invention provides a multicellular structure comprising one or more plant cells of the invention (i.e. the multicellular structure comprises one or more plant cells which may have any of the combinations of the genes of interest or the transgenes described above for the engineered or transgenic plants). The multicellular structure can be a whole plant, plant tissue, plant organ, plant part, plant reproductive material, or cultured plant tissue comprising one or more plant cells described herein. The multicellular structure can be a leaf, a shoot, a seed, a callus, a plantlet, a flower, or an in vitro-cultured bud comprising one or more plant cells described herein. As used herein, the term “callus” is generally intended to include regenerable plant tissue such as an embryogenic callus. As used herein, “plantlet” generally includes young or small plants used as propagules. Plantlets may be produced asexually by tissue culture or cell culture. As used herein, “in vitro-cultured bud” generally includes in vitro-propagated apical and axillary buds. Plant apical and axillary buds are small terminal or lateral protuberances on the stem of a vascular plant that may develop into a flower, leaf, or shoot. Plant buds arise from meristem tissue and may consist of overlapping immature leaves or petals. The multicellular structure can be a leaf, a shoot, a seed, a callus, a plantlet, a flower, or an in vitro-cultured bud derived from a plant described herein. The multicellular structure can be a whole plant, plant tissue, plant organ, plant part, plant reproductive material or cultured plant tissue derived from a plant described herein.

In some embodiments, the multicellular structure comprises one or more plant cells, wherein the one or more plant cells comprise at least four genes of interest, wherein the genes of interest encode OsSOS1, OsNHX1, OsHKT1 and OsSODA1, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92, wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element.

In some embodiments, the multicellular structure comprises one or more plant cells, wherein the one or more plant cells comprise at least four genes of interest, wherein the genes of interest encode OsSOS1, OsNHX1, OsHKT1 and OsAHA3, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsAHA3 gene comprises a polynucleotide sequence that encodes a polypeptide comprising the amino acid sequence of SEQ ID NO: 101, wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

In some embodiments, the multicellular structure comprises one or more plant cells, wherein the one or more plant cells comprise at least four genes of interest, wherein the genes of interest encode OsSOS1, OsNHX1, OsSOS2 and OsVHA-A, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsSOS2 gene comprises a polynucleotide sequence that encodes a polypeptide comprising the amino acid sequence of SEQ ID NO: 100; wherein the OsVHA-A gene comprises a polynucleotide sequence that encodes a polypeptide comprising the amino acid sequence of SEQ ID NO: 102, wherein the gene that encodes OsSOS1 is operatively linked to an enhancer element; wherein the gene that encodes OsNHX1 is operatively linked to an enhancer element; wherein the gene that encodes OsSOS2 is operatively linked to an enhancer element and wherein the gene that encodes OsVHA-A is operatively linked to an enhancer element.

In some embodiments, the multicellular structure comprises one or more plant cells, wherein the one or more plant cells comprise at least four transgenes, wherein the transgenes encode OsSOS1, OsNHX1, OsHKT1 and OsSODA1, wherein the OsSOS1 transgene comprises a polynucleotide sequence that encodes a polypeptide comprising an amino acid sequence that has at least 95% sequence identity to SEQ ID NO: 95 and/or 96; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92.

In certain embodiments, the multicellular structure comprises one or more plant cells, wherein the one or more plant cells comprise at least eight genes of interest, wherein the genes of interest encode OsSOS1, OsSOS2, OsAHA3, OsVHA-A, OsNHX1, OsHKT1, OsSODA1 and OsSOD2;

wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising a the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene is a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93;

wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element, wherein the gene that encodes OsSOS2 is operatively linked to an enhancer element; wherein the gene that encodes OsVHA-A is operatively linked to an enhancer element; wherein the gene that encodes OsAHA3 is operatively linked to an enhancer element and wherein the gene that encodes OsSOD2 is operatively linked to an enhancer element.

In certain embodiments, the multicellular structure comprises one or more plant cells, wherein the one or more plant cells comprise at least eight transgenes, wherein the transgenes encode OsSOS1, OsSOS2, OsAHA3, OsVHA-A, OsNHX1, OsHKT1, OsSODA1 and OsSOD2;

wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101;

wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93.

In certain embodiments, the multicellular structure comprises one or more plant cells, wherein the one or more plant cells comprise at least eight genes of interest, wherein the genes of interest encode OsSOS1, OsSOS2, OsAHA3, OsVHA-A, OsNHX1, OsHKT1, OsSODA1 and OsSODCC1;

wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising a the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene is a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94;

wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element, wherein the gene that encodes OsSOS2 is operatively linked to an enhancer element; wherein the gene that encodes OsVHA-A is operatively linked to an enhancer element; wherein the gene that encodes OsAHA3 is operatively linked to an enhancer element and wherein the gene that encodes OsSODCC1 is operatively linked to an enhancer element.

In other embodiments, the multicellular structure comprises one or more plant cells, wherein the one or more plant cells comprise at least eight transgenes wherein the transgenes encode OsSOS1, OsSOS2, OsAHA3, OsVHA A, OsNHX1, OsHKT1, OsSODA1 and OsSODCC1;

wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSODCC1 transgene comprises a polynucleotide sequence that encodes a polypeptide comprising an amino acid sequence that has at least 95% sequence identity to SEQ ID NO: 94.

### Methods of Producing Engineered Plants

The invention provides methods of making engineered plants, plant parts or multicellular structures according to the invention, the method comprising the steps of: i) introducing at least two enhancer elements as described herein into a cell of a plant, wherein the enhancer elements integrate into the genome of the cell of the plant such that they are operatively linked to the genes of interest, and ii) regenerating the cell to form an engineered plant, a plant part or a multicellular structure from the cell. In some embodiments, the enhancer elements are introduced into the cell by particle bombardment, Agrobacterium mediated transformation or by protoplast transfection. In certain embodiments, the enhancer elements are introduced into the cell by particle bombardment.

Programmable nucleases such as Cas endonucleases (e.g., Cas9, CpfI), transcription activator-like effector nucleases (TALENs), and zinc finger nucleases (ZFNs) allow precise genomic edits to be made. Therefore, in certain embodiments, methods of making engineered plants, plant parts or multicellular structures according to the invention, comprise the steps of:
(a) inducing callus formation from a seed;
(b) precipitating enhancer elements as described herein, guide RNAs and one or more nuclease(s) onto a microcarrier;
(c) transforming the callus with the microcarriers using particle bombardment to generate a transformed callus wherein the enhancer elements integrate into the genome of the engineered plant, the plant part or the multicellular structure such that they are operatively linked to the genes of interest;
(d) recovering the transformed callus to generate a multicellular structure which comprises the at least two genes of interest, wherein each gene of interest is operatively linked to an enhancer element.

In some embodiments, the multicellular structure is regenerated into an engineered plant, plant tissue, a plant organ, a plant part, plant reproductive material, or cultured plant tissue comprising one or more plant cells described herein. In certain embodiments the multicellular structure is regenerated into an engineered plant. In some embodiments, the enhancer element is stably integrated into the genome of the plant.

In some embodiments, the enhancer element is RNA, DNA or a plasmid. In certain embodiments, the enhancer element is DNA. DNA may be used rather than RNA, because it has greater stability both extracellular and within the cytoplasm and nucleus, it is less prone to errors during manufacturing and is less prone to errors during repairing Cas cut sites. DNA may be used rather than a plasmid, because DNA offers stable integration into the genome. DNA can often be integrated with smaller insertions which allows it to remain undetected for many generations and thus become a permanent feature of the new organism and its subsequent generations. When combined with CRISPR-associated (Cas) enzymes, DNA insertions safely integrate an insertion into the host genome with the remarkable absence of any vehicular DNA, plasmid DNA integration or any signatures to indicate a cut has been made in the genome. The safety of using these smaller DNA insertions as opposed to the much larger plasmid structures results precise and accurate insertions producing the exact intended output of the insertion/affected gene compared to some unpredictable consequences of introducing plasmid DNA into a cell.

In some embodiments, the nuclease is a Cas nuclease, Cpf1 nuclease, a TALEN or a zinc finger nucleases. In certain embodiments, the nuclease is Cpf1.

In some embodiments, the engineered plant, the plant part or the multicellular structure is not produced by a process that involves homologous recombination or are not produced by an essentially biological process.

### Methods of Producing Transgenic Plants

The invention provides methods of making transgenic plants, plant parts or multicellular structures according to the invention, the method comprising the steps of: i) introducing the at least two transgenes as described herein into a cell of a plant, wherein the transgenes stably integrate into the genome of the cell of the plant, and ii) regenerating the cell to form a transgenic plant, a plant part or a multicellular structure from the cell. In some embodiments, the transgenes are introduced into the cell by particle bombardment, Agrobacterium mediated transformation or by protoplast transfection. In certain embodiments, the transgenes are introduced into the cell by particle bombardment.

Programmable nucleases such as Cas endonucleases (e.g., Cas9, CpfI), transcription activator-like effector nucleases (TALENs), and zinc finger nucleases (ZFNs) allow precise genomic edits to be made. Therefore, in certain embodiments, methods of making transgenic plants, plant parts or multicellular structures according to the invention, comprise the steps of:
(a) inducing callus formation from a seed;
(b) precipitating a polynucleotide sequence, a guide RNA and a nuclease onto a microcarrier; wherein the polynucleotide sequence comprises the at least two transgenes as described herein;
(c) transforming the callus with the microcarriers using particle bombardment to generate a transformed callus wherein the polynucleotide sequence integrates into the genome of the transgenic plant, the plant part or the multicellular structure;
(d) recovering the transformed callus to generate a multicellular structure which comprises the at least two transgenes.

In some embodiments, the multicellular structure is regenerated into a transgenic plant, plant tissue, a plant organ, a plant part, plant reproductive material, or cultured plant tissue comprising one or more plant cells described herein. In certain embodiments the multicellular structure is regenerated into a transgenic plant. In some embodiments, the polynucleotide sequence is stably integrated into the genome of the plant.

In some embodiments, the polynucleotide is RNA, DNA or a plasmid. In certain embodiments, the polynucleotide is DNA. DNA may be used rather than RNA, because it has greater stability both extracellular and within the cytoplasm and nucleus, it is less prone to errors during manufacturing and is less prone to errors during repairing Cas cut sites. DNA may be used rather than a plasmid, because DNA offers stable integration into the genome. DNA can often be integrated with smaller insertions which allows it to remain undetected for many generations and thus become a permanent feature of the new organism and its subsequent generations. When combined with CRISPR-associated (Cas) enzymes, DNA insertions safely integrate an insertion into the host genome with the remarkable absence of any vehicular DNA, plasmid DNA integration or any signatures to indicate a cut has been made in the genome. The safety of using these smaller DNA insertions as opposed to the much larger plasmid structures results precise and accurate insertions producing the exact intended output of the insertion/affected gene compared to some unpredictable consequences of introducing plasmid DNA into a cell.

In some embodiments, the nuclease is a Cas nuclease, Cpf1 nuclease, a TALEN or a zinc finger nucleases. In certain embodiments, the nuclease is Cas9 or Cpf1.

In some embodiments, the transgenic plant, the plant part or the multicellular structure is not produced by a process that involves homologous recombination or are not produced by an essentially biological process.

### Use of the Engineered Plants and Transgenic Plants

In another embodiment, the present invention provides a method of producing flour, wholemeal, starch or other product obtained from seed, the method comprising; a) obtaining seed of the invention, and b) extracting the flour, wholemeal, starch or other product.

In another embodiment, the present invention provides a method of processing rice, the method comprising; a) obtaining seed of the invention, b) removing the husks, c) milling the shelled rice to remove the bran layer. In some embodiments, the method involves the step of whitening the rice. In some embodiments, the method involves the step of polishing the rice.

In another embodiment, the present invention provides the use of an engineered plant of the invention, or part thereof, as animal feed, or to produce feed for animal consumption or food for human consumption. In another embodiment, the present invention provides the use of a transgenic plant of the invention, or part thereof, as animal feed, or to produce feed for animal consumption or food for human consumption.

### MODES FOR CARRYING OUT THE INVENTION

The following examples are offered for illustrative purposes only, and are not intended to limit the scope of the present invention in any way.

### Example 1—Production of Genetic Inserts for Engineered Rice

Genetic inserts were designed to stably introduce enhancer elements into the genome of rice using Cas9 and Cpf1 nucleases. These enhancer elements were introduced such that they were operatively linked to genes of interest which encode a variety of proteins with different functions including a plasma membrane ion transporter; a plasma membrane hydrogen exporting ATPase, a tonoplast hydrogen exporting ATPase, a tonoplast ion transporter, a potassium transporter, a protein kinase and an antioxidant.

The guide RNAs (gRNAs) used for each of the genes of interest are listed in Table 3. A summary of the enhancer elements and the corresponding genes of interest are listed in Table 4. Cas9 homology arms were added to the 5′ and 3′ end of some of the inserts as summarised in Table 5. The enhancer elements were designed to have two purposes: the first is to increase the expression of the genes of interest in order to provide immediate relief from salt stress. The second is to dilute the gene expression of other stress-induced genes. The stress-induced genes that are being diluted are a collective group that prevent plant growth and prevent the plant metabolism from full functioning when under stress. Without wishing to be bound by any particular theory, by diluting the effective expression of the stress-induced genes this will weaken their initial effect, while the increased expression of the genes of interest into the plants should establish a fair and beneficial salt distribution.

The genes of interest that encode antioxidants were operably linked to the enhancer elements that had the same polynucleotide sequence to ensure that they are expressed in concert. The enhancer elements for the antioxidants were design to ensure that antioxidants were expressed at critical moments in the development of a plant to ‘clean up’ reactive oxygen species, while also providing a heightened baseline level across the lifetime and tissues of the plant. The baseline expression of the antioxidants was achieved by the presence of a TATA box in the enhancer element, which is a constitutive and ubiquitously expressed motif, which encourages plant wide expression. The enhancer element for the antioxidant also includes a gibberellic acid responsive element. Gibberellic acid (GA) promotes expression within the early developmental stages and focuses the expression of the antioxidant genes in the epidermal roots. The presence of an ethylene responsive element in the enhancer element results in the expression of the antioxidant genes through the later developmental stages of the plant, with specific expression during the flowering and fruit development stages. The enhancer element for the antioxidant also contained a DREB2A element, which is a stress-inducible promoter element reactive to ethylene.

Auxin begins to express after the initial germination and predominantly at the axial locations of both stems and roots (root cap & central root). It continues to express throughout the life cycle of the plant and is supported by ethylene at the flowering and fruit development stage (as seen FIG. 7). Auxin elements were used in the enhancer elements for the SOS1/SOS2/AHA group rather gibberellin, as auxin expresses heavily at the end of roots, especially where roots begin to grow. This can be exploited to create a consistent production of these key proteins that can then implement into the cell membrane of roots as they grow. Over time these roots will slow their growth and the intensity of expression will create a gradient from tip to further back along the root. This allows a gradient to be established that shuttles salt to the tip of the root and propels it out of the root.

TABLE 3
Guide RNAs for each of the genes of interest. The functions
of the proteins that these genes of interest encode and the
rice database accession number of the gene are provided.

| Gene | Function | Accession No. | Guide RNA sequence |
|---|---|---|---|
| NHX1 | Tonoplast Na+/H+ | Os07t0666900 | TTTACCGGGAATTGTCGAATTAGGC |
| | antiporters | | (SEQ ID NO: 1) |
| VHA-A | Tonoplast H+-ATPase | Os06g0662000 | GTGATCTGACCGCCGG |
| | | | (SEQ ID NO: 2) |
| SOS1 | Na+/H+ antiporter | Os12g0641100 | TTTGCCTTGGATTTTCCACTTGCA |
| | | | (SEQ ID NO: 3) |
| SOS2 | Serine/threonine | Os06g0606000 | GAGTTGGATTCGTGGCCGCGCGG |
| | protein kinase | | (SEQ ID NO: 4) |
| AHA3 | Plasma membrane H+- | Os12g0638700 | TGGTGGGAGATCCATCTGCGAGG |
| | ATPase | | (SEQ ID NO: 5) |
| HKT1 | High-affinity K+ | Os06g0701700 | TTTCTCATACTCGTTGGCTCGTTGC |
| | transporter | | (SEQ ID NO: 6) |
| SODA1 | mitochondrial | Os05t0323900 | CCCATCCTCCAGTGCTACGG |
| | mangansese superoxide | | (SEQ ID NO: 7) |
| | dismutase | | |
| SODCC1 | cytoplasmic and ECM | Os03g0351500 | TTTAGGACCTCTAGAGGCTACTTCTG |
| | CuZn superoxide | | CC (SEQ ID NO: 8) |
| | dismutase | | |
| SOD2 | cytoplasmic CuZn | Os03g0219200 | GCGAAGCGGCAGAGAATGGCAGGGA |
| | superoxide dismutase | | AA (SEQ ID NO: 9) |

TABLE 4
The sequences for the enhancer elements introduced
to the 5′ end of the genes of interest

| Gene | Promoter elements | Enhancer element sequence |
|---|---|---|
| NHX1 | DREB.GA.TAF.TATATA | GCCGACCCTTTGGCCACGTGGCAATATATA |
| | | (SEQ ID NO: 10) |
| VHA-A | TATA/TAF-1 + ETH | GCCGACGCCACGTGGCAAGCCGCCTATATA |
| | | (SEQ ID NO: 11) |
| SOS1 | AUX.ETH.E2F.DREB.E2F | TGTCTCGCCGCCGCGGGAAA |
| | | (SEQ ID NO: 12) |
| SOS2 | AUX/ETH + E2F-1 (*2) + | TGTCTCGCCGCCGCGGGAAAAGCCGACGCGGGA |
| | TATA/DREB?? | AAA (SEQ ID NO: 13) |
| AHA3 | AUX/ETH + E2F-1 (*2) + | TGTCTCGCCGCCGCGGGAAAAGCCGACGCGGGA |
| | TATA/DREB?? | AAA (SEQ ID NO: 14) |
| HKT1 | DREB2Ax2 + E2F-1 | GCTCAAGCCGACGCGGGAAAGGCCGACGCCGCC |
| | | GCCGAC (SEQ ID NO: 15) |
| SODA1 | GA + TATA + DREB2A + ETH | CCTTTGTATATAGCCGACGCCGACGCCGCC |
| | | (SEQ ID NO: 16) |
| SODCC1 | GA + TATA + DREB2A + ETH | CCTTTGTATATAGCCGACGCCGAC |
| | | (SEQ ID NO: 17) |
| SOD2 | GA + TATA + DREB2A + ETH | CCTTTGTATATAGCCGACGCCGACGCCGCC |
| | | (SEQ ID NO: 18) |

TABLE 5

| Gene | Enzyme | Location | DNA Inserts COMPLETE w/homology arms |
|---|---|---|---|
| NHX1 | Cpf1 | 5′ | GCAT/GCCGACCCTTTGGCCACGTGGCAATATATAtt |
| | | | (SEQ ID NO: 19) |
| | | 3′ | ATCGaaTATATATTGCCACGTGGCCAAAGGGTCGGC |
| | | | (SEQ ID NO: 20) |
| VHA-A | Cas9 | 5′ | CGAGAGAGCCGTCTCCCTCTTCGCTTCTCCTCTCCCCCC |
| | | | CGTGATCTGACGCCGACGCCACGTGGCAAGCCGCCTAT |
| | | | ATACGCCGGCGACGACCCCCTCCCACCACCCGCCGCCG |
| | | | CCGCCGCGCCCCCGC (SEQ ID NO: 21) |
| SOS1 | Cpf1 | 5′ | ACTTaa/TGTCTCGCCGCCGCGGGAAAAGCCGACGCGGGAAAA |
| | | | (SEQ ID NO: 22) |
| | | 3′ | AAGT . . . TTTTCCCGCGTCGGCTTTTCCCGCGGCGGCGAGA |
| | | | CAtt (SEQ ID NO: 23) |
| SOS2 | Cas9 | 5′ | CGGCGGCGTCGTCGTCTTCCTCCTCTTCCCCCCGAGTTG |
| | | | GATTCGTGGCCTGTCTCGCCGCCGCGGGAAAAGCCGAC |
| | | | GCGGGAAAACGGCGGATGGGAGGGGAGGAGGGAATGG |
| | | | CGGCGGGGAGGAAGAAGCGGGT (SEQ ID NO: 24) |
| AHA3 | Cas9 | 5′ | GATACAGGTAGAGAGAGGTGAGAAGGCAGTGGTGGGA |
| | | | GATCCATCTTGTCTCGCCGCCGCGGGAAAAGCCGACGC |
| | | | GGGAAAAGCGAGGTGCCTCCATGGCTGAGAAGGAGGG |
| | | | CAACCTCGACGCCGTCCTCAAGGA (SEQ ID NO: 25) |
| HKT1 | Cpf1 | 5′ | GCTCAAGCCGACGCGGGAAAGGCCGACgccgccGCCGAC |
| | | | (SEQ ID NO: 26) |
| | | 3′ | GAGC . . . GTCGGCGGCGGCGTCGGCCTTTCCCGCGTCGGCtt |
| | | | (SEQ ID NO: 27) |
| SODA1 | Cas9 | 5′ | ACCTGCCACTCGCCCACTCCTCCTCCTCCCCCATCCTCC |
| | | | AGTGCCTTTGTATATAGCCGACGCCGACGCCGCCCTAC |
| | | | GGTGTCACGCAGCCATGGCGCTCCGCACGCTGGCCTCG |
| | | | AGGAAAACC (SEQ ID NO: 28) |
| SODCC1 | Cpf1 | 5′ | CTGCaaCCTTTGTATATAGCCGACGCCGACGCCGCC |
| | | | (SEQ ID NO: 29) |
| | | 3′ | gcagGGCGGCGTCGGCGTCGGCTATATACAAAGGtt |
| | | | (SEQ ID NO: 30) |
| OsSOD2 | Cpf1 | 5′ | CCTTTGTATATAGCCGACGCCGACGCCGCCggGCGA |
| | | | (SEQ ID NO: 31) |
| | | 3′ | ccGGCGGCGTCGGCGTCGGCTATATACAAAGGtcgc |
| | | | (SEQ ID NO: 32) |

The 5′ and 3′ sequences of the DNA inserts. The residues that are in lower case were included in the design to ensure that the gene was in frame. The /denotes residues that form the 4-5 base pair overhang for the Cpf1 nucleases

### Example 2—Production of Engineered Plants

### Step 1—Callus Induction

Seeds were de-hulled by hand or using tweezers. The surface of the seeds was then sterilized with 75% ethanol for 1 min, followed by 2.5% sodium hypochlorite for 15 mins and then washed with distilled water 8 times. The seeds husks were then autoclaved. The seeds were then placed on rice Callus Induction Media. The components of this media are listed in Table 6 below. The seeds were incubated in the dark for 14 hrs at 25° C. This produced embryogenic calli, which looked white or yellowish and had a compact & nodular structure.

TABLE 6
The components of Rice Callus Induction Media
Rice Callus Induction Media

| | |
|---|---|
| 4.3 g L−1 | Murashige & Skoog Basal Salts and Vitamins |
| 30 g L−1 | Maltose |
| 0.3 g L−1 | Casein Hydrolysate |
| 0.6 g L−1 | L-Proline |
| 3 mg L−1 | 2,4-Dichlorophenoxyacetic acid (2,4-D) |
| 0.25 mg L−1 | 6-Benzylaminopurine (BAP) |
| 3 g L−1 | Phytagel |
| 50 g L−1/90 mg L−1 | Kanamycin/Cefotaxime (either antibiotic) |
| 20 mg L−1 | Carbendazim (antifungal) |

After 14 days the embryogenic calli were cut from the seed and further split into equal segments no larger than 5 mm in diameter. These calli were transferred onto fresh on Rice Callus Induction Media and incubated in the same conditions (24 h dark @25° C.) for 4 days.

### Step 2—Particle Bombardment

The DNA inserts designed in Example 1 were transformed into rice using partial bombardment. Each particle bombardment consisted of a maximum number of 4 DNA inserts per bombardment. Therefore, to bombard the embryonic calli with eight DNA inserts multiple sets of bombardments were required. The embryogenic calli were bombarded with the following sets of DNA inserts, which comprise the sequences as described in Table 5 to produce engineered plants as summarized in the table below. The variety of rice used to create the embryogenic calli is also indicated in the table.

### Experiment 1

TABLE 7
The combinations of genes of interest targeted in each rice plant.

| Plant | Genes of interest |
|---|---|
| Genotype 1 | OsNHX1, OsSOS1, OsHKT1, OsSODA1 |
| Genotype 2 | OsNHX1, OsSOS1, OsHKT1, OsSODA1 |
| Genotype 9 | OsNHX1, OsSOS1, OsHKT1, OsSODA1 |
| Genotype 10 | OsNHX1, OsSOS1, OsHKT1, OsSODA1 |

### Experiment 2

SET ONE: NHX1, SOS1, AHA3 and HKT1
SET TWO: VHA-A, SOS2, SODA1 and SOD2

TABLE 8
The combinations of genes of interest targeted in each
rice plant. The rice variety used is also indicated.

| Plant | | |
|---|---|---|
| Sample | Rice variety | Genes of interest |
| A | Hayayuki | OsNHX1, OsSOS1, OsAHA3, OsHKT1 |
| B | Hayayuki | OsNHX1, OsSOS1, OsAHA3, OsHKT1 |
| C | Hayayuki | OsNHX1, OsVHA-A, OsSOS1, OsSOS2, |
| | | OsAHA3, OsHKT1, OsSODA1, OsSOD2 |
| D | Truong | OsNHX1, OsSOS1, OsAHA3, OsHKT1 |
| | Giang | |
| E1 | Hayayuki | OsNHX1, OsVHA-A, OsSOS1, OsSOS2, |
| | | OsAHA3, OsHKT1, OsSODA1, OsSOD2 |
| F | Hayayuki | OsNHX1, OsVHA-A, OsSOS1, OsSOS2, |
| | | OsAHA3, OsHKT1, OsSODA1, OsSOD2 |
| I | Hayayuki | OsNHX1, OsSOS1, OsAHA3, OsHKT1 |
| G | Hayayuki | OsNHX1, OsVHA-A, OsSOS1, OsSOS2, |
| | | OsAHA3, OsHKT1, OsSODA1, OsSOD2 |
| 1 | Hayayuki | OsNHX1, OsVHA-A, OsSOS1, OsSOS2, |
| | | OsAHA3, OsHKT1, OsSODA1, OsSOD2 |
| 9 | Hayayuki | OsNHX1, OsVHA-A, OsSOS1, OsSOS2, |
| | | OsAHA3, OsHKT1, OsSODA1, OsSOD2 |

In order for the DNA inserts to stably integrate into the genome they need to be bombarded in combination with a programmable nuclease (Cas9 or Cpf1) and at least one RNA guide as shown in Table 3. Cas9 requires two RNA guides, crRNA and tracrRNA to stably insert the DNA insert that comprises the enhancer element into the plant genome, whereas Cpf1 requires only a single guide RNA. The ratio of nuclease enzyme to RNA guide to genetic DNA insert was 1:1:2 per gene. The DNA/enzyme mixture was created as outlined below.

| | Per bombardment | |
|---|---|---|
| Enzyme (Cpf1 or Cas9) | 0.5 μl (5 ug) | |
| RNA Guide 1 | 0.5 | μl |
| RNA Guide 2 | 0.5 | μl |
| RNA Guide 3 | 0.5 | μl |
| RNA Guide 4 | 0.5 | μl |

Incubated 15 mins at room temperature

| | | |
|---|---|---|
| DNA Insert 1 | 1 | μl |
| DNA Insert 2 | 1 | μl |
| DNA Insert 3 | 1 | μl |
| DNA Insert 4 | 1 | μl |

| | |
|---|---|
| Buffer | up to 20 ul |

The DNA/enzyme mixture was precipitated onto gold particles by mixing the gold particles with the DNA/enzyme mixture, spermidine, and calcium chloride. The resulting mixture was incubated on ice for 10 minutes. The DNA coated particles were centrifuged, the supernatant removed and replaced with 75% ethanol, and the gold particles were re-suspended.

The embryogenic calli were placed on a petri dish with rice osmotic medium for 4 hours before bombardment took place. The components of the rice osmotic medium are summarised in Table 9 below. Alternatively, the calli were transferred to Whatman™ filter paper and desiccated for 24 hr in the dark at 25° C. The calli were then transferred to a gene gun system, where under a vacuum pressure of at least −5 Hg, 10 μl of prepared DNA-coated gold particles are fired at least 1100 psi.

After bombardment the calli were placed on rice Osmotic Medium at 26° C. in the dark for 16 hr-20 hr. The calli were then transferred to rice Callus Induction Media and incubated in the dark for 7-14 days at 25° C.

TABLE 9
The components Rice osmotic medium and Rice callus induction media

| Rice Osmotic Media (ROM) | | Rice Callus Induction Media (RCIM) | |
|---|---|---|---|
| 4.3 g L−1 | Murashige & Skoog Basal | 4.3 g L−1 | Murashige & Skoog Basal |
| | Salts and Vitamins | | Salts and Vitamins |
| 30 g L−1 | Maltose | 30 g L−1 | Maltose |
| 90 g L−1 | Mannitol | 0.3 g L−1 | Casein Hydrolysate |
| 0.3 g L−1 | Casein Hydrolysate | 0.6 g L−1 | L-Proline |
| 0.6 g L−1 | L-Proline | 3 mg L−1 | 2,4-Dichlorophenoxyacetic |
| | | | acid (2,4-D) |
| 3 mg L−1 | 2,4- Dichlorophenoxyacetic | 0.25 mg L−1 | 6-Benzylaminopurine |
| | acid (2,4-D) | | (BAP) |
| 0.25 mg L−1 | 6-Benzylaminopurine | 50 g L−1/90 mg L−1 | Kanamycin/Cefotaxime |
| | (BAP) | | (either antibiotic) |
| 3 g L−1 | Phytagel | 20 mg L−1 | Carbendazim (antifungal) |
| 50 mg L−1/90 mg L−1 | Kanamycin/Cefotaxime | | |
| | (either antibiotic) | | |
| 20 mg L−1 | Carbendazim (antifungal) | | |

### Step 3—Regeneration of Embryogenic Calli

The calli were then transferred to ice Regeneration Media I (RRM I) and incubated under light conditions (16 h light: 8 h dark at 25° C.) for one to three weeks. After around 7 days green spots started to appear on some calli, which developed into shoot and root growth to form a platelet. After 3 weeks, or before the plantlets got too big for transfer, the platelets and remaining calli were transferred onto Rice Regeneration Media II (as described in Table 10 below) and incubated under light conditions (16 h light: 8 h dark at 25° C.).

TABLE 10
The components of Rice Regeneration Media I and II)

| Rice Regeneration Media I (RRM I) | | Rice Regeneration Media II (RRM II) | |
|---|---|---|---|
| 4.3 g L−1 | Murashige & Skoog Basal | 4.3 g L−1 | Murashige & Skoog Basal |
| | Salts | | Salts |
| 30 g L−1 | Maltose | 30 g L−1 | Maltose |
| 2 g L−1 | Casein Hydrolysate | 100 mg L−1 | Myo-inositol |
| 30 g L−1 | Sorbitol | 60 mg L−1 | Calcium Silicate |
| 0.5 mg L−1 | Naphthaleneacetic acid | 3 g L−1 | Gelzan |
| | (NAA) | | |
| 1.5 mg L−1 | Kinetin | 50 mg L−1 | Cefotaxime or Vancomycin |
| 0.5 mg L−1 | BAP | 20 mg L−1 | Carbendazim (antifungal) |
| 0.5 mg L−1 | TDZ | | |
| 4 g L−1 | Phytagel | | |
| 50 mg L−1 | Cefotaxime or Vancomycin | | |
| 20 mg L−1 | Carbendazim (antifungal) | | |

In order to generate more root tissue growth the platelets were transferred Rice Root Proliferation Media (RRPM) (described in Table 11) and incubated under light conditions (16h light: 8 h dark at 25° C.) until the root mass was sufficient to ensure healthy plant growth.

TABLE 11
The components of Rice Root Proliferation Media
Rice Root Proliferation Media (RRPM)

| | |
|---|---|
| 2.15 g L−1 | Murashige & Skoog Basal Salts and Vitamins |
| 20 g L−1 | Maltose Note don't add sugar for growth in hydroponics or |
| | with bacteria |
| 60 mg L−1 | Calcium Silicate |
| 2 g L−1 | Gelzan Note - Gelzan & Phytogel are interchangeable |
| 50 mg L−1 | Vancomycin/Cefotaxime (either antibiotic) dissolved in |
| | distilled water |
| 20 mg L−1 | Carbendazim (antifungal) dissolved in methanol/ethanol |

### Example 3—Optimisation of Callus Induction Rates

The callus induction rates for different rice varieties was tested using the methods described in Example 3. The effect of different concentrations of the hormones 2,4-dichlorophenoxyacetic acid (2,4-D) and 6-benzylaminopurine (BAP) on callus induction rates was investigated.

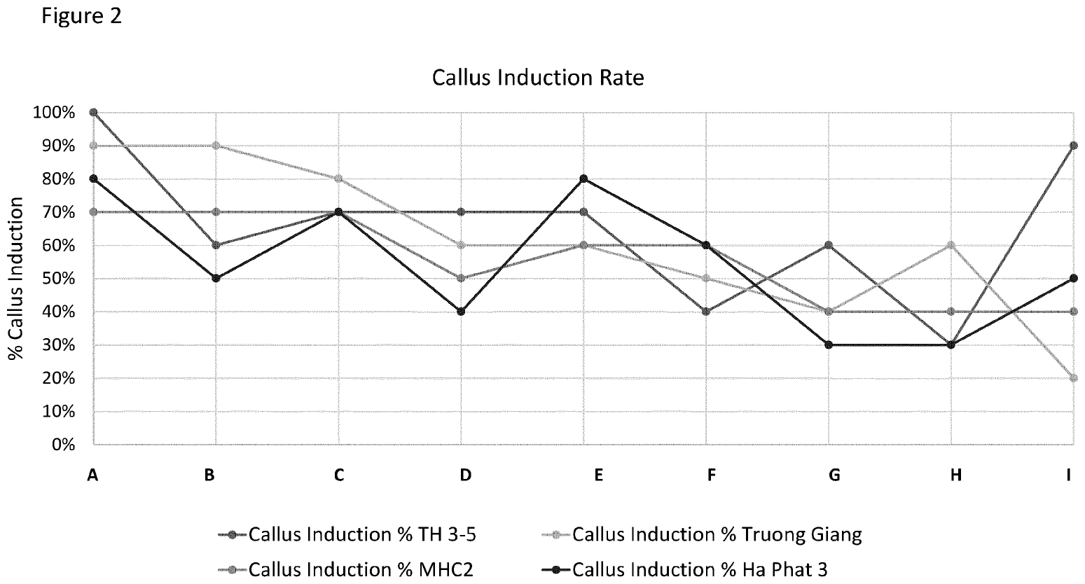

Four different rice varieties were tested: TH 3-5, Truong Giang, MHC2 and Ha Phat 3. The calli were induced as described in step 1 of Example 3. The concentrations of 2,4-D and BAP tested in the rice callus induction media are summarised in Table 12 below. As shown in FIG. 2, calli were successfully produced from all four rice varieties. Media comprising 2 mg/L of 2,4-D and 0.1 mg/l of BAP produced the highest induction rates.

TABLE 12
The concentrations of 2,4-D and BAP tested
in the Rice callus induction media

2,4 D mg/L

| | | | |
|---|---|---|---|
| Media Chart | 2 | 2.5 | 3 |

| | | | | |
|---|---|---|---|---|
| BAP | 0.1 | A | B | C |
| mg/L | 0.25 | D | E | F |
| | 0.5 | G | H | I |

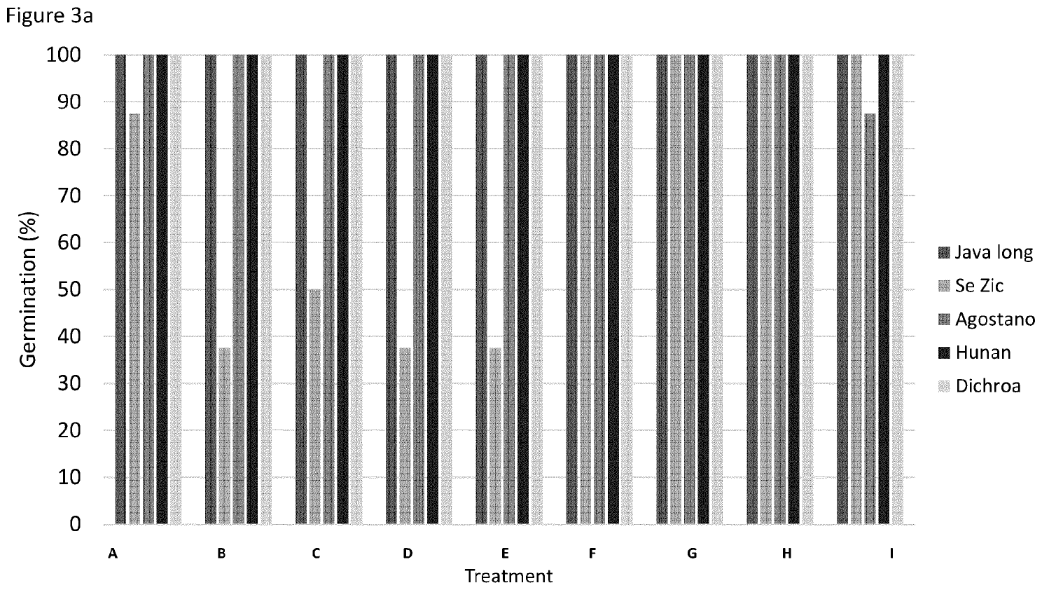

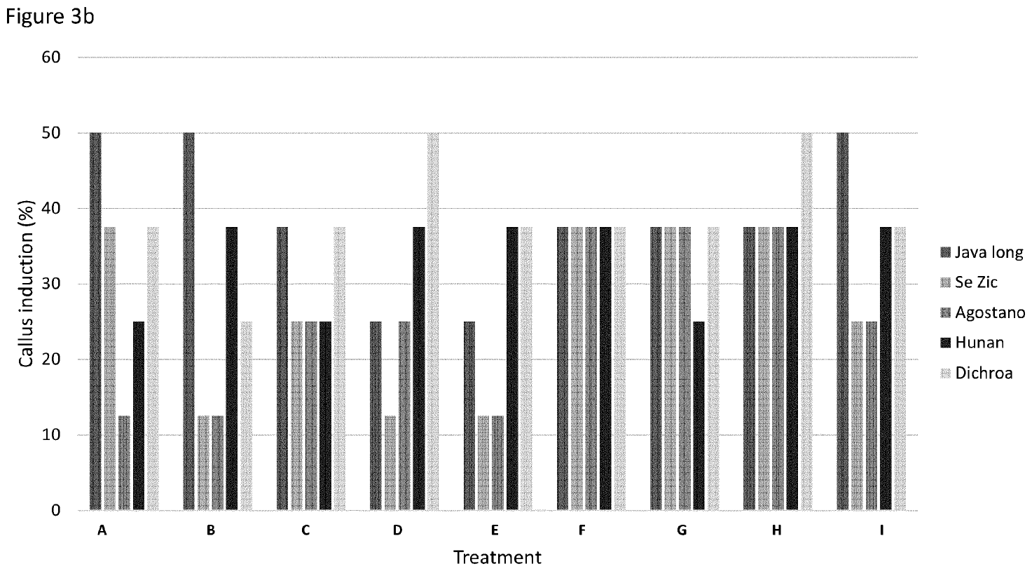

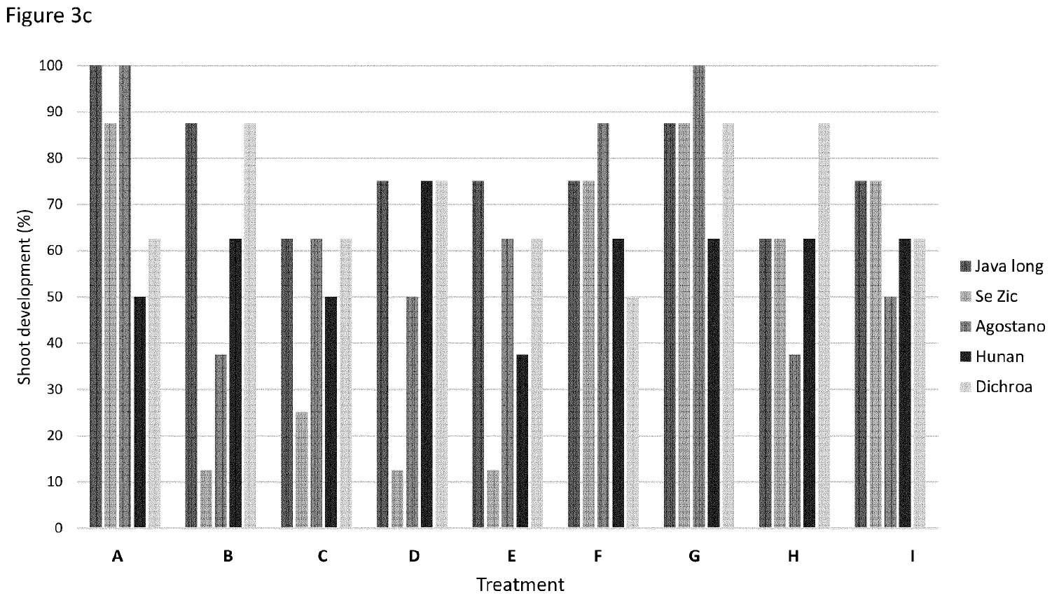

The callus induction rate in five further rice varieties were tested: Java long, Se Zic, Agostano, Hunan and Dichroa. FIG. 3a demonstrates the percentage germination of each of the rice varieties and FIG. 3b demonstrates the percentage of callus induction. These data demonstrate that calli could be produced from all of the varieties tested. FIG. 3c demonstrates that shoot development from these induced calli can also occur all of the varieties tested. These data demonstrate the utility of the callus induction methods for different plant varieties.

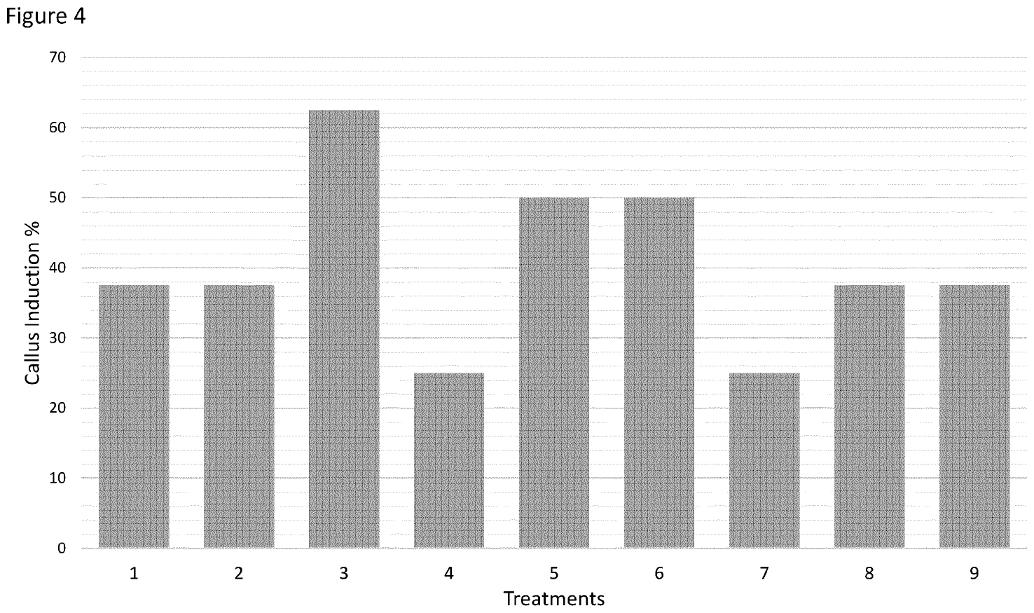

The rate of callus induction was also determined for the rice variety Hayayuki. Calli were induced as described in step 1 of Example 3. The concentrations of 2,4-D and BAP tested in the Rice callus induction media are summarised in Table 13 below. FIG. 4 demonstrates that all of the treatments tested resulted in calli induction of Hayayuki. The combination of 3 mg/L of 2,4-D and 0.1 mg/l of BAP produced the highest induction rates.

TABLE 13
The concentrations of 2,4-D and BAP tested
in the Rice callus induction media

| | | | | | | | | | |
|---|---|---|---|---|---|---|---|---|---|
| BAP | 0.10 | 0.10 | 0.10 | 0.25 | 0.25 | 0.25 | 0.50 | 0.50 | 0.50 |
| (mg/L) | | | | | | | | | |
| 2,4-D | 2.00 | 2.50 | 3.00 | 2.00 | 2.50 | 3.00 | 2.00 | 2.50 | 3.00 |
| (mg/L) | | | | | | | | | |
| Treat- | 1 | 2 | 3 | 4 | 5 | 6 | 7 | 8 | 9 |
| ment | | | | | | | | | |

### Example 4—Analysis of Regenerated Engineered Plants

The engineered plants produced in Example 2 were analysed using qRT-PCT to determine the expression levels of certain genes compared to wild type plants.

### RNA Extraction

Root (5-10 mg) and shoot (˜15 mg) tissue were collected from the regenerated plants from experiment 1 in Example 2 and flash frozen in liquid nitrogen and stored at −80° C. until required. In addition, samples were collected which contained tissue from the whole regenerated engineered plant from experiment 2 in Example 2. These samples predominantly contained leaf tissue.

These samples were subsequently ground in a mortar and pestle with liquid nitrogen. Total RNA was extracted using a GeneJet Plant RNA Purification Kit or Qiagen Plant RNEasy kit, following the manufacturer's protocol. The concentration of the RNA was analysed using a NanoDrop OneC microvolume UV-Vis Spectrophotometer. All the extracted RNAs with a 260/230 ratio of above 2.0 (2.01-2.14) were used for downstream processes. The DNA was removed from the sample by treating with RQ1 RNase-Free DNase at 37° C. for 30 minutes. The DNase was then inactivated by incubating the mixtures at 65° C. for 10 minutes.

qRT-PCR Analysis

qRT-PCR was used to analyze the gene expression of various salt tolerance genes within the engineered plant. Two sets of probe/dye sets were tested. Both sets included a 5th primer/probe of the housekeeping gene Actin to act as a control.
SET ONE: NHX1, SOS1, AHA3 and HKT1
SET TWO: VHA-A, SOS2, SODA1 and SOD2/SODCC1

The probes attached to each gene were: FAM: NHX1 and VHA-A; SUN: SOS1 and SOS2; ROX: AHA3 and SODA1; and Cy5′: HKT1, SODCC1 and SOD2. A mixture of 12.5 μl iTaq Reaction Mix, 0.5 μl iScript Reverse, 5×1 μl each of a forward and reverse primer for each genes of interest (provided in table X), 5×0.8 μl Fluorogenic Probe and 1 μl of RNA was placed in a PCT machine with the following cycle: 1 cycle 50° C. for 30 mins, 1 cycle 94° C. for 2 mins, 40 cycles 94° C. for 15 secs, 59° C. for 15 secs, 68° C. for 15 secs, 1 cycle 68° C. for 5 mins. The primers used for each genes of interest were:

NHX1
(SEQ ID NO: 50)
TGCAATTGGAGCCATCTTTTCTGCG
VHA-A
(SEQ ID NO: 51)
AATGCCTGCGGATAGTGGTTACCC
SOS1
(SEQ ID NO: 52)
TGTTACATTCCCTCAGGTGCTTCGTG
SOS2
(SEQ ID NO: 53)
TGTCACCAGCAACCTTTCGAACATCA
AHA3
(SEQ ID NO: 54)
TGGCAATTGGAAAAGAAACAGGGCG
HKT1
(SEQ ID NO: 55)
TGGGAATGTAGGGCTATCCACTGGT
SODA1
(SEQ ID NO: 56)
CCAAAATCCTCATCAATGGCCCAGC
SODCC1
(SEQ ID NO: 57)
ACTGGGCCACACTACAATCCTGC
SOD2
(SEQ ID NO: 58)
ACTACAACTCACAGGATGCAGCAGC

The fluorescence from probes was analyzed using Bio-Rad CFX Maestro software. The relative transcript abundances of genes in regenerated independent-event Rice plants were analyzed compared to the Cq values in wild type plants. All reactions were performed with two technical replicates per sample. Transcript levels were first analysed relative to Actin and/or 25S ribosomal RNA (25S rRNA) housekeeping genes, with the design based on Rice genome sequence to get the ΔCt values. The relative fold-change gene expressions in regenerated independent-event Rice plants were then analyzed relative to wild type by calculating the ΔΔCt values.

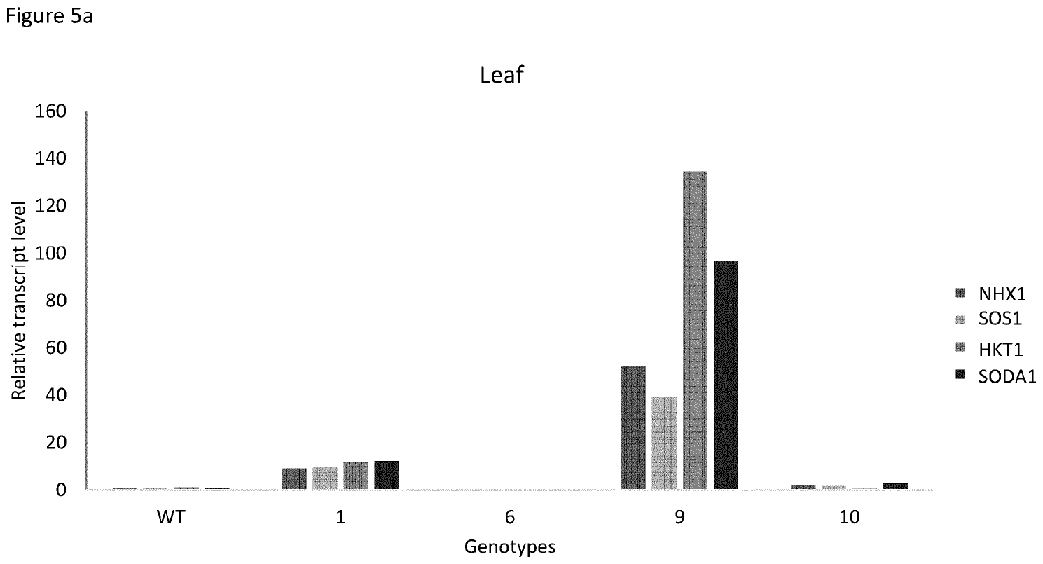

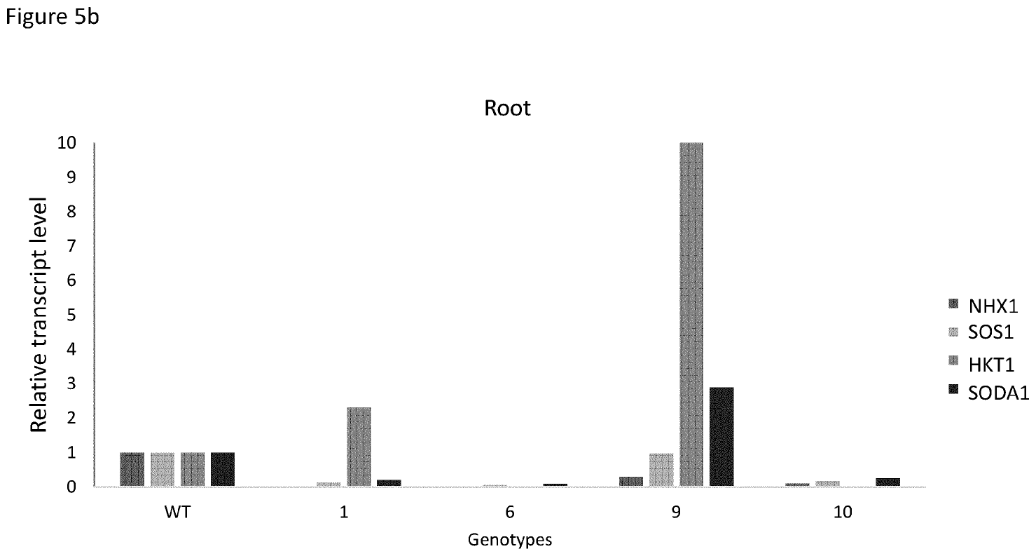

FIGS. 5a and 5b demonstrate that the RNA expression levels of NHX1, SOS1, HKT1 and SODA1 in either the shoot or the root of the engineered rice were significantly higher than compared to the wild type rice. These data demonstrate that the RNA expression levels of NHX1, SOS1, HKT1 and SODA1 were increased compared to a wild type plant. The expression level of the genes of interest varied between the root and the shoot. For example, NHX1 had a higher level of expression in the leaf compared to the root.

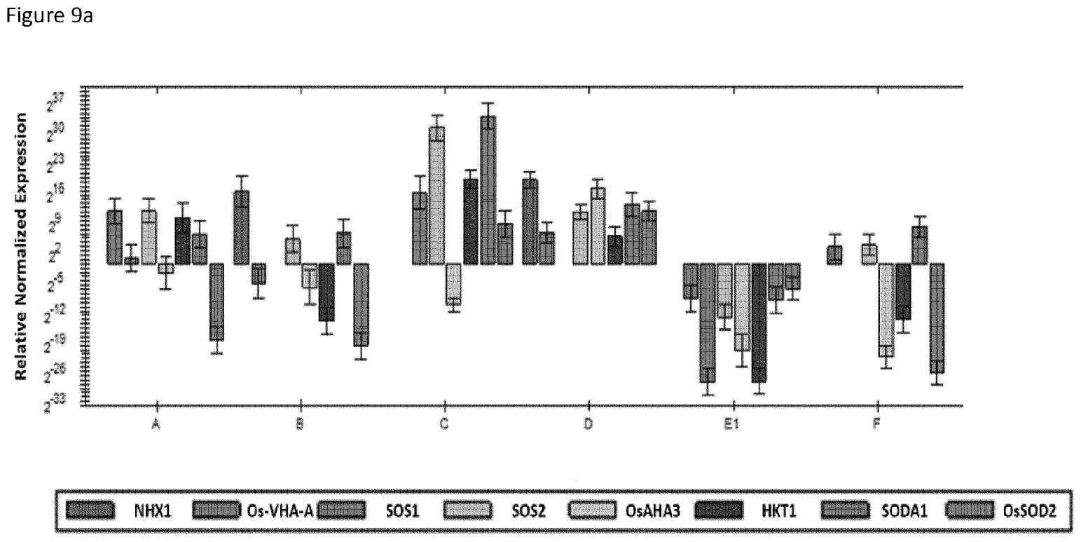

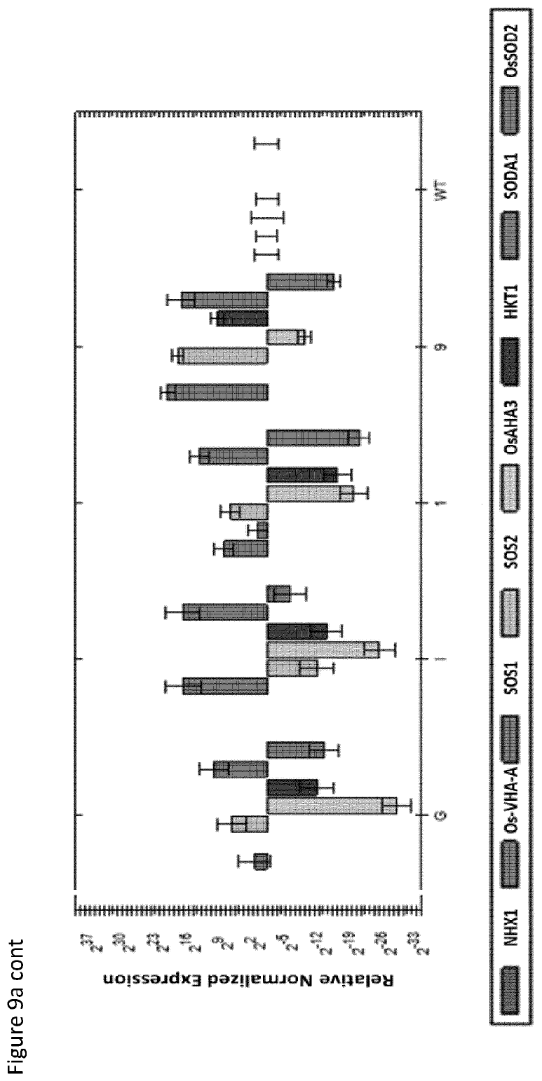

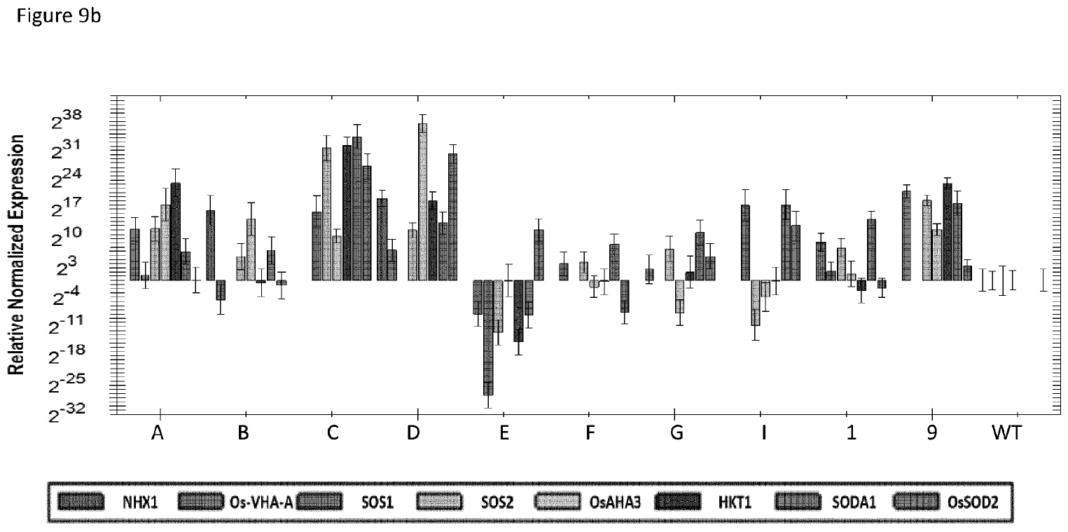

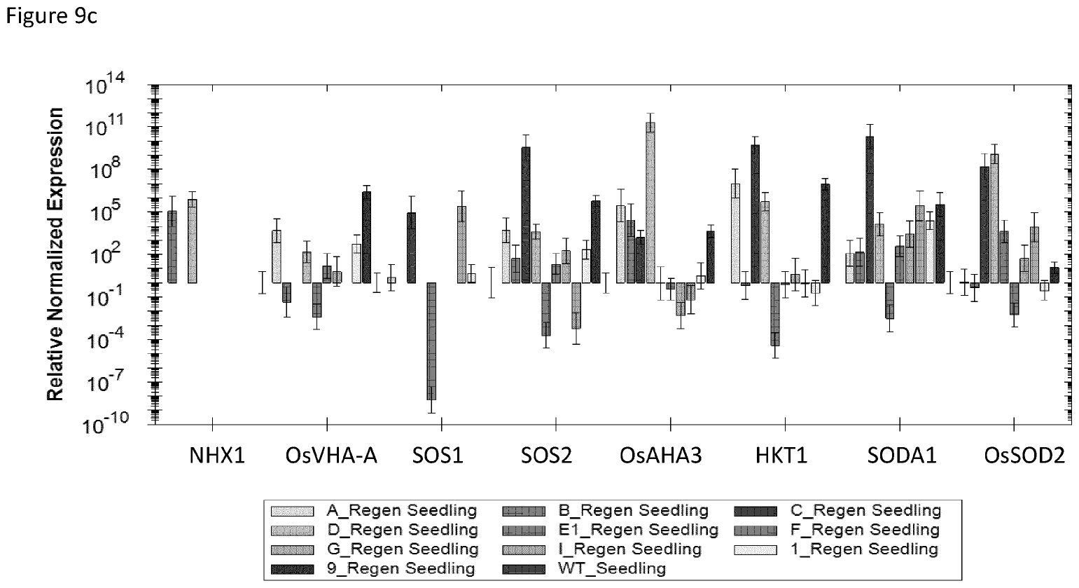

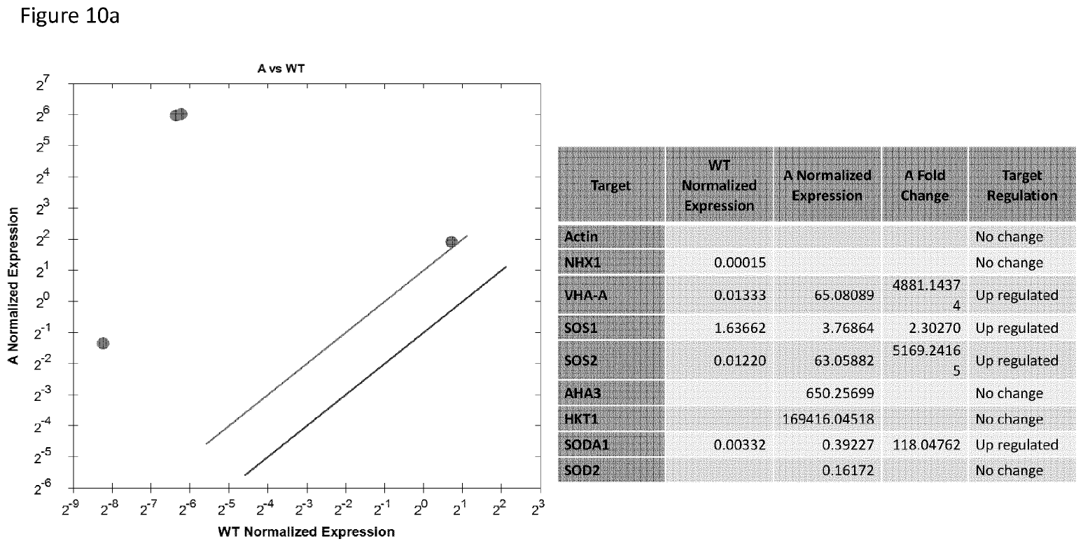

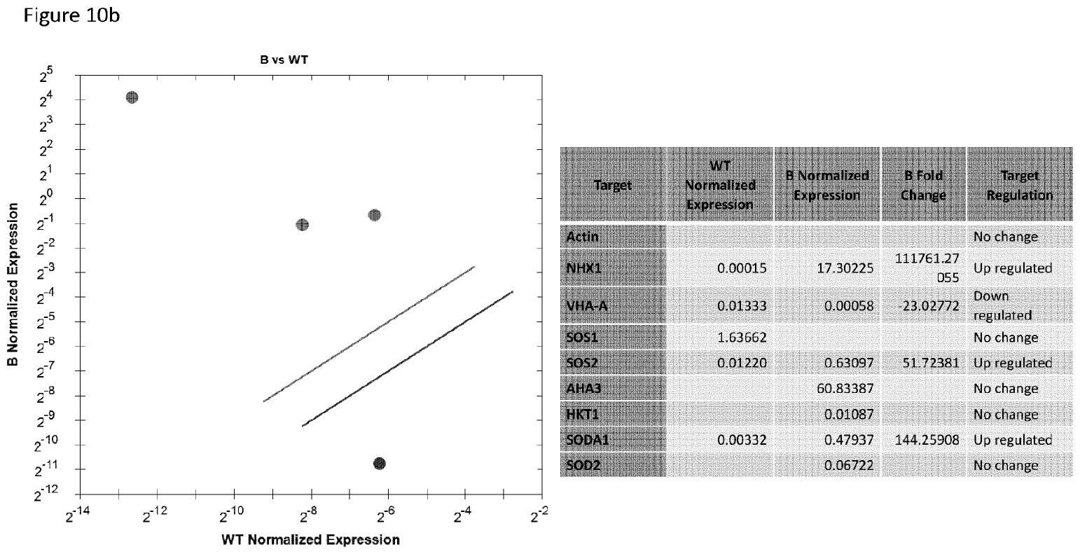

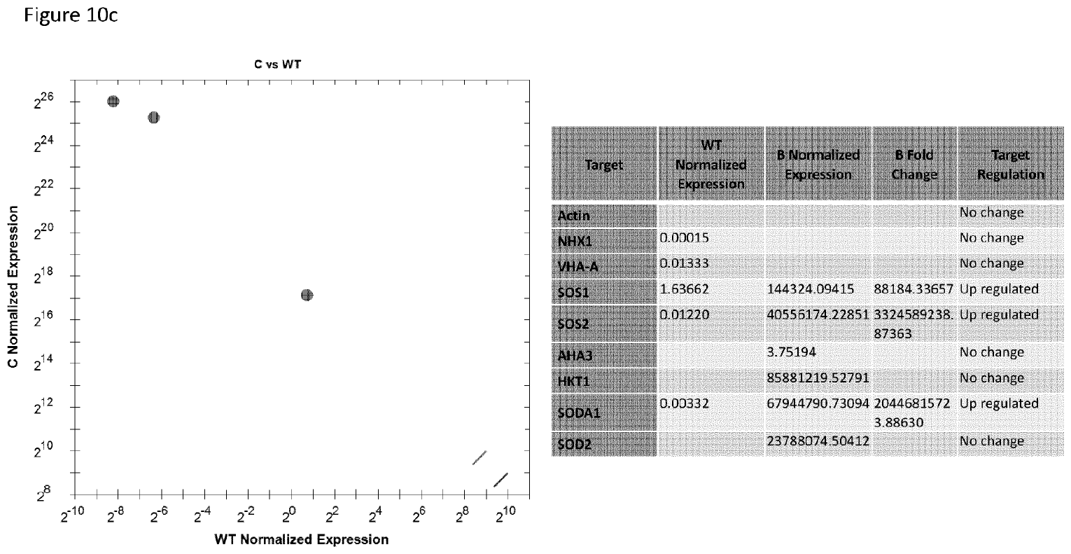

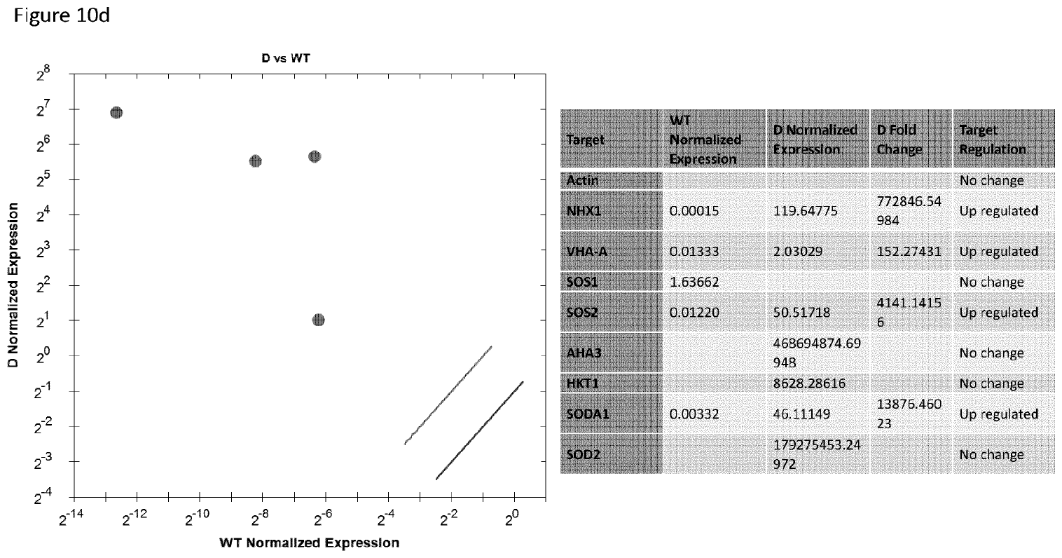

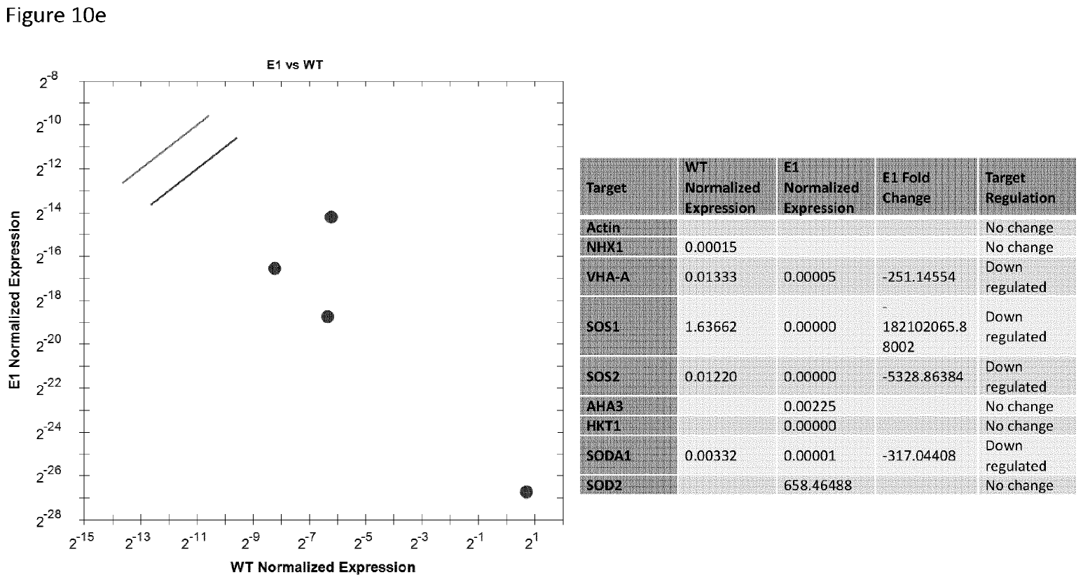

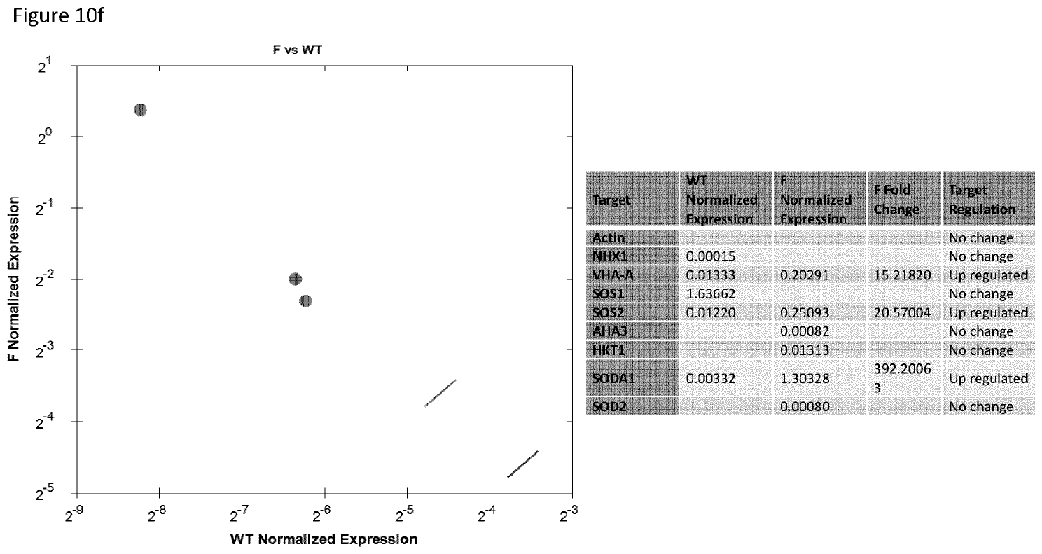

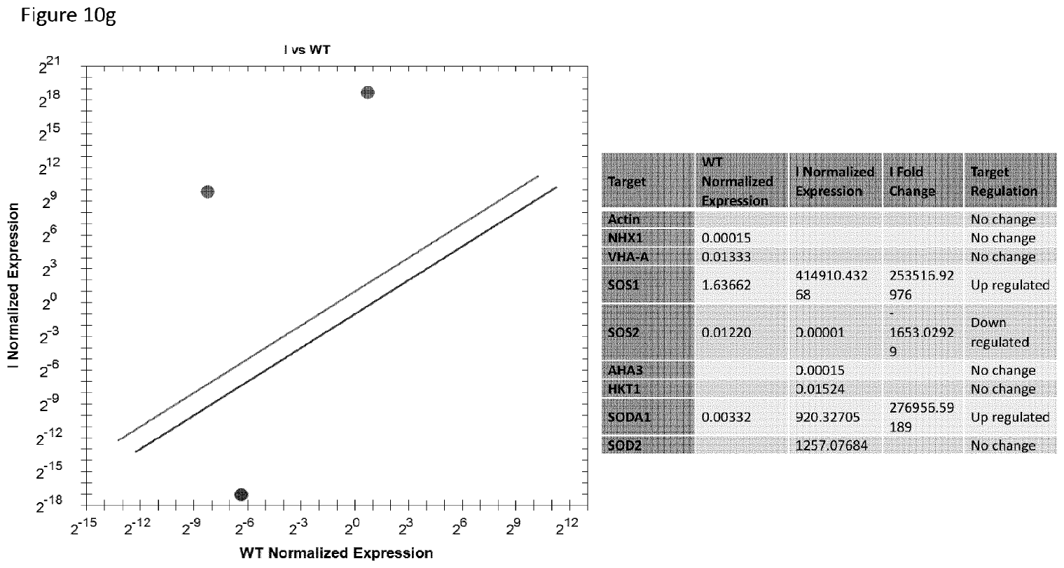

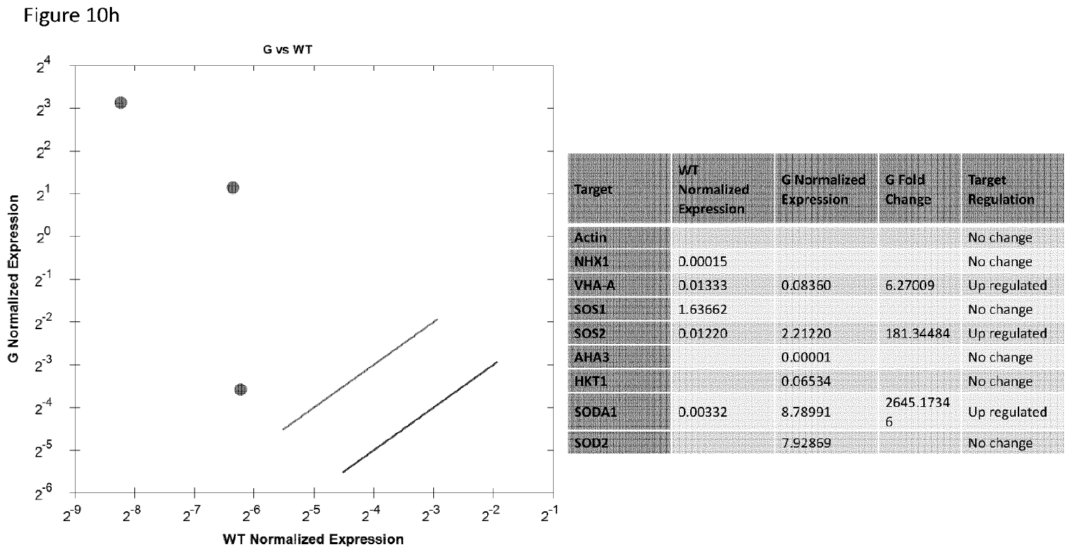

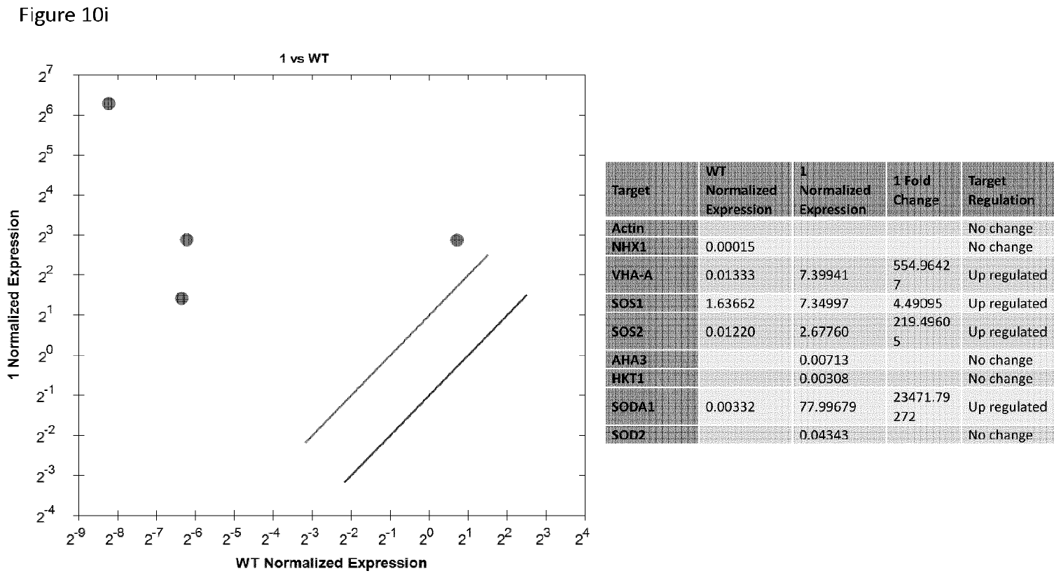

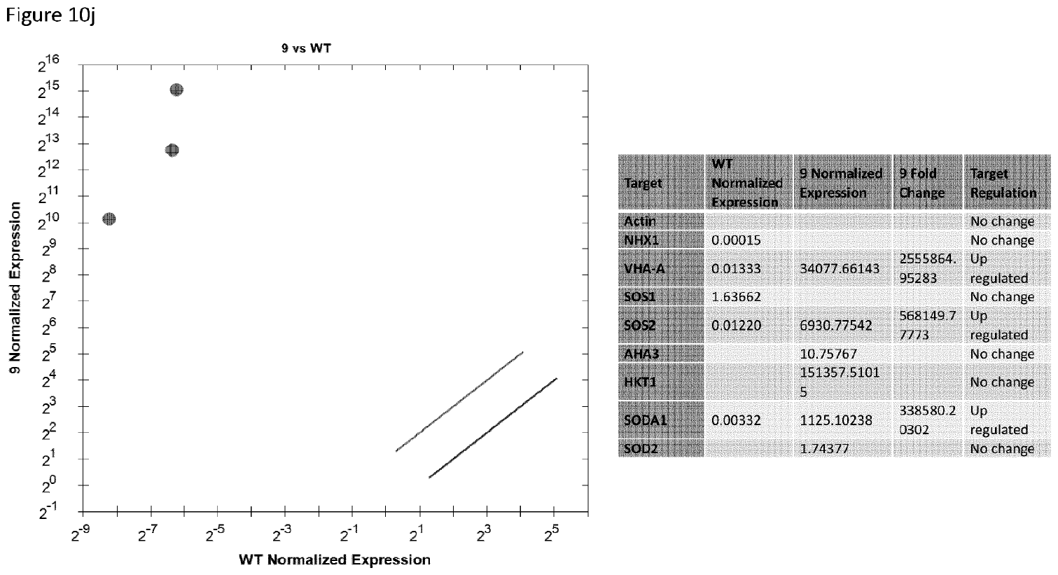

FIGS. 9a, 9b and 9c show the RNA expression levels of NHX1, SOS1, AHA3, HKT1, VHA-A, SOS2, SODA1, SOD2 and SODCC1 in tissue obtained from whole engineered plants. These data demonstrate that these genes were differentially expressed in the engineered plants compared to wild type plants. This difference is further evaluated in the scatter plots in FIGS. 10a-10j. The variation in the expression of these genes between the different engineered plants, seen in FIGS. 9 and 10, may be due to differences in the types of tissue that were collected from each engineered plant. As mentioned previously, the expression of the genes of interest can vary spatially across the plant. For example, HKT1 is expressed only in the vasculature of the plant, therefore if the sample collected did not contain vasculature tissue then little to no HKT1 expression would be observed. SOD2 had reduced expression in a number of the engineered plants tested. A possible reason for this reduced expression may be that the wild type plant used as a control was a different age compared to the engineered plants.

### Example 5—Salinity Tolerance of the Engineered Plants

The salt tolerance of the engineered plants from Example 2 were tested by partially submerging the roots of the plantlets or a plant in Hydroponics Media initially with Og of salt.

Hydroponics Media

| | |
|---|---|
| 4.3 g L−1 | Murashige & Skoog Basal Salts and Vitamins |
| 100 mg L−1 | Myo-inositol |
| 60 mg L−1 | Calcium Silicate |
| 50 mg L−1/90 mg L−1 | Kanamycin/Cefotaxime (either antibiotic) |
| 20 mg L−1 | Carbendazim (antifungal) |

The salinity of the hydroponics media was then increased incrementally up to 45 g L⁻¹, which is full oceanic salinity. The incremental changes in salt concentration and spacing between each change can be subject to change depending on performance of varieties. The growth rates of both shoots and roots was monitored and the overall health of the plant and colour of the leaves analysed.

| Day | NaCl gL−1 |
|---|---|
| 0 | 0 |
| 3 | 2 |
| 6 | 4 |
| 9 | 6 |
| 12 | 8 |
| 15 | 10 |
| 18 | 12 |
| 21 | 14 |
| 24 | 16 |
| 27 | 18 |
| 30 | 20 |
| 33 | 23 |
| 36 | 26 |
| 39 | 29 |
| 42 | 32 |
| 45 | 35 |

### Example 6—Salinity Tolerance of the Engineered Plants

The salt tolerance of the engineered plants from Example 2 were tested by growing the plants in media that had been supplemented with sodium chloride. Engineered plants A, B, C, D, E1, F, G, H, & I from Example 2 were placed directly into RRM II Media supplemented with an initial 8 g/l NaCl and later an increased 16 g/L NaCl. The engineered plants continued to grow under these high salinity conditions. Wild-type plants were initially placed in MS media supplemented with 2 g/L NaCl. The salinity was increased by 2 g/L every two days until 10 g/L NaCl.

MS Media

| | |
|---|---|
| 4.3 g L⁻¹ | Murashige & Skoog Basal Salts and Vitamins |
| 2, 4, 6, 8, | Maltose |
| 10 g L⁻¹ | |
| 50 mg L⁻¹ | Vancomycin/Cefotaxime (either antibiotic) |
| 20 mg L⁻¹ | Carbendazim (antifungal) |

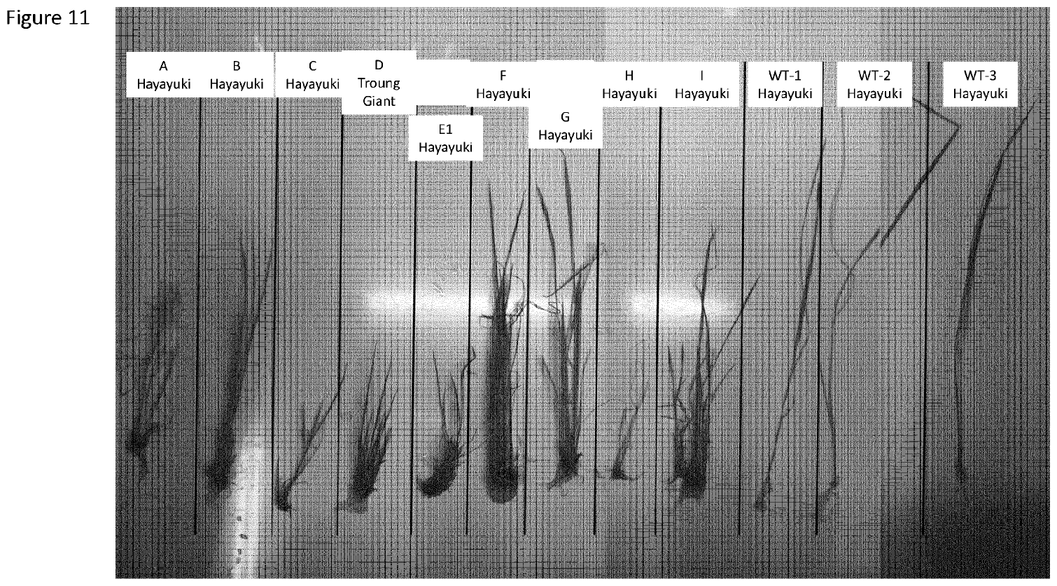

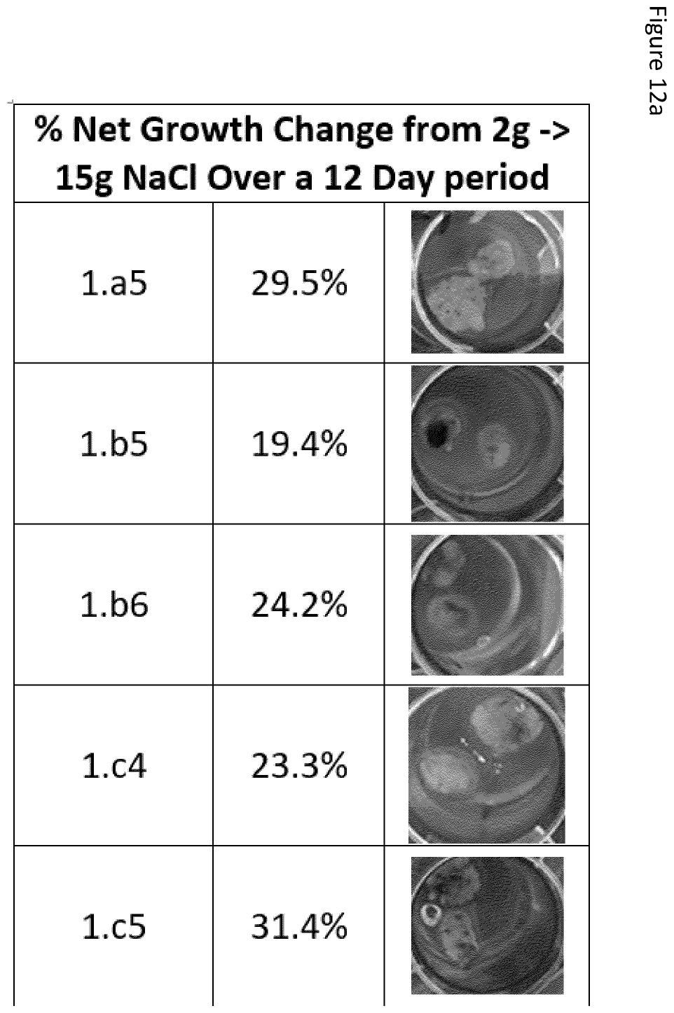

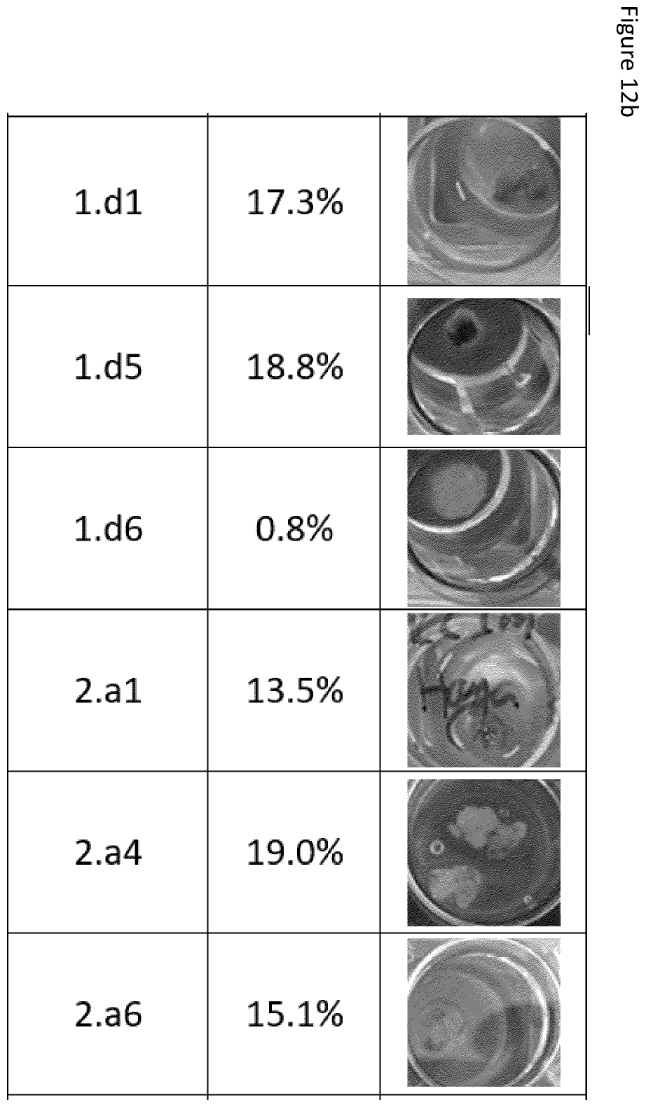

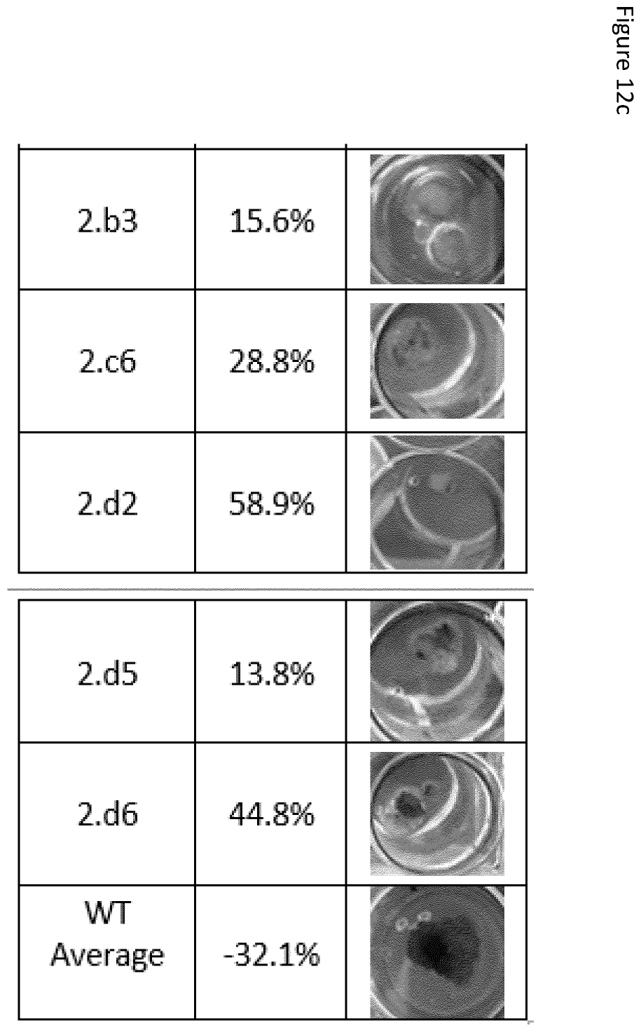

FIG. 11 provides images of the engineered and wild-type plants on the final day of the experiment. The images in FIG. 11 show that the wild-type plants all exhibit significant yellowing of at least half the leaves. Starting at the leaf tip and often spreading throughout the entire length of the leaf. This yellowing is not observed in the engineered plants. The insertion of the enhancer elements so that they are operatively linked to the genes of interested has enabled the engineered plants to tolerate much higher concentrations of salt without showing physical signs of stress. The data demonstrate that the engineered plants according to the invention have increased salt tolerance compared to wild-type plants.

Some of the engineered plants appear white (A & H). These plants have exhibited this colour throughout their entire post regenerated life cycle. Without wishing to by bound to any particular theory, the inventors hypothesize that is due to the substantial increase in gene expression causing a reduction in chlorophyll production. These plants have still behaved similarly to their green counterparts.

The wild-type controls show significantly reduced root and leaf quantity. Their structure is also thinner and unable to support the weight of the plant unaided. These characteristics show that the increased salinity has affected the health of the wild-type plants. In contrast the engineered plants do not exhibit these characteristics further suggesting that the edits that have been made to the genome not only allow the plants to survive, but also to thrive in highly saline environments that wild-type rice are unable to grow in. This improvement is seen in multiple rice varieties demonstrating that inventors designed can be applied to various rice varieties.

Overall, these data demonstrate the plants that have been engineered to have enhancer elements operatively linked to particular genes of interest can grow at high salinities and thus have improved salt tolerance compared to wild-type plants.

### Example 7—Improved Design of Genetic Inserts

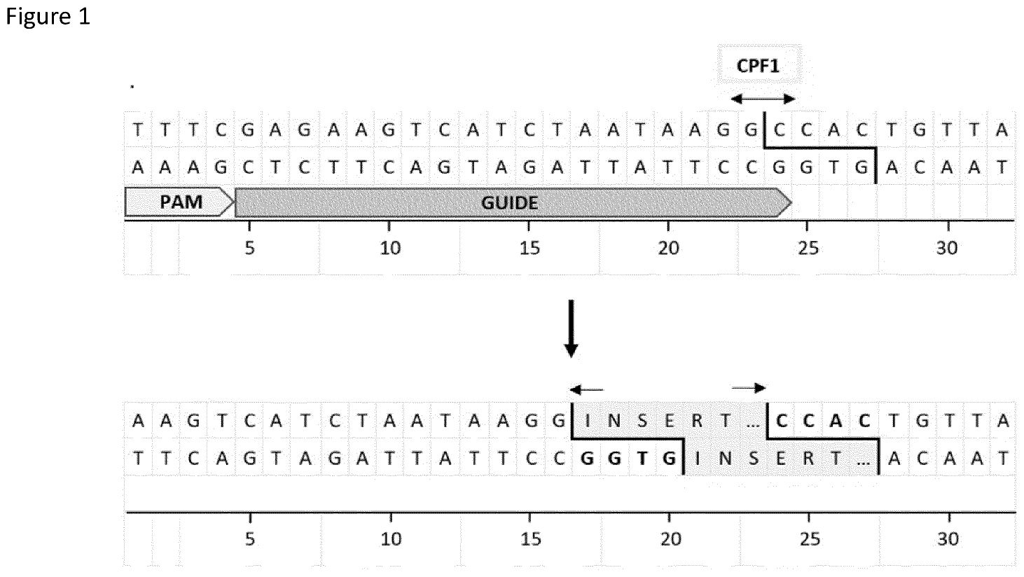

The design of the genetic inserts was altered to improve the editing efficiency and minimize the amount of DNA required to make a successful edit. The main focus for this improved design was to use Cpf1 (Cas12a) to make incisions within the genomic sequence where Cas9 was previously used. Cas9 makes blunt-ended cuts that require large homology arms (˜125 bases including the genetic inserts) to successfully edit and insert any amount of DNA into a genomic sequence, this entire section of DNA is incorporated into the plants resulting in a ˜120 base pair edit per gene. In comparison, Cpf1 uses staggered cuts, making incisions 19 base pairs after the PAM site on the sense strand and 23 base pairs after the PAM site on the anti-sense strand, as shown in FIG. 1. Cpf1 edits do not require homology arms and instead rely on 4-5 base pair overhangs that can be seen in bold in FIG. 1.

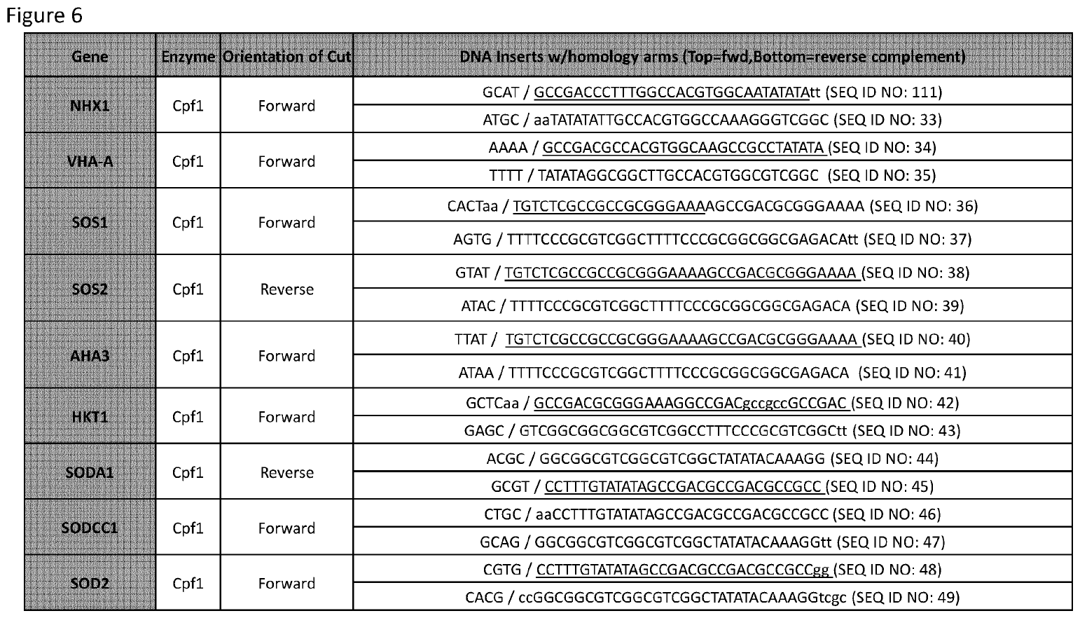

These overhangs were included at the ends of the genetic inserts to enable a smaller piece of DNA to be integrated into the genome using the sequences in FIG. 6. The edit sites were designed to ensure normal function of the gene is left unchanged, only changing the expression intensity and expression location pattern. In this updated design, the gene of interest VHA-A, SOS2, AHA3, and SODA1 have all been converted to accommodate Cpf1 instead of Cas9. The updated guide RNA (gRNA) and updated forward and reverse insert sequences with relevant overhang sequences are listed in Table 14 and FIG. 6.

In combination with Example 1, these data demonstrate that the enhancer elements can be integrated into the genome of the plant using a variety of nucleases. Cpf1 nuclease may be used to integrate the enhancer elements into the genome because it results in a more efficient method of generating the engineered plants.

TABLE 14
The guide RNAs for Cpf1 nucleases

| Gene | Guide RNA sequence |
|---|---|
| NHX1 | TTTACCGGGAATTGTCGAATTAGGC (SEQ ID NO: 115) |
| VHA-A | TTTAAAATTCGCTGGAGGAAAAAAA (SEQ ID NO: 116) |
| SOS1 | TTTGCCTTGGATTTTCCACTTGCA (SEQ ID NO: 117) |
| SOS2 | TTTGGGGGGGCTTCGCTGCCTGAAT (SEQ ID NO: 118) |
| AHA3 | TTTGCAAGCAAGATTTCCTTTGCTT (SEQ ID NO: 119) |
| HKT1 | TTTCTCATACTCGTTGGCTCGTTGC (SEQ ID NO: 120) |
| SODA1 | TTTATGCGGCATCTGTCTCGTGAAC (SEQ ID NO: 121) |
| SODCC1 | TTTAGGACCTCTAGAGGCTACTTCT (SEQ ID NO: 122) |
| SOD2 | TTTGGTGGACTACTCGATCTCTGCG (SEQ ID NO: 123) |

### Example 8—Production of Engineered Calli

Calli were produced as described in Example 3. These calli were bombarded with the DNA inserts described in Example 7 which use Cpf1 nucleases only to make incisions within the genomic sequence. The DNA inserts targeted four genes of interest—OsNHX1, OsVHA-A, OsSOS1 & OsSOS2 and their sequences are provided in FIG. 6 Error! Reference source not found. The OsSOS1 gene is operably linked to an enhancer element comprising SEQ ID NO: 12, the OsSOS2 gene is operably linked to an enhancer element comprising SEQ ID NO: 13, the OsVHA-A gene is operably linked to an enhancer element comprising SEQ ID NO: 11 and the OsNHX1 gene is operably linked to an enhancer element comprising SEQ ID NO: 10.

These four genes of interest were chosen as they are most likely be expressed within calli, considering the difference in physiology, hormone and transcription factor abundance compared to a regenerated engineered plant. This set of genes of interest was also chosen because they act synergistically to drive salt exclusion and isolation within any plant cell to drive salt tolerance.

### Example 9—Salinity Tolerance of the Calli

The salt tolerance of the engineered calli from Example 8 were tested by transferring the calli to Rice Callus Induction Media supplemented with 2 gL⁻¹ of sodium chloride. Every two days the calli were transferred to a higher concentration of salt. The concentrations tested are shown below:

| Day | NaCl gL⁻¹ |
|---|---|
| 0 | 2 |
| 2 | 4 |
| 4 | 6 |
| 6 | 8 |
| 8 | 10 |
| 10 | 12.5 |
| 12 | 15 |

The maximum of 15 gL⁻¹ sodium chloride is just below 50% of full ocean salinity, because the calli are not expected to perform optimally compared to a whole plant. However, calli that can grow in 15 gL⁻¹ will have a significantly high salt tolerance, well beyond the tolerance of the rice varieties currently in use commercially and beyond the salinity of paddies in the Mekong (for example). Overall callus health will also be recorded including: Growth rates; Colour; Texture and Structural integrity.

FIGS. 12a-c demonstrate that the engineered calli were able to grow in media that contained up to 15 gL⁻¹ of sodium chloride, whereas wild-type calli were not able to grow under the same conditions. Identifiers of callus health included colour (white/yellowish as opposed to black) and texture (friable and not mushy). The images in FIGS. 12a-c demonstrate that in general the engineered calli appeared whiter with more defined edges than the WT, which indicates that these calli had higher tolerance.

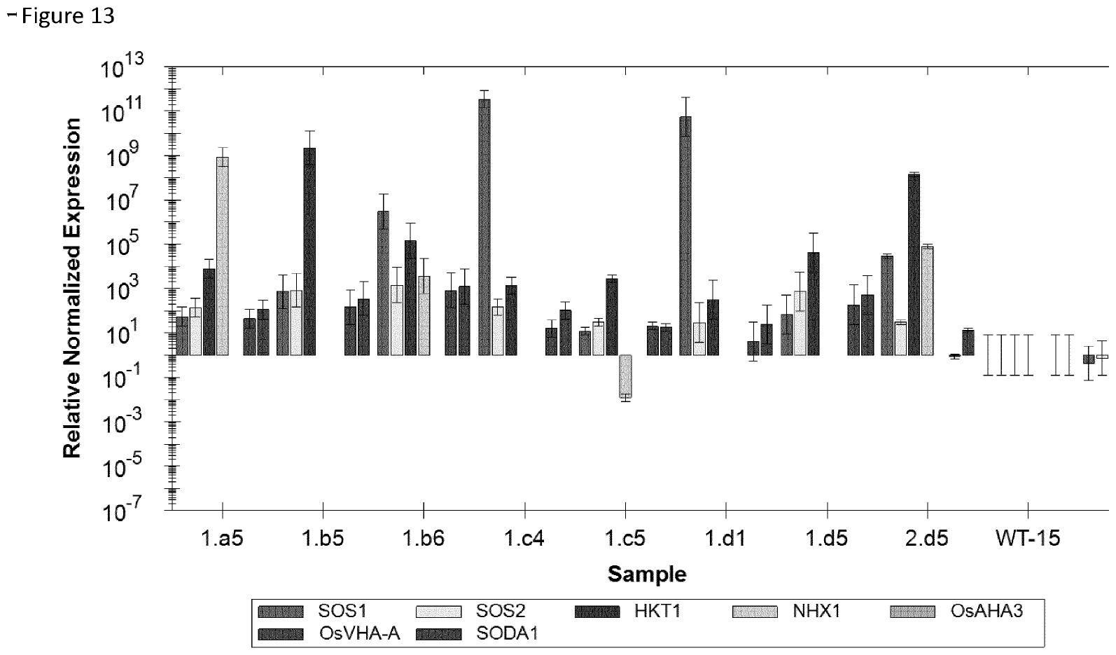

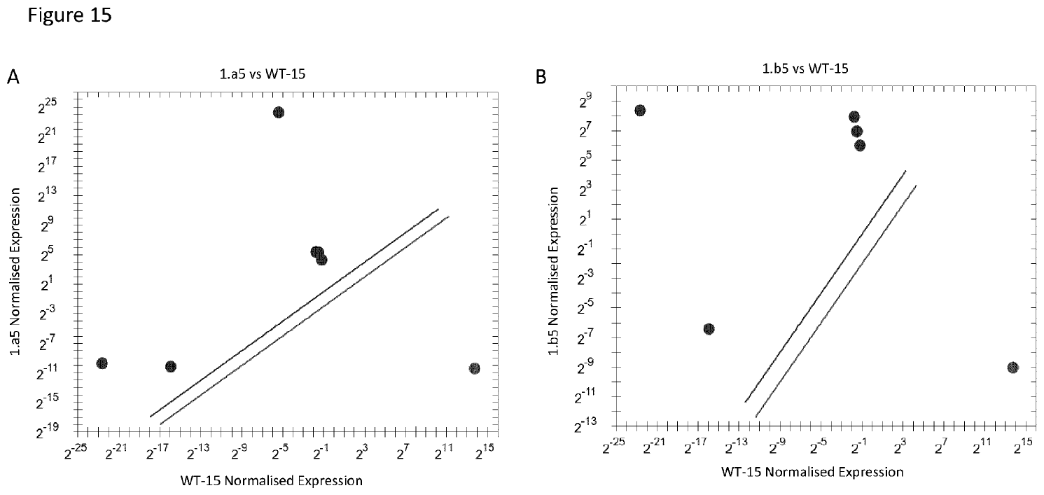

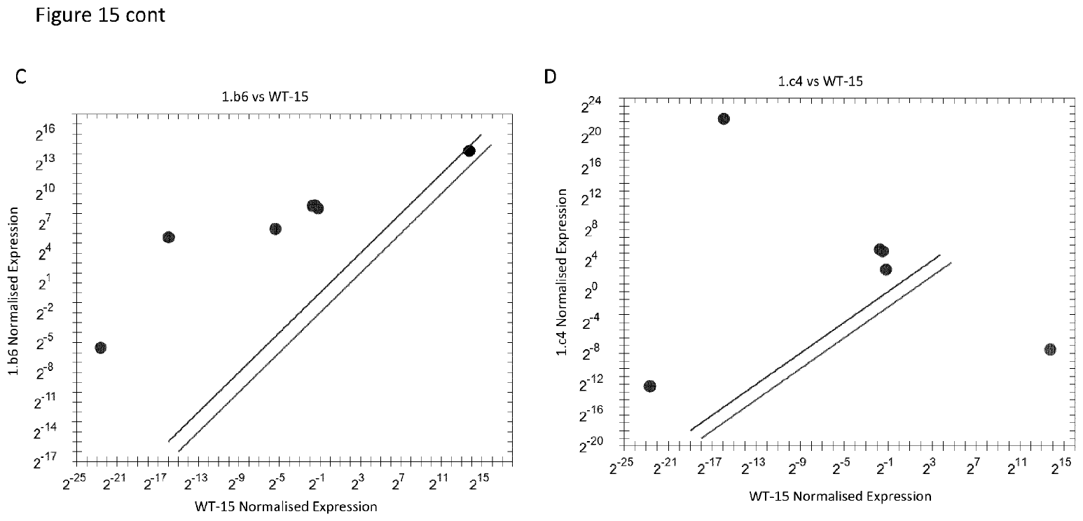

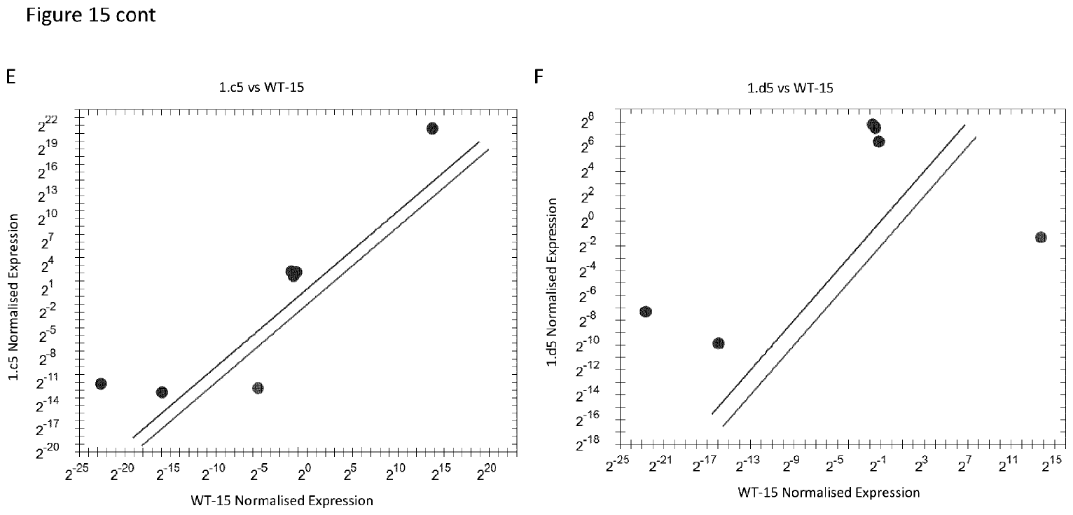

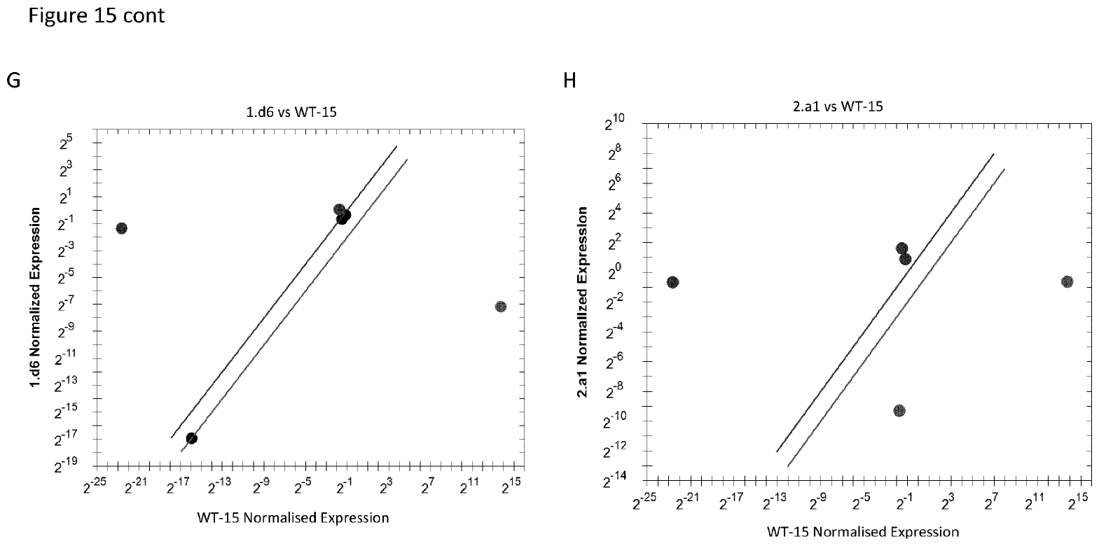

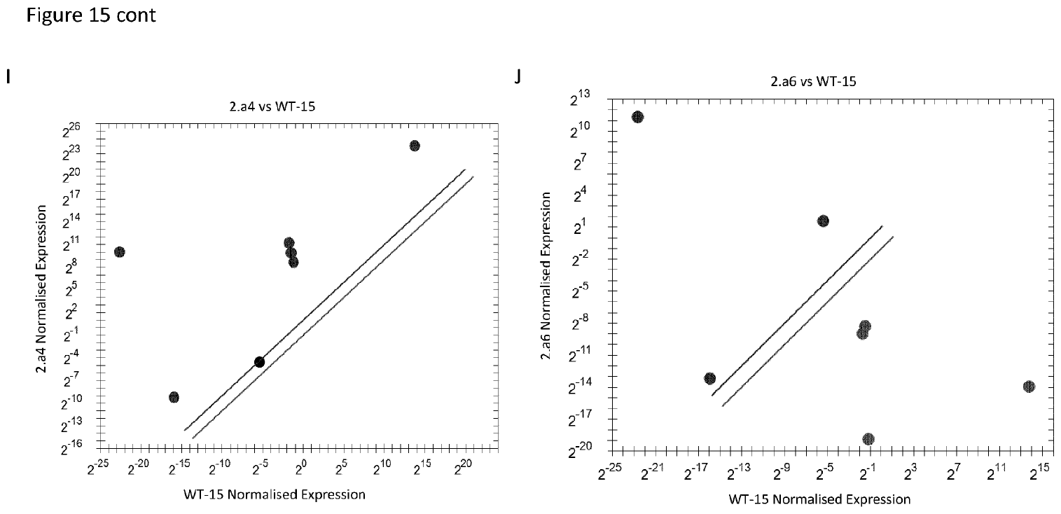

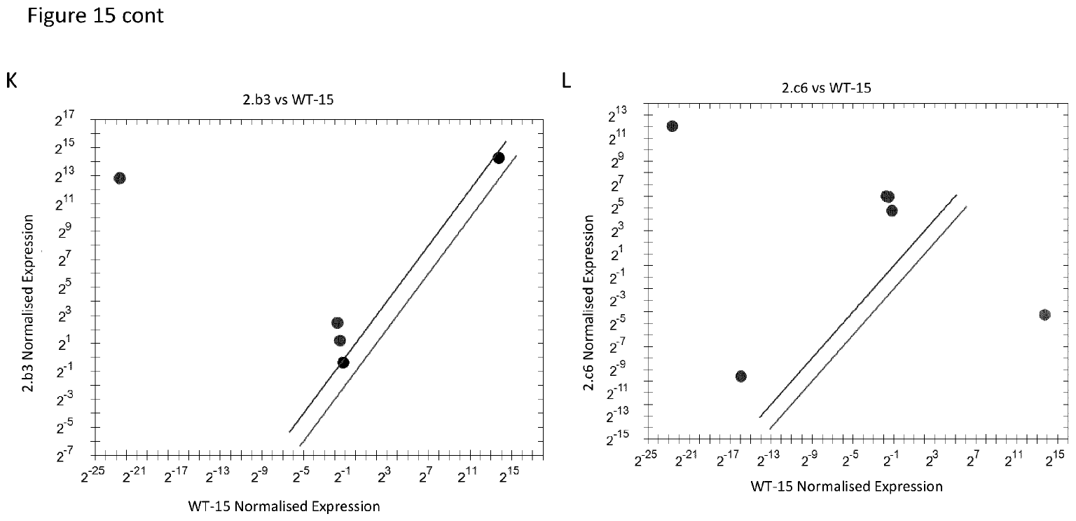

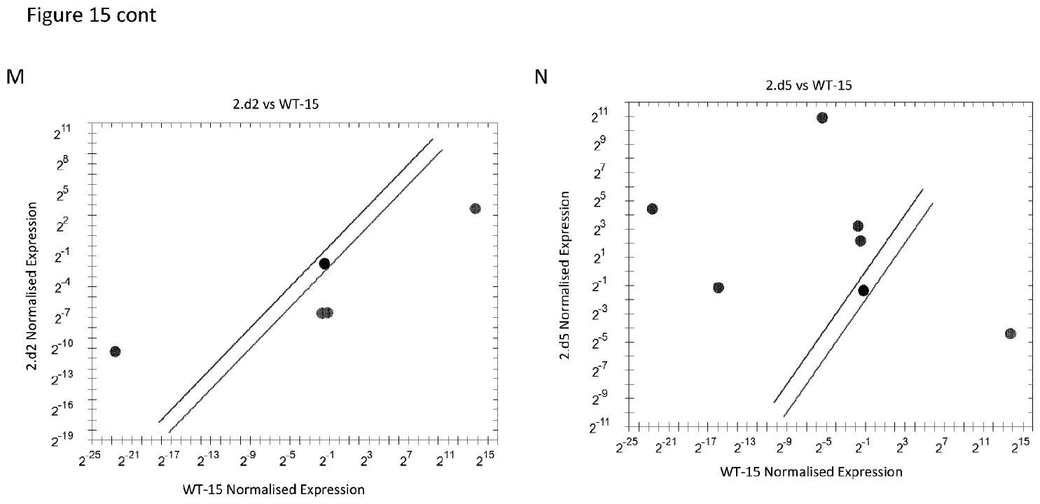

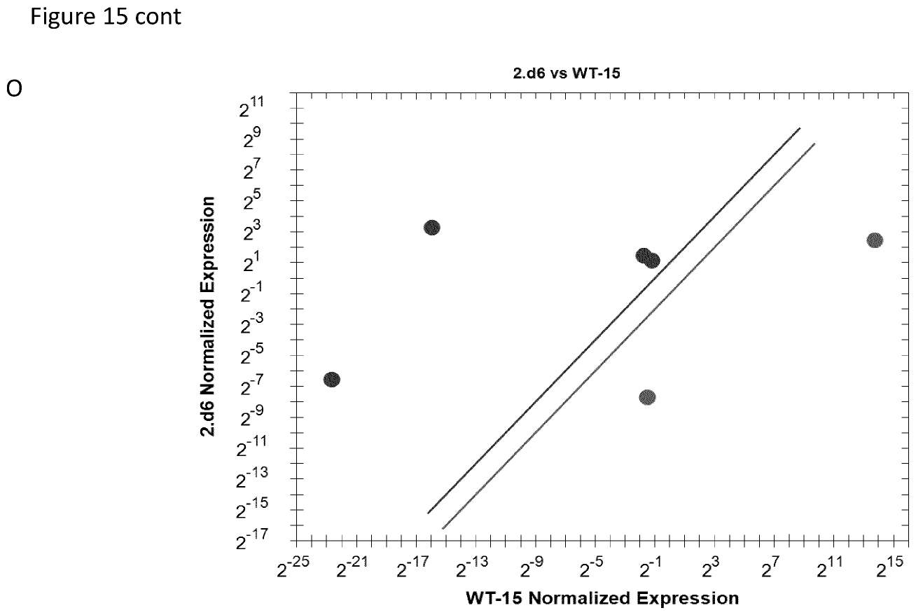

q-RT-PCR was performed on the engineered calli as described in Example 4. FIG. 13 shows the gene expression of SOS1, SOS2, HKT1, NHX1, AHA-3, VHA-A, and SODA1 in the engineered calli. FIG. 13 demonstrates that the majority of engineered calli had increased SOS1, SOS2, HKT1, NHX1, AHA-3, VHA-A, and SODA1 expression compared to wild-type calli when grown at 15 g/L of sodium chloride. This difference is further evaluated in the scatter plots in FIGS. 15a-15o. These data demonstrates that the bombardment process has successful integrated enhancer elements into the genome of the plant so that they are operatively linked to the genes of interest, which enabled the engineered calli to grow under increased saline stress compared to the wild-type.

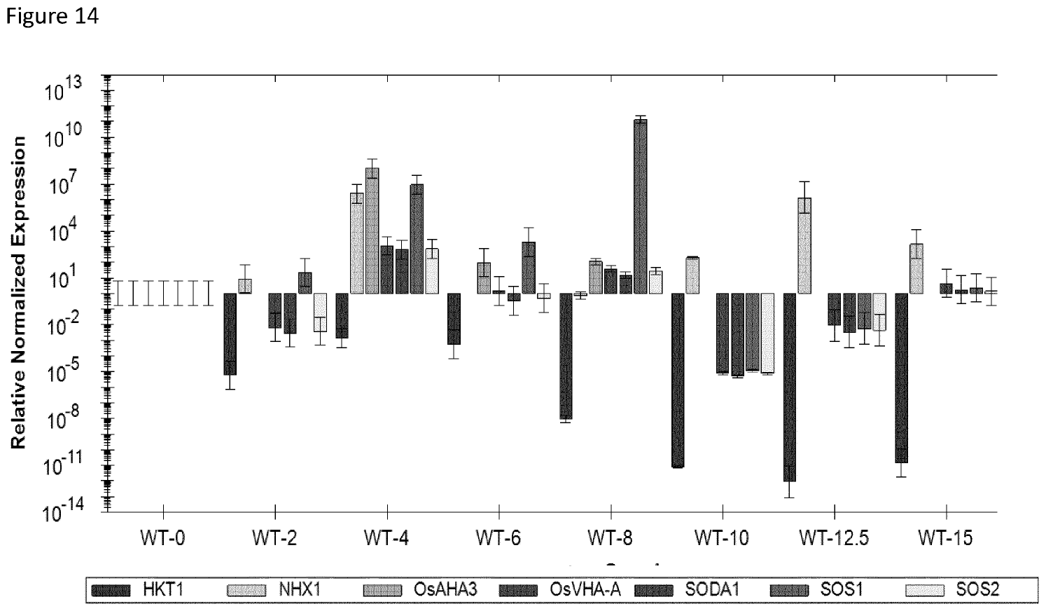

In previous studies it was observed that rice calli show a sharp decrease in fresh weight at 8.7 g/L NaCl [27], as our transformed calli exhibit continued growth through to 15 g/L NaCl we have observed a huge increase in callus salinity tolerance. qRT PCR analysis was conducted on the wild-type calli taken at 0, 2, 4, 6, 8, 10, 12.5 & 15 g/L NaCl. As seen in FIG. 14, in the interval between 8 g/L and 10 g/L there is a drastic change in the expression of NHX1, OsAHA3, OsVHA-A, SODA1, SOS1 and SOS2, indicating a similar shift where calli become unable to tolerate such high concentrations of NaCl. Overall, the data is similar to the physical characteristics observed in the literature of calli grown in salt.

### Example 10—Production of Genetic Inserts for Engineered Kale (Brassica oleracea)

Genetic inserts were designed to stably introduce enhancer elements into the genome of kale using Cpf1 nucleases. These genes of interest encode a variety of proteins with different functions including a plasma membrane ion transporter; a plasma membrane hydrogen exporting ATPase, a tonoplast hydrogen exporting ATPase, a tonoplast ion transporter, a potassium transporter, a protein kinase and an antioxidant.

TABLE 15
The sequences added to the 5′ and 3′ ends of the DNA inserts for engineered
Kale. Underlining signifies adapter sequences cleaved by restriction enzymes

| | Kale | DNA insert | cpf1 DNA insert |
|---|---|---|---|
| Rice gene | orthologue | forward primer | reverse primer |
| NHX1 | Bo9g010200 | ATAGCACAATGCCGACCCT | CTATTATATATTGCCACGTGG |
| | | TTGGCCACGTGGCAATATA | CCAAAGGGTCGGCATTGTG |
| | | TA (SEQ ID NO: 59) | (SEQ ID NO: 60) |
| VHA-A | Bo6g122400 | TCGGCACAATGCCGACGCC | CCGATATATAGGCGGCTTGC |
| | | ACGTGGCAAGCCGCCTATA | CACGTGGCGTCGGCATTGTG |
| | | TA (SEQ ID NO: 61) | (SEQ ID NO: 62) |
| SOS1 | Bo9g071470 | CTCTTGACGCACAATCCCA | AGAGTTATATATTTTCCCGCG |
| | | CTTGTCTCTATATAGCCGC | TCGGCTTTTCCCGCGGCGGCT |
| | | CGCGGGAAAAGCCGACGC | ATATAGAGACAAGTGGGATT |
| | | GGGAAAATATATAA(SEQ | GTG(SEQ ID NO: 64) |
| | | ID NO: 63) | |
| SOS2 | Bo4g139520.1 | GTAACTGACGCACAATCCC | TTACGCTTATATATTTTCCCG |
| | | ACTTGTCTCTATATAGCCG | CGTCGGCTTTTCCCGCGGCGG |
| | | CCGCGGGAAAAGCCGACG | CTATATAGAGACAAGTGGGA |
| | | CGGGAAAATATATAAGC | TTGTGCGTCAG (SEQ ID NO: |
| | | (SEQ ID NO: 65) | 66) |
| OsAHA3 | Bo9g133110 | CATCCACAATCCCACTACC | GATGTATATATTTTCCCGCGT |
| | | TCGTGTCTCGCCGCCGCGG | CGGCTTTTCCCGCGGCGGCG |
| | | GAAAAGCCGACGCGGGAA | AGACACGAGGTAGTGGGATT |
| | | AATATATA | GTG (SEQ ID NO: 68) |
| | | (SEQ ID NO: 67) | |
| HKT1 | Bo3g044660 | ACACCACAATACCTCGTGT | GTGTGTCGGCGGCGGCGTCG |
| | | CTCGCCGACGCGGGAAAG | GCCTTTCCCGCGTCGGCGAG |
| | | GCCGACGCCGCCGCCGAC | ACACGAGGTATTGTG |
| | | (SEQ ID NO: 69) | (SEQ ID NO: 70) |
| [CuZn]SOD2 | Bo5g137020 | ATGACACAATCCCACTCCT | TCATGAGACACGAGGTGGCG |
| | | TTGTATATAGCCGACGCCG | GCGTCGGCGTCGGCTATATA |
| | | ACGCCGCCACCTCGTGTCT | CAAAGGAGTGGGATTGTG |
| | | C (SEQ ID NO: 71) | (SEQ ID NO: 72) |
| SODA1 | Bolg141360 | TCTCCACAATCCCACTCCT | GAGAGAGACACGAGGTGGCG |
| | | TTGTATATAGCCGACGCCG | GCGTCGGCGTCGGCTATATA |
| | | ACGCCGCCACCTCGTGTCT | CAAAGGAGTGGGATTGTG |
| | | C (SEQ ID NO: 73) | (SEQ ID NO: 74) |
| CuZn SOD | Bo4g165150 | ACGCCACAATCCCACTCCT | GCGTGAGACACGAGGTGGCG |
| chloroplast | | TTGTATATAGCCGACGCCG | GCGTCGGCGTCGGCTATATA |
| | | ACGCCGCCACCTCGTGTCT | CAAAGGAGTGGGATTGTG |
| | | C (SEQ ID NO: 75) | (SEQ ID NO: 76) |
| OSK1 | Bo4g138000 | TTTCCCTCGTGTCTCATGAC | GAAATATATACGGCGGCGTC |
| | | GCACAATCCCACTCCTTTG | GGCGTCGGCTATATACAAAG |
| | | TATATAGCCGACGCCGACG | GAGTGGGATTGTGCGTCATG |
| | | CCGCCGTATATA (SEQ ID | AGACACGAGG (SEQ ID NO: |
| | | NO: 77) | 78) |

### Example 11—Genomic Sequencing of the Engineered Plants

The genomic DNA of the engineered plants produced in Example 2 were sequenced to determine whether the genes of interest had been introduced successfully into the plant after bombardment.

The genomic DNA was isolated and purified using a Wizard® Genomic DNA Purification Kit. Briefly, 40 mg of lead material was harvested from the target plant. The leaf tissue was frozen in liquid nitrogen and ground into a fine powder using a pestle and mortar. 600 μl of Nuclei Lysis Solution was added to the sample, which was then vortexed for 1-3 seconds. The sample was then incubated at 65° C. for 15 minutes. 3 μl of RNase Solution was added to the cell lysate and mixed by inverting the tube 2-5 times. The mixture was incubated at 37° C. for 15 minutes. The sample was cooled at room temperature for 5 minutes and then 200 μl of Protein Precipitation Solution was added. The sample was vortexed vigorously at high speed for 20 seconds and then centrifuged for 3 minutes at 13,000-16,000×g. The precipitated proteins formed a tight pellet. The supernatant was transferred to a tube containing 600 μl of room temperature isopropanol. The sample was mixed by inverting the tube and then centrifuged at 13,000-16,000×g for 1 minute at room temperature. 600 μl of room temperature 70% ethanol was added and the tube gently inverted several times to wash the DNA. The sample was then centrifuged at 13,000-16,000×g for 1 minute at room temperature. The ethanol was removed the pellet containing the DNA was air-dried for 15 minutes. 100 μl of DNA Rehydration Solution was added and the sample incubated at 65° C. for 1 hour. The concentration of each DNA sample was measured using a Nanodrop spectrophotometer. The resulting DNA was sequenced by Eurofins Genomics lab.

### Example 12—Stable Inheritance of Salt Tolerance in Engineering Plants

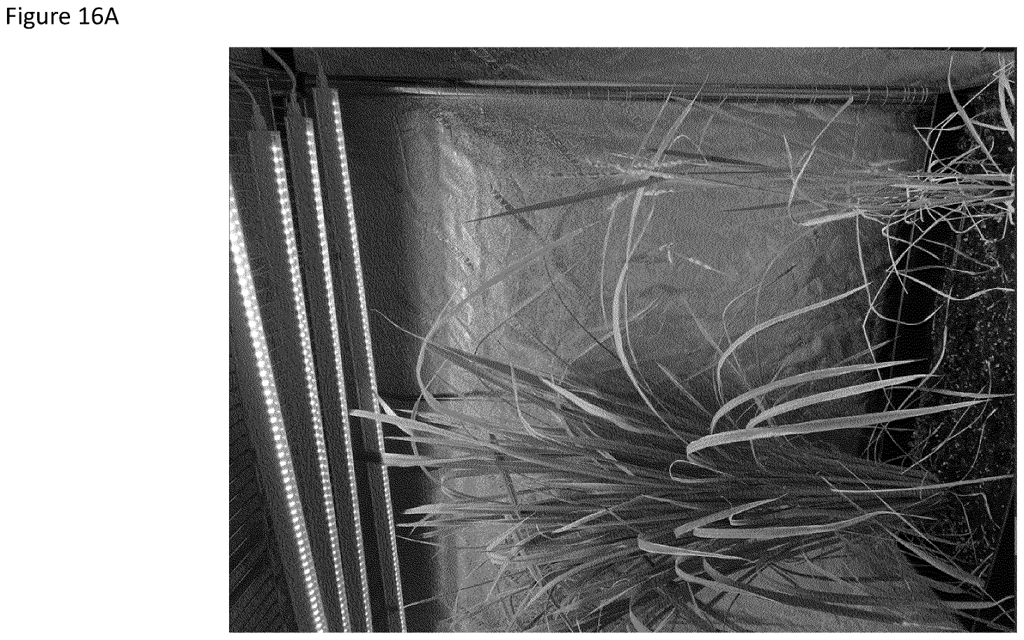

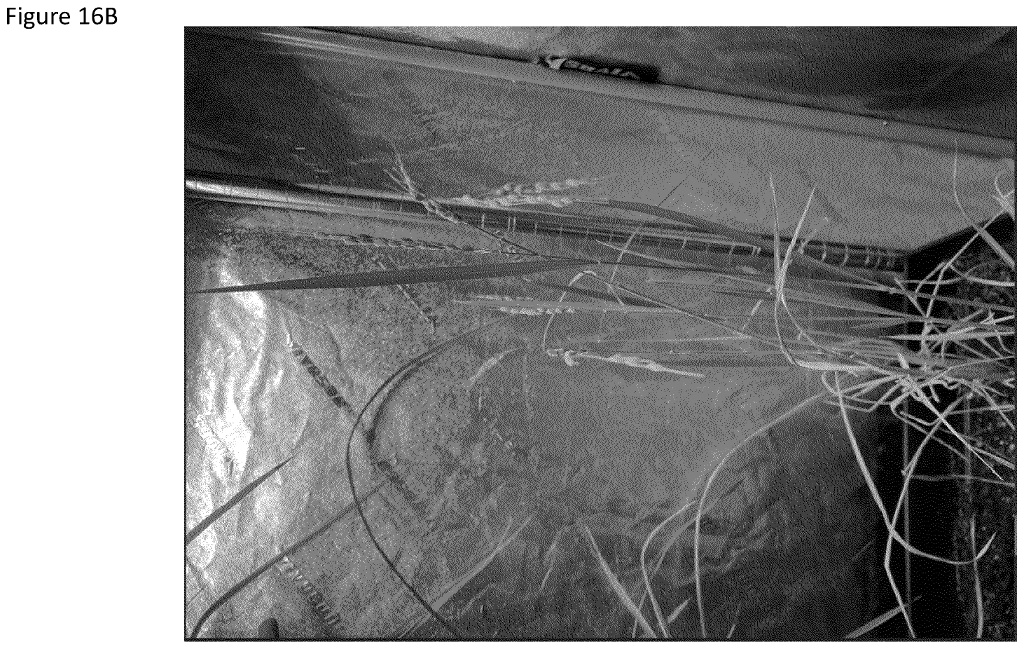

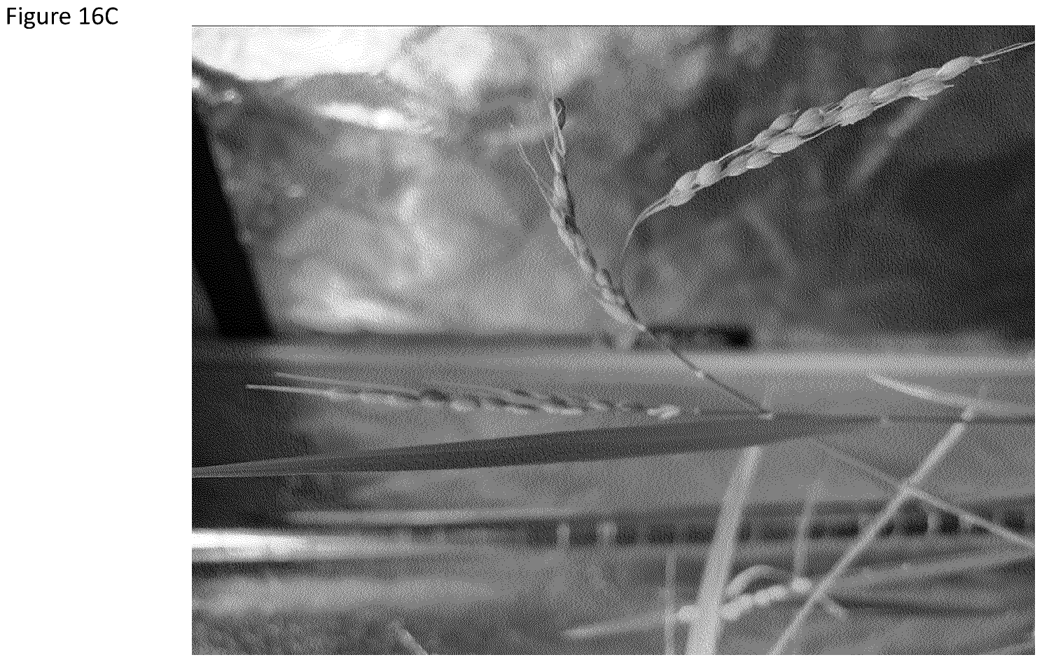

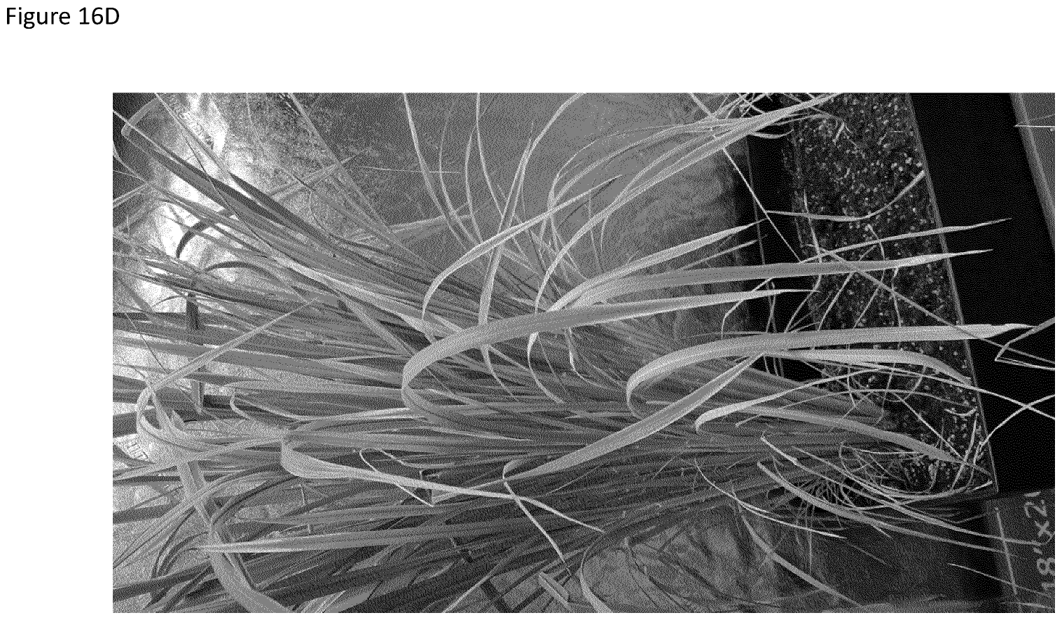

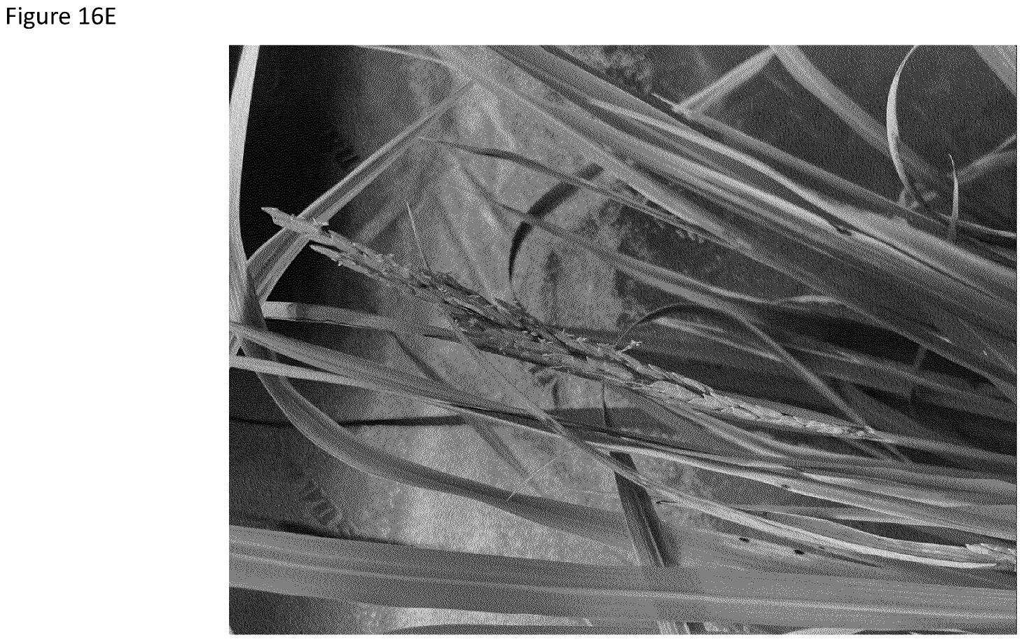

Engineered rice plants of the Hayayuki and Truong Giang varieties were produced as described in Example 2. These engineered rice plants comprised the following genes of interest, OsNHX1, OsVHA-A, OsSOS1, OsSOS2, OsAHA3, OsHKT1, and either OsSODA1 or OsSOD2, wherein each gene of interest is operatively linked to an enhancer element. These engineered rice plants were grown to maturity so that they produced seeds. FIGS. 16a-e are images of these engineered rice plants showing the panicles and seeds.

Seeds were harvested from these three plants (Hayayuki 1, Hayayuki 2 and Truong Giang) and germinated to seedlings. The gene expression in the leaves of the progeny plants was analyzed using qRT-PCR as performed in Example 4 to observe whether salt tolerance had been successfully inherited.

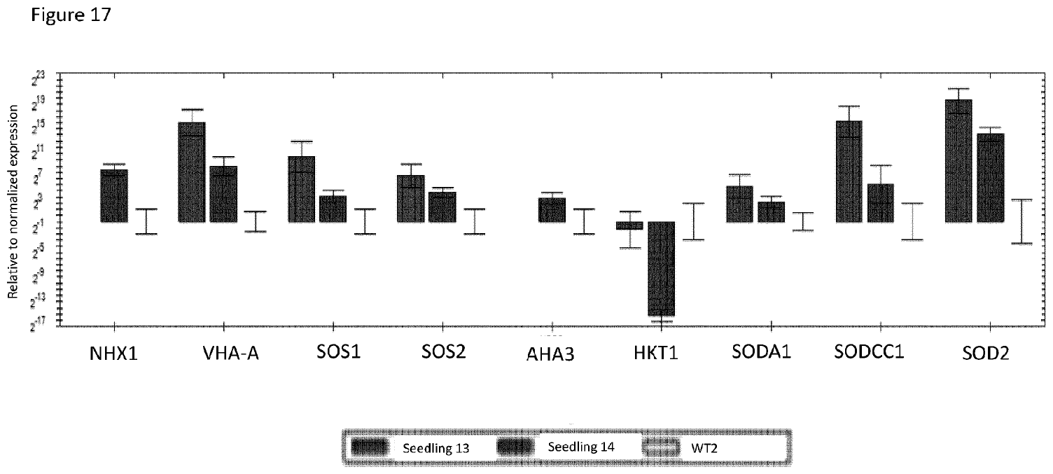

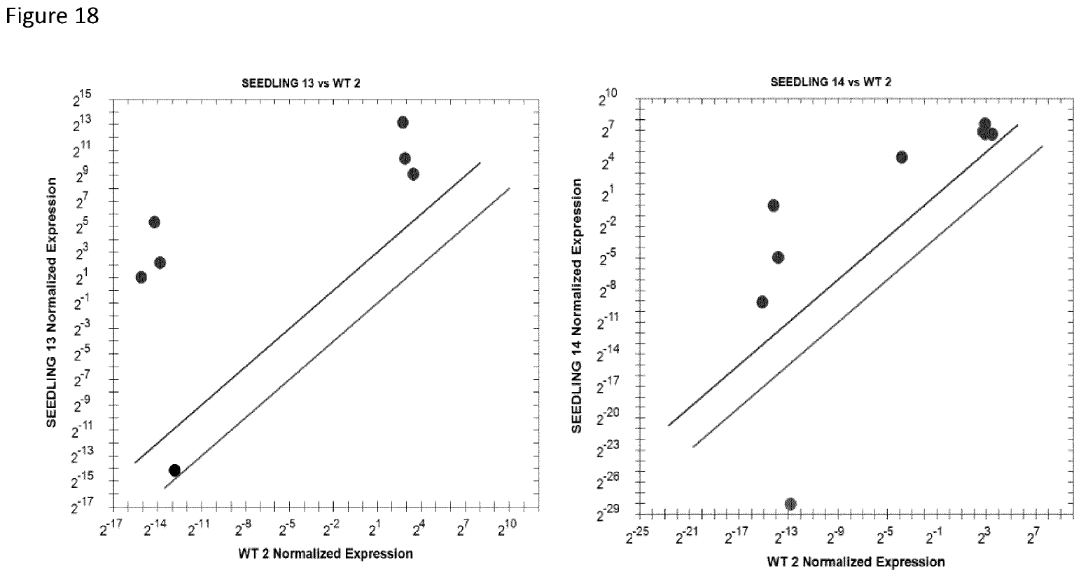

Seedlings 13 and 14 are two seedlings that were grown from seeds collected from Hayayuki Plant 1. FIG. 17 shows that the RNA expression levels of OsNHX1, OsVHA-A, OsSOS1, OsSOS2, OsAHA3, OsSODA1, SODCC1 and OsSOD2 in these seedlings was significantly higher than compared to the wild type rice. These data demonstrate that these genes were differentially expressed in the progeny compared to wild type plants. This difference is further evaluated in the scatter plots provided in FIG. 18. The gene expression observed in the two seedlings is similar to the gene expression observed in the parent plant (Hayayuki 1) and to other engineered rice plants according to the invention. These data demonstrate that the salt-tolerance traits transformed into the engineered plants of the invention are successfully inherited.

The invention provides the following numbered embodiments:
1. A transgenic plant comprising at least two transgenes wherein the at least two transgenes comprise a transgene that encodes a protein that controls the intracellular ion concentration and a transgene that encodes an antioxidant.
2. The transgenic plant according to embodiment 1, wherein the protein that controls the intracellular ion concentration is an ion transporter, a hydrogen exporting ATPase, a hydrogen exporting pyrophosphatase or a protein kinase.
3. The transgenic plant according to embodiment 2, wherein the ion transporter, the hydrogen exporting ATPase and/or the hydrogen exporting pyrophosphatase are a plasma membrane ion transporter, a plasma membrane hydrogen exporting ATPase and/or a plasma membrane hydrogen exporting pyrophosphatase.
4. The transgenic plant according to embodiment 3, wherein the plasma membrane ion transporter is SOS1 and/or the plasma membrane hydrogen exporting ATPase is AHA3.
5. The transgenic plant according to embodiment 4, wherein:
   (a) the SOS1 is OsSOS1, optionally wherein the OsSOS1 transgene comprises a polynucleotide sequence that encodes a polypeptide comprising an amino acid sequence that has at least 95% sequence identity to SEQ ID NO: 95 and/or 96; and/or
   (b) the AHA3 is OsAHA3, optionally wherein the OsAHA3 transgene comprises a polynucleotide sequence that encodes a polypeptide comprising an amino acid sequence that has at least 95% sequence identity to SEQ ID NO: 101.
6. The transgenic plant according to embodiment 2, wherein the ion transporter, the hydrogen exporting ATPase and/or the hydrogen exporting pyrophosphatase are a tonoplast ion transporter, a tonoplast hydrogen exporting ATPase and/or a tonoplast hydrogen exporting pyrophosphatase.
7. The transgenic plant according to embodiment 6, wherein:
   (a) the tonoplast ion transporter is NHX1; and/or
   (b) the tonoplast hydrogen exporting ATPase is VHA-A.
8. The transgenic plant according to embodiment 7, wherein:
   (a) the NHX1 is OsNHX1, optionally wherein the OsNHX1 transgene comprises a polynucleotide sequence that encodes a polypeptide comprising an amino acid sequence that has at least 95% sequence identity to SEQ ID NO: 97 and/or 98; and/or
   (b) the VHA-A is OsVHA-A, optionally wherein the OsVHA-A transgene comprises a polynucleotide sequence that encodes a polypeptide comprising an amino acid sequence that has at least 95% sequence identity to SEQ ID NO: 102.
9. The transgenic plant according to any one of embodiments 2-8, wherein the protein kinase is a serine/threonine kinase, optionally wherein the serine/threonine kinase is SOS2.
10. The transgenic plant according to embodiment 9, wherein the SOS2 is OsSOS2, optionally wherein the OsSOS2 transgene comprises a polynucleotide sequence that encodes a polypeptide comprising an amino acid sequence that has at least 95% sequence identity to SEQ ID NO: 100.
11. The transgenic plant according to any one of embodiments 1-10, wherein the antioxidant is a mitochondrial antioxidant or a cytoplasmic antioxidant.
12. The transgenic plant according to any one of embodiments 1-11, wherein the transgenic plant comprises at least two transgenes that encode antioxidants.
13. The transgenic plant according to embodiment 12, wherein the antioxidants comprise a mitochondrial antioxidant and a cytoplasmic antioxidant.
14. The transgenic plant according to any one of embodiments 1-13, wherein the transgenic plant comprises at least three transgenes that encode antioxidants.
15. The transgenic plant according to any one of embodiments 1-14, wherein the antioxidant(s) comprise(s) SODA1, SOD2 and/or SODCC1.
16. The transgenic plant according to embodiment 15, wherein:
   (a) the SODA1 is OsSODA1, optionally wherein the OsSODA1 transgene comprises a polynucleotide sequence that encodes a polypeptide comprising an amino acid sequence that has at least 95% sequence identity to SEQ ID NO: 92; and/or
   (b) the SOD2 is OsSOD2, optionally wherein the OsSOD2 transgene comprises a polynucleotide sequence that encodes a polypeptide comprising an amino acid sequence that has at least 95% sequence identity to SEQ ID NO: 93; and/or
   (c) the SODCC1 is OsSODCC1, optionally wherein the OsSODCC1 transgene comprises a polynucleotide sequence that encodes a polypeptide comprising an amino acid sequence that has at least 95% sequence identity to SEQ ID NO: 94.
17. A transgenic plant comprising at least two transgenes wherein the at least two transgenes comprise a transgene that encodes a plasma membrane protein that controls the intracellular ion concentration and a transgene that encodes a tonoplast protein that controls the intracellular ion concentration.
18. The transgenic plant according to embodiment 17, wherein the plasma membrane protein that controls the intracellular ion concentration is a plasma membrane ion transporter, a plasma membrane hydrogen exporting ATPase or a plasma membrane hydrogen exporting pyrophosphatase, optionally wherein the plasma membrane ion transporter is SOS1 and/or the plasma membrane hydrogen exporting ATPase is AHA3.
19. The transgenic plant according to embodiment 18, wherein:
   (a) the SOS1 is OsSOS1, optionally wherein the OsSOS1 transgene comprises a polynucleotide sequence that encodes a polypeptide comprising an amino acid sequence that has at least 95% sequence identity to SEQ ID NO: 95 and/or 96; and/or
   (b) the AHA3 is OsAHA3, optionally wherein the OsAHA3 transgene comprises a polynucleotide sequence that encodes a polypeptide comprising an amino acid sequence that has at least 95% sequence identity to SEQ ID NO: 101.
20. The transgenic plant according to any one of embodiments 17-19, wherein the tonoplast protein that controls the intracellular ion concentration is a tonoplast ion transporter, a tonoplast hydrogen exporting ATPase or a tonoplast hydrogen exporting pyrophosphatase.
21. The transgenic plant according to embodiment 20, wherein the tonoplast ion transporter is NHX1 and/or the tonoplast hydrogen exporting ATPase is VHA-A.
22. The transgenic plant according to embodiment 21, wherein:
   (a) the NHX1 is OsNHX1, optionally wherein the OsNHX1 transgene comprises a polynucleotide sequence that encodes a polypeptide comprising an amino acid sequence that has at least 95% sequence identity to SEQ ID NO: 97 and/or 98; and/or
   (b) the VHA-A is OsVHA-A, optionally wherein the OsVHA-A transgene comprises a polynucleotide sequence that encodes a polypeptide comprising an amino acid sequence that has at least 95% sequence identity to SEQ ID NO: 102.
23. The transgenic plant according to any one of embodiments 17-22, wherein the transgenic plant further comprises a transgene that encodes a protein kinase and/or a transgene that encodes an antioxidant.
24. The transgenic plant according to embodiment 23, wherein the protein kinase is a serine/threonine kinase, optionally wherein the serine/threonine kinase is SOS2.
25. The transgenic plant according to embodiment 24, wherein the SOS2 is OsSOS2, optionally wherein the OsSOS2 transgene comprises a polynucleotide sequence that encodes a polypeptide comprising an amino acid sequence that has at least 95% sequence identity to SEQ ID NO: 100.
26. The transgenic plant according to any one of embodiments 23-25, wherein the antioxidant is a mitochondrial antioxidant or a cytoplasmic antioxidant.
27. The transgenic plant according to any one of embodiments 23-26, wherein the antioxidant comprises any one of SODA1, SOD2 and SODCC1.
28. The transgenic plant according to embodiment 27, wherein:
   (a) the SODA1 is OsSODA1, optionally wherein the OsSODA1 transgene comprises a polynucleotide sequence that encodes a polypeptide comprising an amino acid sequence that has at least 95% sequence identity to SEQ ID NO: 92; and/or
   (b) the SOD2 is OsSOD2, optionally wherein the OsSOD2 transgene comprises a polynucleotide sequence that encodes a polypeptide comprising an amino acid sequence that has at least 95% sequence identity to SEQ ID NO: 93; and/or
   (c) the SODCC1 is OsSODCC1, optionally wherein the OsSODCC1 transgene comprises a polynucleotide sequence that encodes a polypeptide comprising an amino acid sequence that has at least 95% sequence identity to SEQ ID NO: 94.
29. A transgenic plant comprising at least three transgenes, wherein the transgenic plant has increased salt tolerance compared to a plant of a same species without said genome modifications.
30. A transgenic plant comprising at least three transgenes wherein the at least three transgenes comprise a transgene that encodes a plasma membrane protein that controls the intracellular ion concentration, a transgene that encodes a tonoplast protein that controls the intracellular ion concentration and a transgene that encodes an antioxidant.
31. The transgenic plant according to embodiment 30, wherein the plasma membrane protein and/or the tonoplast protein that controls the intracellular ion concentration is an ion transporter, a hydrogen exporting ATPase or a hydrogen exporting pyrophosphatase.
32. The transgenic plant according to embodiment 30 or embodiment 31, wherein the transgenic plant further comprises a transgene that encodes a protein kinase.
33. The transgenic plant according to embodiment 32, wherein the protein kinase is a serine/threonine kinase, optionally wherein the serine/threonine kinase is SOS2.
34. A transgenic rice plant comprises at least four transgenes, wherein the at least four transgenes comprise transgenes that encode OsSOS1, OsNHX1, OsHKT1 and OsSODA1.
35. A transgenic rice plant comprising at least four transgenes, wherein the at least four transgenes comprise transgenes that encode OsSOS1, OsNHX1, OsHKT1 and OsSODA1, wherein the OsSOS1 transgene comprises a polynucleotide sequence that encodes a polypeptide comprising an amino acid sequence that has at least 95% sequence identity to SEQ ID NO: 95 and/or 96; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92.
36. The transgenic plant according to any one of embodiments 1-35, wherein the transgenic plant comprises at least seven transgenes, wherein the at least seven transgenes comprise
   (a) a transgene that encodes a plasma membrane ion transporter;
   (b) a transgene that encodes a plasma membrane hydrogen exporting ATPase
   (c) a transgene that encodes a protein kinase
   (d) a transgene that encodes a vacuolar hydrogen exporting ATPase
   (e) a transgene that encodes a vacuolar sodium/proton transporter
   (f) a transgene that encodes a potassium transporter; and
   (g) a transgene that encodes an antioxidant.
37. The transgenic plant according to any one of embodiments 1-36, wherein the transgenic plant comprises at least eight transgenes, wherein the at least eight transgenes comprise
   (a) a transgene that encodes a plasma membrane ion transporter;
   (b) a transgene that encodes a plasma membrane hydrogen exporting ATPase
   (c) a transgene that encodes a protein kinase
   (d) a transgene that encodes a vacuolar hydrogen exporting ATPase
   (e) a transgene that encodes a vacuolar sodium/proton transporter
   (f) a transgene that encodes a potassium transporter; and
   (g) a transgene that encodes a first antioxidant and a second antioxidant.
38. The transgenic plant according to any one of embodiments 36-37, wherein:
   (a) the plasma membrane ion transporter is SOS1; and/or
   (b) the plasma membrane hydrogen exporting ATPase is AHA3; and/or
   (c) the protein kinase is SOS2; and/or
   (d) the vacuolar hydrogen exporting ATPase is VHA-A; and/or
   (e) the vacuolar ion transporter is NHX1; and/or
   (f) the potassium transporter is HKT1; and/or
   (g) the first and the second antioxidants are SODA1, SOD2 and/or SODCC1.
39. The transgenic plant according to embodiment 38, wherein:
   (a) the SOS1 is OsSOS1, optionally wherein the OsSOS1 transgene comprises a polynucleotide sequence that encodes a polypeptide comprising an amino acid sequence that has at least 95% sequence identity to SEQ ID NO: 95 and/or 96; and/or
   (b) the AHA3 is OsAHA3, optionally wherein the OsAHA3 transgene comprises a polynucleotide sequence that encodes a polypeptide comprising an amino acid sequence that has at least 95% sequence identity to SEQ ID NO: 101; and/or
   (c) the SOS2 is OsSOS2, optionally wherein the OsSOS2 transgene comprises a polynucleotide sequence that encodes a polypeptide comprising an amino acid sequence that has at least 95% sequence identity to SEQ ID NO: 100; and/or
   (d) the VHA-A is OsVHA-A, optionally wherein the OsVHA-A transgene comprises a polynucleotide sequence that encodes a polypeptide comprising an amino acid sequence that has at least 95% sequence identity to SEQ ID NO: 102; and/or
   (e) the NHX1 is OsNHX1, optionally wherein the OsNHX1 transgene comprises a polynucleotide sequence that encodes a polypeptide comprising an amino acid sequence that has at least 95% sequence identity to SEQ ID NO: 97 and/or 98; and/or
   (f) the HKT1 is OsHKT1, optionally wherein the OsHKT1 transgene comprises a polynucleotide sequence that encodes a polypeptide comprising an amino acid sequence that has at least 95% sequence identity to SEQ ID NO: 99;
   (g) the SODA1 is OsSODA1, optionally wherein the OsSODA1 transgene comprises a polynucleotide sequence that encodes a polypeptide comprising an amino acid sequence that has at least 95% sequence identity to SEQ ID NO: 92; and/or
   (h) the SOD2 is OsSOD2, optionally wherein the OsSOD2 transgene comprises a polynucleotide sequence that encodes a polypeptide comprising an amino acid sequence that has at least 95% sequence identity to SEQ ID NO: 93; and/or
   (i) the SODCC1 is OsSODCC1, optionally wherein the OsSODCC1 transgene comprises a polynucleotide sequence that encodes a polypeptide comprising an amino acid sequence that has at least 95% sequence identity to SEQ ID NO: 94.
40. A transgenic rice plant comprises at least eight transgenes, wherein the at least eight transgenes comprise transgenes that encode OsSOS1, OsSOS2, OsAHA3, OsVHA-A, OsNHX1, OsHKT1, OsSODA1 and OsSOD2.
41. A transgenic rice plant comprising at least eight transgenes, wherein the at least eight transgenes comprise transgenes that encode OsSOS1, OsSOS2, OsAHA3, OsVHA-A, OsNHX1, OsHKT1, OsSODA1 and OsSOD2;
   wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93.
42. A transgenic rice plant comprises at least eight transgenes, wherein the at least eight transgenes comprise transgenes that encode OsSOS1, OsSOS2, OsAHA3, OsVHA-A, OsNHX1, OsHKT1, OsSODA1 and OsSODCC1.
43. A transgenic rice plant comprising at least eight transgenes wherein the at least eight transgenes comprise transgenes that encode OsSOS1, OsSOS2, OsAHA3, OsVHA-A, OsNHX1, OsHKT1, OsSODA1 and OsSODCC1;
   wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA-A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSODCC1 transgene comprises a polynucleotide sequence that encodes a polypeptide comprising an amino acid sequence that has at least 95% sequence identity to SEQ ID NO: 94.
44. The transgenic plant according to any one of the preceding embodiments, wherein the transgenic plant further comprises at least one additional transgene, wherein the at least one additional transgene encodes a protein that is:
   (a) a cytochrome p450 (P450); and/or
   (b) an oxygen-evolving complex; and/or
   (c) a sucrose phosphate synthase; and/or
   (d) pyrroline carboxylate synthase.
45. The transgenic plant according to embodiment 44, wherein the oxygen-evolving complex is formed of the proteins PsbO, PsbP and PsbQ.
46. The transgenic plant according to embodiment 44 or embodiment 45, wherein the oxygen-evolving complex is formed of the proteins PsbO, PsbP, PsbQ, PsbU and PsbV.
47. The transgenic plant according to any one of embodiments 44-46, wherein the sucrose phosphate synthase is sucrose phosphate synthase 1, sucrose phosphate synthase 2 or sucrose phosphate synthase 3.
48. The transgenic plant according to any one of embodiments 44-47, wherein the pyrroline carboxylate synthase is delta-1-pyrroline-5-carboxylate synthase 1, or delta-1-pyrroline-5-carboxylate synthase 2.
49. The transgenic plant according to any one of the preceding embodiments, wherein the transgenic plant further comprises a transgene that encodes OSK1.
50. The transgenic plant according to any one of the preceding embodiments, wherein the transgenic plant does not contain a transgene that encodes PERK13.
51. The transgenic plant according to any one of the preceding embodiments, wherein at least one of the transgenes is operably linked to at least one promoter, optionally wherein all of the transgenes are operably linked to at least one promoter.
52. The transgenic plant according to embodiment 51, wherein the at least one promoter comprises at least 10, at least 20, or at least 30 nucleotides.
53. The transgenic plant according to embodiment 51 or embodiment 52, wherein the at least one promoter is within 150-500 nucleotides of the 5′ end of an open reading frame of the transgene.
54. The transgenic plant according to any one of embodiments 51-53, wherein the at least one promoter is a root-specific promoter, optionally wherein all of the transgenes are operably linked to a root-specific promoter.
55. The transgenic plant according to any one of the embodiments 51-54, wherein the at least one promoter comprises a DREB2A, gibberellin, ethylene or auxin promoter sequence or a combination thereof, optionally wherein all of the transgenes are operably linked to a promoter comprising a DREB2A, gibberellin, ethylene or auxin promoter sequence or a combination thereof.
56. The transgenic plant according to any one of the embodiments 51-55, wherein the at least one promoter comprises at least 6 nucleotides from an promoter element from a DREB2A, gibberellin, ethylene or auxin promoter sequence or a combination thereof, optionally wherein all of the transgenes are operably linked to a promoter comprising an promoter element from a DREB2A, gibberellin, ethylene or auxin promoter sequence.
57. The transgenic plant according to any one of the embodiments 51-56, wherein the at least one promoter comprises a TAF-1, TATA, E2F, G-BOX, or CAAT promoter sequence or a combination thereof, optionally wherein all of the transgenes are operably linked to a promoter comprising a TAF-1, TATA, E2F, G-BOX, or CAAT promoter sequence.
58. The transgenic plant according to any one of the embodiments 51-57, wherein the at least one promoter comprises a sequence having at least 95% sequence identity to any one of SEQ ID NO: 10-18.
59. The transgenic plant according to any one of the preceding embodiments, wherein each transgene encodes a separate protein.
60. The transgenic plant according to any one of the preceding embodiments, wherein the transgenic plant is an angiosperm.
61. The transgenic plant according to embodiment 60, wherein the transgenic plant is monocotyledonous or dicotyledonous.
62. The transgenic plant according to embodiment 60 or embodiment 61, wherein the transgenic plant is a cereal crop.
63. The transgenic plant according to embodiment 62, wherein the transgenic plant is maize, rice, soybean, sugar cane, mung bean, quinoa, barley, oat, rye, sorghum, or wheat.
64. The transgenic plant according to any one of embodiments 60-63, wherein transgenic plant is a transgenic rice plant.
65. The transgenic plant according to embodiment 60 or embodiment 61, wherein the transgenic plant is a vegetable crop.
66. The transgenic plant according to embodiment 65, wherein the transgenic plant is from the genus a Brassica, Glycine, or Soja.
67. A plant part of the transgenic plant according to any one of embodiments 1-66.
68. The plant part according to embodiment 67, wherein the plant part is a cell, a seed, a leaf, a shoot, a stem or a root.
69. The plant part according to embodiment 68, wherein the plant part is a seed.
70. The plant part according to embodiment 68, wherein the plant part is a cell.
71. A multicellular structure comprising one or more plant cells according to embodiment 70.
72. The multicellular structure of embodiment 71, wherein the multicellular structure is a callus.
73. A method of making the transgenic plant, the plant part or the multicellular structure according to embodiments 1-72, the method comprising the steps of
   i) introducing the at least two transgenes as defined in the transgenic plants according to embodiments 1-66 into a cell of a plant, wherein the transgenes integrate into the genome of the cell of the plant, and
   ii) regenerating the cell to form a transgenic plant, a plant part or a multicellular structure from the cell.
74. The method according to embodiment 73, wherein the transgenes are introduced into the cell by particle bombardment, Agrobacterium mediated transformation or by protoplast transfection.
75. The method according to any one of embodiments 73-74, wherein the methods involves:
   (a) inducing callus formation from a seed;
   (b) precipitating a polynucleotide sequence, a guide RNA and a nuclease onto a microcarrier; wherein the polynucleotide sequence comprises the at least two transgenes as defined according to any one of embodiments 1-72
   (c) transforming the callus with the microcarriers using particle bombardment to generate a transformed callus wherein the polynucleotide sequence integrates into the genome of the transgenic plant, the plant part or the multicellular structure;
   (d) recovering the transformed callus to generate a multicellular structure according to embodiment 71 or embodiment 72.
76. The method according to embodiment 75, wherein the multicellular structure is regenerated into a transgenic plant.
77. The method according to embodiment 75 or embodiment 76, wherein the polynucleotide sequence is stably integrated into the genome of the plant.
78. The method according to any one of embodiments 75-77, wherein the nuclease is a Cas nuclease, Cpf1 nuclease, a TALEN or a zinc finger nucleases, optionally wherein the nuclease is Cas9 or Cpf1.
79. The method according to any one of embodiments 75-78, wherein the polynucleotide is RNA, DNA or a plasmid, optionally wherein the polynucleotide is DNA.
80. The transgenic plant, plant part or multicellular structure according to embodiments 1-72, wherein the transgenic plant, the plant part or the multicellular structure is not produced by a process that involves homologous recombination and/or is not produced by an essentially biological process.
81. A method of producing flour, wholemeal, starch or other product obtained from a seed according to embodiment 69.
82. Use of a transgenic plant according to any one of embodiments 1 to 66, plant part according to any one of embodiments 67-70 or multicellular structure according to embodiment 71 or embodiment 72, as animal feed, or to produce feed for animal consumption or food for human consumption.
83. A transgenic plant as defined in any preceding embodiment wherein one or more of the transgenes is replaced with a corresponding gene of interest that is operatively linked to an enhancer element as disclosed anywhere herein, optionally as disclosed in any of the following embodiments.
   The invention also provides the following numbered embodiments:
1. An engineered plant comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element.
2. The engineered plant according to embodiment 1, wherein the protein that controls the intracellular ion concentration is an ion transporter, a hydrogen exporting ATPase, a hydrogen exporting pyrophosphatase or a protein kinase.
3. The engineered plant according to embodiment 2, wherein the ion transporter, the hydrogen exporting ATPase and/or the hydrogen exporting pyrophosphatase are a plasma membrane ion transporter, a plasma membrane hydrogen exporting ATPase and/or a plasma membrane hydrogen exporting pyrophosphatase.
4. The engineered plant according to embodiment 3, wherein the plasma membrane ion transporter is SOS1 and/or the plasma membrane hydrogen exporting ATPase is AHA3.
5. The engineered plant according to embodiment 4, wherein:
   (a) the SOS1 is OsSOS1, optionally wherein the OsSOS1 gene comprises a polynucleotide sequence that encodes a polypeptide comprising the amino acid sequence of SEQ ID NO: 95 and/or 96; and/or
   (b) the AHA3 is OsAHA3, optionally wherein the OsAHA3 gene comprises a polynucleotide sequence that encodes a polypeptide comprising the amino acid sequence of SEQ ID NO: 101.
6. The engineered plant according to embodiment 2, wherein the ion transporter, the hydrogen exporting ATPase and/or the hydrogen exporting pyrophosphatase are a tonoplast ion transporter, a tonoplast hydrogen exporting ATPase and/or a tonoplast hydrogen exporting pyrophosphatase.
7. The engineered plant according to embodiment 6, wherein:
   (a) the tonoplast ion transporter is NHX1; and/or
   (b) the tonoplast hydrogen exporting ATPase is VHA-A.
8. The engineered plant according to embodiment 7, wherein:
   (a) the NHX1 is OsNHX1, optionally wherein the OsNHX1 gene comprises a polynucleotide sequence that encodes a polypeptide comprising the amino acid sequence of SEQ ID NO: 97 and/or 98; and/or
   (b) the VHA-A is OsVHA-A, optionally wherein the OsVHA-A gene comprises a polynucleotide sequence that encodes a polypeptide comprising the amino acid sequence of SEQ ID NO: 102.
9. The engineered plant according to any one of embodiments 2-8, wherein the protein kinase is a serine/threonine kinase, optionally wherein the serine/threonine kinase is SOS2.
10. The engineered plant according to embodiment 9, wherein the SOS2 is OsSOS2, optionally wherein the OsSOS2 gene comprises a polynucleotide sequence that encodes a polypeptide comprising the amino acid sequence of SEQ ID NO: 100.
11. The engineered plant according to any one of embodiments 1-10, wherein the antioxidant is a mitochondrial antioxidant or a cytoplasmic antioxidant.
12. The engineered plant according to any one of embodiments 1-11, wherein the engineered plant comprises at least two genes of interest that encode antioxidants.
13. The engineered plant according to embodiment 12, wherein the antioxidants comprise a mitochondrial antioxidant and a cytoplasmic antioxidant.
14. The engineered plant according to any one of embodiments 1-13, wherein the engineered plant comprises at least three gene that encode antioxidants.
15. The engineered plant according to any one of embodiments 1-14, wherein the antioxidant(s) comprise(s) SODA1, SOD2 and/or SODCC1.
16. The engineered plant according to embodiment 15, wherein:
   (a) the SODA1 is OsSODA1, optionally wherein the OsSODA1 gene comprises a polynucleotide sequence that encodes a polypeptide comprising the amino acid sequence of SEQ ID NO: 92; and/or
   (b) the SOD2 is OsSOD2, optionally wherein the OsSOD2 gene comprises a polynucleotide sequence that encodes a polypeptide comprising the amino acid sequence of SEQ ID NO: 93; and/or
   (c) the SODCC1 is OsSODCC1, optionally wherein the OsSODCC1 gene comprises a polynucleotide sequence that encodes a polypeptide comprising the amino acid sequence of SEQ ID NO: 94.
17. An engineered plant comprising at least two genes of interest, wherein the genes of interest comprise a gene that encodes a plasma membrane protein that controls the intracellular ion concentration and a gene that encodes a tonoplast protein that controls the intracellular ion concentration, wherein the gene that encodes a plasma membrane protein that controls the intracellular ion concentration is operatively linked to an enhancer element and the a gene that encodes a tonoplast protein that controls the intracellular ion concentration is operatively linked to an enhancer element.
18. The engineered plant according to embodiment 17, wherein the plasma membrane protein that controls the intracellular ion concentration is a plasma membrane ion transporter, a plasma membrane hydrogen exporting ATPase or a plasma membrane hydrogen exporting pyrophosphatase, optionally wherein the plasma membrane ion transporter is SOS1 and/or the plasma membrane hydrogen exporting ATPase is AHA3.
19. The engineered plant according to embodiment 18, wherein:
   (a) the SOS1 is OsSOS1, optionally wherein the OsSOS1 gene comprises a polynucleotide sequence that encodes a polypeptide comprising the amino acid sequence of SEQ ID NO: 95 and/or 96; and/or
   (b) the AHA3 is OsAHA3, optionally wherein the OsAHA3 gene comprises a polynucleotide sequence that encodes a polypeptide comprising the amino acid sequence of SEQ ID NO: 101.
20. The engineered plant according to any one of embodiments 17-19, wherein the tonoplast protein that controls the intracellular ion concentration is a tonoplast ion transporter, a tonoplast hydrogen exporting ATPase or a tonoplast hydrogen exporting pyrophosphatase.
21. The engineered plant according to embodiment 20, wherein the tonoplast ion transporter is NHX1 and/or the tonoplast hydrogen exporting ATPase is VHA-A.
22. The engineered plant according to embodiment 21, wherein:
   (a) the NHX1 is OsNHX1, optionally wherein the OsNHX1 gene comprises a polynucleotide sequence that encodes a polypeptide comprising the amino acid sequence of SEQ ID NO: 97 and/or 98; and/or
   (b) the VHA-A is OsVHA-A, optionally wherein the OsVHA-A gene comprises a polynucleotide sequence that encodes a polypeptide comprising the amino acid sequence of SEQ ID NO: 102.
23. The engineered plant according to any one of embodiments 17-22, wherein the engineered plant further comprises a gene that encodes a protein kinase and/or a gene that encodes an antioxidant.
24. The engineered plant according to embodiment 23, wherein the protein kinase is a serine/threonine kinase, optionally wherein the serine/threonine kinase is SOS2.
25. The engineered plant according to embodiment 24, wherein the SOS2 is OsSOS2, optionally wherein the OsSOS2 gene comprises a polynucleotide sequence that encodes a polypeptide comprising the amino acid sequence of SEQ ID NO: 100.
26. The engineered plant according to any one of embodiments 23-25, wherein the antioxidant is a mitochondrial antioxidant or a cytoplasmic antioxidant.
27. The engineered plant according to any one of embodiments 23-26, wherein the antioxidant comprises any one of SODA1, SOD2 and SODCC1.
28. The engineered plant according to embodiment 27, wherein:
   (a) the SODA1 is OsSODA1, optionally wherein the OsSODA1 gene comprises a polynucleotide sequence that encodes a polypeptide comprising the amino acid sequence of SEQ ID NO: 92; and/or
   (b) the SOD2 is OsSOD2, optionally wherein the OsSOD2 gene comprises a polynucleotide sequence that encodes a polypeptide comprising the amino acid sequence of SEQ ID NO: 93; and/or
   (c) the SODCC1 is OsSODCC1, optionally wherein the OsSODCC1 gene comprises a polynucleotide sequence that encodes a polypeptide comprising the amino acid sequence of SEQ ID NO: 94.
29. An engineered plant comprising at least three genes of interest, wherein each gene of interest is operatively linked to an enhancer element, wherein the engineered plant has increased salt tolerance compared to a plant of a same species without said genome modifications.
30. An engineered plant comprising at least three genes of interest wherein the genes of interest comprise a gene that encodes a plasma membrane protein that controls the intracellular ion concentration, a gene that encodes a tonoplast protein that controls the intracellular ion concentration and a gene that encodes an antioxidant, and
   wherein the gene that encodes a plasma membrane protein that controls the intracellular ion concentration is operatively linked to an enhancer element, the gene that encodes a tonoplast protein that controls the intracellular ion concentration is operatively linked to an enhancer element and the gene that encodes an antioxidant is operatively linked to an enhancer element.
31. The engineered plant according to embodiment 30, wherein the plasma membrane protein and/or the tonoplast protein that controls the intracellular ion concentration is an ion transporter, a hydrogen exporting ATPase or a hydrogen exporting pyrophosphatase.
32. The engineered plant according to embodiment 30 or embodiment 31, wherein the engineered plant further comprises a gene that encodes a protein kinase.
33. The engineered plant according to embodiment 32, wherein the protein kinase is a serine/threonine kinase, optionally wherein the serine/threonine kinase is SOS2.
34. An engineered rice plant comprising at least four genes of interest, wherein the at least four genes of interest encode OsSOS1, OsNHX1, OsHKT1 and OsSODA1, wherein each gene of interest is operatively linked to an enhancer element.
35. An engineered rice plant comprising at least four genes of interest, wherein the at least four genes of interest encode OsSOS1, OsNHX1, OsHKT1 and OsSODA1, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising an amino acid sequence that has at least 95% sequence identity to SEQ ID NO: 92, and
   wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element
36. The engineered plant according to any one of embodiments 1-35, wherein the engineered plant comprises at least seven genes of interest, wherein the at least seven genes of interest comprise
   (a) a gene that encodes a plasma membrane ion transporter and wherein the gene that encodes a plasma membrane ion transporter is operatively linked to an enhancer element;
   (b) a gene that encodes a plasma membrane hydrogen exporting ATPase and wherein the gene that encodes a plasma membrane hydrogen exporting ATPase is operatively linked to an enhancer element;
   (c) a gene that encodes a protein kinase and wherein the gene that encodes a protein kinase is operatively linked to an enhancer element;
   (d) a gene that encodes a vacuolar hydrogen exporting ATPase and wherein the gene that encodes a vacuolar hydrogen exporting ATPase is operatively linked to an enhancer element;
   (e) a gene that encodes a vacuolar sodium/proton transporter and wherein the gene that encodes a vacuolar sodium/proton transporter is operatively linked to an enhancer element;
   (f) a gene that encodes a potassium transporter and wherein the gene that encodes a potassium transporter is operatively linked to an enhancer element; and
   (g) a gene that encodes an antioxidant and the gene that encodes an antioxidant is operatively linked to an enhancer element.
37. The engineered plant according to any one of embodiments 1-36, wherein the engineered plant comprises at least eight genes of interest, wherein the at least eight genes of interest comprise
   (a) a gene that encodes a plasma membrane ion transporter and wherein the gene that encodes a plasma membrane ion transporter is operatively linked to an enhancer element;
   (b) a gene that encodes a plasma membrane hydrogen exporting ATPase and wherein the gene that encodes a plasma membrane hydrogen exporting ATPase is operatively linked to an enhancer element;
   (c) a gene that encodes a protein kinase and wherein the gene that encodes a protein kinase is operatively linked to an enhancer element;
   (d) a gene that encodes a vacuolar hydrogen exporting ATPase and wherein the gene that encodes a vacuolar hydrogen exporting ATPase is operatively linked to an enhancer element;
   (e) a gene that encodes a vacuolar sodium/proton transporter and wherein the gene that encodes a vacuolar sodium/proton transporter is operatively linked to an enhancer element;
   (f) a gene that encodes a potassium transporter and wherein the gene that encodes a potassium transporter is operatively linked to an enhancer element;
   (g) a gene that encodes a first antioxidant and the gene that encodes a first antioxidant is operatively linked to an enhancer element; and
   (h) a gene that encodes a second antioxidant and the gene that encodes a second antioxidant is operatively linked to an enhancer element.
38. The engineered plant according to any one of embodiments 36-37, wherein:
   (a) the plasma membrane ion transporter is SOS1; and/or
   (b) the plasma membrane hydrogen exporting ATPase is AHA3; and/or
   (c) the protein kinase is SOS2; and/or
   (d) the vacuolar hydrogen exporting ATPase is VHA-A; and/or
   (e) the vacuolar ion transporter is NHX1; and/or
   (f) the potassium transporter is HKT1; and/or
   (g) the first and the second antioxidants are SODA1, SOD2 and/or SODCC1.
39. The engineered plant according to embodiment 38, wherein:
   (a) the SOS1 is OsSOS1, optionally wherein the OsSOS1 gene comprises a polynucleotide sequence that encodes a polypeptide comprising the amino acid sequence of SEQ ID NO: 95 and/or 96; and/or
   (b) the AHA3 is OsAHA3, optionally wherein the OsAHA3 gene comprises a polynucleotide sequence that encodes a polypeptide comprising the amino acid sequence of SEQ ID NO: 101; and/or
   (c) the SOS2 is OsSOS2, optionally wherein the OsSOS2 gene comprises a polynucleotide sequence that encodes a polypeptide comprising the amino acid sequence of SEQ ID NO: 100; and/or
   (d) the VHA-A is OsVHA-A, optionally wherein the OsVHA-A gene comprises a polynucleotide sequence that encodes a polypeptide comprising the amino acid sequence of SEQ ID NO: 102; and/or
   (e) the NHX1 is OsNHX1, optionally wherein the OsNHX1 gene comprises a polynucleotide sequence that encodes a polypeptide comprising the amino acid sequence of SEQ ID NO: 97 and/or 98; and/or
   (f) the HKT1 is OsHKT1, optionally wherein the OsHKT1 gene comprises a polynucleotide sequence that encodes a polypeptide comprising the amino acid sequence of SEQ ID NO: 99;
   (g) the SODA1 is OsSODA1, optionally wherein the OsSODA1 gene comprises a polynucleotide sequence that encodes a polypeptide comprising the amino acid sequence of SEQ ID NO: 92; and/or
   (h) the SOD2 is OsSOD2, optionally wherein the OsSOD2 gene comprises a polynucleotide sequence that encodes a polypeptide comprising the amino acid sequence of SEQ ID NO: 93; and/or
   (i) the SODCC1 is OsSODCC1, optionally wherein the OsSODCC1 gene comprises a polynucleotide sequence that encodes a polypeptide comprising the amino acid sequence of SEQ ID NO: 94.
40. An engineered rice plant comprises at least eight genes of interest, wherein the at least eight genes of interest encode OsSOS1, OsSOS2, OsAHA3, OsVHA-A, OsNHX1, OsHKT1, OsSODA1 and OsSOD2;
   wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.
41. An engineered rice plant comprising at least eight genes of interest, wherein the at least eight genes of interest encode OsSOS1, OsSOS2, OsAHA3, OsVHA-A, OsNHX1, OsHKT1, OsSODA1 and OsSOD2
   wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; and
   wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.
42. An engineered rice plant comprises at least eight genes of interest, wherein the at least eight genes of interest encode OsSOS1, OsSOS2, OsAHA3, OsVHA-A, OsNHX1, OsHKT1, OsSODA1 and OsSODCC1;
   wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.
43. An engineered rice plant comprising at least eight genes of interest wherein the at least eight genes of interest encode OsSOS1, OsSOS2, OsAHA3, OsVHA-A, OsNHX1, OsHKT1, OsSODA1 and OsSODCC1;
   wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94; and
   wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.
44. The engineered plant according to any one of the preceding embodiments, wherein the engineered plant further comprises at least one additional gene of interest operatively linked to an enhancer element, wherein the at least one additional gene of interest (that is operatively linked to an enhancer element) encodes a protein that is:
   (a) a cytochrome p450 (P450); and/or
   (b) an oxygen-evolving complex; and/or
   (c) a sucrose phosphate synthase; and/or
   (d) pyrroline carboxylate synthase.
45. The engineered plant according to embodiment 44, wherein the oxygen-evolving complex is formed of the proteins PsbO, PsbP and PsbQ.
46. The engineered plant according to embodiment 44 or embodiment 45, wherein the oxygen-evolving complex is formed of the proteins PsbO, PsbP, PsbQ, PsbU and PsbV.
47. The engineered plant according to any one of embodiments 44-46, wherein the sucrose phosphate synthase is sucrose phosphate synthase 1, sucrose phosphate synthase 2 or sucrose phosphate synthase 3.
48. The engineered plant according to any one of embodiments 44-47, wherein the pyrroline carboxylate synthase is delta-1-pyrroline-5-carboxylate synthase 1, or delta-1-pyrroline-5-carboxylate synthase 2.
49. The engineered plant according to any one of the preceding embodiments, wherein the engineered plant further comprises a gene of interest that encodes OSK1 that is operatively linked to an enhancer element.
50. The engineered plant according to any one of the preceding embodiments, wherein the engineered plant does not contain a gene of interest that encodes PERK13 or said gene of interest is not operatively linked to an enhancer element.
51. The engineered plant according to any one of the preceding embodiments, wherein the enhancer elements have the same polynucleotide sequence or wherein the enhancer elements have different polynucleotide sequences.
52. The engineered plant according to any one of the preceding embodiments, wherein the enhancer element alters the expression level of the gene of interest compared to the expression level of the gene of interest in a wild-type plant.
53. The engineered plant according to any one of the preceding embodiments, wherein the enhancer element increases or decreases the expression level of the gene of interest compared to the expression level of the gene of interest in a wild-type plant, optionally wherein the enhancer element increases the expression level of the gene of interest, compared to the expression level of the gene of interest in a wild-type plant, in at least one part of the plant during at least one developmental stage of the plant.
54. The engineered plant according to any one of the preceding embodiments, wherein the enhancer element alters the expression level of the gene of interest in the root, shoot, leaf, seed or stem.
55. The engineered plant according to embodiment 54, wherein the enhancer element alters the expression level of the gene of interest in the root.
56. The engineered plant according to any one of the preceding embodiments, wherein the enhancer element alters the expression level of the gene of interest during germination, when the plant is growing, when the plant is flowering and/or when the plant is developing fruit.
57. The engineered plant according to any one of the preceding embodiments, wherein the enhancer element comprises at least 10, at least 20, or at least 30 nucleotides.
58. The engineered plant according to any one of the preceding embodiments, wherein the enhancer element is within 150-500 nucleotides of the 5′ end of an open reading frame of the gene of interest.
59. The engineered plant according to any one of the preceding embodiments, wherein enhancer element comprises a root-specific promoter
60. The engineered plant according to embodiment 59, wherein all of the genes of interest are operably linked to enhancer elements comprising a root-specific promoter.
61. The engineered plant according to any one of the preceding embodiments, wherein the enhancer element comprises a DREB2A, gibberellin, ethylene or auxin promoter sequence or a combination thereof.
62. The engineered plant according to embodiment 61, wherein all of the genes of interest are operably linked to enhancer elements comprising a DREB2A, gibberellin, ethylene or auxin promoter sequence or a combination thereof.
63. The engineered plant according to any one of the preceding embodiments, wherein the enhancer element comprises at least 6 nucleotides from a promoter element from a DREB2A, gibberellin, ethylene or auxin promoter sequence or a combination thereof.
64. The engineered plant according to embodiment 63, wherein all of the genes of interest are operably linked to an enhancer element comprising a promoter element from a DREB2A, gibberellin, ethylene or auxin promoter sequence.
65. The engineered plant according to any one of the preceding embodiments, wherein the enhancer element comprises a TAF-1, TATA, E2F, G-BOX, or CAAT promoter sequence or a combination thereof.
66. The engineered plant according to embodiment 65, wherein all of the genes of interest are operably linked to an enhancer element comprising a TAF-1, TATA, E2F, G-BOX, or CAAT promoter sequence.
67. The engineered plant according to any one of the preceding embodiments, wherein the enhancer element comprises a sequence having at least 95% sequence identity to any one of SEQ ID NO: 10-18.
68. The engineered plant according to any one of the preceding embodiments, wherein:
   (a) the OsSOS1 gene is operably linked to an enhancer element comprising SEQ ID NO: 12, and/or
   (b) the OsSOS2 gene is operably linked to an enhancer element comprising SEQ ID NO: 13, and/or
   (c) the OsAHA3 gene is operably linked to an enhancer element comprising SEQ ID NO: 14, and/or
   (d) the OsVHA-A gene is operably linked to an enhancer element comprising SEQ ID NO: 11, and/or
   (e) the OsNHX1 gene is operably linked to an enhancer element comprising SEQ ID NO: 10, and/or
   (f) the OsHKT1 gene is operably linked to an enhancer element comprising SEQ ID NO: 15, and/or
   (g) the OsSODA1 gene is operably linked to an enhancer element comprising SEQ ID NO: 16 and/or
   (h) the OsSODCC1 gene is operably linked to an enhancer element comprising SEQ ID NO: 17 and/or
   (i) the OsSOD2 gene is operably linked to an enhancer element comprising SEQ ID NO: 18.
69. An engineered rice plant comprising at least eight genes of interest, wherein the eight genes of interest encode OsSOS1, OsSOS2, OsAHA3, OsVHA-A, OsNHX1, OsHKT1, OsSODA1 and OsSOD2;
   wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93;
   wherein the OsSOS1 gene is operably linked to an enhancer element comprising SEQ ID NO: 12, the OsSOS2 gene is operably linked to an enhancer element comprising SEQ ID NO: 13, the OsAHA3 gene is operably linked to an enhancer element comprising SEQ ID NO: 14, the OsVHA-A gene is operably linked to an enhancer element comprising SEQ ID NO: 11, the OsNHX1 gene is operably linked to an enhancer element comprising SEQ ID NO: 10, the OsHKT1 gene is operably linked to an enhancer element comprising SEQ ID NO: 15, the OsSODA1 gene is operably linked to an enhancer element comprising SEQ ID NO: 16 and the OsSOD2 gene is operably linked to an enhancer element comprising SEQ ID NO: 18.
70. The engineered plant according to any one of the preceding embodiments, wherein each gene of interest encodes a separate protein.
71. The engineered plant according to any one of the preceding embodiments, wherein the engineered plant is an angiosperm.
72. The engineered plant according to embodiment 71, wherein the engineered plant is monocotyledonous or dicotyledonous.
73. The engineered plant according to embodiment 71 or embodiment 72, wherein the engineered plant is a cereal crop.
74. The engineered plant according to embodiment 73, wherein the engineered plant is maize, rice, soybean, sugar cane, mung bean, quinoa, barley, oat, rye, sorghum, or wheat.
75. The engineered plant according to any one of embodiments 71-74, wherein engineered plant is an engineered rice plant.
76. The engineered plant according to embodiment 71 or embodiment 72, wherein the engineered plant is a vegetable crop.
77. The engineered plant according to embodiment 76, wherein the engineered plant is from the genus a Brassica, Glycine, or Soja.
78. A plant part of the engineered plant according to any one of embodiments 1-77.
79. The plant part according to embodiment 78, wherein the plant part is a cell, a seed, a leaf, a shoot, a stem or a root.
80. The plant part according to embodiment 78, wherein the plant part is a seed.
81. The plant part according to embodiment 78, wherein the plant part is a cell.
82. A multicellular structure comprising one or more plant cells according to embodiment 81.
83. The multicellular structure of embodiment 82, wherein the multicellular structure is a callus.
84. A method of making the engineered plant, the plant part or the multicellular structure according to embodiments 1-83, the method comprising the steps of
   i) introducing at least two enhancer elements as defined in the engineered plants according to embodiments 1-77 into a cell of a plant, wherein the enhancer elements integrate into the genome of the cell of the plant such that they are operatively linked to the genes of interest, and
   ii) regenerating the cell to form an engineered plant, a plant part or a multicellular structure from the cell.
85. The method according to embodiment 84, wherein the enhancer elements are introduced into the cell by particle bombardment, Agrobacterium mediated transformation or by protoplast transfection.
86. The method according to any one of embodiments 84-85, wherein the methods involves:
   (a) inducing callus formation from a seed;
   (b) precipitating the enhancer elements as defined in the engineered plants according to embodiments 1-77, two or more guide RNAs and one or more nucleases onto a microcarrier;
   (c) transforming the callus with the microcarriers using particle bombardment to generate a transformed callus wherein the enhancer elements integrate into the genome of the engineered plant, the plant part or the multicellular structure such that they are operatively linked to the genes of interest;
   (d) recovering the transformed callus to generate a multicellular structure according to embodiment 82 or embodiment 83.
87. The method according to embodiment 86, wherein the multicellular structure is regenerated into an engineered plant.
88. The method according to embodiment 86 or embodiment 87, wherein the polynucleotide sequence is stably integrated into the genome of the plant.
89. The method according to any one of embodiments 86-88, wherein the nuclease is a Cas nuclease, Cpf1 nuclease, a TALEN or a zinc finger nucleases, optionally wherein the nuclease is Cas9 or Cpf1.
90. The engineered plant, plant part or multicellular structure according to embodiments 1-83, wherein the engineered plant, the plant part or the multicellular structure is not produced by a process that involves homologous recombination and/or is not produced by an essentially biological process.
91. A method of producing flour, wholemeal, starch or other product obtained from a seed according to embodiment 80.
92. Use of an engineered plant according to any one of embodiments 1 to 77, plant part according to any one of embodiments 78-81 or multicellular structure according to embodiment 82 or embodiment 83, as animal feed, or to produce feed for animal consumption or food for human consumption.

Additional sequences
OsSODA1 - Q43121
SEQ ID NO: 92
MALRTLASRKTLAAAALPLAAAAAARGVTTVALPDLPYDYGALEPAISGEIMRLHHQKHHATYVANYNKALEQLD
AAVAKGDAPAIVHLQSAIKFNGGGHVNHSIFWNNLKPISEGGGDPPHAKLGWAIDEDFGSFEALVKKMSAEGAAL
QGSGWVWLALDKEAKKLSVETTANQDPLVTKGANLVPLLGIDVWEHAYYLQYKNVRPDYLSNIWKVMNWKYAGEV
YENATA
OsSOD2 - Q10PW4
SEQ ID NO: 93
MAGKAGGLKGVALIGGAGGNSAVAGALHFFQDPSTGYTEVRGRVTGLAPGLHGFHIHSFGDTINGCNSTGPHENP
HNKSHGAPSDDERHVGDLGNIVANKDGVADIFIKDLQISLSGPHSILGRAVVVHADSDDLGRGGHELSKTTGNAG
ARIGCGIIGLRSAV
OsSODCC1 - Q0DRV6
SEQ ID NO: 94
MVKAVVVLGSSEIVKGTIHFVQEGDGPTTVTGSVSGLKPGLHGFHIHALGDTINGCMSTGPHYNPAGKEHGAPED
ETRHAGDLGNVTAGEDGVANIHVVDSQIPLTGPNSIIGRAVVVHADPDDLGKGGHELSKTTGNAGGRVACGIIGL
QG
OsSOS1 - Q5ICN3
SEQ ID NO: 95
MDNPEAEPDDAVLFVGVSLVLGIASRHLLRGTRVPYTVALLVLGVALGSLEFGTKHGMGKLGAGIRIWANINPDL
LLAVFLPALLFESSFSMEIHQIKKCMAQMVLLAGPGVLISTFFLGSALKLTFPYNWNWKTSLLLGGLLSATDPVA
VVALLKELGASKKLSTIIEGESLMNDGTAIVVYQLFYRMVLGRIFDAGSIIKFLSEVSLGAVALGLAFGIASVLW
LGFIFNDTIIEIALTLAVSYIAFFTAQDALEVSGVLTVMTLGMFYAAFAKTAFKGDSQQSLHHFWEMVAYIANTL
IFILSGVVIADGVLENNVHFERHGASWGFLLLLYVFVQISRILVVVILYPLLRHFGYGLDLKEATILVWAGLRGA
VALSLSLSVKRASDAVQTHLKPVDGTMFVFFTGGIVELTLIFNGSTTQFLLHLLGMDRLAATKLRILNYTKYEML
NKALEAFGDLRDDEELGPPADWVTVKKYITCLNDLDDEPVHPHAVSDRNDRMHTMNLRDIRVRLLNGVQAAYWGM
LEEGRITQTTANILMRSVDEAMDLVPTQELCDWKGLRSNVHFPNYYRFLOMSRLPRRLITYFTVERLESGCYICA
AFLRAHRIARRQLHDFLGDSEVARIVIDESNAEGEEARKFLEDVRVTFPQVLRVLKTRQVTYSVLTHLSEYIQNL
QKTGLLEEKEMAHLDDALQTDLKKFKRNPPLVKMPRVSDLLNTHPLVGALPAAMRDPLLSSTKETVKGHGTILYR
EGSRPTGIWLVSIGVVKWTSQRLSSRHSLDPILSHGSTLGLYEVLIGKPYICDMITDSVVHCFFIEAEKIEQLRQ
SDPSIEIFLWQESALVVARLLLPMMFEKMATHELRVLITERSTMNIYIKGEEIELEQNFIGILLEGFLKTKNQTL
ITPPGLLLPPNADLNLFGLESSAINRIDYCYTAPSYQVEARARILFVEIGRPEIEADLORSASLISQTLELPRTQ
SKEHSGLLSWPESFRKSRGAQNGASLTEIRDHPASFSARALQLSMYGSMINDMKSGOGQGQRRORHRHTKASSNK
AHSSSYPRVPSRSSNTQRPLLSVQSEGANMITARQAAAAGASLPPEPEEAGRRRRRQRKAIEEDEDNSSDESAGE
EVIVRVDSPSMLTFRQPSSAADR
OsSOS1 - Q7XBF9
SEQ ID NO: 96
MDNPEAEPDDAVLFVGVSLVLGIASRHLLRGTRVPYTVALLVLGVALGSLEFGTKHGMGKLGAGIRIWANINPDL
LLAVFLPALLFESSFSMEIHQIKKCMAQMVLLAGPGVLISTFFLGSALKLTFPYNWNWKTSLLLGGLLSATDPVA
VVALLKELGASKKLSTIIEGESLMNDGTAIVVYQLFYRMVLGRTFDAGSIIKFLSEVSLGAVALGLAFGIASVLW
LGFIFNDTIIEIALTLAVSYIAFFTAQDALEVSGVLTVMTLGMFYAAFAKTAFKGDSQQSLHHFWEMVAYIANTL
IFILSGVVIADGVLENNVHFERHGASWGFLLLLYVFVQISRILVVVILYPLLRHFGYGLDLKEATILVWAGLRGA
VALSLSLSVKRASDAVQTHLKPVDGTMFVFFTGGIVELTLIFNGSTTQFLLHLLGMDRLAATKLRILNYTKYEML
NKALEAFGDLRDDEELGPPADWVTVKKYITCLNGLDDEPVHPHAVSDRNDRMHTMNLRDIRVRLLNGVQAAYWGM
LEEGRITQTTANILMRSVDEAMDLVPTQELCDWKGLRSNVHFPNYYRFLOMSRLPRRLITYFTVERLESGCYICA
AFLRAHRIARROLHDFLGDSEVARIVIDESNAEGEEARKFLEDVRVTFPQVLRVLKTRQVTYSVLTHLSEYIQNL
QKTGLLEEKEMAHLDDALQTDLKKFKRNPPLVKMPRVSDLLNTHPLVGALPAAMRDPLLSSTKETVKGHGTILYR
EGSRPTGIWLVSIGVVKWTSQRLSSRHSLDPILSHGSTLGLYEVLIGKPYICDMITDSVVHCFFIEAEKIEQLRQ
SDPSIEIFLWQESALVVARLLLPMMFEKMATHELRVLITERSTMNIYIKGEEIELEQNFIGILLEGFLKTKNQTL
ITPPGLLLPPNADLNLFGLESSAINRIDYCYTAPSYQVEARARILFVEIGRPEIEADLORSASLISQTLELPRTQ
SKEHSETIQQLSGAQNGASLTEIRDHPASFSARALQLSMYGSMINDMKSGQGQGORRORHRHTKASSNKAHSSSY
PRVPSSSSNTQRPLLSVQSEGANMTTARQAAAAGASLPPEPEEAGRRRRRORKAIEEDEDNSSDESAGEEVIVRV
DSPSMLTFRQPSSAADR
SEQ ID NO: 97 - OsNHX1 - Q9SXJ8
MGMEVAAARLGALYTTSDYASVVSINLFVALLCACIVLGHLLEENRWVNESITALIIGLCTGVVILLMTKGKSSH
LFVFSEDLFFIYLLPPIIFNAGFQVKKKQFFRNFMTITLFGAVGTMISFFTISIAAIAIFSRMNIGTLDVGDFLA
IGAIFSATDSVCTLQVLNQDETPFLYSLVFGEGVVNDATSIVLFNALQNFDLVHIDAAVVLKFLGNFFYLFLSST
FLGVFAGLLSAYIIKKLYIGRHSTDREVALMMLMAYLSYMLAELLDLSGILTVFFCGIVMSHYTWHNVTESSRVT
TKHAFATLSFIAETFLFLYVGMDALDIEKWEFASDRPGKSIGISSILLGLVLIGRAAFVFPLSFLSNLTKKAPNE
KITWRQQVVIWWAGLMRGAVSIALAYNKFTRSGHTQLHGNAIMITSTITVVLFSTMVFGMMTKPLIRLLLPASGH
PVTSEPSSPKSLHSPLLTSMQGSDLESTTNIVRPSSLRMLLTKPTHTVHYYWRKFDDALMRPMFGGRGFVPFSPG
SPTEQSHGGR
OsNHX1 - Q6VVA7
SEQ ID NO: 98
MGMEVAAARLGPLYTTSDYASVVSINLFVALLCACIVLGHLLEENRWVNESITALIIGLCTGVVILLMTKGKSSH
LFVFSEDLFFIYLLPPIIFNAGFQVKKKQFFRNFMTITLFGAVGTMISFFTISIAAIAIFSRMNIGTLDVGDFLA
IRAIFPATDSVCTLQVLNQDETPFLYSLVFGEGVVNDATSIVLFNALQNFDLVHIDAAVVLKFLGNFFYLFLSST
FLGVFAGLLSAYIIKKLYIGRHSTDREVALMMLMAYLSYMLAELLDLSGILTVFFCGIVMSHYTWHNVTESSRVT
TKHAFATLSFIAETFLFLYVGMDALDIEKWEFASDRPGKSIGISSILLGLVLIGRAAFVFPLSFLSNLTKKAPNE
KRTWRQQVVIWWAGLMRGAVSIALAYNKFTRSGHTQLHGNAIMITSTITVVLWSTMVFGMMTKPLIRLLLPASGH
PVTSEPSSPKSLHSPLLTRMQGSDLESTTNIVRPSSLRMLLTKPTHTVHYYWRKFDDALMRPMFGGRGFVPFSPG
SPTEQSHGGR
OsHKT1 - Q0D9S3
SEQ ID NO: 99
MTSIYHDFIHNKLQSFGRIGRYFVNFVVLAHRFIALHIHPFWIQLSYFLLISILGSVLLMFLKPSNPEFRPGYID
MLFLSTSALTLSSLITIEMEVLSSSQIVVITLLMLLGGEVFVSFLGLMLRLNHKHNPEFSGDKVSSVPIELDTIN
SASTVISCEELQLEAAIPEVPSSTIKDLKRSKRLRWFLGFVVFSYFVVIHVAGFLLVLWYISRVSSAKAPLKKKG
INIALFSFSVTVSSFANVGLVPTNENMAIFSKNPGLLLLFIGQILAGNTLYPLFLRLLIWFLGKVTKLRELKLMI
KNPEELQYDYLLPKLPTAFLASTVIGLMASLVTLFGAVDWNSSVEDGLSSYQKIINALFMAVNARHSGENSIDCS
LIAPAVLVLFIILMYLPPSTTFALSNGDEKTANKKAKRKLGLVVONLAFSQLACISVEVIVAFITERSRLRNDPL
NFSALNMIFEIISAYGNVGLSTGYSCSRLQKLHPGSICQDKPYSLSGWWSDEGKLLLVFVMLYGRLKAFTKGTGE
YWRLW
OsSOS2 - Q69Q47
SEQ ID NO: 100
MGGEEGMAAGRKKRVGRYEVGRTIGQGTFAKVKFAVDADTGAAVAMKVLDKDTILNHRMLHQIKREISIMKIVRH
PNIVRLNEVLAGKTKIYIILELITGGELFDKIARQGKLRENEARKYFQQLIDAINYCHSKGVYHRDLKPENLLLD
SRGNLKVSDFGLSTLAQKGVGLLHTTCGTPNYVAPEVLSNNGYDGSAADVWSCGVILYVLMAGYLPFEEDDLPTL
YDKITAGQFSCPYWFSPGATSLIHRILDPNPKTRITIEQIREDTWFKKTYVAIKRGEDENVDLDDVQAVEDNIED
KYVSEQVTHNDGGPLVMNAFEMITLSQGLDLSALFDRQQEFVKRQTRFVSRKPAKTIVATIEVVAETMGLKVHSQ
NYKLRLEGVSSNRMSPFAVVLQVFEVAPSLFMVDVRKVAGDTLEYHRFYKNLCNKMESIIWRPIEVSAKSALLRT
ATC
OsAHA3 - Q8L613
SEQ ID NO: 101
MAEDKGGLDAVLKESVDLENIPIEEVFQNLKCCRQGLTSEEAQLRLOLFGPNKLEEKEESKFLKFLGFMWNPLSW
VMEAAAIMAIALANGGGKPPDWQDFVGIITLLLINSTISFIEENNAGNAAAALMARLAPKAKVLRNGSWTEEEAA
ILVPGDIISIKLGDIIPADARLLEGDPLKIDQSALTGESLPATKGPGDGVYSGSTVKQGEIEAVVIATGVHTFFG
KAAHLVDSTNQVGHFQKVLTAIGNFCICSIAVGMFVEIIVMYPIQHRPYRPGIDNLLVLLIGGIPIAMPTVLSVT
MAIGSHRLSQQGAITKRMTAIEEMAGMDVLCSDKTGTLTLNKLTVDKNLIEIFERGVTQDQVILMAARASRTENQ
DAIDTAIVGMLADPKEARAGIQEVHFLPFNPTDKRTALTYIDSDGKMYRVSKGAPEQILNLAHNKTQIERRVHAV
IDKFAERGLRSLAVAYQEVPDGRKESPGGPWRFVALLPLFDPPRHDSAETIRRALNLGVNVKMITGDQLAIGKET
GRRLGMGTNMYPSSALLGONKDESVAALPVDDLIEKADGFAGVFPEHKYEIVKRLQARKHICGMTGDGVNDAPAL
KKADIGIAVADATDAARSASDIVLTEPGLSVIISAVLTSRAIFORMKNYTIYAVSITIRIVFGFMLLALIWEFDF
PPFMVLIIAILNDGTIMTISKDLVKPSPLPDSWKLAEIFTTGVVLGGYLAMMTVIFFWAAYKTNFFPRIFHVESL
EKTAQDDYQKLASAVYLQVSTISQALIFVTRSRSWSFIERPGFLLVFAFFVAQLIATLIAVYANWAFTSIKGIGW
GWAGIVWLYNLVFYFPLDIIKFLIRYALSGKAWDLVIEQRIAFTRKKDFGKEERELKWAHAHRTLHGLQPPDAKP
FPEKTGYSELNQMAEEAKRRAEIARLRELHTLKGHVESVVKLKGLDIDTIHQSYTV
OsVHA-A - Q651T8
SEQ ID NO: 102
MSYDRVTTFEDSEKESEYGYVRKVSGPVVVADGMGGAAMYELVRVGNDNLIGEIIRLEGDSATIQVYEETAGLMV
NDPVLRTRKPLSVELGPGILGNIFDGIQRPLKTIAIKSGDVYIPRGVSVPALDKDQLWDFEPKKLGVGDAITGGD
LYATVFENTLMKHHVALPPGSMGKISYIAPAGQYSLQDTVLELEFQGIKKQFTMLQTWPVRSPRPVSSKLAADTP
LLTGQRVLDALFPSVLGGTCAIPGAFGCGKTVISQALSKYSNSEAVVYVGCGERGNEMAEVLMDFPQLTMTLPDG
REESVMKRTTLVANTSNMPVAAREASIYTGITIAEYFRDMGYNVSMMADSTSRWAEALREISGRLAEMPADSGYP
AYLAARLASFYERAGKVKCLGSPDRTGSVTIVGAVSPPGGDFSDPVTSATLSIVQVFWGLDKKLAQRKHFPSVNW
LISYSKYSKALESFYEKFDQDFIDIRTKAREVLOREDDLNEIVOLVGKDALAESDKITLETAKLLREDYLAQNAF
TPYDKFCPFYKSVWMMRNIIHFNTLANQAVERAANADGQKITYSVIKHRMGDLFYRLVSQKFEDPAEGEDVLVAK
FQKLYDDLTTGERNLEDEAR
OsVHA-B - Q7FV25
SEQ ID NO: 103
MGLVKDGAADLEEGTLEIGMEYRTVSGVAGPLVILDKVKGPKYQEIVNIRLGDGTTRRGQVLEVDGEKAVVQVFE
GTSGIDNKYTTVQFTGEVLKTPVSLDMLGRIFNGSGKPIDNGPPILPEAYLDISGSSINPSERTYPEEMIQTGIS
TIDVMNSIARGQKIPLFSAAGLPHNEIAAQICRQAGLVKSLEKGKHAEGGEDDNFAIVFAAMGVNMETAQFFKRD
FEENGSMERVTLFINLANDPTIERIITPRIALTTAEYLAYECGKHVLVILTDMSSYADALREVSAAREEVPGRRG
YPGYMYTDLATIYERAGRIEGRSGSITQIPILTMPNDDITHPTPDLTGYITEGQIYIDRQLHNRQIYPPINVLPS
LSRLMKSAIGEGMTRRDHSDVSNQLYANYAIGKDVQAMKAVVGEEALSSEDLLYLEFLDKFERKFVTQGAYDTRN
IFQSLDLAWTLLRIFPRELLHRIPAKTLDQYYSRDATH
OsPsbO - A5JV93
SEQ ID NO: 104
MAASLQAAATLMPAKIGGRASSARPSSHVARAFGVDAGARITCSLOSDIREVASKCAEAAKMAGFALATSALLVS
GASAEGAPKRLTFDEIQSKTYMEVKGTGTANQCPTIDGGVDSFPFKAGKYEMKKFCLEPTSFTVKAEGIQKNEPP
AFQKTKLMTRLTYTLDEMEGPLEVGADGTLKFEEKDGIDYAAVTVQLPGGERVPFLFTVKQLVATGKPESFSGPF
LVPSYRGSSFLDPKGRGGSTGYDNAVALPAGGRGDEEELAKENVKNASSSTGNITLSVTKSKPETGEVIGVFERV
QPSDTDLGAKAPKDVKIQGVWYAQLESN
OsPsbP - XP_002876377.1/Q0KIW5 fragment
SEQ ID NO: 105
VVNASSSEASSDEKNVTRRRLALLGAGALATGLLKSSSAYAEEVPKNYKSYVDSKDGYSYLYPADWRDFDFLGHD
SAFKDRNVALQCVRVGFIPTTKTDIRDLGPMDEAIFNLVNNVYAAPNQIPTVYDMQERTVHGKNYWT
OsPsbQ - P83646
SEQ ID NO: 106
MAQAMASMTGLSQGVQLPAGPRRAGGRSRLAVVRADAAAADVQTGRRAVLGLVATGIAGGALAQAALAEAAKPIK
LGPPPPPSGGLPGTLNSDQARDTDLPLRERFYLQPLPPAEAAARAKESAQDIINLKPLIEKKOWPFVRDDLRLRA
SYLRYDLKTVINSKPKDEKKGLKDLTGKLFATIDGLDHAAKIKSPEEAEKYYTLTKSALGDVLAKLG
OsOSK1 - Q852Q2
SEQ ID NO: 107
MEGAGRDGNPLGGYRIGKTLGIGSFGKVKIAEHILTGHKVAIKILNRRKIKSMEMEEKVKREIKILRLFMHPHII
RLYEVIDTPADIYVVMEYVKSGELFDYIVEKGRLQEEEARRFFQQIISGVEYCHRNMVVHRDLKPENLLLDSKCN
VKIADFGLSNVMRDGHFLKTSCGSPNYAAPEVISGKLYAGPEVDVWSCGVILYALLCGTLPFDDENIPNLFKKIK
GGIYTLPSHLSPLARDLIPRMLVVDPMKRITIREIREHQWFTVGLPRYLAVPPPDTAQQVKKLDDETLNDVINMG
FDKNQLIESLHKRLQNEATVAYYLLLDNRLRTTSGYLGAEFHESMESSLAQVTPAETPNSATDHRQHGHMESPGF
GLRHHFAADRKWALGLOSRAHPREIITEVLKALQELNVCWKKIGHYNMKCRWSPSFPSHESMMHNNHGFGAESAI
IETDDSEKSTHTVKFEIQLYKTRDEKYLLDLORVSGPQLLFLDLCSAFLTQLRVL
OsOSK1 -Q0DGI1
SEQ ID NO: 108
MEGAGRDGNPLGGYRIGKTLGIGSFGKVKIAEHILTGHKVAIKILNRRKIKSMEMEEKVKREIKILRLFMHPHII
RLYEVIDTPADIYVVMEYVKSGELFDYIVEKGRLQEEEARRFFQQIISGVEYCHRNMVVHRDLKPENLLLDSKCN
VKIADFGLSNVMRDGHFLKTSCGSPNYAAPEVISGKLYAGPEVDVWSCGVILYALLCGTLPFDDENIPNLFKKIK
GGIYTLPSHLSPLARDLIPRMLVVDPMKRITIREIREHQWFTVGLPRYLAVPPPDTAQQVKKLDDETLNDVINMG
FDKNQLIESLHKRLQNEATVAYYLLLDNRLRTTSGYLGAEFHESMESSLAQVTPAETPNSATDHRQHGHMESPGF
GLRHHFAADRKWALGLOSRAHPREIITEVLKALQELNVCWKKIGHYNMKCRWSPSFPSHESMMHNNHGFGAESAI
IETDDSEKSTHTVKFEIQLYKTRDEKYLLDLQRVSGPQLLFLDLCSAFLTQLRVL
OsOSK1 -Q9ZTF6
SEQ ID NO: 109
MEGAGSDGNPLGGYRIGKTLGIGSFGKVKIAEHILTGHKVAIKILNRRKSMEMEEKVKREIKILRLFMHPHIIRL
YEVIDTPADIYVVMEYVKSGELFDYDVEKGRLQEEEARRFFQQIISGVEYCHRNMVVRRDLKPENLLLDSKCNVK
IADFGLSNVMRDGHFLKTSCGSPNYAAPEVISGKLYAGPEVDVWSCGVILYALLCGTLPFDDENIPNLFKNIKGG
IYTLPSHLSPLGRDLIPRMLVVDPMKRITIREIREHQWFTVGLPRYLAVPPPDTAQQVKKLDDETQNDVINMGED
KNQLIESLHKRLQNEATVAYYLLLDNRLRTTSGYLGAEFHESMVSSLAQVTPAETPNSATDHRQHGHMESPGFGL
RHHFAADRKWALGLQSRAHPREIITEVLKALQELNVCWKKIGHHNMKCRWSPSFPSHESMMHNNHGFGAASAMIE
TDDSEKSTHTVKFEIQLYKTRDEKYFLDLQRLSGPQLLFLDLCSAFLTQLRVL
OsOSK1 -Q9ZRJ1
SEQ ID NO: 110
MEGAGRDGNPLGGYRIGKTLGIGSFGKVKIAEHILTGHKVAIKILNRRKIKSMEMEEKVKREIKILRLFMHPHII
RLYEVIDTPADIYVVMEYVKSGELFDYIVEKGRLQEEEARRFFQQIISGVEYCHRNMVVHRDLKPENLLLDSKCN
VKIADFGLSNVMRDGHFLKTSCGSPNYAAPEVISGKLYAGPEVDVWSCGVILYALLCGTLPFDDENIPNLEKKIK
GGIYTLPSHLSPLARDLIPRMLVVDPMKRITIREIREHQWFTVGLPRYLAVPPPDTAQQVKKLDDETLNDVINMG
FDKNQLIESLHKRLQNEATVAYYLLLDNRLRTTSGYLGAEFHESMESSLAQVTPAETPNSATDHRQHGHMESPGF
GLRHHFAADRKWALGLOSRAHPREIITEVLKALQELNVCWKKIGHYNMKCRWSPSFPSHESMMHNNHGFGAESAI
IETDDSEKSTHTVKFEIQLYKTRDEKYLLDLQRVSGPQLLFLDLCSAFLTQLRVL
P450 - Q9FVS9
SEQ ID NO: 112
MAMLGFYVTFIFFLVCLFTYFFLQKKPQGQPILKNWPFLRMLPGMLHQIPRIYDWTVEVLEATNLTFYFKGPWLS
GTDMLFTADPRNIHHILSSNFGNYPKGPEFKKIFDVLGEGILTVDFELWEEMRKSNHALFHNQDFIELSVSSNKS
KLKEGLVPFLDNAAQKNIIIELQDVFQRFMEDTSSILMTGYDPMSLSIEMLEVEFGEAADIGEEAIYYRHFKPVI
LWRLQNWIGIGLERKMRTALATVNRMFAKIISSRRKEEISRAKTEPYSKDALTYYMNVDTSKYKLLKPNKDKFIR
DVIFSLVLAGRDTTSSVLTWFFWLLSKHPQVMAKLRHEINTKFDNEDLEKLVYLHAALSESMRLYPPLPENHKSP
AKPDVLPSGHKVDANSKIVICIYALGRMRSVWGEDALDFKPERWISDNGGLRHEPSYKFMAFNSGPRTCLGKNLA
LLQMKMVALEIIRNYDFKVIEGHKVEPIPSILLRMKHGLKVTVTKKI
Oryza sativa sucrose-phosphate synthase 1- QOJGK4
SEQ ID NO: 113
MAGNEWINGYLEAILDSGGAAGGGGGGGGGGGGGGGGGGGGGGGGVDPRSPAAGAASPRGPHMNENPTHYFVEEV
VKGVDESDLHRTWIKVVATRNARERSTRLENMCWRIWHLARKKKOLELEGILRISARRKEQEQVRRETSEDLAED
LFEGEKADTVGELAQQDTPMKKKFORNFSELTVSWSDENKEKKLYIVLISLHGLVRGDNMELGRDSDTGGQVKYV
VELARALAMMPGVYRVDLFTRQVSSPEVDWSYGEPTEMLTSGSTDGEGSGESAGAYIVRIPCGPRDKYLRKEALW
PYLQEFVDGALAHILNMSKALGEQVSNGKLVLPYVIHGHYADAGDVAALLSGALNVPMVLTGHSLGRNKLEQIMK
QGRMSKEEIDSTYKIMRRIEGEELALDAAELVITSTRQEIDEQWGLYDGFDVKLEKVLRARARRGVSCHGREMPR
MVVIPPGMDFSSVVVPEDTSDGDDGKDFEIASPRSLPPIWAEVMRFLINPHKPMILALSRPDPKKNITTLVKAFG
ECRPLRELANLILIMGNRDDIDEMSAGNASVLTTVLKLIDKYDLYGSVAFPKHHKQSDVPEIYRLTGKMKGVFIN
PALVEPFGLTLIEAAAHGLPIVATKNGGPVDIKNALNNGLLVDPHDQHAIADALLKLVADKNLWQECRKNGLRNI
QLYSWPEHCRTYLTRIAGCRIRNPRWLMDTPADAAAEEEEALEDSLMDVQDLSLRLSIDGERGSSMNDAPSSDPQ
DSVQRIMNKIKRSSPADTDGAKIPAEAAATATSGAMNKYPLLRRRRRLFVIAVDCYGDDGSASKRMLQVIQEVER
AVRSDSQMSRISGFALSTAMPLPETLKLLOLGKIPPTDFDALICGSGSEVYYPSTAQCVDAGGRLRPDQDYLLHI
NHRWSHDGAKQTIAKLAHDGSGTNVEPDVESCNPHCVSFFIKDPNKVRTIDEMRERVRMRGLRCHLMYCRNATRL
QVVPLLASRSQALRYLFVRWGLSVGNMYLIVGEHGDTDHEEMLSGLHKTVIIRGVTEKGSEQLVRSSGSYQREDV
VPSESPLIAFTKGDLKADEIMRALKEVTKAASGM
Oryza sativa delta-1-pyrroline-5-carboxylate synthase 1 004226
SEQ ID NO: 114
MASVDPSRSFVRDVKRVIIKVGTAVVSRQDGRLALGRVGALCEQVKELNSLGYEVILVTSGAVGVGRQRLRYRKL
VNSSFADLQKPQMELDGKACAAVGQSGLMALYDMLFNQLDVSSSQLLVTDSDFENPKFREQLTETVESLLDLKVI
PIFNENDAISTRKAPYEDSSGIFWDNDSLAGLLALELKADLLILLSDVDGLYSGPPSEPSSKIIHTYIKEKHQQE
ITFGDKSRVGRGGMTAKVKAAVLASNSGTPVVITSGFENRSILKVLHGEKIGTLFHKNANLWESSKDVSTREMAV
AARDCSRHLONLSSEERKKILLDVADALEANEDLIRSENEADVAAAQVAGYEKPLVARLTIKPGKIASLAKSIRT
LANMEDPINQILKKTEVADDLVLEKTSCPLGVLLIVFESRPDALVQIASLAIRSGNGLLLKGGKEAIRSNTILHK
VITDAIPRNVGEKLIGLVTTRDEIADLLKLDDVIDLVIPRGSNKLVSQIKASTKIPVLGHADGICHVYIDKSADM
DMAKHIVMDAKIDYPAACNAMETLLVHKDLMKSPGLDDILVALKTEGVNIYGGPIAHKALGFPKAVSFHHEYSSM
ACTVEFVDDVQSAIDHIHRYGSAHTDCIVTTDDKVAETFLRRVDSAAVFHNASTRFSDGARFGLGAEVGISTGRI
HARGPVGVEGLLITRWILRGRGQVVNGDKDVVYTHKSLPLQ

### REFERENCES

[1] Silva and Geros (2009) Plant Signal Behav (8): 718-726.
[2] Fan et al. (2019) BMC Plant Biology, 19: 74.
[3] Khan et al. (2015) Biotechnology and Biotechnological equipment, 29, 5, 815-825.
[4] Hossian et al. (2006) Plant Biotechnology, 23, 275-281.
[5] Current Protocols in Molecular Biology (F. M. Ausubel et al., eds., 1987) Supplement 30
[6] Smith & Waterman (1981) Adv. Appl. Math. 2: 482-489.
[7] Liu et al. (2013) Molecular plant, 6, 2, 275-286
[8] Shi et al. (2000) PNAS, 97(12): 6896-6901.
[9] Huertas et al. (2012) Plant, Cell & Environment, 5, Issue 8/p. 1467-1482
[10] Abdel-latif (2007) Research Journal of Agriculture and Biological Sciences, 3(6): 930-933
[11] Hmida-Sayari et al. (2005) Plant Science 169, 746-75
[12] Higo et al. (1990) Nucleic Acids Research, 27, 1, 297-300.
[13] Dubouze et al. (2003) The Plant Journal 33, 751-763
[14] Odell et al. (1988) Plant Molecular Biology, 10; 263-272
[15] Odell et al. (1985) Nature, 313, p810
[16] Nakashima et al. (2012) Biochimica et Biophysica Acta, 1819 97-103
[17] Fang et al (1989) The Plant Cell, Vol. 1, 141-150
[18] Oeda et al (1001) The EMBO Journal vol. 10 no. 7 pp. 1793-1802
[19] Cserhati (2015) Genomics Data 3 128-136
[20] Bilas et al. (2016) Plant Cell Tiss Organ Cult, 127:269-287
[21] Xu et al (1996) Plant Molecular Biology 31:1117-1127.
[22] Salinas et al (1992) The Plant Cell, Vol. 4, 1485-1493
[23] Kaplan-Levy et al (2012) Trends in Plant Science vol. 17, No. 3
[24] Shinshi et al (1995) Plant Molecular Biology 27: 923-932,
[25] Schlabach et al. (2010) PNAS, 107, 6, 2538-2543
[26] Rathus et al (1993) Plant Molecular Biology 23: 613-618.
[27] Wani et al 2010 The Asian and Australasian Journal of Plant Science and Biotechnology, 4(1), pp. 57-61.

1-71. (canceled)

72. An engineered plant comprising at least four genes of interest, wherein the genes of interest comprise a gene that encodes a plasma membrane ion transporter and a gene that encodes a vacuolar ion transporter, wherein the genes of interest further comprise two genes selected from the group consisting of: a gene that encodes a potassium transporter, a hydrogen exporting ATPase, a protein kinase and/or an antioxidant, wherein each gene of interest is operatively linked to an enhancer element and wherein the enhancer elements alter the expression level of the genes of interest compared to the expression level of the genes of interest in a wild-type plant.

73. The engineered plant of claim 72, wherein:
(a) the at least four genes of interest encode OsSOS1, OsNHX1, OsHKT1 and OsSODA1; optionally wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising an amino acid sequence that has at least 95% sequence identity to SEQ ID NO: 92; or
(b) the at least four genes of interest encode OsSOS1, OsNHX1, OsHKT1 and OsAHA3, optionally wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsAHA3 gene comprises a polynucleotide sequence that encodes a polypeptide comprising an amino acid sequence that has at least 95% sequence identity to SEQ ID NO: 101, or
(c) the at least four genes of interest encode OsSOS1, OsNHX1, OsSOS2 and OsVHA-A, optionally wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsSOS2 gene comprises a polynucleotide sequence that encodes a polypeptide comprising the amino acid sequence of SEQ ID NO: 100; wherein the OsVHA-A gene comprises a polynucleotide sequence that encodes a polypeptide comprising the amino acid sequence of SEQ ID NO: 102.

74. The engineered plant according to claim 72, wherein the engineered plant comprises at least seven genes of interest, wherein the at least seven genes of interest comprise
(a) a gene that encodes a plasma membrane ion transporter and wherein the gene that encodes a plasma membrane ion transporter is operatively linked to an enhancer element;
(b) a gene that encodes a plasma membrane hydrogen exporting ATPase and wherein the gene that encodes a plasma membrane hydrogen exporting ATPase is operatively linked to an enhancer element;
(c) a gene that encodes a protein kinase and wherein the gene that encodes a protein kinase is operatively linked to an enhancer element;
(d) a gene that encodes a vacuolar hydrogen exporting ATPase and wherein the gene that encodes a vacuolar hydrogen exporting ATPase is operatively linked to an enhancer element;
(e) a gene that encodes a vacuolar sodium/proton transporter and wherein the gene that encodes a vacuolar sodium/proton transporter is operatively linked to an enhancer element;
(f) a gene that encodes a potassium transporter and wherein the gene that encodes a potassium transporter is operatively linked to an enhancer element; and
(g) a gene that encodes an antioxidant and the gene that encodes an antioxidant is operatively linked to an enhancer element.

75. The engineered plant according to claim 72, wherein the engineered plant comprises at least eight genes of interest, wherein the at least eight genes of interest comprise
(a) a gene that encodes a plasma membrane ion transporter and wherein the gene that encodes a plasma membrane ion transporter is operatively linked to an enhancer element;
(b) a gene that encodes a plasma membrane hydrogen exporting ATPase and wherein the gene that encodes a plasma membrane hydrogen exporting ATPase is operatively linked to an enhancer element;
(c) a gene that encodes a protein kinase and wherein the gene that encodes a protein kinase is operatively linked to an enhancer element;
(d) a gene that encodes a vacuolar hydrogen exporting ATPase and wherein the gene that encodes a vacuolar hydrogen exporting ATPase is operatively linked to an enhancer element;
(e) a gene that encodes a vacuolar sodium/proton transporter and wherein the gene that encodes a vacuolar sodium/proton transporter is operatively linked to an enhancer element;
(f) a gene that encodes a potassium transporter and wherein the gene that encodes a potassium transporter is operatively linked to an enhancer element;
(g) a gene that encodes a first antioxidant and the gene that encodes a first antioxidant is operatively linked to an enhancer element; and
(h) a gene that encodes a second antioxidant and the gene that encodes a second antioxidant is operatively linked to an enhancer element.

76. The engineered plant according to claim 74, wherein:
(a) the plasma membrane ion transporter is SOS1; and/or
(b) the plasma membrane hydrogen exporting ATPase is AHA3; and/or
(c) the protein kinase is SOS2; and/or
(d) the vacuolar hydrogen exporting ATPase is VHA-A; and/or
(e) the vacuolar ion transporter is NHX1; and/or
(f) the potassium transporter is HKT1; and/or
(g) the antioxidant(s) is (are) SODA1, SOD2 and/or SODCC1.
optionally wherein:
(a) the SOS1 is OsSOS1, optionally wherein the OsSOS1 gene comprises a polynucleotide sequence that encodes a polypeptide comprising the amino acid sequence of SEQ ID NO: 95 and/or 96; and/or
(b) the AHA3 is OsAHA3, optionally wherein the OsAHA3 gene comprises a polynucleotide sequence that encodes a polypeptide comprising the amino acid sequence of SEQ ID NO: 101; and/or
(c) the SOS2 is OsSOS2, optionally wherein the OsSOS2 gene comprises a polynucleotide sequence that encodes a polypeptide comprising the amino acid sequence of SEQ ID NO: 100; and/or
(d) the VHA-A is OsVHA-A, optionally wherein the OsVHA-A gene comprises a polynucleotide sequence that encodes a polypeptide comprising the amino acid sequence of SEQ ID NO: 102; and/or
(e) the NHX1 is OsNHX1, optionally wherein the OsNHX1 gene comprises a polynucleotide sequence that encodes a polypeptide comprising the amino acid sequence of SEQ ID NO: 97 and/or 98; and/or
(f) the HKT1 is OsHKT1, optionally wherein the OsHKT1 gene comprises a polynucleotide sequence that encodes a polypeptide comprising the amino acid sequence of SEQ ID NO: 99;
(g) the SODA1 is OsSODA1, optionally wherein the OsSODA1 gene comprises a polynucleotide sequence that encodes a polypeptide comprising the amino acid sequence of SEQ ID NO: 92; and/or
(h) the SOD2 is OsSOD2, optionally wherein the OsSOD2 gene comprises a polynucleotide sequence that encodes a polypeptide comprising the amino acid sequence of SEQ ID NO: 93; and/or
(i) the SODCC1 is OsSODCC1, optionally wherein the OsSODCC1 gene comprises a polynucleotide sequence that encodes a polypeptide comprising the amino acid sequence of SEQ ID NO: 94.

77. An engineered rice plant comprises at least eight genes of interest, wherein the at least eight genes of interest encode OsSOS1, OsSOS2, OsAHA3, OsVHA-A, OsNHX1, OsHKT1, OsSODA1 and OsSOD2/OsSODCC1; wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA-A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2/OsSODCC1 is operatively linked to an enhancer element;
optionally wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; and wherein the OsSOD2 gene comprises a polynucleotide sequence that encodes a polypeptide comprising the amino acid sequence of SEQ ID NO: 93 or the OsSODCC1 gene comprises a polynucleotide sequence that encodes a polypeptide comprising the amino acid sequence of SEQ ID NO: 94.

78. The engineered plant according to claim 72, wherein:
(a) the enhancer elements have the same polynucleotide sequence or wherein the enhancer elements have different polynucleotide sequences;
(b) the enhancer element increases or decreases the expression level of the gene of interest compared to the expression level of the gene of interest in a wild-type plant, optionally wherein the enhancer element increases the expression level of the gene of interest, compared to the expression level of the gene of interest in a wild-type plant, in at least one part of the plant during at least one developmental stage of the plant;
(c) the enhancer element alters the expression level of the gene of interest in the root, shoot, leaf, seed or stem, optionally wherein the enhancer element alters the expression level of the gene of interest in the root;
(d) the enhancer element alters the expression level of the gene of interest during germination, when the plant is growing, when the plant is flowering and/or when the plant is developing fruit;
(e) the enhancer element comprises at least 10, at least 20, or at least 30 nucleotides;
(f) the enhancer element is within 150-500 nucleotides of the 5′ end of an open reading frame of the gene of interest;
(g) the enhancer element comprises a root-specific promoter;
(h) all of the genes of interest are operably linked to enhancer elements comprising a root-specific promoter;
(i) the enhancer element comprises a DREB2A, gibberellin, ethylene or auxin promoter sequence or a combination thereof, optionally wherein all of the genes of interest are operably linked to enhancer elements comprising a DREB2A, gibberellin, ethylene or auxin promoter sequence or a combination thereof;
(j) the enhancer element comprises at least 6 nucleotides from a promoter element from a DREB2A, gibberellin, ethylene or auxin promoter sequence or a combination thereof, optionally wherein all of the genes of interest are operably linked to an enhancer element comprising a promoter element from a DREB2A, gibberellin, ethylene or auxin promoter sequence;
(k) the enhancer element comprises a TAF-1, TATA, E2F, G-BOX, or CAAT promoter sequence or a combination thereof;
(l) all of the genes of interest are operably linked to an enhancer element comprising a TAF-1, TATA, E2F, G-BOX, or CAAT promoter sequence;
(m) the enhancer element comprises a sequence having at least 95% sequence identity to any one of SEQ ID NO: 10-18;
(n) each gene of interest encodes a separate protein; and/or
(o) the engineered plant is an angiosperm, optionally wherein the engineered plant is monocotyledonous or dicotyledonous, further optionally wherein:
(i) the engineered plant is a cereal crop, optionally wherein the engineered plant is maize, rice, soybean, sugar cane, mung bean, quinoa, barley, oat, rye, sorghum, or wheat, further optionally wherein engineered plant is an engineered rice plant; or
(ii) the engineered plant is a vegetable crop, optionally wherein the engineered plant is from the genus a Brassica, Glycine, or Soja.

79. The engineered plant according to claim 77, wherein:
(a) the OsSOS1 gene is operably linked to an enhancer element comprising SEQ ID NO: 12, and/or
(b) the OsSOS2 gene is operably linked to an enhancer element comprising SEQ ID NO: 13, and/or
(c) the OsAHA3 gene is operably linked to an enhancer element comprising SEQ ID NO: 14, and/or
(d) the OsVHA-A gene is operably linked to an enhancer element comprising SEQ ID NO: 11, and/or
(e) the OsNHX1 gene is operably linked to an enhancer element comprising SEQ ID NO: 10, and/or
(f) the OsHKT1 gene is operably linked to an enhancer element comprising SEQ ID NO: 15, and/or
(g) the OsSODA1 gene is operably linked to an enhancer element comprising SEQ ID NO: 16, and/or
(h) the OsSODCC1 gene is operably linked to an enhancer element comprising SEQ ID NO: 17, and/or
(i) the OsSOD2 gene is operably linked to an enhancer element comprising SEQ ID NO: 18.

80. An engineered rice plant comprising at least eight genes of interest, wherein the eight genes of interest encode OsSOS1, OsSOS2, OsAHA3, OsVHA-A, OsNHX1, OsHKT1, OsSODA1 and OsSOD2; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA-A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; wherein the OsSOS1 gene is operably linked to an enhancer element comprising SEQ ID NO: 12, the OsSOS2 gene is operably linked to an enhancer element comprising SEQ ID NO: 13, the OsAHA3 gene is operably linked to an enhancer element comprising SEQ ID NO: 14, the OsVHA-A gene is operably linked to an enhancer element comprising SEQ ID NO: 11, the OsNHX1 gene is operably linked to an enhancer element comprising SEQ ID NO: 10, the OsHKT1 gene is operably linked to an enhancer element comprising SEQ ID NO: 15, the OsSODA1 gene is operably linked to an enhancer element comprising SEQ ID NO: 16 and the OsSOD2 gene is operably linked to an enhancer element comprising SEQ ID NO: 18.

81. A plant part of the engineered plant according to claim 72, optionally wherein the plant part is a cell, a seed, a leaf, a shoot, a stem or a root, optionally wherein the plant part is a seed or a cell.

82. A multicellular structure comprising one or more plant cells according to claim 81, optionally wherein the multicellular structure is a callus.

83. A method of making the engineered plant according to claim 72, the method comprising the steps of
i) introducing at least two enhancer elements into a cell of a plant,
wherein the cell comprises at least four genes of interest, wherein the genes of interest comprise a gene that encodes a plasma membrane ion transporter and a gene that encodes a vacuolar ion transporter, wherein the genes of interest further comprise two genes selected from the group consisting of: a gene that encodes a potassium transporter, a hydrogen exporting ATPase, a protein kinase and/or an antioxidant, wherein each gene of interest is operatively linked to an enhancer element;
wherein the at least two enhancer elements alter the expression level of the genes of interest compared to the expression level of the genes of interest in a wild-type plant; and
wherein the enhancer elements integrate into the genome of the cell of the plant such that they are operatively linked to the genes of interest, and
ii) regenerating the cell to form an engineered plant, a plant part or a multicellular structure from the cell.

84. The method according to claim 83, wherein the methods involves:
(a) inducing callus formation from a seed;
(b) precipitating the enhancer elements, two or more guide RNAs and one or more nucleases onto a microcarrier;
(c) transforming the callus with the microcarriers using particle bombardment to generate a transformed callus wherein the enhancer elements integrate into the genome of the engineered plant, the plant part or the multicellular structure such that they are operatively linked to the genes of interest;
(d) recovering the transformed callus to generate a multicellular; optionally wherein:
(i) the multicellular structure is regenerated into an engineered plant;
(ii) the polynucleotide sequence is stably integrated into the genome of the plant; and/or
(iii) the nuclease is a Cas nuclease, Cpf1 nuclease, a TALEN or a zinc finger nucleases, optionally wherein the nuclease is Cas9 or Cpf1.

85. The engineered plant according to claim 72, wherein the engineered plant is not produced by a process that involves homologous recombination and/or is not produced by an essentially biological process.

86. A feed or food comprising an engineered plant according to claim 72.
